(19) United States
(12) Patent Application Publication
(10) Pub. No.: US 2020/0171068 A1
(43) Pub. Date: Jun. 4, 2020

(54) COMPOSITIONS COMPRISING APTAMERS AND NUCLEIC ACID PAYLOADS AND METHODS OF USING THE SAME

(71) Applicant: Children's National Medical Center, Washington, DC (US)

(72) Inventors: Yang Liu, Washington, DC (US); Pan Zheng, Washington, DC (US); Toshihiko Tanno, Olney, MD (US)

(73) Assignee: Children's National Medical Center, Washington, DC (US)

(21) Appl. No.: 16/614,505

(22) PCT Filed: May 18, 2018
(86) PCT No.: PCT/US2018/033521

§ 371 Date: Nov. 18, 2019

Related U.S. Application Data
(60) Provisional application No. 62/508,237, filed on May 18, 2017.

(51) Int. Cl. A61K 31/713; C12N 15/115; C12N 15/113; A61K 45/06; A61P 7/00; A61P 35/00

(52) U.S. Cl. CPC A61K 31/713; C12N 15/115; C12N 15/1135; A61K 45/06; C12N 2310/3519; A61P 35/00; C12N 2310/16; C12N 2310/141; C12N 2310/20; A61P 7/00

(57) ABSTRACT

The disclosure relates to compositions comprising a nucleic acid sequence having two domains: a cell targeting domain and a microRNA domain, wherein the cell targeting domain comprises an aptamer sequence that targets cancer cells and the microRNA domain comprises a microRNA sequence that binds to an endogenous mRNA sequence within a cancer cell and disrupts normal function of the cell.

### CROSS REFERENCE TO RELATED APPLICATIONS

The present application claims priority to and the benefit of U.S. Provisional Application Ser. No. 62/508,237, filed on May 18, 2017, the entire contents of which is incorporated herein by reference.

### FIELD OF DISCLOSURE

The disclosure relates to compositions comprising a chimeric molecule comprising an aptamer and methods of making, using and administering such chimeric molecules for, among other things, delivery of nucleic acid sequences to one or a plurality of cancer cells.

### BACKGROUND

Dysregulation of miRNA has been implicated in cancer pathogenesis (15-17). Accumulating data have shown that alternation of miRNAs is involved in cancer initiation and progression (15-18), and that manipulating expression level of miRNAs in cancer cells may offer potential therapeutic effect (19). However, it remains exceedingly difficult to deliver miRNA to confer significant therapeutic effect in vivo.

### SUMMARY OF EMBODIMENTS

The efficacy of traditional chemotherapy is limited by their toxicity, especially toxicity of hematopoiesis in the subject. miR-26a is shown to play a critical role in protecting mice against chemotherapy-induced myeloid suppression by targeting a pro-apoptotic protein (Bak1) in hematopoietic stem/progenitor cells (HSPC). Since c-Kit is expressed at high levels in HSPC, an miRNA-aptamer chimera that contains miR-26a mimic and c-Kit-targeting aptamer was designed and successfully delivered miR-26a into HSPC to attenuate toxicity of 5′ fluorouracil (5-FU) and carboplatin. Meanwhile, in silico analysis revealed wide-spread and prognosis-associated down regulation of miR-26a in advanced breast cancer, that KIT is over-expressed among basal-like breast cancer cells, and that such expression associates with poor prognosis. Importantly, the miR-26a-Aptamer effectively repressed tumor growth in vivo and synergize with 5-FU in cancer therapy in the mouse breast cancer models. Thus, targeted delivery of miR-26a suppresses tumor growth while protecting host against myelosupression by chemotherapy.

The present disclosure relates to a nucleic acid sequence comprising at least one or a combination of domains in either the 5′ to 3′ or the 3′ to 5′ orientation: an aptamer domain and a miRNA domain. The disclosure relates to a nucleic acid sequence comprising at least one or a combination of domains in either the 5′ to 3′ orientation: an aptamer domain and a miRNA domain, wherein the aptamer domain comprises a secondary structure that directs the nucleic acid sequence into a cancer cell and wherein the miRNA domain comprises a complementarity sufficient to bind an mRNA in the cancer cell thereby reducing or eliminating translation of the mRNA in the cancer cell. The disclosure relates to a nucleic acid sequence comprising at least one or a combination of domains in either the 5′ to 3′ orientation: an aptamer domain and a miRNA domain, wherein the aptamer domain comprises a secondary structure that directs the nucleic acid sequence into a cancer cell and wherein the miRNA domain comprises a complementarity sufficient to bind an mRNA with at least 70% homology to an mRNA in the cancer cell thereby reducing or eliminating translation of the mRNA in the cancer cell. The disclosure relates to a nucleic acid sequence comprising at least one or a combination of domains in either the 5′ to 3′ orientation: an aptamer domain and a miRNA domain, wherein the aptamer domain comprises a secondary structure that directs the nucleic acid sequence into a cancer cell and wherein the miRNA domain comprises a complementarity sufficient to bind an mRNA with at least 70% homology to an mRNA in the cancer cell thereby reducing or eliminating translation of the mRNA in the cancer cell.

The present disclosure relates to a nucleic acid sequence comprising at least one or a combination of domains in either the 5′ to 3′ or the 3′ to 5′ orientation: an aptamer domain and a miRNA domain, wherein the aptamer domain comprises and/or the miRNA domain comprises from about 1% to about 99% modified nucleotides. The present disclosure relates to a nucleic acid sequence comprising at least one or a combination of domains in either the 5′ to 3′ or the 3′ to 5′ orientation: an aptamer domain and a miRNA domain, wherein the aptamer domain comprises and/or the miRINA domain comprises from about 1% to about 99% modified ribonucleotides. The present disclosure relates to a nucleic acid sequence comprising at least one or a combination of domains in either the 5′ to 3′ or the 3′ to 5′ orientation: an aptamer domain and a miRNA domain, wherein the aptamer domain comprises and/or the miRINA domain comprises from about 1% to about 99% modified ribonucleotides and/or deoxyribonucleotides. The present disclosure also relates to a nucleic acid sequence comprising at least one or a combination of domains in either the 5′ to 3′ or the 3′ to 5′ orientation: an aptamer domain and a miRNA domain, wherein the aptamer domain comprises and/or the miRINA domain comprises from about 1% to about 99% modified ribonucleotides. The present disclosure relates to a nucleic acid sequence comprising at least one or a combination of domains in either the 5′ to 3′ or the 3′ to 5′ orientation: an aptamer domain and a miRNA domain, wherein the aptamer domain comprises any one or combination of sequences that are at least 70%, 80%, 85%, 90%, 95% homologous to any sequence set forth in the Examples or Figures. In some embodiments, the miRINA domain comprises from about 1% to about 99% modified ribonucleotides and/or deoxyribonucleic acids.

The present disclosure relates to any disclosed nucleic acid sequence or molecule as a component of a composition or individual with or without a number of disclosed modifications. Any of the modifications listed in this application may be incorporated into a modified nucleotide, either a deoxyribonucleotide or ribonucleotide. In one non-limiting example, any of the nucleotides identified as positions set forth in the sequence identifiers comprise a conserved substituent (oxygen atom or hydroxyl or hydrogen) at the 2′ pentose sugar but may contain a modified functional group at the 3′carbon position of the pentose sugar. Similarly, nucleic acids that have one or a plurality of modification of the phosphodiester bind between one or a plurality of contiguous or non-contiguous nucleotides. In some embodiments, the modification of the one or plurality of nucleotides is a phosphorothioate bond.

In some embodiments, the disclosure relates to composition comprising a nucleic acid sequence, wherein the nucleic acid sequence comprises an aptamer domain and a miRNA domain, wherein the nucleic acid sequence consists of from about 25 to about 250 ribonucleotides. In some embodiments, the nucleic acid sequence consists of from about 25 to about 200 ribonucleotides. In some embodiments the nucleic acid consists of from about 25 to about 150 nucleotides, wherein at least one or pluralities of nucleotides are modified. In some embodiments, the nucleic acid sequence consists of from about 25 to about 140 ribonucleotides. In some embodiments, the nucleic acid sequence consists of from about 25 to about 130 ribonucleotides. In some embodiments, the nucleic acid sequence consists of from about 25 to about 120 ribonucleotides. In some embodiments, the nucleic acid sequence consists of from about 25 to about 90 ribonucleotides. In some embodiments, the nucleic acid sequence consists of from about 25 to about 80 ribonucleotides.

The present disclosure also relates to a composition comprising: (a) a nucleic acid sequence disclosed herein; and (b) any one or plurality delivery agents. In some embodiments, the composition is a pharmaceutical composition that comprises: (a) a nucleic acid sequence disclosed herein or a pharmaceutically acceptable salt thereof; and (b) one or a plurality of pharmaceutically acceptable excipients. In some embodiments, the pharmaceutical composition comprises a delivery agent, such as a nanoparticle that encapsulates any one or plurality of unmodified or modified nucleic acid sequences disclosed herein. In some embodiments, the pharmaceutical composition comprises two or more nucleic acid sequences disclosed herein, modified or unmodified, wherein each nucleic acid sequence is at a therapeutically effective concentration. In some embodiments, the composition further comprises a lipid or polymer that encapsulates any of the nucleic acids disclosed herein, including any ribonucleotide described herein. In some embodiments, the composition further comprises a pharmaceutically acceptable carrier.

The present disclosure also relates to a kit comprising: (a) one or a plurality of nucleic acid sequences disclosed herein; and (b) a vehicle for administration of the one or plurality of nucleic acids to a subject, such as a human. In some embodiments, the one or more nucleic acid sequences described herein are lyophilized or desiccated. In some embodiments, the kit further comprises at least one container comprising a reconstitution fluid.

The present disclosure relates to a cell comprising any one or plurality of nucleic acid sequences disclosed herein or salts thereof. In some embodiments, the cell is a eukaryotic or prokaryotic cell.

The disclosure also relates to a method transforming or transfecting any cell with the one or plurality of nucleic acids disclosed herein or salts thereof. In some embodiments, such methods are a step in a method of treatment or a method of preventing cancer such that entry of the nucleic acid or acids or salts thereof into the cell is performed for a period of time sufficient to transfer a therapeutically effective amount of nucleic acid into the cell. In some embodiments, the cell is a human cancer cell in a subject suspected of having or diagnosed with cancer. In some embodiments, the cancer is characterized as a cancer caused by overexpression of any one or plurality of genes identified herein, the expression of which causes upregulated expression of mRNA that is complementary to or substantially complementary to any of the miRNA domains disclosed herein.

The present disclosure also relates to a method of chemically synthesizing any one or plurality of nucleic acid sequences disclosed herein comprising integrating a modification into a nucleic acid or chemically synthesizing one or a plurality of nucleotide acids in sequence.

The present disclosure also relates to a method of altering expression of at least one gene product in a cancer cell comprising introducing into a cell any one or plurality of disclosed nucleic acid sequences or salts thereof; wherein the cell contains and expresses a mRNA molecule having a target sequence; and wherein the disclosed nucleic acid sequence or sequences or salts thereof are introduced at a concentration sufficient to hybridize the mRNA target sequence, thereby preventing translation of the mRNA or expression of the at least one gene product and altering expression of the gene product.

The present disclosure also relates to a method of treating and/or preventing cancer or cancer progression in a subject in need thereof comprising contacting or administering to the subject a therapeutically effective amount of one or a plurality of any of the disclosed nucleic acid sequences or salts thereof. In some embodiments the step of administering comprises any one or plurality of pharmaceutical compositions disclosed herein. In some embodiments, the step of contacting is performed in vitro, ex vivo, or in vivo. In some embodiments, the subject has breast cancer or is suspected of having breast cancer. In some embodiments, the step of administering comprises administering a therapeutically effective amount of modified cells, (autologous or heterologous cells in respect to the subject) comprising the one or plurality of nucleic acids disclosed herein or salts thereof. In some embodiments, the method comprises administration of a modified cell lymphocyte isolated from a culture, the subject or a donor subject. In some embodiments, the cell is a cultured T-cell or CAR T cell. In some embodiments, the cell is a cell from the liver, lung, neuron, skin, intestine, stomach, breast, or colon. In some embodiments, the cell is cancerous, pre-cancerous or neoplastic.

The present disclosure relates to a method of reducing the toxicity of chemotherapeutic agent by administering or co-administering, sequentially or simultaneously, one of the disclosed pharmaceutical compositions and a chemotherapeutic agent. In some embodiments, the chemotherapeutic agent is at a concentration that would be toxic to the subject if it were administered without the pharmaceutical composition, and the pharmaceutical composition comprises a therapeutically effective amount of one or a combination of nucleic acid sequences disclosed herein or salts thereof.

The present disclosure also relates to a method of preventing leukopenia and/or myelosuppression by administering or co-administering, sequentially or simultaneously, one of the disclosed pharmaceutical compositions.

### BRIEF DESCRIPTION OF THE DRAWINGS

FIG. 1A-FIG. 1H show identification and validation of KIT-targeting miR-26a chimera that inhibits human breast cancer growth in vitro and in vivo. FIG. 1A is a graph showing the overall survival of basal-like breast cancer patients with higher or lower expression of miR-26a-2 based on the expression scores comparing to the mean value in TCGA cohort. FIG. 1B is a graph showing the binding of anti-KIT antibody and KIT DNA aptamer to MDA-MB-231 cell line. Other cell targeting aptamer is specific for Ramos cells. FIG. 1C depicts the secondary structure of KIT aptamer-miR-26a chimera. KIT DNA aptamer was linked with C3 linker to RNA passenger sequence that was complimentary binds to miR-26a mimic sequence. Another RNA passenger sequence binding to the 3′ of miR-26 mimic was conjugated with TEG-cholesterol. FIG. 1D is a graph showing specific miR-26a delivery by the miR-26a chimera. Two days after incubation with the miR-26a chimera, significant increase of miR-26a expression in the MDA-MB-231 cells compared to control chimera treatment was detected by qPCR. Data (mean+s.d.) were pooled from three experiments. FIG. 1E is a series of graphs showing that miR-26a chimera suppressed the growth and induced apoptosis of MDA-MB-231 cells in a dose-dependent manner. After 3 days of culture with miR-26a chimera or control chimera, MDA-MB-231 cell numbers (left) were counted using hemocytometer and Annexin V positive cells (%) (right) was determined by flow cytometry. Data (mean±s.d.) were pooled from two experiments. FIG. 1F is a picture showing significant suppression of EZH2 protein in miR-26a chimera-treated MDA-MB-231 cells detected by Immunoblot. FIG. 1G is a graph showing the relative expression of miR-26a in tumor harvested from NSG mice bearing human breast tumor with MDA-MB-231 cells. Significant increase of miR-26a was observed in KIT⁺ tumor cells 3 days after intravenous injection with 670 pmol/20 g miR-26a chimera. Data (mean+s.d.) were pooled from two experiments, involving a total of 6 mice per group. FIG. 1H is a series of graphs showing the therapeutic effect of miR-26a chimera. The tumor bearing mice were treated daily with miR-26a chimera (670 pmol/20 g) for 5 or 10 days (First injection defined as day 0). Data (mean+s.d.) were pooled from two experiments, involving a total of 5 mice per group. Left, tumor volume over time. Significant difference between miR-26 chimera (×5) versus miR26 chimera (×10) (Two way repeated-measures ANOVA followed by Bonferroni post-test for day 0 to day18 detected the significant difference (interaction p-value 0.0012). Right, Kaplan-Myer survival curve. Log-rank test detected significant differences between control chimera group and miR-26a chimera groups (control chimera versus miR-26 chimera (×5); P=0.0020, control chimera versus miR-26 chimera (×10); P=0.0020). The difference in survival between miR-26 chimera (×5) and miR26 chimera (×10) did not reach statistical significance (P=0.077). *P<0.05, **P<0.01.

FIG. 2A-FIG. 2J demonstrate that miR-26a protects hematopoiesis from chemotherapeutic agent-induced myelosuppression. The mice received intravenous injection of 670 pmol/20 g miR-26a or control chimera daily for 3 days. 150 mg/kg 5-FU was injected on day 2 of the chimera treatment. FIG. 2A is a graph showing the numbers of white blood cells (WBC) 10 days after 5-FU treatment. Data (mean+s.d.) were pooled from two experiments, involving a total of 6 mice per group. FIG. 2B is a graph showing the number of bone marrow (BM) cells 10 days after 5-FU treatment. Data (mean+s.d.) were pooled from two experiments, involving a total of 6 mice per group. FIG. 2C is (left) a representative picture of LSK population 5 days after 5-FU treatment with or without miR-26a chimera treatment; and (right) a graph showing the percentages of LSK population. Data (mean+s.d.) were pooled from two experiments, involving a total of 6 mice per group. FIG. 2D is a series of graphs showing apoptosis. Left, representative picture of Annexin V⁺ in LSK population 5 days after 5-FU treatment. Right, the percentages of Annexin V⁺ in the LSK population. Data (mean+s.d.) were pooled from two experiments, involving a total of 6 mice per group. *P<0.05, **P<0.01. FIG. 2E is a picture showing a putative miR-26a target site in the 3′UTR of mouse Bak1 (SEQ ID NO:19). Base pairs CU-GA -UACUUGAA (SEQ ID NO:20) of the Bak Sequence and its complementary basepairs are the sites with highly probability preferential conservation between mammals. The miR-26a sequence depicted is SEQ ID NO:21. FIG. 2F is a graph showing relative luciferase activity of reporter constructs containing the wild-type or mutant (mut) 3′-UTR of mouse Bak1 in HEK293 cells co-transfected with either miR-26a precursor (OE), miR-26a TuD inhibitor (TuD), or negative control (ctrl). Data were pooled from two experiments. FIG. 2G is a graph showing relative expression level of Bak1 mRNA in LSK population 5 days after 5-FU treatment. Data (mean+s.d.) were pooled from two experiments, involving a total of 6 mice per group. FIG. 2H is a graph showing that targeted mutation of the Bak1 gene increased survival of LSK in BM at day 5 after 150 mg/kg 5-FU treatments. Data shown are percent of LSK in BM (n=3 for Bak1^{+/+} mice and n=4 for Bak1^{-/-} mice). FIG. 2J is a graph showing percent survival of Bak1^{+/+} and Bak1^{-/-} LSK in BM. Data shown percent of untreated, after normalization using means of LSK % from BM of two untreated Bak1^{+/+} and Bak1^{-/-} mice as 100%. FIG. 2J is a graph showing the numbers of WBC 10 days after 150 mg/kg 5-FU treatments between Bak1^{-/-} mice and wild type mice. Data (mean+s.d.) were pooled from two experiments, involving a total of 7 mice per group. *P<0.05, **P<0.01.

FIG. 3A-FIG. 3G demonstrate how miR-26a plays an essential role in hematopoietic reconstitution after BM transplantation. BM cells (5×10⁵) transduced with miR-26a TuD inhibitor (miR-26 TuD) or control (ctrl) were transplanted into lethally irradiated congenic recipients. FIG. 3A is a graph showing percent survival of recipients after the BM transplant (BMT). Data were pooled from two experiments, involving a total of 7 mice per group. FIG. 3B is a series of pictures showing BM cells (CD45.2) transduced with ctrl or miR-26a TuD inhibitor (miR-26 TuD) were harvested and mixed with equal number of recipient-type BM cells (CD45.1), prior to transplantation into lethally irradiated congenic recipients (CD45.1). Left, representative plots of recipient peripheral blood leukocytes for control (ctrl) or miR-26a TuD cells at 8 weeks after BM transplantation. Right, the reconstitution ratio of ctrl or miR-26a TuD donor cells in the recipients' peripheral blood (PB) at 4, 8 and 20-30 weeks after transplantation. Data (mean+s.d.) were pooled from two experiments, involving a total of 10 mice per group. FIG. 3C is a series of graphs showing the reconstitution ratio of ctrl or miR-26a TuD donor cells in the recipients' peripheral blood B220⁺, CD3⁺ and Mac-1⁺ populations at 4, 8 and 20-30 weeks after BMT. Data (mean+s.d.) were pooled from two experiments, involving a total of 10 mice per group. FIG. 3D is a series of graphs showing the reconstitution ratio of ctrl or miR-26 TuD donor cells in the BM, spleen and thymus of recipients at 20-30 weeks after transplantation. Data (mean+s.d.) were pooled from two experiments, involving a total of 10 mice per group. FIG. 3E is a series of graphs showing the reconstitution ratio of ctrl or miR-26 TuD donor cells in the LSK, HSC populations of the recipients' BM at 20-30 weeks after BMT. Data (mean+s.d.) were pooled from two experiments, involving a total of 10 mice per group. FIG. 3F is a series of graphs showing the percentages of Annexin V⁺ in donor-derived LSK and HSC populations 5 days after BMT. Data (mean+s.d.) were pooled from two experiments, involving a total of 6 mice per group. FIG. 3G is a series of graphs showing the relative expression level of Bak1 mRNA in donor-derived LSK and HSC populations. Data (mean+s.d.) were pooled from two experiments, involving a total of 6 mice per group. Error bars stand for standard deviation. *P<0.05, **P<0.01.

FIG. 4A-FIG. 4J show how miR-26a chimera inhibits mouse breast cancer growth and protects from chemo-induced myelosuppression. FIG. 4A is a graph showing the binding of c-Kit-aptamer to TUBO cells. FIG. 4B is a series of graphs showing how the miR-26a chimera suppressed the growth and induced apoptosis of TUBO cells. Additional 5-FU treatment (1 μg/ml) enhanced these effects of miR-26a chimera. Cell count (left) and Annexin V⁺ cells (%) (right) of the cells cultured with miR-26a chimera or the control (ctrl) were determined by flow cytometry. Asterisks denote the significant difference between ctrl chimera versus miR-26a chimera, and between ctrl chimera+5-FU versus miR-26a chimera+5FU. Data (mean±s.d.) were pooled from two experiments. FIG. 4C is a graph showing miR-26a expression in c-Kit⁺ or c-Kit cells harvested from TUBO-derived tumors in BALB/c mice treated with miR-26a chimera (670 pmol/20 g) for 3 days. Data (mean+s.d.) were pooled from two experiments, involving a total of 6 mice per group. FIG. 4D is a graph showing Ezh2 expression in c-Kit⁺ or c-Kit− cells harvested from TUBO-derived tumors in BALB/c mice treated with miR-26a chimera (670 pmol/20 g) for 3 days. Data (mean+s.d.) were pooled from two experiments, involving a total of 6 mice per group. FIG. 4E is a graph showing Bak1 expression in c-Kit⁺ or c-Kit-cells harvested from TUBO-derived tumors in BALB/c mice treated with miR-26a chimera (670 pmol/20 g) for 3 days. Data (mean+s.d.) were pooled from two experiments, involving a total of 6 mice per group. FIG. 4F is a graph showing miR-26a expression in bone marrow (BM) detected by qPCR at 3 days after intravenous injection with the miR-26a chimera (670 pmol/20 g). Data (mean+s.d.) were pooled from two experiments, involving a total of 6 mice per group. FIG. 4G is a graph showing Bak expression in bone marrow (BM) detected by qPCR at 3 days after intravenous injection with the miR-26a chimera (670 pmol/20 g). Data (mean+s.d.) were pooled from two experiments, involving a total of 6 mice per group. FIG. 4H is a graph showing tumor volume over time. BALB/c mice bearing TUBO cells were treated with miR-26a chimera (670 pmol/20 g) (5 times, gray arrows) and 50 mg/kg 5-FU (3 times, dark grey arrows). Data (mean+s.d.) were pooled from two experiments, involving a total of 6 mice per group. There was significant difference between 5-FU only group versus miR-26a chimera only group (two way repeated-measures ANOVA followed by Bonferroni post-test for day0 to day18 (interaction p-value<0.001). FIG. 4I is a graph showing tumor volume over time after combinational treatment with 5-FU and chimeras. Data (mean+s.d.) were pooled from two experiments, involving a total of 6 mice per group. There was significant difference between 5-FU+ctrl chimera group versus 5-FU+miR-26a chimera group (two way repeated-measures ANOVA followed by Bonferroni post-test for day0 to day21, interaction p-value<0.0001). FIG. 4J is a graph showing the numbers of WBC and PLT in the tumor bearing mice 5 days after 5-FU treatment. Data (mean+s.d.) pooled from two experiments, involving a total of 6 mice per group. *P<0.05, **P<0.01.

FIG. 5A-FIG. 5C show oncogenomic screening of targeting genes in basal-like breast cancer. FIG. 5A is a picture of a workflow using TCGA database for screening target miRNAs that were significantly increased or decreased in basal-like breast cancer and also associated with overall survival of the patients. FIG. 5B is a picture of a workflow using TCGA database for cell membrane protein-coding genes that were significantly increased in basal-like breast cancer and also associated with overall survival of the patients. FIG. 5C is a graph showing the Kaplan-Meier curves for breast cancer patients with higher or lower expression level of KIT that were obtained from TCGA cohort. Higher expression group (z-score>2), lower expression group (z-score<2).

FIG. 6A-FIG. 6B show how the miR-26a chimera protects mice against carboplatin-induced myelosuppression. C57BL/6 mice were treated with miR-26a chimera intravenously (670 pmol/20 g) daily for 3 days. At day 2, 150 mg/kg 5-FU was injected intravenously. Peripheral blood was collected 5 and 10 days after the 5-FU treatment. FIG. 6A is a series of graphs showing the numbers of WBC, RBC, and PLT 5 days after 5-FU treatment. FIG. 6B is a series of graphs showing the numbers of WBC, RBC, and PLT 10 days after 5-FU treatment. Data (mean+s.d.) were pooled from two experiments, involving a total of 6 mice. *, P<0.05, **, P<0.01.

FIG. 7 is a series of graphs showing the number of RBC and PLT from Bak1^{-/-} mice and wild type mice (Bak1^{+/+}) treated with 150 mg/kg 5-FU. Bak1^{-/-} mice were more resistant against 5-FU-induced myelosuppression. Peripheral blood was collected 0, 5, and 10 days after the 5-FU treatment. Data (mean+s.d.) were pooled from two experiments, involving a total of 7 mice per group. *, P<0.05. A target sequence to cleave (SEQ ID NO:21)

FIG. 8A is a graph showing the pharmacokinetics of plasma levels of miR-26a chimera and the single dose administration in BALB/c mice bearing mouse TUBO breast cancer cell line. miR-26a chimera (670 pmol/20 g) was injected intravenously. Blood samples were collected at the indicated time points. The concentrations of miR-26a were determined by quantitative PCR using standard curve generated with dose-titrated miR-26a chimera. Data were pooled from three experiments, involving a total of 3 mice. FIG. 8B is a series of pictures showing in vivo imaging of miR-26a chimera conjugated with AF647 dye in BALB/c mice bearing tumor with TUBO cell line. A single dose of miR-26a chimera (670 pmol/20 g) was intravenously injected and followed by serial fluorescence imaging at the indicated time points by IVIS spectrum (Caliper LifeSciences, Waltham, Mass.). Data shown are series images from one experiment. Similar data were obtained from another experiment.

FIG. 9A-FIG. 9B show how miR-26a ameliorates 5-FU induced myelosuppression in breast cancer-bearing mice. BALB/c mice bearing TUBO tumors were treated intravenously with miR-26a chimera (670 pmol/20 g) daily for 5 days. At days 2, 3, and 4, 50 mg/kg 5-FU was intravenously injected. Peripheral blood was collected 5 and 10 days after the initial 5-FU treatment. FIG. 9A is a series of graphs showing the numbers of WBC, RBC, and PLT 5 days after 5-FU treatment. FIG. 9B is a series of graphs showing the numbers of WBC, RBC, and PLT 10 days after 5-FU treatment. Data (mean+s.d.) were pooled from two experiments, involving a total of 6 mice per group. *, P<0.05, **, P<0.01.

FIG. 10 is a series of graphs showing that a mixture of miR-26a chimera with 5-FU protects mice against 5-FU-induced myelosuppression. The mice received intravenous injection of 150 mg/kg 5-FU with 2 nmol/20 g miR-26a or control chimera at same time. Graphs show the numbers of white blood cells (WBC), red blood cells (RBC), and platelet (PLT) 5 days and 10 days after carboplatin treatment. n=3.

FIG. 11A is a diagram of the cKit-CD63 aptamer chimera with GFP-loaded exosome. FIG. 11B shows representative cultured cells with GFP plasmid delivery into cells by ckit-targeting exosome (top) and a graph showing measured fluorescence (bottom).

FIG. 12A is a diagram of the cKit-CD63 aptamer chimera with Luciferase enzyme-loaded exosome. FIG. 12B is a graph showing the luminescence measured after delivery of the luciferase expression vector by the ckit-targeting exosome.

FIG. 13A is a diagram of the cKit-CD63 aptamer chimera with a payload of multiple small RNAs. FIG. 13B shows expression levels of mi-R26a from fluorescein-conjugated miR-26a-5p delivered by the ekit-targeting exosome to ckit-overexpressing MEF cells.

FIG. 14 shows expression levels of mi-R26a from fluorescein-conjugated miR-26a-5p, transfected into HEK293 cells and delivered by the ckit-targeting exosome to in vitro cultured bone marrow cells, measured by flow cytometry analysis.

FIG. 15 is a graph showing expression levels of mi-R26a from fluorescein-conjugated miR-26a-5p transfected into JAWSII cell-derived exosome and delivered in vivo to mouse by the ckit-targeting exosome via i.v. injection. 2 days later, bone marrow cells were harvested, sorted by MACS for ckit+, and measured by qPCR. Bars within the rectangle show the exosome with more payload capacity.

FIG. 16 is a diagram of the cKit-CD63 aptamer chimera with CRISPR components loaded into the exosome. Template DNA can also be loaded for homologous recombination.

FIG. 17 is a diagram showing genome editing possibilities with the CRISPR-Cas9 system. The target sequence to cleave is depicted as SEQ ID NO:22. The guide sequence, indicated by an arrow and framed in grey is SEQ ID NO:23. The entire sgRNA sequence including the guide sequence is SEQ ID NO:24.

FIG. 18A shows a diagram of the Cas9-Rosa26 gRNA plasmid pCRISPR-CG01 that was used for gene knock-out in MEF cells by the targeting exosome (top), and the Indel detection assay (bottom). FIG. 18B shows results of the assay.

FIG. 19 shows the donor vector DC-DON-SH02 (RFP/GFP/Puro) (top) used in combination with the Cas9-Rosa26 gRNA plasmid for homologous recombination, delivered to ckit-overexpressing MEF cells in 293T exosome by ckit-CD63 aptamer. Junction PCR was used to detect template sequence integration (bottom).

FIG. 20 shows results of MACS sorting of lineage-depleted bone marrow cells from a BL6 mouse. The mouse was injected 2× i.v. with ckit-targeting JAWSII dendritic cell-derived exosome loaded with Cas9-Rosa26 gRNA vector and template DNA vector (GFP/RFP/Puro).

FIG. 21 shows Rosa locus junction PCR of ex vivo cultured bone marrow cells from FIG. 20 after selection with puromycin. Rectangles how successful detection of template sequence integration.

FIG. 22 shows pictures of organs from a mouse model of lymphoma (p53/Tsc1 double knock-out, top), and depiction of the p53 pX330 plasmid that was used for gene knock-out by targeting exosome to mouse bone marrow cells with ckit-CD63 aptamer (bottom).

FIG. 23 shows results of next generation sequencing for CRISPR genome editing of mouse p53. The arrow shows the CRISPR cutting site with a region of chromosome 11 corresponding to: tatgctccatacagtacacaatctcttctctctacaGATGACTGCCATGGAGGAGTCACAGTCGGATATCAG CCTCGAGCTCCCTCTGAGCCAGGAGACATTTTCAGGCTTATGGAAACTGTGAGTGGA TCTTTTTTGGGG (SEQ ID NO:25). An amino acid sequence is indentified below the cutting site and encoded by a portion of SEQ ID NO:25. That encoded portion is MTAMEESQSDISLELPLSQETFSGLWKI (SEQ ID NO:26).

FIG. 24 shows an enlargement of several genomic sequences in the same portion of the chr11 mouse p53 site in the middle panel (the sequences from top to bottom in the middle panel are SEQ ID NO: 27, SEQ ID NO: 28; SEQ ID NO: 29; SEQ ID NO: 30; SEQ ID NO: 31; SEQ ID NO:32; SEQ ID NO: 33; SEQ ID NO: 34; SEQ ID NO: 35; SEQ ID NO: 36; SEQ ID NO: 37; SEQ ID NO: 38; SEQ ID NO: 39; SEQ ID NO: 40; SEQ ID NO: 41; SEQ ID NO: 42; SEQ ID NO: 43; SEQ ID NO: 44; SEQ ID NO: 45; SEQ ID NO: 46; SEQ ID NO:47; SEQ ID NO: 48; SEQ ID NO: 49; SEQ ID NO: 50; SEQ ID NO: 51; SEQ ID NO: 52; SEQ ID NO: 53; SEQ ID NO: 54; SEQ ID NO: 55; SEQ ID NO: 56; SEQ ID NO: 57; SEQ ID NO: 58; SEQ ID NO: 59; SEQ ID NO: 60; SEQ ID NO: 61; SEQ ID NO: 62; SEQ ID NO: 63; SEQ ID NO: 64; SEQ ID NO: 65; SEQ ID NO: 66; SEQ ID NO:67). The middle panel sequences are all variants of SEQ ID NO:68. The top line sequence of the bottom panel shows the CRISPR cutting site on chromosome 11 (depicted within the sequence GAG) within the chromosome 11 consensus region sequence (SEQ ID NO:68) which is agttatgctccatacagtacacaatctcttctctctacaGATGACTGCCATGGAGGAGTCACAGTCGGATATC AGCCTCGAGCTCCCTCTGAGCCAGGAGACATTTTCAGGCTTATGGAAACTGTGAGTG GATCTTTTGGGGCC. The arrow points to the site of successful indel induction by the exosome CRISPR system. The encoded amino acid from a portion of SEQ ID NO:68 above (encoded by the basepairs beginning at ATGACT) is SEQ ID NO:26.

FIG. 25 shows results of next generation sequencing for CRISPR genome editing of mouse Tsc1. Alignment of several variant sequences is depicted in the middle panel a consensus sequence of the mouse Tsc1 gene is on the topline of the bottom panel. It is: GACGATGGACACTGATGTTGTGGTCCTCACAACTGGTGTCTTGGTGGTGATCACCAT GCTCCCGATGATCCCGCAGTCAGGGAAGCAGCACCTTCTCGACTTCTTTGACATCTT TGGCCG (SEQ ID NO:69). The arrow points to the site of successful indel induction by the exosome CRISPR system within the sequence CAG. The encoded protein of the Tsc1 gene is MDTDVVVLTTGVLVLITMLPMIPQSGKQHLLDFFDIIFGR (SEQ ID NO:70).

FIG. 26A is a diagram of a Seq6-CD63 aptamer chimera with tumor-specific antigens loaded into the exosome. FIG. 26B shows results of cell sorting of dendritic cells with Seq6 and other aptamers bound to exosome via CD63 aptamer-linker.

### DETAILED DESCRIPTION OF EMBODIMENTS

The term “about” as used herein when referring to a measurable value such as an amount, a temporal duration, and the like, is meant to encompass variations of ±20%, ±10%, ±5%, ±1%, ±0.9%, ±0.8%, ±0.7%, ±0.6%, ±0.5%, ±0.4%, 0.3%, ±0.2% or ±0.1% from the specified value, as such variations are appropriate to perform the disclosed methods.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, the term “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, “either,” “one of,” “only one of,” or “exactly one of” “Consisting essentially of,” when used in the claims, shall have its ordinary meaning as used in the field of patent law.

As used herein, “activity” in the context of aptamer activity mi-RNA activity refers to the ability of a nucleic acid and to bind to a target sequence and/or bind a cellular receptor or binding partner to a degree and for a period of time sufficient to allow entry of the nucleic acid sequence into a target cell, such as a cancer cell. Such activity can be measured in a variety of ways as known in the art. For example, mRNA or protein expression, activity, or level of a gene sequence can be measured, and targeting the gene sequence can be assayed for their ability to reduce the expression, activity, or level of the gene. For example, a cell can be transfected with, transformed with, or contacted with a nucleic acid sequence disclosed herein. The activity can be measured by monitoring the expression of the target nucleic acid sequence and comparing expression to a cell not transfected, transformed or contacted with disclosed nucleic acid sequences.

The term “analog” as used herein refers to compounds that are similar but not identical in chemical formula and share the same or substantially similar function of the compound with the similar chemical formula. In some embodiments, the analog is a mutant, variant or modified sequence as compared to the non-modified or wild-type sequence upon which it is based. In some embodiments, compositions of the disclosure include modifications or analogs that are at least about 70%, about 75%, about 80%, about 85%, about 90% about 91%, about 92%, about 93%, about 94%, about 95%, about 96%, about 97%, about 98% or about 99% homology to any of the disclosed nucleic acids disclosed herein. In some embodiments the analog is a functional fragment of any of the disclosed nucleic acid sequences. In some embodiments, the analog is a salt of any of the disclosed nucleic acid sequences. In such embodiments, the analog may retain about 99%, 98%, 97%, 96%, 95%, 94%, 93%, 92%, 91%, 90%, 85%, 80%, 75%, 70% or less biological activity as compared to the natural or wild-type sequences upon which it is based.

The terms “biophysically effective amount” refers to an amount of nucleic acid in a system under one or a plurality of physiological conditions (such as temperature, pH, exposure to percent oxygen, etc.) sufficient for a nucleic acid sequence disclosed herein or an analog thereof to associate with an aptamer domain target or a microRNA target. In some embodiments, the nucleic acid sequence of the disclosure is in a biophysically effective amount.

As used herein, “conservative” amino acid substitutions may be defined as set out in Tables A, B, or C below. The polypeptides of the disclosure include those wherein conservative substitutions (from either nucleic acid or amino acid sequences) have been introduced by modification of polynucleotides encoding polypeptides. In some embodiments, these polypeptides comprise or consist of amino acids that are receptors, such as those receptors which are aptamer domain targets (capable of forming a complex with one or a plurality of nucleic acid sequences of the disclosure when in contact with an aptamer domain) or functional fragments thereof. Amino acids can be classified according to physical properties and contribution to secondary and tertiary protein structure. A conservative substitution is recognized in the art as a substitution of one amino acid for another amino acid that has similar properties. In some embodiments, the conservative substitution is recognized in the art as a substitution of one nucleic acid for another nucleic acid that has similar properties, or, when encoded, has similar binding affinities. Exemplary conservative substitutions are set out in Table A.

TABLE A
Conservative Substitutions I

| Side Chain | |
|---|---|
| Characteristics | Amino Acid |
| Aliphatic | |
| Non-polar | G A P I L V F |
| Polar - uncharged | C S T M N Q |
| Polar - Charged | D E K R |
| Aromatic | H F W Y |
| Other | N Q D E |

Alternately, conservative amino acids can be grouped as described in Lehninger, (Biochemistry, Second Edition; Worth Publishers, Inc. NY, N.Y. (1975), pp. 71-77) as set forth in Table B.

TABLE B
Conservative Substitutions II

| | |
|---|---|
| Side Chain | |
| Characteristic | Amino Acid |

Non-polar (hydrophobic)

| | |
|---|---|
| Aliphatic: | A L I V P |
| Aromatic: | F W Y |
| Sulfur-containing: | M |
| Borderline: | G Y |

Uncharged-polar

| | |
|---|---|
| Hydroxyl: | S T Y |
| Amides: | N Q |
| Sulfhydryl: | C |
| Borderline: | G Y |
| Positively Charged (Basic): | K R H |
| Negatively Charged (Acidic): | D E |

Alternately, exemplary conservative substitutions are set out in Table C.

TABLE C
Conservative Substitutions III

| Original Residue | Exemplary Substitution |
|---|---|
| Ala (A) | Val Leu Ile Met |
| Arg (R) | Lys His |
| Asn (N) | Gln |
| Asp (D) | Glu |
| Cys (C) | Set Thr |
| Gln (Q) | Asn |
| Glu (E) | Asp |
| Gly(G) | Ala Val Leu Pro |
| His (H) | Lys Arg |
| Ile (I) | Leu Val Met Ala Phe |
| Leu (L) | Ile Val Met Ala Phe |
| Lys (K) | Arg His |
| Met (M) | Leu Ile Val Ala |
| Phe (F) | Trp Tyr Ile |
| Pro (P) | Gly Ala Val Leu Ile |
| Ser (S) | Thr |
| Thr (T) | Ser |
| Trp (W) | Tyr Phe Ile |
| Tyr (Y) | Trp Phe Thr Ser |
| Val (V) | Ile Leu Met Ala |

It should be understood that some amino acid sequences (such as aptamer domain targets) or any analog thereof described herein are intended to include amino acid sequences comprising polypeptides bearing one or more insertions, deletions, or substitutions, or any combination thereof, of amino acid residues as well as modifications other than insertions, deletions, or substitutions of amino acid residues.

The terms “aptamer domain” refers to a nucleic acid element or domain within a nucleic acid sequence or polynucleotide sequence that, at a biophysically effective amount, will bind or have an affinity for one or a plurality of aptamer target domains presented within or on a cell. In some embodiments, in the presence of one or a plurality of proteins (or functional fragments thereof) and a target sequence, the one or plurality of proteins and the nucleic acid element forms a biologically active complex and/or can be enzymatically active on a target sequence.

The terms “CRISPR-associated genes” refer to any nucleic acid that encodes a regulatory or expressible gene that regulates a component or encodes a component of the CRISPR system. In some embodiments, the terms “CRISPR-associated genes” refer to any nucleic acid sequence that encodes any of the proteins in Table D or Table E (or functional fragments or analogs thereof that are at least about 70, about 75, about 80, about 85, about 90, about 95, about 96, about 97, about 98, or about 99% homologous to the sequences disclosed in either Table). In some embodiments, the terms “Cas-binding domain” or “Cas protein-binding domain” refers to a nucleic acid element or domain within a nucleic acid sequence or polynucleotide sequence that, in a biophysically effective amount, will bind to or have an affinity for one or a plurality of proteins in Table D or Table E (or functional fragments or variants thereof that are at least about 70, 75, 80, 85, 90, 95, 96, 97, 98, or 99% homologous to the sequences disclosed in either Table). In some embodiments, the Cas binding domain consists of no more than about 10, 11, 12, 13, 14, 15, 16, 17 18, 19, 20, 25, 30, 35, 40, 41, 42, 43, 44, 45, 50, 60, 70, 80, 90, 100, 110, 120, 130, 140, 150, 160, 170, 180, 190, 200, 210, 220, 230, 240, 250 or more nucleotides in length and comprises at least one sequence that is capable of forming a hairpin or duplex that partially associates or binds to a biologically active CRISPR system at a concentration and within microenvironment suitable for CRISPR system formation. In some embodiments, the composition or pharmaceutical compositions comprises one or a combination of sgRNA, crRNA, and tracrRNA that consists of no more than about 25, 30, 35, 40, 45, 50, 60, 70, 80, 90, 100, 110, 120, 130, 140, 150, 160, 170, 180, 190, 200, 210, 220, 230, 240, 250 or more nucleotides in length and comprises at least one sequence that is capable of forming a hairpin or duplex that partially associates or binds to a biologically active amino acid sequence (or functional fragment disclosed herein) disclosed in Table E at a concentration and within microenvironment suitable for CRISPR system formation and CRISPR enzymatic activity on a target sequence. In some embodiments, the Cas protein derived from the Cas9 family of Cas proteins or a functional fragment thereof.

TABLE D
CRISPR enzymes (Cas proteins, or Cas-like proteins) organized by Family

| | | | | Structure of | | |
|---|---|---|---|---|---|---|
| | | | Name | encoded | Families (and | |
| | | Name | from | protein | superfamily) | |
| Proposed | System type or | from Haft | Brouns | (PDB | of encoded | |
| gene name^{‡} | subtype | et al.^{§} | et al.^{\| \|} | accessions)^{¶} | protein^{#}** | Representatives |
| cas1 | Type I | cas1 | cas1 | 3GOD, 3LFX | COG1518 | SERP2463, |
| | Type II | | | and 2YZ5 | | SPy1047 and |
| | Type III | | | | | ygbT |
| cas2 | Type I | cas2 | cas1 | 2IVY, 2I8E | COG1343 | SERP2462, |
| | Type II | | | and 3EXC | and | SPy1048, |
| | Type III | | | | COG3512 | SPy1723 (N- |
| | | | | | | terminal |
| | | | | | | domain) and |
| | | | | | | ygbF |
| cas3′ | Type I^{‡‡} | cas3 | cas3 | NA | COG1203 | APE1232 and |
| | | | | | | ygcB |
| cas3″ | Subtype I-A | NA | NA | NA | COG2254 | APE1231 and |
| | Subtype I-B | | | | | BH0336 |
| cas4 | Subtype I-A | cas4 and | NA | NA | COG1468 | APE1239 and |
| | Subtype I-B | csa1 | | | | BH0340 |
| | Subtype I-C | | | | | |
| | Subtype I-D | | | | | |
| | Subtype II-B | | | | | |
| cas5 | Subtype I-A | cas5a; | casD | 3KG4 | COG1688 | APE1234, |
| | Subtype I-B | cas5d, | | | (RAMP) | BH0337, dev5 |
| | Subtype I-C | cas5e, | | | | and ygcl |
| | Subtype I-E | cas5h, | | | | |
| | | cas5p, | | | | |
| | | cas5t and | | | | |
| | | cmx5 | | | | |
| cas6 | Subtype I-A | cas6 and | NA | 3I4H | COG1583 | PF1131 and |
| | Subtype I-B | cmx6 | | | and | slr7014 |
| | Subtype I-D | | | | COG5551 | |
| | Subtype III-A | | | | (RAMP) | |
| | Subtype III-B | | | | | |
| cas6e | Subtype I-E | cse3 | casE | 1WJ9 | (RAMP) | ygcH |
| cas6f | Subtype I-F | csy4 | NA | 2XIJ | (RAMP) | y1727 |
| cas7 | Subtype I-A | csa2, | casC | NA | COG1857 and | devR and ygcJ |
| | Subtype I-B | csd2, | | | COG3649 | |
| | Subtype I-C | cse4, | | | (RAMP) | |
| | Subtype I-E | csh2, csp1 | | | | |
| | | and cst2 | | | | |
| cas8a1 | Subtype I-A^{‡‡} | cmx1, | NA | NA | BH0338-like | LA3191^{§§} and |
| | | cst1, csx8, | | | | PG2018^{§§} |
| | | csx13 and | | | | |
| | | CXXC- | | | | |
| | | CXXC | | | | |
| cas8a2 | Subtype I-A^{‡‡} | csa4 and | NA | NA | PH0918 | AF0070, AF1873, |
| | | csx9 | | | | MJ0385, PF0637, |
| | | | | | | PH0918 and |
| | | | | | | SSO1401 |
| cas8b | Subtype I-B^{‡‡} | csh1 and | NA | NA | BH0338-like | MTH11090 and |
| | | TM1802 | | | | TM1802 |
| css8c | Subtype I-C^{‡‡} | csd1 and | NA | NA | BH0338-like | BH0338 |
| | | csp2 | | | | |
| cas9 | Type II^{‡‡} | csn1 and | NA | NA | COG3513 | FTN_0757 and |
| | | csx12 | | | | SPy1046 |
| cas10 | Type III^{‡‡} | cmr2, | NA | NA | COG1353 | MTH326, |
| | | csm1 and | | | | Rv2823c§§ and |
| | | csx11 | | | | TM1794§§ |
| cas10d | Subtype I-D^{‡‡} | csc3 | NA | NA | COG1353 | slr7011 |
| csy1 | Subtype I-F^{‡‡} | csy1 | NA | NA | y1724-like | y1724 |
| csy2 | Subtype I-F | csy2 | NA | NA | (RAMP) | y1725 |
| csy3 | Subtype I-F | csy3 | NA | NA | (RAMP) | y1726 |
| cse1 | Subtype I-E^{‡‡} | cse1 | casA | NA | YgcL-like | ygcL |
| cse2 | Subtype I-E | cse2 | casB | 2ZCA | YgcL-like | ygcK |
| csc1 | Subtype I-D | csc1 | NA | NA | alr1563-like | alr1563 |
| | | | | | (RAMP) | |
| csc2 | Subtype I-D | csc1 and | NA | NA | COG1337 | slr7012 |
| | | csc2 | | | (RAMP) | |
| csa5 | Subtype I-A | csa5 | NA | NA | AF1870 | AF1870, |
| | | | | | | MJ0380, PF0643 |
| | | | | | | and SSO1398 |
| csn2 | Subtype II-A | csn2 | NA | NA | SPy1049-like | SPy1049 |
| csm2 | Subtype III-A^{‡‡} | csm2 | NA | NA | COG1421 | MTJH1081 and |
| | | | | | | SERP2460 |
| csm3 | Subtype III-A | csc2 and | NA | NA | COG1337 | MTH1080 and |
| | | csm3 | | | (RAMP) | SERP2459 |
| csm4 | Subtype III-A | csm4 | NA | NA | COG1567 | MTH1079 and |
| | | | | | (RAMP) | SERP2458 |
| csm5 | Subtype III-A | csm5 | NA | NA | COG1332 | MTH1078 and |
| | | | | | (RAMP) | SERP2457 |
| csm6 | Subtype III-A | APE2256 | NA | 2WTE | COG1517 | APE2256 and |
| | | and csm6 | | | | SSO1445 |
| cmr1 | Subtype III-B | cmr1 | NA | NA | COG1367 | PF1130 |
| | | | | | (RAMP) | |
| cmr3 | Subtype III-B | cmr3 | NA | NA | COG1769 | PF1128 |
| | | | | | (RAMP) | |
| cmr4 | Subtype III-B | cmr4 | NA | NA | COG1336 | PF1126 |
| | | | | | (RAMP) | |
| cmr5 | Subtype III-B^{‡‡} | cmr5 | NA | 2ZOP and | COG3337 | MTH324 and |
| | | | | 2OEB | | PF1125 |
| cmr6 | Subtype III-B | cmr6 | NA | NA | COG1604 | PF1124 |
| | | | | | (RAMP) | |
| csb1 | Subtype I-U | GSU0053 | NA | NA | (RAMP) | Balac_1306 and |
| | | | | | | GSU0053 |
| csb2 | Subtype I-U^{§§} | NA | NA | NA | (RAMP) | Balac_1305 and |
| | | | | | | GSU0054 |
| csb3 | Subtype I-U | NA | NA | NA | (RAMP) | Balac_1303^{§§} |
| csx17 | Subtype I-U | NA | NA | NA | NA | Btus_2683 |
| csx14 | Subtype I-U | NA | NA | NA | NA | GSU0052 |
| csx10 | Subtype I-U | csx10 | NA | NA | (RAMP) | Caur_2274 |
| csx16 | Subtype III-U | VVA1548 | NA | NA | NA | VVA1548 |
| csaX | Subtype III-U | csaX | NA | NA | NA | SSO1438 |
| csx3 | Subtype III-U | csx3 | NA | NA | NA | AF1864 |
| csx1 | Subtype III-U | csa3, | NA | 1XMX and | COG1517 | MJ1666, |
| | | csx1, csx2, | | 2I71 | and | NE0113, PF1127 |
| | | DXTHG, | | | COG4006 | and TM1812 |
| | | NE0113 | | | | |
| | | and | | | | |
| | | TIGR02710 | | | | |
| csx15 | Unknown | NA | NA | NA | TTE2665 | TTE2665 |

All amino acid and nucleic acid sequences associated with the Accession Numbers below as of May 18, 2017, are incorporated by reference in their entireties. Any mutants or variants that are at least 70, 75, 80, 85, 90, 95, 96, 97, 98, 99% homologous to the encoded nucleic acids or amino acids set forth in the Accession Numbers below are also incorporated by reference in their entireties.

TABLE E
Accession Numbers of Cas proteins (or those related with
Cas-like function) and Nucleic Acids encoding the same.

NC_014644.1; NC_002967.9; NC_007929.1; NC_000913.3; NC_004547.2; NC_009380.1;
NC_011661.1; NC_010175.1; NC_010175.1; NC_010175.1; NC_003413.1; NC_000917.1;
NC_002939.5; NC_0182.27.2; NC_004829.2; NC_021921.1; NC_014160.1; NC_011766.1;
NC_007681.1; NC_021592.1; NC_021592.1; NC_021169.1; NC_020517.1; NC_018656.1;
NC_018015.1; NC_018015.1; NC_017946.1; NC_017576.1; NC_017576.1; NC_015865.1;
NC_015865.1; NC_015680.1; NC_015680.1; NC_015474.1; NC_026150.1; NC_003552.1;
NC_025263.1; NC_016112.1; NC_008526.1; NC_015474.1; NC_012804.1; NC_015518.1;
NC_017276.1; NC_017275.1; NC_012632.1; NC_012623.1; NC_012588.1; NC_015636.1;
NC_015562.1; NC_013769.1; NC_014374.1; NC_009776.1; NC_005877.1; NC_005877.1

The terms “aptamer target domain” refers to a amino acid sequence or nucleic acid element or domain within a nucleic acid sequence (or polynucleotide sequence) that binds to an aptamer domain either covalently or non-covalently when the aptamer domain is in contact with the aptamer target domain in a biophysically effective amount. In some embodiments, the aptamer target domain consists of no more than about 5, 10, 15, 20, 25, 30, 35, 40, 45, 50, 60, 70, 80, 90, 100, 110, 120, 130, 140, 150, 160, 170, 180, 190, 200, 210, 220, 230, 240, 250 or more amino acids or nucleotides in length and comprises at least one sequence that is capable of forming a hairpin or duplex that partially drives association of the nucleic acid sequence to an aptamer domain at a concentration and microenvironment sufficient for association. In some embodiments, the aptamer target domain is expressed by a cancer cell, such as a breast cancer cell. In some embodiments, the aptamer target domain is expressed by a hematopoietic stem cell. In some embodiments, the aptamer target domain is expressed by a cancer stem cell.

In some embodiments, an aptamer target domain or sequence is located in the nucleus or cytoplasm of a cell. In some embodiments, the target sequence may be within an organelle of a eukaryotic cell, for example, mitochondrion or chloroplast. In some embodiments, the compostions of the disclosure comprises one or a plurality of nucleic acid sequences comprising at least one aptamer domain that recognize one or a plurality of aptamer target domains, wherein the aptamer target domain or domains are expressed on the surface of a cell.

One or a plurality of vectors may also be components in any system or composition provided herein. In some embodiments, the disclosure comprises a composition comprising a vector comprising any nucleic acid sequence disclosed herein optionally comprising a regulatory sequence that is operably connected to the nucleic acid sequence disclosed herein such that the nucleic acid sequence is expressible under conditions sufficient to induce expression of the nucleic acid. In some embodiments, the vector comprises one or more insertion sites, such as a restriction endonuclease recognition sequence (also referred to as a “cloning site”). In some embodiments, one or more insertion sites (e.g. about or more than about 1, 2, 3, 4, 5, 6, 7, 8, 9, 10, or more insertion sites) are located upstream and/or downstream of one or more sequence elements of one or more vectors.

When multiple, different nucleic acid sequences disclosed herein are used together, a single expression construct may be used to target an aptamer domain to multiple, different, corresponding aptamer target domains sequences within and/or on a cell. In some embodiments, the disclosure erelates to a composition with one or a plurality of vectors expressing a first, second, third, and/or fourth or more nucleic acid sequence disclosed herein, For example, a single vector may comprise about or more than about 1, 2, 3, 4, 5, 6, 7, 8, 9, 10, 15, 20, or more nucleic acid sequences disclosed herein. In some embodiments, about or more than about 1, 2, 3, 4, 5, 6, 7, 8, 9, 10, or more such nucleic acid-sequence-containing vectors may be provided, and optionally delivered to a cell. The disclosure relates to any composition comprising any of the aforementioned elements and one or more nucleic acid molecules (for instance a first and second) each comprising one or more nucleic acid sequences disclosed herein.

Another aspect of the disclosure relates to a CRISPR system comprising a nucleic acid sequence disclosed herein further comprising one or more CRISPR domains. The CRISPR domain comprises a nucleic acid that expresses a wild type, natural or modified CRISPR enzyme (or “Cas protein”) or a nucleotide sequence encoding one or more Cas proteins. Any protein capable of enzymatic activity in cooperation with a guide sequence is a Cas protein. In some embodiments, the disclosure relates to a system comprises a vector comprising a regulatory element operably linked to an enzyme-coding sequence encoding a CRISPR enzyme, such as a Cas protein from the Cas family of enzymes. In some embodiments, the nucleic acid of the disclosure comprises a CRISPR sgRNA sequence contiguously or non-contiguously upstream or downstream from one or more aptamer domains. In some embodiments, the nucleic acid of the disclosure comprises a CRISPR sgRNA sequence contiguously or non-contiguously upstream or downstream from one or more aptamer domains and/or one or more miRNA domains.

In some embodiments, the disclosure relates to a system, composition, or pharmaceutical composition comprising any one or plurality of Cas proteins either individually or in combination with one or a plurality of nucleic acid sequences disclosed herein. Compositions of one or a plurality of Cas proteins may be administered to a subject with any of the disclosed guide sequences sequentially or contemporaneously. Non-limiting examples of Cas proteins include Cas1, Cas1B, Cas2, Cas3, Cas4, Cas5, Cas6. Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3. Csf4, type V CRISPR-Cas systems, variants and fragments thereof, or modified versions thereof having at least 70% homology to the sequences of Table E. These enzymes are known; for example, the amino acid sequence of S. pyogenes Cas9 protein may be found in the SwissProt database under accession number Q99ZW2. In some embodiments, the unmodified CRISPR enzyme has DNA cleavage activity, such as Cas9. In some embodiments the CRISPR enzyme is Cas9, and may be Cas9 from S. pyogenes or S. pneumoniae. In some embodiments, the CRISPR enzyme directs cleavage of one or both strands at the location of a target sequence, such as within the target sequence and/or within the complement of the target sequence. In some embodiments, the CRISPR enzyme directs cleavage of one or both strands within about 1, 2, 3, 4, 5, 6, 7, 8, 9, 10, 15, 20, 25, 50, 100, 200, 500, or more base pairs from the first or last nucleotide of a target sequence. In some embodiments, a vector encodes a CRISPR enzyme or Cas protein that is mutated to with respect to a corresponding wild-type enzyme such that the mutated CRISPR enzyme lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). Other examples of mutations that render Cas9 a nickase include, without limitation, H840A, N854A, and N863A. In some embodiments, a Cas9 nickase may be used in combination with guide sequenc(es), e.g., two guide sequences, which target respectively sense and antisense strands of the DNA target. This combination allows both strands to be nicked and used to induce NHEJ.

As a further example, two or more catalytic domains of Cas9 (RuvC I, RuvC II, and RuvC III) may be mutated to produce a mutated Cas9 substantially lacking all DNA cleavage activity. In some embodiments, a D10A mutation is combined with one or more of H840A, N854A, or N863A mutations to produce a Cas9 enzyme substantially lacking all DNA cleavage activity. In some embodiments, a CRISPR enzyme is considered to substantially lack all DNA cleavage activity when the DNA cleavage activity of the mutated enzyme is less than about 25%, 10%, 5%, 1%, 0.1%, 0.01%, or lower with respect to its non-mutated form. Other mutations may be useful; where the Cas9 or other CRISPR enzyme is from a species other than S. pyogenes, mutations in corresponding amino acids may be made to achieve similar effects.

In some embodiments, an enzyme coding sequence encoding a CRISPR enzyme is codon optimized for expression in particular cells, such as eukaryotic cells. The eukaryotic cells may be those of or derived from a particular organism or a particular subject, such as a mammal, including but not limited to human, mouse, rat, rabbit, dog, or non-human primate. In general, codon optimization refers to a process of modifying a nucleic acid sequence for enhanced expression in the host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database”, and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, Pa.), are also available. In some embodiments, one or more codons (e.g. 1, 2, 3, 4, 5, 10, 15, 20, 25, 50, or more, or all codons) in a sequence encoding a CRISPR enzyme correspond to the most frequently used codon for a particular amino acid.

As used herein, “expression” refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

The terms “functional fragment” means any portion of a polypeptide or nucleic acid sequence from which the respective full-length polypeptide or nucleic acid relates that is of a sufficient length and has a sufficient structure to confer a biological affect that is at least similar or substantially similar to the full-length polypeptide or nucleic acid upon which the fragment is based. In some embodiments, a functional fragment is a portion of a full-length or wild-type nucleic acid sequence that encodes any one of the nucleic acid sequences disclosed herein, and said portion encodes a polypeptide of a certain length and/or structure that is less than full-length but encodes a domain that still biologically functional as compared to the full-length or wild-type protein. In some embodiments, the functional fragment may have a reduced biological activity, about equivalent biological activity, or an enhanced biological activity as compared to the wild-type or full-length polypeptide sequence upon which the fragment is based. In some embodiments, the functional fragment is derived from the sequence of an organism, such as a human. In such embodiments, the functional fragment may retain 99%, 98%, 97%, 96%, 95%, 94%, 93%, 92%, 91%, or 90% sequence identity to the wild-type human sequence upon which the sequence is derived. In some embodiments, the functional fragment may retain 85%, 80%, 75%, 70%, 65%, or 60% sequence homology to the wild-type sequence upon which the sequence is derived. In such embodiments, the functional fragment may retain about 99%, 98%, 97%, 96%, 95%, 94%, 93%, 92%, 91%, 90%, 85%. 80%, 75%, 70% or less biological activity as compared to the natural or wild-type sequences upon which it is based. In some embodiments, the composition provided comprises one, two, three or more a nucleic acid sequences or salts thereof that is a functional fragment retaining 99%, 98%, 97%, 96%, 95%. 94%, 93%, 92%, 91%, 90%, 85%, 80%, 75%, or 70% sequence identity to any sequence identified in Table 4. In some embodiments, the composition provided comprises a therapeutically effective amount of a nucleic acid molecule or multiple nucleic acid molecules or salts thereof that comprise one, two, three or more a nucleic acid sequences or salts thereof that is a variant having 99%, 98%, 97%. 96%, 95%, 94%, 93%, 92%, 91%. 90%, 85%, 80%, 75%, or 70% sequence identity to any sequences identified in Table 4. In the case of bispecific aptamers, such embodiments comprise a composition comprising a therapeutically effective amount of a nucleic acid molecule or multiple nucleic acid molecules or salts thereof, wherein each nucleic acid molecule or salt thereof comprises a first and a second nucleic acid sequences that comprise at least one aptamer domain that is a variant having 99%, 98%, 97%, 96%, 95%, 94%, 93%, 92%, 91%, 90%, 85%, 80%, 75%, or 70% sequence identity to any sequence identified in Table 4 or any sequence capable of binding the aptamer targeting domain identified in Table 1.

“Hybridization” refers to a reaction in which one or more polynucleotides react to form a complex that is stabilized via hydrogen bonding between the bases of the nucleotide residues. The hydrogen bonding may occur by Watson Crick base pairing, Hoogstein binding, or in any other sequence specific manner. The complex may comprise two strands forming a duplex structure, three or more strands forming a multi stranded complex, a single self-hybridizing strand, or any combination of these. A hybridization reaction may constitute a step in a more extensive process, such as the initiation of PCR, or the cleavage of a polynucleotide by an enzyme. A sequence capable of hybridizing with a given sequence is referred to as the “complement” of the given sequence. In some embodiments, association or binding of a disclosed nucleic acid sequence is hybridizing with a nucleic acid sequence or molecule within a target cell.

The present disclosure also relates to isotopically-enriched compounds, which are structurally similar to the nucleic acid sequences disclosed herein, but for the fact that one or more atoms of the nucleic acid sequence are replaced by an atom having an atomic mass or mass number different from the atomic mass or mass number usually found in nature. Examples of isotopes that can be incorporated into compounds of the disclosure include isotopes of hydrogen, carbon, nitrogen, oxygen, phosphorous, fluorine and chlorine, such as ²H, ³H, ¹³C, ¹⁴C, ¹⁴C, N, ¹⁶O, ¹⁷O, ³¹P, ³²p, ³⁵S, ¹⁸F, and ³⁶Cl. Nucleic acids of the present disclosures that contain the aforementioned isotopes and/or other isotopes of other atoms are within the scope of this disclosure. Certain isotopically-labelled compounds of the present disclosure, for example those into which radioactive isotopes such as 3H and 14C are incorporated, are useful in drug and/or substrate tissue distribution assays. Tritiated, i.e., 3H, and carbon-14. i.e., 14C, isotopes are particularly preferred for their ease of preparation and detection. Further, substitution with heavier isotopes such as deuterium. i.e., 2H, can afford certain therapeutic advantages resulting from greater metabolic stability, for example increased in vivo half-life or reduced dosage requirements and, hence, may be preferred in some circumstances. Isotopically enriched compounds of this disclosure can generally be prepared by substituting a readily available isotopically labeled reagent for a non-isotopically enriched reagent. In some embodiments, the compositions of the disclosure comprise one or more nucleic acid sequences disclosed herein comprising an aptamer domain and a miRNA domain with one or more atoms replaced with a radioisotope. In some embodiments, such radioactive nucleic acid sequences may be a component in a pharmaceutical composition that delivers a radioisotope to a cancer cell after administration to a subject in need of the treatment. In some embodiments, the radioactive nucleic acid sequence can be used as a targeted imaging agent whereupon, after administration to a subject, one or more imaging techniques may be used to detect where within a subject one or a plurality of cancer cells may exist within the subject. Such imaging techniques include PET scanning or CT scanning.

The disclosure relates to nucleic acids disclosed herein unsolvated forms as well as solvated forms, including hydrated forms. The compounds of the disclosure also are capable of forming both pharmaceutically acceptable salts, including but not limited to acid addition and/or base addition salts. Furthermore, compounds of the present disclosure may exist in various solid states including an amorphous form (noncrystalline form), and in the form of clathrates, prodrugs, polymorphs, bio-hydrolyzable esters, racemic mixtures, non-racemic mixtures, or as purified stereoisomers including, but not limited to, optically pure enantiomers and diastereomers. In general, all of these forms can be used as an alternative form to the free base or free acid forms of the compounds, as described above and are intended to be encompassed within the scope of the present disclosure.

“Nucleobase” means a heterocyclic moiety capable of non-covalently pairing with another nucleobase.

“Nucleoside” means a nucleobase linked to a sugar moiety.

“Nucleotide” means a nucleoside having a phosphate group covalently linked to the sugar portion of a nucleoside. In some embodiments, the nucleotide is characterized as being modified if the 3′ phosphate group is covalently linked to a contiguous nucleotide by any linkage other than a phosphodiester bond.

The disclosure relates to any nucleic acid sequence disclosed herein also comprising one or a plurality of modified nucleotides. In some embodiments, the compositions of the disclosure comprise a nucleic acid sequence disclosed herein comprising one or a plurality of modified oligonucleotides. In some embodiments, the composition comprises any one, two, three or more nucleic acid sequences disclosed herein comprising a modified oligonucleotide consisting of a number of linked nucleosides. Thus, the compound or compounds may include additional substituents or conjugates. Unless otherwise indicated, the compound does not include any additional nucleosides beyond those of the modified oligonucleotide.

“Modified oligonucleotide” means an oligonucleotide having one or more modifications relative to a naturally occurring terminus, sugar, nucleobase, and/or internucleoside linkage. A modified oligonucleotide may comprise unmodified nucleosides at one or a plurality of any of the positions of the disclosed nucleic acids.

“Single-stranded modified oligonucleotide” means a modified oligonucleotide which is not hybridized to a complementary strand. In some embodiments, the compositions of the disclosure relate to a nucleic acid molecule that is a single-stranded modified oligonucleotide comprising any one or more domains disclosed herein.

The nucleic acid sequences of the disclosure can comprise one or more modified nucleosides. The terms “modified nucleoside” mean a nucleoside having any change from a naturally occurring nucleoside. A modified nucleoside may have a modified sugar, and an unmodified nucleobase. A modified nucleoside may have a modified sugar and a modified nucleobase. A modified nucleoside may have a natural sugar and a modified nucleobase. In certain embodiments, a modified nucleoside is a bicyclic nucleoside. In certain embodiments, a modified nucleoside is a non-bicyclic nucleoside.

A “polymorph” refers to solid crystalline forms of the one or more nucleic acid sequences disclosed herein. In some embodiments, one or more nucleic acids disclosed herein are in a polymorph form. Different polymorphs of the same compound can exhibit different physical, chemical and/or spectroscopic properties. Different physical properties include, but are not limited to stability (e.g., to heat or light), compressibility and density (important in formulation and product manufacturing), and dissolution rates (which can affect bioavailability). Different physical properties of polymorphs can affect their processing.

The nucleic acid sequences, proteins or other agents of the present disclosure can be administered, inter alia, as pharmaceutically acceptable salts, esters, or amides. The term “salts” refers to inorganic and organic salts of compounds of the present disclosure. The salts can be prepared in situ during the final isolation and purification of a compound, or by separately reacting a purified compound in its free base or acid form with a suitable organic or inorganic base or acid and isolating the salt thus formed. Representative salts include the hydrobromide, hydrochloride, sulfate, bisulfate, nitrate, acetate, oxalate, palmitate, stearate, laurate, borate, benzoate, lactate, phosphate, tosylate, citrate, maleate, fumarate, succinate, tartrate, naphthylate, mesylate, glucoheptonate, lactobionate, and laurylsulphonate salts, and the like. The salts may include cations based on the alkali and alkaline earth metals, such as sodium, lithium, potassium, calcium, magnesium, and the like, as well as non-toxic ammonium, quaternary ammonium, and amine cations including, but not limited to, ammonium, tetramethylammonium, tetraethylammonium, methylamine, dimethylamine, trimethylamine, triethylamine, ethylamine, and the like. See, for example, S. M. Berge, et al., “Pharmaceutical Salts,” J Pharm Sci, 66: 1-19 (1977), which discloses salt forms of nucleic acids and which is incorporated by reference in its entirety.

The terms “polynucleotide”, “nucleotide sequence”, “nucleic acid” and “oligonucleotide” are used interchangeably. They refer to a polymeric form of nucleotides of any length, either deoxyribonucleotides or ribonucleotides, or analogs thereof. Polynucleotides may have any three dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after synthesis or polymerization, such as by conjugation with a labeling component.

The oligonucleotides of the disclosure also include those nucleic acid sequences disclosed herein that comprise nucleosides connected by charged linkages, and/or whose sequences are divided into at least two subsequences. In some embodiments, a first, second, and third subsequence or domains include an aptamer domain and a miRNA domain. In some embodiments the nucleic acid sequence comprises a sgRNA guide sequence with a nucleotide binding domain (or DNA-binding domain), a Cas-binding domain, and a transcription terminator domain. In some embodiments, a first, second, third, fourth, and/or fifth subsequence or domains include a nucleotide binding domain, a Cas-binding domain, and a transcription terminator sequence, but, if any two domains are present they must be oriented such that the aptamer domain precedes the miRNA domain. If the embodiment includes a sgRNA sequence or sequence elements, such sequences, in some embodiments, the nucleic acid sequence comprises a nucleotide binding domain which precede a Cas-binding domain which, in turn precedes the transcription terminator domain in a 5′ to 3′ orientation. Any of the nucleosides within any of the domains may be 2′-substituted-nucleosides linked by a first type of linkage. The second subsequence includes nucleosides linked by a second type of linkage.

In the context of this disclosure, the term “oligonucleotide” also refers to a plurality of nucleotides joined together in a specific sequence from naturally and non-naturally occurring nucleobases. Nucleobases of the disclosure are joined through a sugar moiety via phosphorus linkages, and may include any one or combination of adenine, guanine, cytosine, uracil, thymine, xanthine, hypoxanthine, 2-aminoadenine. 6-methyl, 2-propyl and other alkyl adenines, 5-halo uracil, 5-halo cytosine, 6-aza uracil, 6-aza cytosine and 6-aza thymine, pseudo uracil, 4-thiouracil, 8-halo adenine, 8-aminoadenine, 8-thiol adenine, 8-thiolalkyl adenines, 8-hydroxyl adenine and other 8-substituted adenines, 8-halo guanines, 8-amino guanine, 8-thiol guanine, 8-thiolalkyl guanines, 8-hydroxyl guanine and other 8-substituted guanines, other aza and deaza uracils, other aza and deaza thymidines, other aza and deaza cytosines, other aza and deaza adenines, other aza and deaza guanines, 5-trifluoromethyl uracil and 5-trifluoro cytosine. The sugar moiety may be deoxyribose or ribose. The sugar moiety may be a modified deoxyribose or ribose with one or more modifications on the C₁, C₂, C₃, C₄, and/or C₅ carbons. The oligonucleotides of the disclosure may also comprise modified nucleobases or nucleobases having other modifications consistent with the spirit of this disclosure, and in particular modifications that increase their nuclease resistance in order to facilitate their use as therapeutic, diagnostic or research reagents.

The terms “polypeptide”, “peptide” and “protein” are used interchangeably herein to refer to polymers of amino acids of any length. The polymer may be linear or branched, it may comprise modified amino acids, and it may be interrupted by non-natural amino acids or chemical groups that are not amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” includes natural and/or unnatural or synthetic amino acids, including glycine and both the D or L optical isomers, and amino acid analogs and peptidomimetics.

As used herein, “more than one” or “two or more” 2, 3, 4, 5, 6, 7, 8, 9, or 10 or more where “more” may be an positive integer above 10 that corresponding to the length of nucleotides in the nucleotide sequences. In some embodiments, “more than one” means 2, 3, 4, or 5 of the amino acids or nucleic acids or mutations described herein. In some embodiments, “more than one” means 2, 3, or 4 of the amino acids or nucleic acids or mutations described herein. In some embodiments, “more than one” means 2 or 3 of the amino acids or nucleic acids or mutations described herein. In some embodiments, “more than one” means 2 of the amino acids or nucleic acids or mutations described herein.

“Sugar moiety” means a naturally occurring furanosyl or a modified sugar moiety.

“Modified sugar moiety” means a substituted sugar moiety or a sugar surrogate.

“Substituted sugar moiety” means a furanosyl that is not a naturally occurring furanosyl. Substituted sugar moieties include, but are not limited to sugar moieties comprising modifications at the 2′-position, the 5′-position and/or the 4′-position of a naturally occurring furanosyl. Certain substituted sugar moieties are bicyclic sugar moieties.

“Sugar surrogate” means a structure that does not comprise a furanosyl and that is capable of replacing the naturally occurring furanosyl of a nucleoside, such that the resulting nucleoside is capable of (1) incorporation into an oligonucleotide and (2) hybridization to a complementary nucleoside. Such structures include relatively simple changes to the furanosyl, such as rings comprising a different number of atoms (e.g., 4, 6, or 7-membered rings); replacement of the oxygen of the furanosyl with a non-oxygen atom (e.g., carbon, sulfur, or nitrogen); or both a change in the number of atoms and a replacement of the oxygen. Such structures may also comprise substitutions corresponding with those described for substituted sugar moieties (e.g., 6-membered carbocyclic bicyclic sugar surrogates optionally comprising additional substituents). Sugar surrogates also include more complex sugar replacements (e.g., the non-ring systems of peptide nucleic acid). Sugar surrogates include without limitation morpholinos, cyclohexenyls and cyclohexitols. In some embodiments, the nucleic acid of the disclosure comprises one or a plurality of sugar surrogates at one or a plurality of nucleotide positions.

The terms “therapeutically effective amount” mean a quantity sufficient to achieve a desired therapeutic and/or prophylactic effect, for example, an amount which results in the prevention or amelioration of or a decrease in the symptoms associated with a disease that is being treated. The amount of composition administered to the subject will depend on the type and severity of the disease and on the characteristics of the individual, such as general health, age, sex, body weight and tolerance to drugs. It will also depend on the degree, severity and type of disease. The skilled artisan will be able to determine appropriate dosages depending on these and other factors. The regimen of administration can affect what constitutes an effective amount. The compound of the disclosure can be administered to the subject either prior to or after the onset of disease or disorder. Further, several divided dosages, as well as staggered dosages, can be administered daily or sequentially, or the dose can be continuously infused, or can be a bolus injection. Further, the dosages of the compound(s) of the disclosure can be proportionally increased or decreased as indicated by the exigencies of the therapeutic or prophylactic situation. Typically, an effective amount of the compounds of the present disclosure, sufficient for achieving a therapeutic or prophylactic effect, range from about 0.000001 mg per kilogram body weight per day to about 10,000 mg per kilogram body weight per day. Preferably, the dosage ranges are from about 0.0001 mg per kilogram body weight per day to about 100 mg per kilogram body weight per day. A therapeutically effective amount of a pharmaceutical composition comprising any one or a plurality of any of the nucleic acid sequences disclosed herein can also be administered in combination with two, three, four or more nucleic acid sequences disclosed herein, or with one or more additional therapeutic compounds. Those skilled in the art will recognize and determine a therapeutically effective amount of any of the nucleic acid sequences disclosed herein whether calculated when administered alone or part of a therapeutic regimen that includes one or more other beta-catenin nuclear translocation inhibitors and/or one or more one or more other therapeutic agents and/or one or more other therapeutic treatments or interventions. Generally, therapeutically effective amount refers to an amount of a nucleic acid sequence that alone or in combination with one or a plurality of other therapeutic compounds causes a transfection of the nucleic acid sequence into a target cell (such as a cancer cell) and/or hybridization of the one or more miRNA domains within the nucleic acid sequences sufficient reduce or inhibit expression of a mRNA sequence with the cell, thereby ameliorating symptoms, or reversing, preventing or reducing the rate of progress of disease, or extend life span of a subject when administered alone or in combination with other therapeutic agents or treatments as compared to the symptoms, rate of progress of disease, or life span of an individual not receiving a therapeutically effective amount the one or plurality of nucleic cells disclosed herein.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include di- and multivalent radicals, having the number of carbon atoms designated (i.e., C₁-C₁₀ means one to ten carbons). Alkyl is not cyclized. Examples of saturated hydrocarbon radicals include, but are not limited to, groups such as methyl, ethyl, n-propyl, isopropyl, n-butyl, t-butyl, isobutyl, sec-butyl, (cyclohexyl)methyl, homologs and isomers of, for example, n-pentyl, n-hexyl, n-heptyl, n-octyl, and the like. An unsaturated alkyl group is one having one or more double bonds or triple bonds (e.g. alkene, alkyne). Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—).

The term “alkylene,” by itself or as part of another substituent, means, unless otherwise stated, a divalent radical derived from an alkyl, as exemplified, but not limited by, —CH₂CH₂—. Typically, an alkyl (or alkylene) group will have from about 1 to about 24 carbon atoms, with those groups having 10 or fewer carbon atoms being preferred in the present disclosure. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene.

The term “heteroalkyl,” by itself or in combination with another term, means, unless otherwise stated, a stable straight or branched chain, or combinations thereof, including at least one carbon atom and at least one heteroatom selected from the group consisting of O, N, P, Si, and S, and wherein the nitrogen and sulfur atoms may optionally be oxidized, and the nitrogen heteroatom may optionally be quaternized. Heteroalkyl is not cyclized. The heteroatom(s) O, N, P, S, and Si may be placed at any interior position of the heteroalkyl group or at the position at which the alkyl group is attached to the remainder of the molecule. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, mean, unless otherwise stated, cyclic versions of “alkyl” and “heteroalkyl,” respectively. Additionally, for heterocycloalkyl, a heteroatom can occupy the position at which the heterocycle is attached to the remainder of the molecule. Cycloalkyl and heterocycloalkyl are non-aromatic. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl. 4-thiazolyl, 5-thiazolyl, 2-furyl, 3-furyl, 2-thienyl, 3-thienyl, 2-pyridyl, 3-pyridyl. 4-pyridyl. 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. Non-limiting examples of heteroaryl groups include pyridinyl, pyrimidinyl, thiophenyl, thienyl, furanyl, indolyl, benzoxadiazolyl, benzodioxolyl, benzodioxanyl, thianaphthanyl, pyrrolopyridinyl, indazolyl, quinolinyl, quinoxalinyl, pyridopyrazinyl, quinazolinonyl, benzoisoxazolyl, imidazopyridinyl, benzofuranyl, benzothienyl, benzothiophenyl, phenyl, naphthyl, biphenyl, pyrrolyl, pyrazolyl, imidazolyl, pyrazinyl, oxazolyl, isoxazolyl, thiazolyl, furylthienyl, pyridyl, pyrimidyl, benzothiazolyl, purinyl, benzimidazolyl, isoquinolyl, thiadiazolyl, oxadiazolyl, pyrrolyl, diazolyl, triazolyl, tetrazolyl, benzothiadiazolyl, isothiazolyl, pyrazolopyrimidinyl, pyrrolopyrimidinyl, benzotriazolyl, benzoxazolyl, or quinolyl. The examples above may be substituted or unsubstituted and divalent radicals of each heteroaryl example above are non-limiting examples of heteroarylene.

A fused ring heterocyloalkyl-aryl is an aryl fused to a heterocycloalkyl. A fused ring heterocycloalkyl-heteroaryl is a heteroaryl fused to a heterocycloalkyl. A fused ring heterocycloalkyl-cycloalkyl is a heterocycloalkyl fused to a cycloalkyl. A fused ring heterocycloalkyl-heterocycloalkyl is a heterocycloalkyl fused to another heterocycloalkyl. Fused ring heterocycloalkyl-aryl, fused ring heterocycloalkyl-heteroaryl, fused ring, heterocycloalkyl-cycloalkyl, or fused ring heterocycloalkyl-heterocycloalkyl may each independently be unsubstituted or substituted with one or more of the substitutents described herein.

The term “oxo,” as used herein, means an oxygen that is double bonded to a carbon atom.

The term “alkylsulfonyl,” as used herein, means a moiety having the formula —S(O₂)—R′, where R′ is a substituted or unsubstituted alkyl group as defined above. R′ may have a specified number of carbons (e.g., “C₁-C₄ alkylsulfonyl”).

In some embodiments, each of the above terms (e.g., “alkyl,” “heteroalkyl,” “aryl,” and “heteroaryl”) includes both substituted and unsubstituted forms of an indicated radical.

Embodiments of the disclosure include radicals of any of those nucleotides within a given sequence. Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR′″, —ONR′R″, —NR′C═(O)NR″NR′″R″″, —CN, —NO₂, monophosphate (or derivatives thereof), diphosphate (or derivatives thereof), triphosphate (or derivatives thereof), in a number ranging from zero to (2m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound of the disclosure includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′. —NR—C(NR′R″R′″)═NR″″, —NR—C(NR′R″)═NR′″, —S(O)R′, —S(O)₂R′, —S(O)₂NR′R″. —NRSO₂R′, —NR′NR″R′″, —ONR′R″, —NR′C═(O)NR″NR′″R″″, —CN, —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound of the disclosure includes more than one R group, for example, each of the R groups is independently selected as are each R′, R″, R′″, and R″″ groups when more than one of these groups is present.

Two or more substituents in a modified nucleic acid sequence disclosed herein may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In some embodiments, the ring-forming substituents are attached to a single member of the base structure. For example, two ring-forming substituents attached to a single member of a cyclic base structure create a spirocyclic structure. In yet another set of embodiments, the ring-forming substituents are attached to non-adjacent members of the base structure.

Two of the substituents on adjacent atoms of the aryl or heteroaryl ring may optionally form a ring of the formula -T-C(O)—(CRR′)q-U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)r-B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)s-X′— (C″R″R′″)d-, where s and d are independently integers of from 0 to 3, and X′ is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include, oxygen (O), nitrogen (N), sulfur (S), phosphorus (P), and silicon (Si).

A “substituent group,” as used herein, means a group selected from the following moieties: (A) oxo, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, —NHSO₂CH₃, —N₃, unsubstituted alkyl, unsubstituted heteroalkyl, unsubstituted cycloalkyl, unsubstituted heterocycloalkyl, unsubstituted aryl, unsubstituted heteroaryl, monophosphate (or derivatives thereof), diphosphate (or derivatives thereof), or triphosphate (or derivatives thereof), and (B) alkyl, heteroalkyl, cycloalkyl, heterocycloalkyl, aryl, heteroaryl, substituted with at least one substituent selected from:

(i) oxo, halogen, —CF₃, —CN, —OH, —NH₂, —COOH, —CONH₂, —NO₂, —S, —SO₂Cl, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, —NHSO₂CH₃, —N₃, unsubstituted alkyl, unsubstituted heteroalkyl, unsubstituted cycloalkyl, unsubstituted heterocycloalkyl, unsubstituted aryl, unsubstituted heteroaryl, and

(ii) alkyl, heteroalkyl, cycloalkyl, heterocycloalkyl, aryl, heteroaryl, substituted with at least one substituent selected from:
(a) oxo, halogen, —CF₃. —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NH—OH, —OCF₃, —OCHF₂, —NHSO₂CH₃, —N₃, unsubstituted alkyl, unsubstituted heteroalkyl, unsubstituted cycloalkyl, unsubstituted heterocycloalkyl, unsubstituted aryl, unsubstituted heteroaryl, and
(b) alkyl, heteroalkyl, cycloalkyl, heterocycloalkyl, aryl, heteroaryl, monophosphate (or derivatives thereof), diphosphate (or derivatives thereof), or triphosphate (or derivatives thereof), substituted with at least one substituent selected from: oxo, halogen, —CF, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, —NHSO₂CH₃, —N₃, unsubstituted alkyl, unsubstituted heteroalkyl, unsubstituted cycloalkyl, unsubstituted heterocycloalkyl, unsubstituted aryl, unsubstituted heteroaryl,

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl, is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and each substituted or unsubstituted heteroaryl is a substituted or unsubstituted 5 to 9 membered heteroaryl.

In embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds, compositions and pharmaceutical compositions disclosed herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl; each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In some embodiments herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene. In embodiments, the compound is a chemical species set forth in the Examples section below.

Embodiments of the present disclosure may possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present disclosure. The compounds of the present disclosure do not include, or free of, those compound which are known in art to be too unstable to synthesize and/or isolate. The present disclosure is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another. In some embodiments, the composition of the disclosure comprises one or a plurality of tautomers of given forms. It will be apparent to one skilled in the art that, in some embodiments, the compositions of this disclosure comprise nucleic acid sequences or molecules with nucleic acids that may exist in tautomeric forms, all such tautomeric forms of the compounds being within the scope of the disclosure.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by 13C- or 14C-enriched carbon are within the scope of this disclosure.

The compounds of the present disclosure may also contain unnatural proportions of atomic isotopes at one or more of the atoms that constitute such compounds. For example, the compounds may be radiolabeled with radioactive isotopes, such as for example tritium (3H), iodine-125 (125I), or carbon-14 (14C) including the radioisotopes of Table 2. All isotopic variations of the compounds of the present disclosure, whether radioactive or not, are encompassed within the scope of the present disclosure.

The symbol “-” denotes the point of attachment of a chemical moiety to the remainder of a molecule or chemical formula.

The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls. Moreover, where a moiety is substituted with an R substituent, the group may be referred to as “R-substituted.” Where a moiety is R-substituted, the moiety is substituted with at least one R substituent and each R substituent is optionally different.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The symbol “ [symbol] ” denotes the point of attachment of a chemical moiety to the remainder of a molecule or chemical formula. The symbol “ [symbol] ” denotes one or more than one modified or unmodified contiguous nucleotide.

A “base,” as used herein, means a group selected from the following: adenine, guanine, cytosine, uracil, thymine, uridine, pyrimidine, purine, pseudouridine, inosine, hypoxanthine, rhodamine, fluroscein, 2-aminopurine, cytidine, 2′-deoxycytidine, 1,3-Diaza-2-oxophenothiazine, dihydrouridine, queuosine, wyosine, cyanophage S-2L diaminopurine, isoguanine, isocytosine, diaminopyrimidine, 2,4-difluorotoluene. 4-methylbenzimidazole, isoquinoline, pyrrolo[2,3-b]pyridine, 2-amino-6-(2-thienyl)purine, pyrrole-2-carbaldehyde, 2,6-bis(ethylthiomethyl)pyridine, pyridine-2,6-dicarboxamide, 2′-deoxyinosine, 2-amino-8-(2-thienyl)purine, pyridine-2-one, 7-(2-thienyl)imidazo[4,5-b]pyridine, pyrrole-2-carbaldehyde, 4-[3-(6-aminohexanamido)-1-propynyl]-2-nitropyrrole, or modified derivative thereof.

The term “phosphodiester,” by itself or as part of another substituent, means, unless otherwise stated, —O—P(O)₂—O—, wherein the phosphate atom is doubly bonded to one oxygen atom and bound to other substituents through the adjacent oxygen atoms.

The term “LNA,” as used herein, means any nucleic acid analog disclosed herein comprising a cyclic structure between the C2 and C4 carbon of the sugar moiety of a nucleic acid. In some embodiments, the LNA has the structure below:

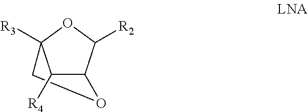

wherein R₂ is independently selected from: any base or nucleobase, adenine, guanine, cytosine, uracil, thymine, uridine, pyrimidine, purine, pseudouridine, inosine, or hypoxanthine; wherein R₃ is independently selected from a: phosphodiester, phosphorothioate, aldehyde, carboxyl, carbonyl, ether, ester, or amino; wherein R₄ is independently selected from a: phosphodiester, phosphorothioate, aldehyde, carboxyl, carbonyl, ether, ester, or amino; or a pharmaceutically active salt thereof.

The terms “subject,” “individual,” and “patient” are used interchangeably herein to refer to a vertebrate, preferably a mammal, more preferably a human. Mammals include, but are not limited to, murines, simians, humans, farm animals, cows, pigs, goats, sheep, horses, dogs, sport animals, and pets. Tissues, cells and their progeny obtained in vivo or cultured in vitro are also encompassed by the definition of the term “subject.” The term “subject” is also used throughout the specification in some embodiments to describe an animal from which a cell sample is taken or an animal to which a disclosed cell or nucleic acid sequences have been administered. In some embodiment, the animal is a human. For treatment of those conditions which are specific for a specific subject, such as a human being, the term “patient” may be interchangeably used. In some instances in the description of the present disclosure, the term “patient” will refer to human patients suffering from a particular disease or disorder. In some embodiments, the subject may be a non-human animal from which an endothelial cell sample is isolated or provided. The term “mammal” encompasses both humans and non-humans and includes but is not limited to humans, non-human primates, canines, felines, murines, bovines, equines, caprines, and porcines.

“Variants” is intended to mean substantially similar sequences. For nucleic acid molecules, a variant comprises a nucleic acid molecule having deletions (i.e., truncations) at the 5′ and/or 3′ end; deletion and/or addition of one or more nucleotides at one or more internal sites in the native polynucleotide; and/or substitution of one or more nucleotides at one or more sites in the native polynucleotide. As used herein, a “native” nucleic acid molecule or polypeptide comprises a naturally occurring nucleotide sequence or amino acid sequence, respectively. For nucleic acid molecules, conservative variants include those sequences that, because of the degeneracy of the genetic code, encode the amino acid sequence of one of the polypeptides of the disclosure. Variant nucleic acid molecules also include synthetically derived nucleic acid molecules, such as those generated, for example, by using site-directed mutagenesis but which still encode a protein of the disclosure. Generally, variants of a particular nucleic acid molecule of the disclosure will have at least about 70%, 75%, 80%, 85%. 90%, 91%, 92%, 93%, 94%, 95%, 96%, 97%, 98%, 99% or more sequence identity to that particular polynucleotide as determined by sequence alignment programs and parameters as described elsewhere herein.

Variants of a particular nucleic acid molecule of the disclosure (i.e., the reference DNA sequence) can also be evaluated by comparison of the percent sequence identity between the polypeptide encoded by a variant nucleic acid molecule and the polypeptide encoded by the reference nucleic acid molecule. Percent sequence identity between any two polypeptides can be calculated using sequence alignment programs and parameters described elsewhere herein. Where any given pair of nucleic acid molecule of the disclosure is evaluated by comparison of the percent sequence identity shared by the two polypeptides that they encode, the percent sequence identity between the two encoded polypeptides is at least about 70%, 75%, 80%, 85%, 90%, 91%, 92%, 93%, 94%, 95%, 96%, 97%, 98%, 99% or more sequence identity. In some embodiments, the term “variant” protein is intended to mean a protein derived from the native protein by deletion (so-called truncation) of one or more amino acids at the N-terminal and/or C-terminal end of the native protein; deletion and/or addition of one or more amino acids at one or more internal sites in the native protein; or substitution of one or more amino acids at one or more sites in the native protein. Variant proteins or polynucleotides encompassed by the present disclosure are biologically active, that is they continue to possess the desired biological activity of the native or claimed protein or polynucleotide as described herein. Such variants may result from, for example, genetic polymorphism or from human manipulation. Biologically active variants of a protein of the disclosure will have at least about 70%, 75%, 80%, 85%, 90%, 91%, 92%, 93%, 94%, 95%, 96%, 97%, 98%, 99% or more sequence identity to the amino acid sequence for the native protein as determined by sequence alignment programs and parameters described elsewhere herein. A biologically active variant of a protein of the disclosure may differ from that protein by as few as 1-15 amino acid residues, as few as 1-10, such as 6-10, as few as 5, as few as 4, 3, 2, or even 1 amino acid residue. The proteins or polypeptides of the disclosure may be altered in various ways including amino acid substitutions, deletions, truncations, and insertions. Methods for such manipulations are generally known in the art. For example, amino acid sequence variants and fragments of the proteins can be prepared by mutations in the nucleic acid sequence that encodes the amino acid sequence recombinantly.

In some embodiments, any natural or non-natural nucleic acid formula may be repeated across 1, 2, 3, 4, 5, 6, 7, 8, 9, 10 or more nucleic acids in contiguous nucleic acids or in a non-contiguous nucleotides across the length of the nucleic acid. In some embodiments, the disclosed nucleic acid sequences comprise 1, 2, 3, 4, 5, 6, 7, 8, 9, 10, 11, 12, 13, 14, 15, 16, 17, 18, 19, 20 or more contiguous or non-contiguous modified nucleic acids across a length of the nucleic acid.

In some embodiments, the composition or pharmaceutical composition disclosed herein comprises a nucleic acid disclosed herein that comprises ribonucleic acid and about 10%, 11%, 12%, 13%, 14%, 15%, 16%, 17%. 18%, 19%, 20%, 22%, 25%, 27%, 30%, 32%, 35%, 37%, 40%, 42%, 45%, 47%, 50%, 52%, 55%, 57%. 60%, 62%, or 65% modified nucleotides.

In some embodiments, any of the forgoing formulae may comprise one or a plurality of LNA molecules positioned between or bound to one or a plurality of modified or unmodified nucleotides.

In some embodiments, the composition or pharmaceutical composition disclosed herein comprises a nucleic acid sequence comprising a total of about 20, 25, 30, 35, 40, 45, 50, 55, 60, 65, 70, 75, 80, 85, 90, 95, 100, 150, or 200 nucleotides in length and comprising in 5′ to 3′ orientation: aptamer domain and a miRNA domain. In some embodiments the composition or pharmaceutical composition disclosed herein comprises a nucleic acid sequence comprising a total of about 20, 25, 30, 35, 40, 45, 50, 55, 60, 65, 70, 75, 80, 85, 90, 95, 100, 150, or 200 nucleotides in length and comprising in 5′ to 3′ orientation: an aptamer domain and a miRNA domain; the nucleic acid sequence further comprises a CRISPR element or complex comprising one, two or three of the following domains: a Cas protein binding domain (or Cas binding domain), and/or a transcription terminator domain and/or a DNA-binding domain; wherein each of the aforementioned domains independently consists of no more than about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 110, 120, 130, 140, 150, 160, 170, 180, or 190 nucleotides.

In some embodiments, the composition or pharmaceutical composition disclosed herein comprises a nucleic acid comprises a total of about 50, 60, 70, 80, 90, 100, 150, or 200 nucleotides in length and comprise in 5′ to 3′ orientation: a DNA-binding domain, a Cas protein binding domain, and, optionally, a transcription terminator domain; wherein each of the aforementioned domains independently consists of no more than about 20, 30, 40, 50, 60, 70, 80, 90, 100, 110, 120, 130, 140, 150, 160, 170, 180, or 190 nucleotides.

In some embodiments, the nucleic acid molecule comprises a Cas-protein binding domain. In certain embodiments, the Cas-protein binding domain comprises about 30, 35, 40, 41, 42, 43, 44, 45, 46, 47, 48, 49, 50, 51, 52, 53, 54 or 55 nucleotides. Any of these values may be used to define a range for the length of the Cas-protein binding domain. For example, in some embodiments, the Cas-protein binding domain comprises about 30 to 55, about 40 to 45, or about 40 to 50 nucleotides. In a particular embodiment, the Cas-protein binding domain comprises about 41 nucleotides.

In certain embodiments, the modification of the nucleotide in the aptamer domain is one or more of 2′-O-methyl, 2′-O-fluoro, or phosphorothioate. In certain embodiments, the nucleotide is modified at the 2′ position of the sugar moiety. In certain embodiments, the modification at the 2′ position of the sugar moiety is 2′-O-methyl or 2′-O-fluoro. In certain embodiments, the nucleotide is modified at the 3′ position of the sugar moiety. In certain embodiments, the modification at the 3′ position of the sugar moiety is phosphorothioate. In certain embodiments, the nucleotide is modified at both the 2′ position of the sugar moiety and at the 3′ position of the sugar moiety. In certain embodiments, the nucleotide is not modified at the 2′ position of the sugar moiety. In certain embodiments, the nucleotide is not modified at the 3′ position of the sugar moiety.

In a particular embodiment, the nucleic acid molecule comprises a miRNA domain comprising from about 17 to 45 nucleotides, wherein the miRNA domain has at least 70% sequence homology to the nucleic acid sequence of SEQ ID NO: 1, and wherein one or more of the nucleotides are modified.

In certain embodiments of the aforementioned nucleic acid molecules, only the aptamer domain comprises one or more modified nucleotides. In certain embodiments, only the miRNA binding domain of the nucleic acid molecule comprises one or more modified nucleotides. In certain embodiments, both the aptamer domain and the mi-RNA domain comprise one or more modified nucleotides.

In certain aspects, the invention also relates to a pharmaceutical composition comprising any of the aforementioned nucleic acid molecules. In certain embodiments, the pharmaceutical composition comprises a nanoparticle comprising any of the aforementioned nucleic acid molecules.

In some embodiments, the nucleic acid sequence comprises one or a plurality of intervening sequences, or linkers, between any one or plurality of domains. In some embodiments, the intervening sequence is no more than about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 60, 61, 62, 63, 63, 64, 65, 66, 67, 68, 69, 70, 71, 72, 73, 74, 75, 76, 77, 78, 79, 80, 81, 82, 83, 84, 85, 86, 87, 88, 89, 90, 91 92, 93, 94, 95, 96, 97, 98, 99, or 100 nucleotides in length.

The aptamer domain can be from about 5 to about 150 nucleotides long, or longer (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 60, 61, 62, 63, 63, 64, 65, 66, 67, 68, 69, 70, 71, 72, 73, 74, 75, 76, 77, 78, 79, 80, 81, 82, 83, 84, 85, 86, 87, 88, 89, 90, 91 92, 93, 94, 95, 96, 97, 98, 99, or 100 nucleotides in length, or longer). In some cases, the aptamer region is from about 15 to about 50 nucleotides in length (e.g., from about 15 to about 34, 15-46, 15-40; 16-35, 16-30, 16-28, 16-25; or about 25-50, 25-55, 25-60, or about 15 to about 65 nucleotides in length).

The miRNA domain can be from about 5 to about 150 nucleotides long, or longer (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 60, 61, 62, 63, 63, 64, 65, 66, 67, 68, 69, 70, 71, 72, 73, 74, 75, 76, 77, 78, 79, 80, 81, 82, 83, 84, 85, 86, 87, 88, 89, 90, 91 92, 93, 94, 95, 96, 97, 98, 99, or about 100 nucleotides in length, or longer). In some cases, the miRNA region is from about 15 to about 50 nucleotides in length (e.g., from about 15 to about 34, 15-46, 15-40; 16-35, 16-30, 16-28, 16-25; or about 25-50, 25-55, 25-60, or 15-65 nucleotides in length). In some embodiments, the nucleic acid molecule provided is free of a miRNA domain.

Generally, the miRNA region is designed to complement or substantially complement the target nucleic acid sequence or sequences, such as an mRNA sequence in a target cell. In some embodiments, the mRNA domain is also called a “nucleotide binding region,” and such terms are used equivalently in this application, because of its ability to bind to complementary or partially complementary target sequences.

The nucleotide binding domain can incorporate wobble or degenerate bases to bind multiple sequences. In some cases, the binding region can be altered to increase stability. For example, non-natural nucleotides, can be incorporated to increase RNA resistance to degradation. In some cases, the binding region can be altered or designed to avoid or reduce secondary structure formation in the binding region. In some cases, the binding region can be designed to optimize G-C content. In some cases, (i-C content is from about 40% and about 60% (e.g., 40%, 45%, 50%, 55%, 60%). In some cases, the nucleotide binding region can contain modified nucleotides such as, without limitation, methylated, phosphorylated, fluorinated, or hydroxylated nucleotides. In some cases, the nucleotide binding region can contain modified nucleotides such as, without limitation, methylated, phosphorylated, fluorinated, or hydroxylated nucleotides; wherein if the nucleotide is fluorinated, the nucleotide may also be bound to one or more adjacent modified or unmodified nucleotides by a phosphorothioate bond, in either R or S orientation.

In some embodiments, the nucleotide binding region binds or is capable of hybridizing with DNA, RNA, or hybrid RNA/DNA sequences, such as any of those target sequences described herein. In some embodiments, any of the domains or elements comprises DNA, RNA, or hybrid RNA/DNA sequences. In some embodiments, the miRNA domain comprises from about 5% to about 100% modified nucleotides based upon the total number of the nucleotides in the entire sequence. In some embodiments, the miRNA domain comprises from about 5% to about 90% modified nucleotides as compared to an unmodified or naturally occurring nucleotide sequence. In some embodiments, the miRNA domain comprises from about 5% to about 80% modified nucleotides. In some embodiments, the miRNA domain comprises from about 5% to about 70% modified nucleotides. In some embodiments, the miRNA domain comprises from about 5% to about 60% modified nucleotides. In some embodiments, the miRNA domain comprises from about 5% to about 50% modified nucleotides. In some embodiments, the miRNA domain comprises from about 5% to about 40% modified nucleotides. In some embodiments, the miRNA domain comprises from about 5% to about 30% modified nucleotides. In some embodiments, the miRNA domain comprises from about 5% to about 20% modified nucleotides. In some embodiments, the miRNA domain comprises from about 5% to about 10% modified nucleotides. In some embodiments, the miRNA domain comprises from about 5% to about 9% modified nucleotides.

In some embodiments, the miRNA domain comprises hybrid RNA/DNA sequences of either unmodified or modified nucleotides. In some embodiments, the DNA-targeting domain comprises no less than about 250, 200, 150, 100, 50, 45, 40, 35, 30, 25, or 20 nucleotides, wherein no more than about 50, 45, 40, 35, 30, 25, 20, 15, 14, 13, 12, 11, 10, 9, 8, 7, 6, 5, 4, 3, 2, or 1 nucleotides is a modified or unmodified deoxyribonucleic acid. In some embodiments, the miRNA domain comprises no less than about 250, 200, 150, 100, 50, 45, 40, 35, 30, 25, or 20 nucleotides, wherein no more than about 50, 45, 40, 35, 30, 25, 20, 15, 14, 13, 12, 11, 10, 9, 8, 7, 6, 5, 4, 3, 2, or 1 nucleotides from the 5′ end of the nucleic acid sequence is a modified or unmodified deoxyribonucleic acid.

Variants of a particular nucleic acid molecule of the disclosure (i.e., the reference DNA sequence) can also be evaluated by comparison of the percent sequence identity between the nucleic acid and the variant nucleic acid molecule and the polypeptide encoded by the reference nucleic acid molecule. In some embodiments the nucleic acid sequence or molecules disclosed herein encompass variants. Percent sequence identity between any two nucleic acid molecules can be calculated using sequence alignment programs and parameters described elsewhere herein. Where any given pair of nucleic acid molecule of the disclosure is evaluated by comparison of the percent sequence identity shared by the two nucleotides such that they encode, the percent sequence identity between the two encoded nucleic acid sequence is at least about 70%, 75%, 80%, 85%, 90%, 91%, 92%, 93%, 94%, 95%, 96%, 97%, 98%, 99% or more sequence identity. In some embodiments, the term “variant” nucleotide sequence is intended to mean a nucleotide sequence derived from the native or disclosed nucleotide by deletion (so-called truncation) of one or more nucleic acid sequences at the 5′ prime and 3′ prime-terminal and/or terminal end of the native or disclosed nucleotide sequence; deletion and/or addition of one or more amino acids at one or more internal sites in the native or disclosed nucleotide sequence; or substitution of one or more bases or modifications at one or more sites in the native or disclosed nucleotide sequence. Variant nucleotide sequences encompassed by the present disclosure are biologically active, that is they continue to possess the desired biological activity of the disclosed nucleotide acid sequence as described herein. Such variants may result from, for example, genetic polymorphism or from human manipulation. Biologically active variants of a nucleic acid sequences of the disclosure will have at least about 70%, 75%, 80%, 85%, 90%, 91%, 92%, 93%, 94%, 95%, 96%, 97%. 98%, 99% or more sequence identity to the nucleic acid sequence for the disclosed or native protein as determined by sequence alignment programs and parameters disclosed herein. A biologically active variant of a nucleotide sequence of the disclosure may differ from the disclosed nucleotide sequence by as few as about 1, 2, 3, 4, 5, 6. 7, 8, 9, 10, 11, 12, 13, 14 or about 15 nucleobases, as few as about 1 to about 10, such as 6-10, as few as 5, as few as 4, 3, 2, or even 1 nucleobase. The nucleotide sequences of the disclosure may be altered in various ways including base substitutions, deletions, truncations, and insertions. Methods for such manipulations are generally known in the art. For example, nucleotide sequence variants and fragments of the proteins can be prepared by standard PCR-induced mutations in the nucleic acid sequence by the designing primers with the mutations to be added or deleted.

“Internucleotide linkage” refers to any group, molecules or atoms that covalently or noncovalently join two nucleosides. Unmodified internucleotide linkages are phosphodiester bonds. In some embodiments, the nucleic acid sequence comprises at least about 10%, 20%, 30%, 40%, 50%, 60%, 70%, 75%, 80%, 85%, 90%, 91%, 92%, 93%, 94%, 95%, 96%, 97%, 98%, 99% or more modified internucleotide linkages. Modified internucleotide linkages are set forth in the U.S. Pat. No. 8,133,669 and WO1994002499, each of which is incorporated herein in its entirety. Examples of such well known modified linkages, for which conventional synthesis schemes are known, include alkylphosphonate, phosphodiester, phosphotriester, phosphorothioate, phosphorodithioate, phosphoramidate, ketone, sulfone, carbonate and thioamidate linkages. In some embodiments, the composition or pharmaceutical compositions disclosed herein comprise a nucleotide acid sequence disclosed herein with one or more internucleotide linkages that are modified or mutated at any one or plurality of positions within the sequence.

“2′-0-methyl sugar” or “2′-OMe sugar” means a sugar having a 0-methyl modification at the 2′ position.

“2′-0-methoxyethyl sugar” or “2′-MOE sugar” means a sugar having a 0-methoxy ethyl modification at the 2′ position.

“2′-0-fluoro” or “2′-F” means a sugar having a fluoro modification of the 2′ position.

### Compositions

The disclosure relates to a nucleic acid molecule or nucleic acid molecules comprising a nucleic acid sequence of two, three, four, five or more domains, each domain comprising or consisting of from about 10 to about 110 nucleic acids; wherein the first domain is an aptamer domain and the second domain is a miRNA domain and the first and second domains appear in the 5′ to 3′ orientation and optionally, the composition comprising from about 1% to about 100% modified nucleic acids. In some embodiments, the composition comprises the nucleic acid sequence with a third, fourth or fifth domain each of the third, fourth, and fifth nucleic acids are elements in a CRISPR/sgRNA system. In some embodiments, the domains are contiguous or non-contiguous with from about 1 to about 100 or more nucleotides in between one or more domains.

In some embodiments, the disclosure relates to a nucleic acid sequence and compositions comprising the same. In another aspect, the disclosure relates to a nucleic acid sequence disclosed herein and compositions comprising the same with or without a vector capable of delivery of the nucleic acid. In some embodiments, the vector is a viral vector or a bacterial vector wherein such vector is attenuated and/or replication deficient such that administration of the vector comprising or encapsulating the disclosed nucleic acid sequence is capable of delivering its payload into a transduced cell but otherwise unable to divide and ro replicate sufficiently to cause an infection due to the absence viral nucleic acid or attenuation of the vector particle.

In some embodiments, the present disclosure provides a composition comprising a compound having Formula W:

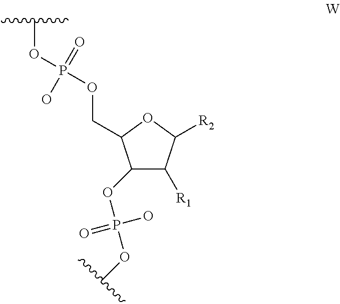

wherein R₁ is independently selected from a halogen, methyl, or methoxy ethyl;

wherein R₂ is independently selected from: hydrogen, hydroxyl, halogen, alkyl, alkenyl, alkynyl, acyl, a base, adenine, guanine, cytosine, uracil, thymine, uridine, pyrimidine, purine, pseudouridine, inosine, or hypoxanthine;

or a pharmaceutically active salt thereof.

In some embodiments, the present disclosure provides a composition comprising a compound having Formula X:

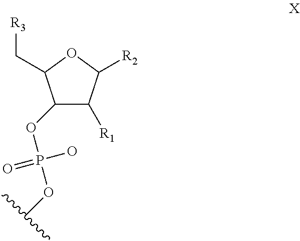

wherein R₁ is independently selected from a halogen, methyl, or methoxy ethyl;

wherein R₂ is independently selected from: any nucleobase, hydrogen, hydroxyl, halogen, alkyl, alkenyl, alkynyl, acyl, adenine, guanine, cytosine, uracil, thymine, uridine, a pyrimidine, a purine, pseudouridine, inosine, or hypoxanthine;

wherein R₃ is independently selected from a: alkylphosphonate, phosphotriester, phosphorodithioate, phosphoramidate, ketone, sulfone, carbonate thioamidate, phosphodiester, aldehyde, carboxyl, carbonyl, ether, ester, or amine; in some embodiments, the phosphodiester, alkylphosphonate, phosphotriester, phosphorodithioate, phosphoramidate, ketone, sulfone, carbonate thioamidate, aldehyde, carboxyl, carbonyl, ether, ester, or amine is bonded to a contiguous nucleic acid or nucleoside, such that the R₃ reads

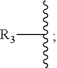

or a pharmaceutically active salt thereof.

In some embodiments, the present disclosure provides a composition comprising a compound having Formula Y:

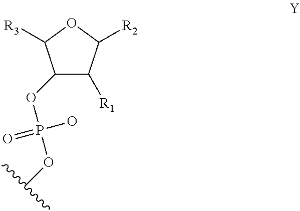

wherein R₁ is independently selected from: hydrogen, hydroxyl, halogen, methyl, or methoxy ethyl;

wherein R₂ is independently selected from: hydrogen, hydroxyl, halogen, alkyl, alkenyl, alkynyl, acyl, any base, adenine, guanine, cytosine, uracil, thymine, uridine, a pyrimidine, a purine, pseudouridine, inosine, or hypoxanthine;

wherein R₃ is independently selected from a: alkylphosphonate, phosphotriester, phosphorodithioate, phosphoramidate, ketone, sulfone, carbonate thioamidate, phosphorothioate, phosphodiester, aldehyde, carboxyl, carbonyl, ether, ester, amine or a CH2-bonded to a phosphodiester, aldehyde, carboxyl, carbonyl, ether, ester, amine;

wherein, in some optional, embodiments, the alkylphosphonate, phosphotriester, phosphorodithioate, phosphoramidate, ketone, sulfone, carbonate thioamidate, phosphodiester, aldehyde, carboxyl, carbonyl, ether, ester, or amine is bonded to a contiguous nucleic acid, such that the R₃ reads

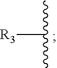

or a pharmaceutically active salt thereof.

In some embodiments, the present disclosure provides a composition comprising a compound having Formula Z:

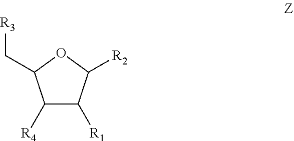

wherein R₁ is independently selected from: a hydrogen, a hydroxyl, a halogen, methyl, or methoxy ethyl;

wherein R₂ is independently selected from: hydrogen, hydroxyl, halogen, alkyl or heteroalkyl, alkenyl, alkynyl, acyl, any base, pyrimidine, purine, adenine, guanine, cytosine, uracil, thymine, uridine, pseudouridine, inosine, or hypoxanthine;

wherein R₃ is independently selected from a: alkylphosphonate, phosphotriester, phosphorodithioate, phosphoramidate, ketone, sulfone, carbonate thioamidate, phosphorothioate, phosphodiester, aldehyde, carboxyl, carbonyl, ether, ester, or amine;

wherein R₄ is independently selected from a one or a combination of: alkylphosphonate, phosphotriester, phosphorodithioate, phosphoramidate, ketone, sulfone, carbonate thioamidate, phosphorothioate, phosphodiester, aldehyde, carboxyl, carbonyl, ether, ester, or amine;

or a pharmaceutically active salt thereof; wherein the compound X is positioned between or bonded to any one or plurality of unmodified or modified nucleotides at R₃ and/or R₄.

The nucleic acid sequence may comprise zero, one or a plurality of nucleotides of any combination or sequence having any of Formulae W, X, Y and Z. As a non-limiting example, compositions of the disclosure can comprise a nucleic acid sequence of N′-[Z]ₙ-N″; wherein N′ is any modified or unmodified 5′ terminal nucleotide; N″ is any modified or unmodified 3′ terminal nucleotide; any n is any positive integer from about 1 to about 250, wherein each position of Z in the formula may have an independently selected positions at their respective R₁, R₂, R₃, and R₄, subgroups. As a non-limiting example, compositions of the disclosure relate to a nucleic acid sequence of N′-[Z]₁₀-N″; wherein N′ is any modified or unmodified 5′ terminal nucleotide; N″ is any modified or unmodified 3′ terminal nucleotide; wherein [Z]₁₀ is [Z₁-Z₂-Z₃-Z₄-Z₅-Z₆-Z₇-Z₈-Z₉-Z₁₀] and each position of Z in the formula may have an independently selected positions at their respective R₁, R₂, R₃, and R₄, subgroups. As a another non-limiting example, compositions of the disclosure may comprise a nucleic acid sequence of N′-[Z]ₙ-N″; wherein N′ is any modified or unmodified 5′ terminal nucleotide; N″ is any modified or unmodified 3′ terminal nucleotide; any n is any positive integer from about 1 to about 100, wherein each position of Z in the sequence may have an independently selected positions at their respective R₁, R₂, R₃, and R₄, subgroups; As a another non-limiting example, compositions of the disclosure may comprise a nucleic acid sequence of N′-[X, W, Y, or Z]ₙ-N″; wherein N′ is any modified or unmodified 5′ terminal nucleotide; N″ is any modified or unmodified 3′ terminal nucleotide; any n is any positive integer from about 1 to about 100, wherein each position of the nucleic acid sequence comprises zero, one or a plurality of nucleotides with Formula X, W, Y, or Z in in 5′ to 3′ order, such that the formula of each nucleotide sequence is independently selectable and the nucleic sequence may have an independently selected positions at their respective R₁, R₂, R₃, and R₄, subgroups, wherein the nucleic acid sequence comprises at least two domains: an aptamer domain and a mi-RNA domain. In some embodiments, any one or plurality of Z of the nucleic acid sequence of N′-[Z]ₙ-N″ may be replaced with one or a plurality of contiguous or noncontiguous, modified or unmodified nucleotides chosen from Formula W, X, and/or Y.

The oligonucleotides of the disclosure may be conveniently synthesized using solid phase synthesis of known methodology, and is designed at least at the nucleotide-binding domain to be complementary to or specifically hybridizable with the preselected nucleotide sequence of the target RNA or DNA. Nucleic acid synthesizers are commercially available and their use is understood by persons of ordinary skill in the art as being effective in generating any desired oligonucleotide of reasonable length. It is also possible to synthesize the sgRNA by use of T7 RNA polymerase and a DNA template added to a mixture with individual dNTPs at an appropriate concentrations so that each nucleotide (whether it be RNA nucleotide or a DNA nucleotide) of the sgRNA is polymerized sequentially by the T7 polymerase catalyzing a reaction linking each base. Methods of making the nucleic acid sequences disclosed herein are contemplated by this application in which such nucleotide sequences may be manufactured by solid phase synthesis, by recombinant expression of one or more nucleotides in an invitro culture, or a combination of both in which modifications may be introduced at one or more positions across the length of the sequences.

In some embodiments, the degree of complementarity between a miRNA sequence and its corresponding target sequence, when optimally aligned using a suitable alignment algorithm, is about or more than about 50%, 60%, 75%, 80%. 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), ClustalW, Clustal X, BLAT, Novoalign (Novocraft Technologies, ELAND (Illumina, San Diego, Calif.), SOAP (available at soap.genomics.org.cn), and Maq (available at maq.sourceforge.net). In some embodiments, a nucleic acid sequence domain is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a nucleic acid sequence is less than about 75, 50, 45, 40, 35, 30, 25, 20, 15, 12, or fewer nucleotides in length. The ability of the miRNA domain of the nucleic acid sequence to direct sequence-specific binding of an mRNA may be assessed by any suitable assay.

In some embodiments, the nucleotide binding domain or aptamer domain consists of from about 15 to about 25 nucleotides; wherein the from 15 to about 25 nucleotides comprises a sequence similarity of about 90, 91, 92, 93, 94, 95, 96, 97, 98, 99%, or 100% sequence homology to any target sequences identified herein or in the table provided above. In some embodiments, the nucleotide binding domain or aptamer consists of from about 15 to about 30 nucleotides; wherein the from 15 to about 25 nucleotides comprises a sequence similarity of about 90, 91, 92, 93, 94, 95, 96, 97, 98, 99%, or 100% sequence homology to any target sequence identified herein. In some embodiments, the nucleotide binding domain or a aptamer domain consists of from about 15 to about 40 nucleotides; wherein the from 15 to about 25 nucleotides comprises a sequence similarity of about 90, 91, 92, 93, 94, 95, 96, 97, 98, 99%, or 100% sequence homology to any target sequence identified herein. In some embodiments, the nucleotide binding domain or a DNA-binding domain consists of from about 15 to about 25 nucleotides; wherein the from 15 to about 25 nucleotides comprises a sequence similarity of about 90, 91, 92, 93, 94, 95, 96, 97, 98, 99%, or about 100% sequence homology to any target sequence identified herein. For instance, one of ordinary skill in art could identify other DNA-binding domains which may be structurally related to those sequences provided in Table 4 to be used in connection with aptamer targeting.

In some embodiments, the aptamer domain is one or more aptamer domains disclosed in: Meyer S, Maufort J P, Nie J, Stewart R, McIntosh B E, Conti L R, et al. Development of an efficient targeted cell-SELEX procedure for DNA aptamer reagents. PLoS One. 2013; 8:e71798. Zhao N, Pei S N, Qi J, Zeng Z, Iyer S P, Lin P, et al. Oligonucleotide aptamer-drug conjugates for targeted therapy of acute myeloid leukemia. Biomaterials. 2015; 67:42-51, each of which is incorporated by reference in their entireties.

In some embodiments, any of the sequences disclosed herein may have a aptamer domain and an mi-RNA domain. Any of the domains of the disclosed oligonucleotides may be in any order from 5′ to 3′ orientation and may be contiguous as to each other or any one or multiple domains or elements may be non-contiguous in relation to one or more of the other domains, such that a different element, amino acid sequence, nucleotide or set of modified nucleotides may precede the 5′ and/or 3′ area of any domain.

In some embodiments, for instance, any one or combination of domains or sequences disclosed herein may comprise a sequence of about 1, 2, 3, 4, 5, 6, 7, 8, 9, 10, 15, 20, 25, 30, 35, 40, 45, or 50 or more modified or unmodified nucleotides flanking the 3′ or 5′ end of each domain. In some embodiments, for instance, any one or combination of domains or sequences disclosed herein may comprise a sequence of about 1, 2, 3, 4, 5, 6, 7, 8, 9, 10, 15, 20, 25, 30, 35, 40, 45, or 50 or more modified or unmodified uracils flanking the 3′ or 5′ end of each domain. Each domain may comprise from about 10 to about 15, 20, 25, 30, 35, 40, 45, 50, 60, 65, 70, 75, 80, 85, 90, 95, 100, 110, 120, 130, 140, 150, 160, 170, 180, 190, 200, 210, 220, 230, 240, or 250 or more modified or unmodified nucleic acids of DNA or RNA.

In some embodiments, the disclosure relates to a composition or pharmaceutical composition comprising a nucleic acid comprising the following domains contiguously oriented in the 5′ to 3′ direction: X₁ domain-DNA-binding domain-Cas binding domain-transcription terminator domain-X₂ domain;

wherein the X₁ domain is from about 0 to about 100 nucleotides in length, the DNA-binding domain is from about 1 to about 20 nucleotides in length, the Cas-binding domain is from about 30 to about 50 nucleotides in length, the transcription terminator domain is from about 30 to about 70 nucleotides in length, and wherein the X₂ domain is from about 0 to about 200 nucleotides in length, and wherein position 1 corresponds to the first nucleotide in the DNA-binding domain and each position thereafter is a successive positive integer; and each nucleotide in the X₁ domain, if not 0 nucleotides in length, is assigned a position of a negative integer beginning with the position −1 at the nucleotide adjacent to position 1 in the 5′ direction. In some embodiments, the disclosure relates to a composition or pharmaceutical composition comprising a nucleic acid that comprises the following domains contiguously oriented in the 5′ to 3′ direction: X₁ domain-DNA-binding domain-Cas binding domain-transcription terminator domain-X₂ domain; wherein the X₁ domain and the X₂ domain are 0 nucleotides in length, the DNA-binding domain is about 20 nucleotides in length, the Cas-binding domain is about 40 nucleotides in length, the transcription terminator domain is about 39 nucleotides in length.

In some embodiments, the disclosure relates to a composition or pharmaceutical composition comprising a nucleic acid comprises the following domains contiguously oriented in the 5′ to 3′ direction: X₁ domain-DNA-binding domain-Cas binding domain-transcription terminator domain-X₂ domain; wherein the X1 domain and the X2 domain are 0 nucleotides in length, the DNA-binding domain is about 20 nucleotides in length, the Cas-binding domain is about 40 nucleotides in length, the transcription terminator domain is about 39 nucleotides in length; and wherein the nucleic acid sequence comprises one or a combination of ribonucleotides at the positions identified in Table 5. In some embodiments, the one or a combination of ribonucleotides at the positions identified in Table 5 comprise 2′ hydroxyl groups within the sugar moieties of the nucleotide.

The disclosure relates to compositions and pharmaceutical compositions comprising one or a plurality of nucleic acid sequences disclosed herein, wherein the one or a plurality of nucleic acid sequences comprises from about 1% to about 99% modified nucleotides, wherein each modified nucleotide comprises at least two modification disclosed herein. The disclosure also relates to compositions and pharmaceutical compositions comprising one or a plurality of nucleic acid sequences disclosed herein, wherein the one or a plurality of nucleic acid sequences comprises from about 1% to about 99% modified nucleotides, wherein each modified nucleotide comprises a 2′ halogen at its 2′ carbon of its sugar moiety. In any embodiment, the one or plurality of nucleic acid sequences may comprise one or more nucleotides having Formula W, X, Y, and/or Z positioned in the sequence either contiguously or noncontiguously.

In some embodiments, the disclosure relates to compositions comprising a nucleic acid sequence comprising a miRNA domain sequence that is 70%, 75%, 80%, 85%, 90%, 91%, 92%, 93%, 94%, 95%, 96%, 97%, 98%, 99% to the RNA sequence: mi-26a UUCAAGUAAUCCAGGAUAGGCU (SEQ ID NO:1).

In some embodiments, the disclosure relates to a compositions comprising a nucleic acid sequence comprising a miRNA domain comprising, consisting essentially of, or consisting of SEQ ID NO:1.

In some embodiments, the disclosure relates to a compositions comprising a nucleic acid sequence comprising a miRNA domain comprising, consisting essentially of, or consisting of a sequence that is 70%, 75%, 80%, 85%, 90%, 91%, 92%, 93%, 94%, 95%, 96%, 97%, 98%, or 99%, homologous to SEQ ID NO:1.

In some embodiments, the disclosure relates to a compositions comprising a nucleic acid sequence comprising, consisting essentially of, or consisting of a sequence that is 70%, 75%, 80%, 85%, 90%, 91%, 92%, 93%, 94%, 95%, 96%, 97%, 98%, 99% homologous to any one or combination of sequences disclosed herein, wherein the nucleic acid sequence comprises a fragment or variant of the sequences disclosed herein but possesses the same or substantially the same function as the full-length sequence disclosed herein. For example, in the case of a fragment or variant of a nucleic acid sequence disclosed herein that comprises modified nucleotides in the DNA-binding domain, in some embodiments, the variant or fragment would be functional insomuch as it would exceed or retain some or all of its capacity to bind DNA at that domain as compared to the full-length sequence.

Any of the disclosed nucleic acid sequences may comprise any one or combination or set of modifications disclosed herein. In some embodiments, the nucleic acid, comprises RNA, DNA, or combinations of both RNA and DNA. In some embodiments, the nucleotide sequence, optionally in respect to one or a plurality of domains, comprises a modified nucleobase or a modified sugar.

Modifications to nucleotides are known in the art but include any of the disclosed modifications in the present application. Oligonucleotides particularly suited for the practice of one or more embodiments of the present disclosure comprise 2′-sugar modified oligonucleotides wherein one or more of the 2′-deoxy ribofuranosyl moieties of the nucleoside is modified with a halo, alkoxy, aminoalkoxy, alkyl, azido, or amino group. For example, the substitutions which may be independently selected from F, CN, CF₃, OCF₃, OCN, O-alkyl, S-alkyl, SMe, SO₂Me, ONO₂; NO₂, NH₃, NH₂, NH-alkyl, OCH₃═CH₂ and OCCH. In each of these, alkyl is a straight or branched chain of C₁ to C₂₀, having unsaturation within the carbon chain. A preferred alkyl group is C₁-C₉ alkyl. A further preferred alkyl group is C₅-C₂₀ alkyl.

A first group of substituents include 2′-deoxy-2′-fluoro substituents. A further preferred group of substituents include C₁ through C₂₀ alkoxyl substituents. An additional group of substituents include cyano, fluoromethyl, thioalkoxyl, fluoroalkoxy, alkylsulfinyl, alkylsulfonyl, allyloxy or alkenoxy substituents.

In further embodiments of the present disclosure, the individual nucleotides of the oligonucleotides of the disclosure are connected via phosphorus linkages. Phosphorus linkages include phosphodiester, phosphorothioate and phosphorodithioate linkages. In one preferred embodiment of this disclosure, nuclease resistance is conferred on the oligonucleotides by utilizing phosphorothioate internucleoside linkages.

In further embodiments of the disclosure, nucleosides can be joined via linkages that substitute for the internucleoside phosphate linkage. Macromolecules of this type have been identified as oligonucleosides. The term “oligonucleoside” thus refers to a plurality of nucleoside units joined by non-phosphorus linkages. In such oligonucleosides the linkages include an —O—CH₂—CH₂—O— linkage (i.e., an ethylene glycol linkage) as well as other novel linkages disclosed in U.S. Pat. No. 5,223,618, issued Jun. 29, 1993, U.S. Pat. No. 5,378,825, issued Jan. 3, 1995 and U.S. patent application Ser. No. 08/395,168, filed Feb. 27, 1995. Other modifications can be made to the sugar, to the base, or to the phosphate group of the nucleotide. Representative modifications are disclosed in International Publication Numbers WO 91/10671, published Jul. 25, 1991, WO 92/02258, published Feb. 20, 1992, WO 92/03568, published Mar. 5, 1992, and U.S. Pat. No. 5,138,045, issued Aug. 11, 1992, all of which are herein incorporated by reference in their entireties.

In some embodiments, a nucleic acid sequence is selected to reduce the degree of secondary structure within the nucleic sequence. Secondary structure may be determined by any suitable polynucleotide folding algorithm. Some programs are based on calculating the minimal Gibbs free energy. An example of one such algorithm is inFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g. A. R. Gruber et al., 2008, Cell 106(1): 23-24; and PA Carr and GM Church, 2009, Nature Biotechnology 27(12): 1151-62). Further algorithms may be found in U.S. application Ser. No. 61/836,080 filed Jun. 17, 2013 (attorney docket 44790.11.2022; Broad Reference BI-2013/004A); incorporated herein by reference in its entirety.

In some embodiments, the disclosure relates to modifications of the nucleic acid sequence that include positions of the sequences disclosed herein replaced by modified nucleotides that include additions of long non-coding RNAs (lncRNAs).

lncRNA has attracted much attention due to their large number and biological significance. Many lncRNAs have been identified as mapping to regulatory elements including gene promoters and enhancers, ultraconserved regions and intergenic regions of protein-coding genes. Yet, the biological function and molecular mechanisms of lncRNA in human diseases in Data from the literature suggest that lncRNA, often via interaction with proteins, functions in specific genomic loci or use their own transcription loci for regulatory activity. In some embodiments, the nucleic acid sequence of the disclosure comprises a length of contiguous lncRNA from about 150 nucleotides to about 250, 300, 350, 400, 450, or 500 nucleotides. In some embodiments, the nucleic acid sequence comprises a nucleotide domain that is at least 70%, 75%, 80%, 85%, 90%, 95%, 96%, 97%, 98%, 99% complementary to a known lncRNA sequence. The nucleic acid sequence may comprise an RNA binding domain that comprises such a complementary sequence or may comprise one or a plurality of RNA binding domains that comprises a at least 70%, 75%, 80%, 85%, 90%, 95%, 96%, 97%, 98%, 99% complementary to a known lncRNA sequence.

In another embodiment, the disclosure provides a cell or a vector comprising one of the nucleic acids of the disclosure or functional fragments thereof. The cell may be an animal cell or a plant cell. In some embodiments, the cell is a mammalian cell, such as a human cell.

In one aspect, the disclosure provides a vector system comprising one or more vectors. In some embodiments, the system comprises: (a) a synthetic nucleic acid sequence comprising at least one of the nucleic acid sequences disclosed herein, wherein the nucleic acid sequence directs sequence-specific portion of the aptamer domain to a target sequence in a eukaryotic cell, In general, and throughout this specification, the term “vector” refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g. circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g. retroviruses, replication defective retroviruses, adenoviruses, replication defective adenoviruses, and adeno-associated viruses). Viral vectors also include polynucleotides carried by a virus for transfection into a host cell. Certain vectors are capable of autonomous replication in a host cell into which they are introduced (e.g. bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Common expression vectors of utility in recombinant DNA techniques are often in the form of plasmids.

Another aspect of the disclosure relates to a composition comprising a nucleic acid disclosed herein and one or a plurality of recombinant expression vectors. Generally, the disclosure relates to composition comprising a synthetic nucleic acid sequence and one or a plurality of recombinant expression vectors. Recombinant expression vectors can comprise a nucleic acid of the disclosure in a form suitable for expression of the nucleic acid in a host cell, which means that the recombinant expression vectors include one or more regulatory elements, which may be selected on the basis of the host cells to be used for expression, that is operatively-linked to the nucleic acid sequence to be expressed. Within a recombinant expression vector, “operably linked” is intended to mean that the nucleotide sequence of interest is linked to the regulator) element(s) in a manner that allows for expression of the nucleotide sequence (e.g. in an in vitro transcription/translation system or in a host cell when the vector is introduced into the host cell).

The term “regulatory element” is intended to include promoters, enhancers, internal ribosomal entry sites (IRES), and other expression control elements (e.g. transcription termination signals, such as polyadenylation signals and poly-U sequences). Such regulatory elements are described, for example, in Goeddel, GENE EXPRESSION TECHNOLOGY: METHODS IN ENZYMOLOGY 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter may direct expression primarily in a desired tissue of interest, such as muscle, neuron, bone, skin, blood, specific organs (e.g. liver, pancreas), or particular cell types (e.g. lymphocytes). Regulatory elements may also direct expression in a temporal-dependent manner, such as in a cell-cycle dependent or developmental stage-dependent manner, which may or may not also be tissue or cell-type specific. In some embodiments, a vector comprises one or more pol III promoter (e.g. 1, 2, 3, 4, 5, or more pol Ill promoters), one or more pol II promoters (e.g. 1, 2, 3, 4, 5, or more pol II promoters), one or more pol I promoters (e.g. 1, 2, 3, 4, 5, or more pol I promoters), or combinations thereof. Examples of pol III promoters include, but are not limited to, U16 and HI promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) (see, e.g., Boshart et al, Cell, 41:521-530 (1985)), the SV40 promoter, the dihydrofoblate reductase promoter, the β-actin promoter, the phosphoglycerol kinase (PGK) promoter, and the EF1α promoter. Also encompassed by the term “regulatory element” are enhancer elements, such as WPRE; CMV enhancers; the R-U5′ segment in LTR of HTLV-1 (Mol. Cell. Biol., Vol. 8(1), p. 466-472, 1988); SV40 enhancer; and the intron sequence between exons 2 and 3 of rabbit 3-globin (Proc. Natl. Acad. Sci. USA., Vol. 78(3), p. 1527-31, 1981). It will be appreciated by those skilled in the art that the design of the expression vector can depend on such factors as the choice of the host cell to be transformed, the level of expression desired, etc. One or more nucleic acid sequences and one or more vectors can be introduced into host cells to form complexes with other cellular or non-natural compounds, produce transcripts, proteins, or peptides, including fusion proteins or peptides, encoded by nucleic acids as described herein (e.g., clustered regularly interspersed short palindromic repeats. (CRISPR) transcripts, proteins, enzymes, mutant forms thereof, fusion proteins thereof, etc.).

The disclosure also relates to pharmaceutical compositions comprising: (i) one or nucleic acid sequences disclosed herein or one or more pharmaceutically acceptable salts thereof; and (ii) a pharmaceutically acceptable carrier. The term “pharmaceutically acceptable salts” refers to physiologically and pharmaceutically acceptable salts of the nucleic acid sequences of the disclosure: i.e., salts that retain the desired biological activity of the nucleic acid sequences and do not impart undesired toxicological effects thereto.

Pharmaceutically acceptable base addition salts are formed with metals or amines, such as alkali and alkaline earth metals or organic amines. Examples of metals used as cations are sodium, potassium, magnesium, calcium, and the like. Examples of suitable amines are N,N′-dibenzylethylenediamine, chloroprocaine, choline, diethanolamine, dicyclohexylamine, ethylenediamine, N-methylglucamine, and procaine (see, for example,

Berge et al., “Pharmaceutical Salts,” J. of Pharnut Sci., 1977, 66:1). The base addition salts of said acidic compounds are prepared by contacting the free acid form with a sufficient amount of the desired base to produce the salt in the conventional manner. The free acid form may be regenerated by contacting the salt form with an acid and isolating the free acid in the conventional manner. The free acid forms differ from their respective salt forms somewhat in certain physical properties such as solubility in polar solvents, but otherwise the salts are equivalent to their respective free acid for purposes of the present disclosure. As used herein, a “pharmaceutical addition salt” includes a pharmaceutically acceptable salt of an acid form of one of the components of the compositions of the disclosure. These include organic or inorganic acid salts of the amines. In some embodiments, a pharmaceutically acceptable salt is selected from one or a combination of hydrochlorides, acetates, salicylates, nitrates and phosphates.

Other suitable pharmaceutically acceptable salts are well known to those skilled in the art and include basic salts of a variety of inorganic and organic acids, such as, for example, with inorganic acids, such as for example hydrochloric acid, hydrobromic acid, sulfuric acid or phosphoric acid; with organic carboxylic, sulfonic, sulfo or phospho acids or N-substituted sulfamic acids; for example acetic acid, propionic acid, glycolic acid, succinic acid, malefic acid, hydroxymaleic acid, methylmaleic acid, fiunaric acid, malic acid, tartaric acid, lactic acid, oxalic acid, gluconic acid, glucaric acid, glucuronic acid, citric acid, benzoic acid, cinnamic acid, mandelic acid, salicylic acid, 4-aminosalicylic acid, 2phenoxybenzoic acid, 2-acetoxybenzoic acid, embonic acid, nicotinic acid or isonicotinic acid; and with amino acids, such as the 20 alpha-amino acids involved in the synthesis of proteins in nature, for example glutamic acid or aspartic acid, and also with phenylacetic acid, methanesulfonic acid, ethanesulfonic acid, 2-hydroxyethanesulfonic acid, ethane-1,2-disulfonic acid, benzenesulfonic acid, 4-methylbenzenesulfonic acid, naphthalene-2-sulfonic acid, naphthalene-1,5-disulfonic acid, 2- or 3-phosphoglycerate, glucose-6phosphate, N-cyclohexylsulfamic acid (with the formation of cyclamates), or with other acid organic compounds, such as ascorbic acid. Pharmaceutically acceptable salts of compounds may also be prepared with a pharmaceutically acceptable cation. Suitable pharmaceutically acceptable cations are well known to those skilled in the art and include alkaline, alkaline earth, ammonium and quaternary ammonium cations. Carbonates or hydrogen carbonates are also possible.

For oligonucleotides, examples of pharmaceutically acceptable salts include but are not limited to (a) salts formed with cations such as sodium, potassium, ammonium, magnesium, calcium, polyamines such as spermine and spermidine, etc.; (b) acid addition salts formed with inorganic acids, for example hydrochloric acid, hydrobromic acid, sulfuric acid, phosphoric acid, nitric acid and the like; (c) salts formed with organic acids such as, for example, acetic acid, oxalic acid, tartaric acid, succinic acid, malefic acid, fumaric acid, glucoruc acid, citric acid, malic acid, ascorbic acid, benzoic acid, tannic acid, palimitic acid, alginic acid, polyglutamic acid, naphthalenesulfonic acid, methanesulfonic acid, p-toluenesulfonic acid, naphthalenedisulfonic acid, polygaiacturonic acid, and the like; and (d) salts formed from elemental anions such as chlorine, bromine, and iodine.

The practice of the present disclosure employs, unless otherwise indicated, conventional techniques of immunology, biochemistry, chemistry, molecular biology, microbiology, cell biology, genomics and recombinant DNA, which are within the skill of the art. See Sambrook. Fritsch and Maniatis, MOLECULAR CLONING: A LABORATORY MANUAL, 2nd edition (1989); CURRENT PROTOCOLS IN MOLECULAR BIOLOGY (F. M. Ausubel, et al. eds., (1987)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)), all of which are incorporated by reference in their entireties.

In some embodiments, the nucleic acid sequence comprises one or a plurality of radioactive moieties. Radioactive moiety means a substituent or component of a compound that comprises at least one radioisotope. Any radioisotope may be used. In some embodiments, the radioisotope is selected from Table 2. In some embodiments, the substituent or component of a compound of the present invention may incorporate any one, two, three, or more radioisotopes disclosed in Table 2.

TABLE 2: Radioisotopes that may be incorporated into pharmaceutical compositions
²H, ³H, ¹³C, ¹⁴C, ¹⁵N, ¹⁶O, ¹⁷O, ³¹P, ³²P, ³⁵S, ¹⁸F, ³⁶Cl, ²²⁵Ac, ²²⁷Ac, ²¹²Bi, ²¹³Bi, ¹⁰⁹Cd, ⁶⁰Co, ⁶⁴Cu, ⁶⁷Cu, ¹⁶⁶Dy ¹⁶⁹Er, ¹⁵²Eu, ⁵⁴Eu, ¹⁵³Gd, ¹⁹⁸Au, ¹⁶⁶Ho, ¹²⁵I, ¹³¹I, ¹⁹²Ir, ¹⁷⁷Lu, ⁹⁹Mo, ¹⁹⁴Os, ¹⁰³Pd, ^{195m}Pt, ³²P, ³³p, ²²³Ra, ¹⁸⁶Re, ¹⁸⁸Re, ¹⁰⁵Rh, ¹⁴⁵Sm, ¹⁵³Sm, ⁴⁷Sc, ⁷⁵Se, ⁸⁵Sr, ⁸⁹Sr, ^{99m}Tc, ²²⁸Th, ²²⁹Th, ¹⁷⁰Tm, ^{117m}Sn, ¹⁸⁸W, ¹²⁷Xe, ¹⁷⁵Yb, ⁹⁰Y, ⁹¹Y

In some embodiments, the composition or pharmaceutical composition comprises any nucleic acid disclosed herein or its salt and one or more therapies listed in Table 3. In some embodiments, the pharmaceutical composition comprises any one or plurality of nucleic acids disclosed herein or its salt or variant thereof and/or one or more therapies from Table 3 is administered to the subject before, contemporaneously with, substantially contemporaneously with, or after administration the pharmaceutical composition.

TABLE 3
Table of Chemotherapeutic Agents

Alkylating agents
Cyclophosphamide
Mechlorethamine
Chlorambucil
Melphalan
Anthracyclines
Daunorubicin
Doxorubicin
Epirubicin
Idarubicin
Mitoxantrone
Valrubicin
Cytoskeletal disruptors (Taxanes)
Paclitaxel
Docetaxel
Epothilones
Histone Deacetylase Inhibitors
Vorinostat
Romidepsin
Inhibitors of Topoisomerase I
Irinotecan
Topotecan
Inhibitors of Topoisomerase II
Etoposide
Teniposide
Tafluposide
Kinase inhibitors
Bortezomib
Erlotinib
Gefitinib
Imatinib
Vemurafenib
Vismodegib
Monoclonal antibodies
Bevacizumab
Cetuximab
Ipilimumab
Ofatumumab
Ocrelizumab
Panitumab
Rituximab
Nucleotide analogs and precursor analogs
Azacitidine
Azathioprine
Capecitabine
Cytarabine
Doxifluridine
Fluorouracil
Gemcitabine
Hydroxyurea
Mercaptopurine
Methotrexate
Tioguanine (formerly Thioguanine)
Peptide antibiotics
Bleomycin
Actinomycin
Platinum-based agents
Carboplatin
Cisplatin
Oxaliplatin
Retinoids
Tretinoin
Alitretinoin
Bexarotene
Vinca alkaloids and derivatives
Vinblastine
Vincristine
Vindesine
Vinorelbine
Actinomycin
All-trans retinoic acid
Azacitidine
Azathioprine
Bleomycin
Bortezomib
Carboplatin
Capecitabine
Cisplatin
Chlorambucil
Cyclophosphamide
Cytarabine
Daunorubicin
Docetaxel
Doxifluridine
Doxorubicin
Epirubicin
Epothilone
Etoposide
Fluorouracil
Gemcitabine
Hydroxyurea
Idarubicin
Imatinib
Irinotecan
Mechlorethamine
Mercaptopurine
Methotrexate
Mitoxantrone
Oxaliplatin
Paclitaxel
Pemetrexed
Teniposide
Tioguanine
Topotecan
Valrubicin
Vinblastine
Vincristine
Vindesine
Vinorelbine

Compositions of the disclosure include pharmaceutical compositions comprising: a particle comprising any of the nucleic acid sequences disclosed herein, or pharmaceutically acceptable salts thereof: and a pharmaceutically acceptable carrier. In some embodiments, the pharmaceutical composition comprise a pharmaceutically effective amount of one or a combination of chemotherapeutic agents chosen from Table 3. Any combination of 1, 2, 3, 4, 5, 6, 7, or more of those agents is capable of being a component in the compositions disclosed herein. Any combination of pharmaceutically effective amounts of 1, 2, 3, 4, 5, 6, 7, or more of those agents may be used or administered simultaneously, prior to or after administration of the pharmaceutical compositions disclosed herein in any of the disclosed methods.

As used herein, a “particle” refers to any entity having a diameter of less than 100 microns (μm). Typically, particles have a longest dimension (e.g. diameter) of 1000 nm or less. In some embodiments, particles have a diameter of 300 nm or less. In some embodiments, nanoparticles have a diameter of 200 nm or less. In some embodiments, nanoparticles have a diameter of 100 nm or less. In general, particles are greater in size than the renal excretion limit, but are small enough to avoid accumulation in the liver. In some embodiments, a population of particles may be relatively uniform in terms of size, shape, and/or composition. In general, inventive particles are biodegradable and/or biocompatible. Inventive particles can be solid or hollow and can comprise one or more layers. In some embodiments, particles are spheres, spheroids, flat, plate-shaped, cubes, cuboids, ovals, ellipses, cylinders, cones, or pyramids. In some embodiments, particles can be a matrix of polymers. In some embodiments, the matrix is cross-linked. In some embodiments, formation of the matrix involves a cross-linking step. In some embodiments, the matrix is not substantially cross-linked. In some embodiments, formation of the matrix does not involve a cross-linking step. In some embodiments, particles can be a non-polymeric particle (e.g. a metal particle, quantum dot, ceramic, inorganic material, bone, etc.). Components of the pharmaceutical compositions disclosed herein may comprise particles or may be microparticles, nanoparticles, liposomes, and/or micelles comprising one or more disclosed nucleic acid sequences or conjugated to one or more disclosed amino acids. As used herein, the term “nanoparticle” refers to any particle having a diameter of less than 1000 nm. Examples of nanoparticles are disclosed in Nature Biotechnology 31, 638-646, which is herein incorporated by reference in its entirety. In some embodiments, the particle is an exosome.

Pharmaceutical “carrier” or “excipient”, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21st Edition, A. R. Gennaro, (Lippincott, Williams & Wilkins, Baltimore, Md., 2006) discloses various excipients used in formulating pharmaceutical compositions and known techniques for the preparation thereof. Except insofar as any conventional excipient is incompatible with a substance or its derivatives, such as by producing any undesirable biological effect or otherwise interacting in a deleterious manner with any other component(s) of the pharmaceutical composition, its use is contemplated to be within the scope of this invention.

In some embodiments, the pharmaceutically acceptable excipient or carrier is at least 95%, 96%. 97%. 98%, 99%, or 100% pure. In some embodiments, the excipient is approved for use in humans and for veterinary use. In some embodiments, the excipient is approved by United States Food and Drug Administration. In some embodiments, the excipient is pharmaceutical grade. In some embodiments, the excipient meets the standards of the United States Pharmacopoeia (USP), the European Pharmacopoeia (EP), the British Pharmacopoeia, and/or the International Pharmacopoeia.

Pharmaceutically acceptable excipients used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in the inventive formulations. Excipients such as cocoa butter and suppository waxes, coloring agents, coating agents, sweetening, flavoring, and perfuming agents can be present in the composition, according to the judgment of the formulator.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and combinations thereof.

Exemplary granulating and/or dispersing agents include, but are not limited to, potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, etc., and combinations thereof.

Exemplary surface active agents and/or emulsifiers include, but are not limited to, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof.

Exemplary binding agents include, but are not limited to, starch (e.g. cornstarch and starch paste); gelatin; sugars (e.g. sucrose, glucose, dextrose, dextrin, molasses, lactose, lactitol, mannitol,); natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan); alginates; polyethylene oxide; polyethylene glycol; inorganic calcium salts; silicic acid; polymethacrylates; waxes; water; alcohol; etc.; and combinations thereof.

In some embodiments, the pharmaceutical composition comprise any one or combination of nucleic acid sequence disclosed here fused, linked or conjugated to a peptide from about 6 to about 100 amino acids long. In some embodiments, the pharmaceutical composition comprises a therapeutically effective amount of an RNA sequence that comprises an aptamer domain fused to a protein or peptide that is an exosome targeting domain. The exosome targeting domain comprises an amino acid sequence capable of binding or associating to a receptor on an exosome. In some embodiments, the pharmaceutical compositions comprise an aptamer domain fused or covalently bound to a peptide via a linker, wherein the peptide is a cancer antigen or exosome targeting domain. In some embodiments, the exosome targeting domain is CD63 or an amino acid sequence variant or truncation mutant that is at least 70% homolgous to the amino acid sequence of CD63 but still functional to bind its natural ligand. In some embodiments, the nucleic acid sequences disclosed herein are conjugated to an exosome via the amino acid sequence that is the exosome targeting domain.

Compositions of the disclosure relate to aptamers bound to exosome via an exosome targeting domain which is a nucleic acid sequence, amino acid sequence, or nucleic acid-amino acid fusion. In some embodiments, the composition comprises a nucleic acid sequence fused to a ligand. The ligand of the fusion typically is a heterologous amino acid sequence (i.e., relative to the engineered glycosylation site and/or relative to the exosome-targeting domain) that binds to a receptor present on the surface of a target cell (e.g., a protein receptor, a carbohydrate receptor, or a lipid receptor present on the surface of a cell). For example, suitable ligands may include a ligand for a cell receptor present on a target cell, or an antibody or binding fragment thereof that binds to a cell receptor or other membrane protein present on a target cell. The ligand of the fusion protein typically is present at the luminal end of the fusion molecule, which optionally may be the N-terminus of the fusion protein. For example, the fusion protein may comprise a structure as follows: nucleotide sequence comprising an aptamer domain—engineered glycosylation site--exosome targeting domain. In some embodiments, the exosome trgatein domain comprises the amino acid sequence for human CD63, or a sequence at least 75% homolgous to the human amino acid sequence of CD63. Sequences of exemplary aptamers are shown in Table 4 below.

TABLE 4
Aptamer sequences

| Apatamer | |
|---|---|
| Target | Aptamer Sequences |
| mouse | GCTCAACGCGGGACGGCTCTCCCATGAC |
| c-Kit | SEQ ID NO: 71 |
| CD117 | GAGGCATACCAGCTTATTATTGGGGCCGGGGCAAG |
| | GGGGGGGTACCGTGGTAGGACAGATAGTAAGTGCA |
| | ATCTGCGAA |
| | SEQ ID NO: 72 |
| CD117 | ATTGGGGCCGGGGCAAGGGGGGGGTACCGTGGTAG |
| (core) | GAC |
| | SEQ ID NO: 73 |
| CD3 | CAGCGTGGAGGATAAAAGTTAGCTGTTTCCTCGTG |
| | GCAGAAGGAACAGACCACCGTACTTTAGGGTGTGT |
| | CGT |
| | SEQ ID NO: 74 |
| CD4 | GTGACGTCCTGATCGATTGTGCATTCGGTGTGACG |
| | ATCT |
| | SEQ ID NO: 75 |
| CD8 | CTACAGCTTGCTATGCTCCCCTTGGGGTA |
| | SEQ ID NO: 76 |
| CD16 | CCACTGCGGGGGTCTATACGTGAGGAAGAAGTGGG |
| | CAGGTC |
| | SEQ ID NO: 77 |
| CD28 | GGGAGAGAGGAAGAGGGAUGGGGAUUAGACCAUAG |
| | GCUCCCAACCCCCAUAACCCAGAGGUCGAUAGUAC |
| | UGGAUCC |
| | SEQ ID NO: 78 |
| CD 40 | GGGAGAGACGAUGCGGCCAACGAGUAGGCGAUAGC |
| | GCGUGGCAGAGCGUCGCUGAGGAUCCGAGA |
| | SEQ ID NO: 79 |
| CD44 | CCAAGGCCTGCAAGGGAACCAAGGACACAG |
| | SEQ ID NO: 80 |
| mouse | GGGAGGACGAUGCGGCAGUCUGCAUCGUAGGAAUC |
| CD134 | GCCACCGUAUACUUUCCCACCAGACGACUCGCUGA |
| (OX40) | GGAUCCGAGA |
| | SEQ ID NO: 81 |
| CD134 | GGGAUGCGGAAAAAAGAACACUUCCGAUUAGGGCC |
| (OX40) | CACCCUAACGGCCGCAGAC |
| | SEQ ID NO: 82 |
| CD133 | CCCUCCUACAUAGGG |
| | SEQ ID NO: 83 |
| CD137 | GGGAGAGAGGAAGAGGGAUGGGCGACCGAACGUGC |
| (4-1BB) | CCUUCAAAGCCGUUCACUAACCAGUGGCAUAACCC |
| | AGAGGUCGAUAGUACUGGAUCCCCCC |
| | SEQ ID NO: 84 |
| mouse | GGGAGAGAGGAAGAGGGAUGGGCCGACGUGCCGCA |
| CD152 | SEQ ID NO: 85 |
| (CTLA-4) | |
| CD205 | GGGAGGUGUGUUAGCACACGAUUCAUAAUCAGCUA |
| | CCCUCCC |
| | SEQ ID NO: 86 |
| mouse | GACGATAGCGGTGACGGCACAGACGGCTACTGTAC |
| CD279 | ATCACGCCTCTCCCC CGTATGCCGCTTCCGTCCG |
| (PD-1) | TCGCTC |
| | SEQ ID NO: 87 |
| mouse | GGGAGAGGACCAUGUAGUCACUAUGGUCUUGGAGC |
| CD366 | UAGCGGCAGAGCGUCGCGGUCCCUCCC |
| (TIM3) | SEQ ID NO: 88 |
| EpCAM | AACAGAGGGACAAACGGGGGAAGATTTGACGTCGA |
| | CGACA |
| | SEQ ID NO: 89 |
| EGFR | TGCCGTTTCTTCTCTTTCGCTTTTTTTGCTTTTGA |
| | GCATG |
| | SEQ ID NO: 90 |
| Seq6 (mouse | GCTGTGTGACTCCTGCAATGTCCAGTCTCGGCCCT |
| macrophage) | ACCTTCGACTTCCTAAGTCGCATCGGCAGCTGTAT |
| | CTTGTCTCC |
| | SEQ ID NO: 91 |
| Nucleolin | GGTGGTGGTGGTTGTGGTGGTGGTGGTTTTTTTTT |
| (NUC) | TTT |
| | SEQ ID NO: 92 |
| PSMA | GGGAGGACGAUGCGGAUCAGCCAUGUUUACGUCAC |
| | UCCUUGUCAAUCCUCAUCGGC |
| | SEQ ID NO: 93 |
| Her2 | AACCGCCCAAATCCCTAAGAGTCTGCACTTGTCAT |
| | TTTGTATATGTTTGGTTTTTGGCTCTCACAGACAC |
| | ACTACACACGCACA |
| | SEQ ID NO: 94 |
| Nestin | GCAGTTGATCCTTTGGATACCCTGG |
| | SEQ ID NO: 95 |
| Mucin1 | GCAGTTGATCCTTTGGATACCCTGG |
| | SEQ ID NO: 96 |
| VEGFR | CGGAAUCAGUGAAUGCUAUACAUCCG |
| | SEQ ID NO: 97 |
| PDGF-BB | CAGGCTACGGCACGTAGAGCATCACCATGATCCTG |
| | TTTTTT |
| | SEQ ID NO: 98 |
| c-Met | GGAGGGAAAAGTTATCAGGCTGGATGGTAGCTCGG |
| | TCGGGGTGGGTGGGTTGGCAAGTCTGATTAGTTTT |
| | GGAGTACTCGCTCC |
| | SEQ ID NO: 99 |

### Methods of Making Compositions and Modifications

Modified oligonucleotides may be made with automated, solid phase synthesis methods known in the art. During solid phase synthesis, phosphoramidite monomers are sequentially coupled to a nucleoside that is covalently linked to a solid support. This nucleoside is the 3′ terminal nucleoside of the modified oligonucleotide. Typically, the coupling cycle comprises four steps: detritylation (removal of a 5′-hydroxyl protecting group with acid), coupling (attachment of an activated phosphoroamidite to the support bound nucleoside or oligonucleotide), oxidation or sulfurization (conversion of a newly formed phosphite trimester with an oxidizing or sulfurizing agent), and capping (acetylation of unreacted 5′-hydroxy 1 groups). After the final coupling cycle, the solid support-bound oligonucleotide is subjected to a detritylation step, followed by a cleavage and deprotection step that simultaneously releases the oligonucleotide from the solid support and removes the protecting groups from the bases. The solid support is removed by filtration, the filtrate is concentrated and the resulting solution is tested for identity and purity. The oligonucleotide is then purified, for example using a column packed with anion-exchange resin.

This term includes oligonucleotides composed of naturally-occurring nucleobases, sugars and covalent internucleoside (backbone) linkages as well as oligonucleotides having non-naturally-occurring portions which function similarly. Such modified or substituted oligonucleotides are often preferred over native forms because of desirable properties such as, for example, enhanced cellular uptake, enhanced affinity for nucleic acid target and increased stability in the presence of nucleases.

In forming oligonucleotides, the phosphate groups covalently link adjacent nucleosides to one another to form a linear polymeric compound. In turn the respective ends of this linear polymeric structure can be further joined to form a circular structure, however, open linear structures are generally preferred. Within the oligonucleotide structure, the phosphate groups are commonly referred to as forming the internucleoside backbone of the oligonucleotide. The normal linkage or backbone of RNA and DNA is a 3′ to 5′ phosphodiester linkage.

As defined in this specification, oligonucleotides having modified backbones include those that retain a phosphorus atom in the backbone and those that do not have a phosphorus atom in the backbone. For the purposes of this specification, and as sometimes referenced in the art, modified oligonucleotides that do not have a phosphorus atom in their internucleoside backbone can also be considered to be oligonucleosides.

Preferred modified oligonucleotide backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included.

Representative United States patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, each of which is herein incorporated by reference in its entirety.

Preferred modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH2 component parts. Any of the olignucleotide backbone modifications here may replace any one of the internucleotide linkages set forth in Formula W, X, Y, and/or Z.

Representative United States patents that teach the preparation of the above oligonucleosides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference in its entirety.

In other preferred oligonucleotide mimetics, both the sugar and the internucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Representative United States patents that teach the preparation of PNA compounds include, but are not limited to, U.S. Pat. Nos. 5,539,082; 5,714,331; and 5,719,262, each of which is herein incorporated by reference. Further teaching of PNA compounds can be found in Nielsen et al., Science, 1991, 254, 1497-1500.

Some embodiments of the disclosure are oligonucleotides with phosphorothioate backbones and oligonucleosides with heteroatom backbones, and in particular —CH2-NH—O—CH2-, —CH2-N(CH3)-O—CH2- [known as a methylene (methylimino) or MMI backbone], —CH2-O—N(CH3)-CH2-, —CH2-N(CH3)-N(CH3)-CH2- and —N(CH3)-CH2-CH2- [wherein the native phosphodiester backbone is represented as —O—P—O—CH2-] of the above referenced U.S. Pat. No. 5,489,677, and the amide backbones of the above referenced U.S. Pat. No. 5,602,240. Also preferred are oligonucleotides having morpholino backbone structures of the above-referenced U.S. Pat. No. 5,034,506.

Modified oligonucleotides may also contain one or more substituted sugar moieties. In some embodiments, oligonucleotides of the disclosure comprise one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Particularly preferred are O[(CH₂)nO]mCH₃, O(CH₂)nOCH₃, O(CH₂)nNH₂, O(CH₂)nCH₃, O(CH₂)nONH₂, and O(CH₂)nON[(CH₂)nCH₃)]₂, where n and m are from 1 to about 10. Other preferred oligonucleotides comprise one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, acetamide, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A preferred modification includes 2′-methoxyethoxy (2′-O—CH2CH2OCH3, also known as 2′-O-(2-methoxyethyl) or 2′-MOE) (Martin et al., Helv. Chim. Acta, 1995, 78, 486-504) i.e., an alkoxyalkoxy group. Another modification includes 2′-dimethylaminooxyethoxy, i.e., a O(CH₂)₂ON(CH₃)₂ group, also known as 2′-DMAOE, and 2′-dimethylamino-ethoxyethoxy (2′-DMAEOE), i.e., 2′-O—CH₂—CH2-N(CH₂h).

Other modifications include 2′-methoxy (2′-O—CH₃), 2′-aminopropoxy (2′-OCH₂CH₂CH₂NH₂) and 2′-fluoro (2′-F). Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar. Representative United States patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, each of which is herein incorporated by reference in its entirety.

Oligonucleotides may also include a modified thioester group on the 2′, 3′ and/or 5′ nucleoside. Such modifications in the 5′ carbon of the ribose sugar also for formation of single 5′-S-thioester linkages between nucleotides in a synthetic nucleotide sequence. In any 3′ or 5′ linkage between nucleotides any one or both positions may create a series of linkages between nucleotides. The linkages at the 2′ or 3′ can create thioester bond, phosphorothioriate linkages between two or a plurality of nucleosides in the oligonucleotide.

Strategically placed sulfur atoms in the backbone of nucleic acids have found widespread utility in probing of specific interactions of proteins, enzymes and metals. Sulfur replacement for oxygen may be carried out at the 2′-position of RNA and in the 3′-5′-positions of RNA and of DNA. Polyribonucleotide containing phosphorothioate linkages were obtained as early as 1967 by Eckstein et al. using DNA-dependent RNA polymerase from E. coli (57). DNA-dependent RNA polymerase is a complex enzyme whose essential function is to transcribe the base sequence in a segment of DNA into a complementary base sequence of a messenger RNA molecule. Nucleoside triphosphates are the substrates that serve as the nucleotide units in RNA. In the polymerization of triphosphates, the enzyme requires a DNA segment that serves as a template for the base sequence in the newly synthesized RNA. In the original procedure, Uridine 5′-O-(1-thiotriphosphate), adenosine 5′-O-triphosphate, and only d (AT) as a template was used. As a result, an alternating copolymer is obtained, in which every other phosphate is replaced by a phosphorothioate group. Using the same approach and uridine 5′-O-(1-thiotriphosphate) and adenosine 5′-O-(1-thiotriphosphate), polyribonucleotide containing an all phosphorothioate backbone can also synthesized. In both cases, nucleoside 5′-O-(1-thiotriphosphates) as a mixture of two diastereomers can be used. In some embodiments, alternating phosphorothioate groups link a DNA or RNA or hybrid sequence of predominantly RNA to form alternating phosphorothioate backbones. Optionally, linkers of any cyclic or acyclic hydrocarbon chains of varying length may be incorporated into the nucleic acid. In some embodiments, linkers of the disclosure comprise one or a plurality of: branched or non-branched alkyl, hydroakyl, hydroxyl, halogen, metal, nitrogen, or other atoms.

Oligonucleotides may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine. 5-propynyl uracil and cytosine, 6-azo uracil, cytosine and thymine, 5-uracil (pseudouracil), 4-thiouracil. 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Certain of these nucleobases are particularly useful for increasing the binding affinity of the oligomeric compounds of the disclosure. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., eds., Antisense Research and Applications, CRC Press, Boca Raton, 1993, pp. 276-278) and are presently preferred base substitutions, even more particularly when combined with 2′-O-methoxyethyl sugar modifications.

Representative United States patents that teach the preparation of certain of the above noted modified nucleobases as well as other modified nucleobases include, but are not limited to, the above noted U.S. Pat. No. 3,687,808, as well as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,681,941, and 5,750,692, each of which is herein incorporated by reference in its entirety.

In some embodiments, the nucleic acids is conjugated to other proteins, polypeptides or molecules. Representative United States patents that teach the preparation of such oligonucleotide conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5.258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, each of which is herein incorporated by reference in its entirety.

It is not necessary for all positions in a given compound to be uniformly modified, and in fact more than one of the aforementioned modifications may be incorporated in a single sequence or compound or even at a single nucleoside or functional group within one or a plurality of posioins within a nucleoside or an oligonucleotide.

For example. GalNAc-conjugated modification are known to direct oligonucleotides to liver cells. Modifications, such as GalNAc-conjugated modification, may be made to any one or combination of oligonucleotides disclosed herein with automated solid phase synthesis, similar to the solid phase synthesis that produced unconjugated oligonucleotides. During the synthesis of GalNAc-conjugated oligonucleotides, the phosphoramidite monomers are sequentially coupled to a GalNAc conjugate which is covalently linked to a solid support. The synthesis of GalNAc conjugates and GalNAc conjugate solid support is described, for example in U.S. Pat. No. 8,106,022, which is herein incorporated by reference in its entirety for the description of the synthesis of carbohydrate-containing conjugates, including conjugates comprising one or more GalNAc moieties, and of the synthesis of conjugate covalently linked to solid support.

The disclosure also relates to synthesizing one or a plurality of oligonucleotides, such as aptamer-miRNA chimeric molecules. 2′-deoxy-2′-modified nucleosides of adenine, guanine, cytosine, thymidine and certain analogs of these nucleobases may be prepared and incorporated into oligonucleotides via solid phase nucleic acid synthesis. Novel oligonucleotides can be assayed for their hybridization properties and their ability to resist degradation by nucleases compared to the unmodified oligonucleotides. Initially, small electronegative atoms or groups can be selected because they would not be expected to sterically interfere with required Watson-Crick base pair hydrogen bonding (hybridization). However, electronic changes due to the electronegativity of the atom or group in the 2′-position may profoundly affect the sugar conformation.

2′-Substituted oligonucleotides can be synthesized by standard solid phase nucleic acid synthesis using an automated synthesizer such as Model 380B (Perkin-Elmer/Applied Biosystems) or MilliGen/Biosearch 7500 or 8800. Triester, phosphoramidite, or hydrogen phosphonate coupling chemistries [Oligonucleotides. Antisense Inhibitors of Gene Expression. M. Caruthers, p. 7, J. S. Cohen (Ed.), CRC Press, Boca Raton. Fla., 1989] are used with these synthesizers to provide the desired oligonucleotides. The Beaucage reagent [J. Amer. Chem. Soc., 112, 1253 (1990)] or elemental sulfur [Beaucage et al., Tet. Lett., 22, 1859 (1981)] is used with phosphoramidite or hydrogen phosphonate chemistries to provide 2′-substituted phosphorothioate oligonucleotides.

2′-substituted nucleosides (A, G, C, T(U), and other modified nucleobases) may be prepared by modification of several literature procedures as described below.

Procedure 1. Nucleophilic Displacement of 2′-Leaving Group in Arabino Purine Nucleosides. Nucleophilic displacement of a leaving group in the 2′-up position (2′-deoxy-2′-(leaving group)arabino sugar) of adenine or guanine or their analog nucleosides. General synthetic procedures of this type have been described by Ikehara et al., Tetrahedron, 34, 1133 (1978); ibid., 31, 1369 (1975); Chemistry and Pharmaceutical Bulletin, 26, 2449 (1978); ibid., 26, 240 (1978); Ikehara, Accounts of Chemical Research, 2, 47 (1969); and Ranganathan, Tetrahedron Letters, 15, 1291 (1977).

Procedure 2. Nucleophilic Displacement of 2,2′-Anhydro Pyrimidines. Nucleosides thymine, uracil, cytosine or their analogs are converted to 2′-substituted nucleosides by the intermediacy of 2,2′-cycloanhydro nucleoside as described by Fox et al., Journal of Organic Chemistry, 29, 558 (1964).

Procedure 3. 2′-Coupling Reactions. Appropriately 3′,5′-sugar and base protected purine and pyrimidine nucleosides having a unprotected 2′-hydroxyl group are coupled with electrophilic reagents such as methyl iodide and diazomethane to provide the mixed sequences containing a 2′-OMe group H. Inoue et al., Nucleic Acids Research, 15, 6131.

Procedure 4. 2-Deoxy-2-substituted Ribosylations. 2-Substituted-2-deoxyribosylation of the appropriately protected nucleic acid bases and nucleic acids base analogs has been reported by Jarvi et al., Nucleosides & Nucleotides, 8, Ill 1-1114 (1989) and Hertel et al., Journal of Organic Chemistry, 53, 2406 (1988).

The disclosure relates to a composition or pharmaceutical composition comprising a therapeutically effective amount of a nucleic acid molecule comprising a first and second aptamer domain oriented in the 5′ to 3′ orientation or the 3′ to 5′ orientation. The aptamer domains may be contiguous on a single nucleic acid molecule or linked by a DNA or RNA linker in a double-stranded preparation, and the one or plurality of aptamer, domains are optionally complementary to the linker sequence such that the nucleic acid molecule is in the structure of: aptamer domain 1-linker-aptamer domain 2 in a partially double-stranded state wherein each aptamer domain is optionally complementary to a DNA linker or RNA linker positioned therebetween. In some embodiments, the structure of the nucleic acid molecule is selected from:

aptamer domain 1-XX′-linker-aptamer domain 2;

aptamer domain 1-XX′-aptamer domain 2; or

aptamer domain 1-XX′-linker-XX″-aptamer domain 2;

wherein each aptamer domain is independently variable in length from about 15 to about 100 modified or unmodified nucleotides and wherein the domains XX′ and/or XX″ and/linker domain have an independently length from about 0 to about 60 modified or unmodified nucleotides in length and wherein if either or both XX′ and XX″ domains are from about 15 to about 60 nucleotides in length at least one of the domains is a therapeutic nucleic acid sequence such as a miRNA sequence at least about 10%, 20%, 30%, 40%, 50%, 60%, 70%, 80%. 90%, 95%, 96%, 97%, 98%, 99%, or 100% complementary to a mRNA target sequence. As disclosed above, the disclosure reads on such nucleic acid sequence comprising any one or multiple nucleotides of Formula W, X, Y, and/or Z in any series or pattern, contiguously positioned or non-contiguously positioned in the nucleotide molecule or salt thereof.

In some embodiments, the nucleic acid molecule further comprises a cholesterol or modified cholesterol molecule covalently or non-covalently bound to the nucleic acid molecule in a molar ratio of 1:1 in terms of moles of cholesterol:moles of aptamer domain or if the nucleic acid molecule comprises a first aptamer domain and a second aptamer domain or a therapeutic RNA sequence such as a miRNA, the molar ratio of modified or unmodified cholesterol covalently or non-covalently bound to the nucleic acid molecule is in a ratio of about 2:1, 2:1:1, 1:1, or 1:1:1 in terms of modified or unmodified cholesterol:aptamer domain 1: aptamer domain 2 or gene silencing domain. Modified cholesterol includes any modified cholesterol disclosed in the Examples section.

In some embodiments, the presence of the nucleic acid molecule comprising a chimeric set of nucleic acid sequences comprising the following structure: aptamer domain 1- linker-aptamer domain 2, wherein the first aptamer domain is positioned at the flank of the nucleic acid molecule and the second aptamer is positioned at the opposing flank of the nucleic acid molecule and one of the two aptamer domain comprises a sequence that associates with an amino acid sequence on the surface of an exosome (such as CD63), and the other aptamer domain comprises a sequence that associates with an amino acid that is expressed by a target cell such as a cancer cell.

Pharmaceutical compositions and compositions of the disclosure relate to a therapeutically effective amounts of nucleic acid molecule or molecules that comprise two contiguous or non-contiguous aptamer domains, one aptamer bound to a receptor or peptide on the surface of an exosome, said exosome comprising a shell and hydrophilic core in which one or plurality of payloads is contained from the environment outside of the exosome. In some embodiments, the composition also comprises a therapeutically effective amount of an exosome bound to one or a plurality of nucleic acid sequence disclosed herein, each nucleic acid sequence bound or associated to an amino acid on the surface of the exosome. In some embodiments, the exosome may be associated with between from about 1 to about 100.

Payloads include: siRNA, miRNA, shRNA, mRNA molecule or molecules that encode one or more DNAs of a therapeutic protein. DNA that encodes a therapeutic protein or immunogen, a therapeutic amino acid sequence or an amino acid sequence that is a gene editing enzyme. In some embodiments, the gene editing enzyme is any enzyme identified in the disclosure related to a CRISPR complex, such as Cas9 of variants at least 70% homologous to Cas9 or any other enzyme with Cas9-like function and disclosed in the application. Payloads comprising combinations of molecules include: (i) an amino acid sequence or functional fragment thereof with a structure of 70% homology to a Cas9 protein or amino acid sequence with Cas9-like (gene editing) function or a nucleic acid encoding the same; (ii) an sgRNA, tracer and/or tracrmate RNA sequence, a RNA/DNA molecule with the same sgRNA function, wherein the sgRNA sequence comprises a nucleotide sequence that is partially complementary to a genomic sequence targeted for mutagenesis. In some embodiments, the exosome comprises between about 1 to about 1×10¹⁰ molecules including any one or combination of the above-identified molecules. In some embodiments, the pharmaceutical composition comprises a therapeutically effective amount of exosomes. In some embodiments, the therapeutically effective amount of exosomes comprise an amount from about 1 to about 1×10¹⁰ particles.

In some embodiments, the one or more aptamer domains of the nucleic acid sequence targets the entire nucleic acid molecule to a cell and/or exosome. In some embodiments, the first aptamer domains flanks a terminus of the nucleic acid molecule and the second aptamer domain flanks the opposite nucleic acid terminus. In some embodiments, the first aptamer domain targets the nucleic acid molecule to a cancer cell and the other aptamer domain directs association between it and a ligand or polypeptide on the surface of an exosome. In such embodiments the bispecific aptamers direct delivery of one or a plurality of payloads to the cell upon which the first aptamer domain associates. In some bispecific aptamers, the first or second aptamer domain binds or associates with any one or combination of the amino acid sequences disclosed in Table 1. In some embodiments, such amino acid sequences are conjugated or displayed on the exosome. In some embodiments, the first and/or second aptamer domain comprises, consist of, or consists essentially of any of the nucleic acid sequences of Table 4 or those sequences that have about 70%, 75%, 80%, 85%, 90%, 91%, 92%, 93%, 94%, 95%, 96%, 97%, 98%, 99% homology to the nucleic acid sequences of Table 4.

The disclosure also relates to a composition or pharmaceutical composition comprising a therapeutically amount of a nucleic acid molecule comprising a first aptamer domain and a second aptamer domain disclosed herein wherein a first aptamer domain is bound to an exosome via association or non-covalently bonding, or covalent bonding to a aptaremer targeting domain on the surface of the exosome; and wherein the exosome comprises payload of a therapeutic nucleic acid sequence and/or amino acid sequence.

### Exosomes and Vaccines

Improved vaccines are disclosed which arise from a strategy to enhance cellular immune responses induced by immunogens. Any modified consensus sequence can be generated and considered payload for encapsulation within an exosome. Genetic modifications including codon optimization, RNA optimization, and the addition of a high efficient immunoglobin leader sequence are also disclosed as part of an expressible nucleic acid sequence operably linked to a regulatory sequence and encapsulated inside of an exosome. The novel construct has been designed to elicit stronger and broader cellular immune responses than a corresponding codon optimized immunogens. Generally exosome for vaccines have been described in WO/2016/193422 and WO/2008/092153, which are incorporated by reference in their entireties.

The improved vaccines are based upon proteins and genetic constructs that encode proteins with epitopes that make them particularly effective as immunogens against which anti-immunogen can be induced. Accordingly, vaccines may induce a therapeutic or prophylactic immune response. In some embodiments, the means to deliver the immunogen is a DNA vaccine, a recombinant vaccine, a protein subunit vaccine, a composition comprising the immunogen, an attenuated vaccine or a killed vaccine. In some embodiments, the vaccine comprises a combination selected from the groups consisting of: one or more DNA vaccines, one or more protein subunit vaccines, one or more compositions comprising the immunogen, one or more attenuated vaccines and/or one or more killed vaccines such vaccines associate with one or more nucleic acid sequences disclosed herein with an aptamer domain directed to an amino acid sequence on the surface of the vaccine particle, cell or exosome encapsulating the payload.

According to some embodiments, a vaccine is delivered to an individual to modulate the activity of the individual's immune system or induce the immune system and thereby enhance the immune response against immunogen. When a nucleic acid molecules that encode the immunogen is taken up by cells of the individual the nucleotide sequence is expressed in the cells and the protein are thereby delivered to the individual. Methods of delivering the coding sequences of the protein on nucleic acid molecule such as plasmid, as part of recombinant vaccines and as part of attenuated vaccines, as isolated proteins or proteins part of a vector are provided. In some embodiments, the association of one or plurality of nucleic acid sequences comprising bispecific aptamer domains enhance the immune response by increasing the efficiency of delivery of the payload into one or more cells. In some embodiments, the cells are antigen-presenting cells, such as a dendritic cells or macrophages and the first or second aptamer domain comprises a sequence that binds or associates to the antigen presenting cell for directed delivery of the exosome, vaccine or cell to the antigen presenting cell thereby causing phagocytosis or contact of the payload with the antigen presenting cell interior and expression of the immunogen. In some embodiments, gene editing machinery such as CRISPR related enzymes or genes are incorporated or encapsulated by the exosomes for targeted genomic DNA modification. The nucleic acid sequences of the disclosure bound or associated to an exosome increases the efficiency with which the mutageneisis or immunogen can be expressed by a target cell, especially a cancer cell disclosed herein.

Vaccines of the disclosure include exosomes bonded to, associated with or noncovalently bonded to one or a plurality of hundreds of nucleic acid molecules disclosed herein, each nucleic acid molecule with at least one independently variable aptamer domain comprising at least one sequence capable of associating with an exosome targeting domain such as CD63 and at least one aptamer domain cpabel of of associating with an antigen presenting cell targeting domain. Exosomes can include any nucleic acid molecule comprising any of the mi-RNA domains disclosed herein.

In some embodiments, the immunogen is a cancer-associated antigen expressed on cancer cells. In some embodiments, the immunogen is a cancer-associated antigen expressed on cells identified in Table 1. In some embodiments, the immunogen is an amino acid sequence that is at least 70%, at least 80%, at least 85%, at least 90%, at least 95%, at least 96%, at least 97%, at least 98%, at least 99%, or 100% homologous to the sequences of Table 1.

### Methods

The disclosure relates to treating a cancer in an subject in need thereof comprising administering a therapeutically effective amount of the pharmaceutical composition disclosed herein. In certain embodiments, the disclosure relates to methods of treating a KIT expressing cancer in an individual in need thereof comprising: (a) identifying the cancer as a KIT expressing cancer; and (b) providing a therapeutically effective amount of the pharmaceutical composition to the subject. In some embodiments, the methods further comprise administering a chemotherapeutic agent to the individual, optionally at a dose typically toxic to a human subject. In some embodiments, the chemotherapeutic agent is 5-FU, a small interfering RNA, or any one or combination of chemotherapeutic agents listed in Table 3. In some embodiments, the pharmaceutical composition and the chemotherapeutic agent are synergistic. In some embodiments, the cancer type s provided as any cancer type disclosed in Table 1.

In some embodiments, the pharmaceutical composition is adminstered before the chemotherapeutic agent. In some embodiments, the methods further comprise providing radiotherapy to the individual. In some embodiments, the pharmaceutical composition nucleotide is provided before the radiotherapy. In some embodiments, the cancer is a solid tumor. In some embodiments, the solid cancer is a melanoma, nasopharyngeal cancer, neuroendocrine tumor, lung cancer, colon cancer, urothelial cancer, bladder cancer, liver cancer, multiple myeloma, ovarian cancer, gastric carcinoma, esophageal cancer, pancreatic cancer, kidney cancer, breast cancer, or lymphoma. In some embodiments, the lung cancer is a non-small cell lung cancer (NSCLC) optionally expressing mouse or hum an or other mammalian variant of KIT. In some embodiments, the lung cancer is a small-cell lung cancer (SCLC). In some embodiments, the liver cancer is a hepatocellular carcinoma (HCC). In some embodiments, the cancer is a leukemia. In some embodiments, the cancer is a lymphoma. In some embodiments, the cancer as a KIT expressing cancer comprises measuring the KIT expression in a cancer c ell from the individual and comparing to a control. In some embodiments, the KIT expression is overexpressed compared to the control. In some embodiments, the methods further comprise selecting the individual having functional p53.

Table 1 below lists various cell surface proteins expressed by cancer cells and example of the types of cancer that are known to express those target proteins. Methods of treating any of the disclosed cancer types are provided, whereby composition of pharmaceutical compositions comprise an aptamer domain targeting the amino acid identified next to subtitling each section.

All amino acid sequences or Accession Numbers below as of May 18, 2017, are incorporated by reference in their entireties. Any mutants or variants that are at least 70, 75, 80, 85, 90, 95, 96, 97, 98, 99% homologous to the encoded amino acids set forth in the sequences or Accession Numbers below are also incorporated by reference in their entireties. The nucleic acid sequences that encode the amino acid sequences are also contemplated by this disclosure as well as plasmid sequences comprising any one or plurality of expressible nucleic acid sequences that are at least 70%, 80%, 85%, 90%, 95%, 96%, 97%, 98%, 99% or 100% homologous to the sequences below. Amino acid variants and full-length protein sequences are contemplated by the disclosure and can be considered payloads for this disclosure in addition to the nucleic acid sequences.

TABLE 1
Cell surface target proteins on cancer cells that can bind to aptamer domains

| Cell surface | | |
|---|---|---|
| protein | Cancer types | Sequence |
| CD117 | Breast cancer | 1 mrgargawdf lcvlllllrv qtgssqpsvs pgepsppsih |
| (AAH71593) | Lung cancer | pgksdlivrv gdeirllctd |
| SEQ ID NO: 100 | Prostate cancer | 61 pgfvkwtfei ldetnenkqn ewitekaeat ntgkytctnk |
| | Ovarian cancer | hglsnsiyvf vrdpaklflv |
| | Gastrointestinal | 121 drslygkedn dtlvrcpltd pevtnyslkg cqgkplpkdl |
| | stromal | rfipdpkagi miksvkrayh |
| | tumors | 181 rlclhcsvdq egksvlsekf ilkvrpafka vpvvsvskas |
| | Testicular | yllregeeft vtctikdvss |
| | seminoma | 241 svystwkren sqtklqekyn swhhgdfnye rqatltissa |
| | Melanoma | rvndsgvfmc yanntfgsan |
| | Acute myeloid | 301 vtttlevvdk gfinifpmin ttvfvndgen vdliveyeaf |
| | leukemia | pkpehqqwiy mnrtftdkwe |
| | | 361 dypksenesn iryvselhlt rlkgteggty tflvsnsdvn |
| | | aaiafnvyvn tkpeiltydr |
| | | 421 lvngmlqcva agfpeptidw yfcpgteqrc sasvlpvdvq |
| | | tlnssgppfg klvvqssids |
| | | 481 safkhngtve ckayndvgkt sayfnfafkg nnkeqihpht |
| | | lftplligfv ivagmmciiv |
| | | 541 miltykylqk pmyevqwkvv eeingnnyvy idptqlpydh |
| | | kwefprnrls fgktlgagaf |
| | | 601 gkvveatayg liksdaamtv avkmlkpsah lterealmse |
| | | lkvlsylgnh mnivnllgac |
| | | 661 tiggptlvit eyccygdlln flrrkrdsfi cskqedhaea |
| | | alyknllhsk esscsdstne |
| | | 721 ymdmkpgvsy vvptkadkrr svrigsyier dvtpaimedd |
| | | elaidledll sfsyqvakgm |
| | | 781 aflaskncih rdlaarnill thgritkicd fglardiknd |
| | | snyvvkgnar lpvkwmahes |
| | | 841 ifncvytfes dvwsygiflw elfslgsspy pgmpvdskfy |
| | | kmikegfrml spehapaemy |
| | | 901 dimktcwdad plkrptfkqi vgliekqise stnhiysnla |
| | | ncspnrqkpv vdhsvrinsv |
| | | 961 gstasssqpl lvhddv |
| Prostate | Prostate cancer | 1 mwnllhetds avatarrprw lcagalvlag gffllgflfg |
| specific | Kidney cancer | wfikssneat nitpkhnmka |
| membrane | Bladder cancer | 61 fldelkaeni kkflynftqi phlagteqnf glakqiqsqw |
| antigen | Pancreatic | kefgldsvel ahydvllsyp |
| (PSMA) | cancer | 121 nkthpnyisi inedgneifn tslfeppppg yenvsdivpp |
| (AAA60209) | | fsafspqgmp egdlvyvnya |
| SEQ ID NO: 101 | | 181 rtedffkler dmkincsgki viarygkvfr gnkvknaqla |
| | | gakgvilysd padyfapgvk |
| | | 241 sypdgwnlpg ggvqrgniln lngagdpltp gypaneyayr |
| | | rgiaeavglp sipvhpigyy |
| | | 301 daqkllekmg gsappdsswr gslkvpynvg pgftgnfstq |
| | | kvkmhihstn evtriynvig |
| | | 361 tlrgavepdr yvilgghrds wvfggidpqs gaavvheivr |
| | | sfgtlkkegw rprrtilfas |
| | | 421 wdaeefgllg stewaeensr llqergvayi nadssiegny |
| | | tlrvdctplm yslvhnltke |
| | | 481 lkspdegfeg kslyeswtkk spspefsgmp risklgsgnd |
| | | fevffqrlgi asgrarytkn |
| | | 541 wetnkfsgyp lyhsvyetye lvekfydpmf kyhltvaqvr |
| | | ggmvfelans ivlpfdcrdy |
| | | 601 avvlrkyadk iysismkhpq emktysvsfd slfsavknft |
| | | eiaskfserl qdfdksnpiv |
| | | 661 lrmmndqlmf lerafidplg lpdrpfyrhv iyapsshnky |
| | | agesfpgiyd alfdieskvd |
| | | 721 pskawgevkr qiyvaaftvq aaaetlseva |
| Human | Breast cancer | 1 melaalcrwg lllallppga astqvctgtd mklrlpaspe |
| epidermal | Gastric cancer | thldmlrhly qgcqvvqgnl |
| growth | Lung cancer | 61 eltylptnas lsflqdiqev qgyvliahnq vrqvplqrlr |
| factor 2 | Bladder cancer | ivrgtqlfed nyalavldng |
| (HER2) | Colorectal cancer | 121 dplnnttpvt gaspgglrel qlrslteilk ggvliqrnpq |
| (NP_004439) | Esophageal cancer | lcyqdtilwk difhknnqla |
| SEQ ID NO: 102 | Ovarian cancer | 181 ltlidtnrsr achpcspmck gsrcwgesse dcqsltrtvc |
| | | aggcarckgp lptdccheqc |
| | | 241 aagctgpkhs dclaclhfnh sgicelhcpa lvtyntdtfe |
| | | smpnpegryt fgascvtacp |
| | | 301 ynylstdvgs ctlvcplhnq evtaedgtqr cekcskpcar |
| | | vcyglgmehl revravtsan |
| | | 361 iqefagckki fgslaflpes fdgdpasnta plqpeqlqvf |
| | | etleeitgyl yisawpdslp |
| | | 421 dlsvfqnlqv irgrilhnga ysltlqglgi swlglrslre |
| | | lgsglalihh nthlcfvhtv |
| | | 481 pwdqlfrnph qallhtanrp edecvgegla chqlcarghc |
| | | wgpgptqcvn csqflrgqec |
| | | 541 veecrvlqgl preyvnarhc lpchpecqpq ngsvtcfgpe |
| | | adqcvacahy kdppfcvarc |
| | | 601 psgvkpdlsy mpiwkfpdee gacqpcpinc thscvdlddk |
| | | gcpaeqrasp ltsiisavvg |
| | | 661 illvvvlgvv fgilikrrqq kirkytmrrl lqetelvepl |
| | | tpsgampnqa qmrilketel |
| | | 721 rkvkvlgsga fgtvykgiwi pdgenvkipv aikvlrents |
| | | pkankeilde ayvmagvgsp |
| | | 781 yvsrllgicl tstvqlvtql mpygclldhv renrgrlgsq |
| | | dllnwcmqia kgmsyledvr |
| | | 841 lvhrdlaarn vlvkspnhvk itdfglarll dideteyhad |
| | | ggkvpikwma lesilrrrft |
| | | 901 hqsdvwsygv tvwelmtfga kpydgipare ipdllekger |
| | | lpqppictid vymimvkcwm |
| | | 961 idsecrprfr elvsefsrma rdpqrfvviq nedlgpaspl |
| | | dstfyrslle dddmgdlvda |
| | | 1021 eeylvpqqgf fcpdpapgag gmvhhrhrss strsgggdlt |
| | | lglepseeea prsplapseg |
| | | 1081 agsdvfdgdl gmgaakglqs lpthdpsplq rysedptvpl |
| | | psetdgyvap ltcspqpeyv |
| | | 1141 nqpdvrpqpp spregplpaa rpagatlerp ktlspgkngv |
| | | vkdvfafgga venpeyltpq |
| | | 1201 ggaapqphpp pafspafdnl yywdqdpper gappstfkgt |
| | | ptaenpeylg ldvpv |
| Epithelial | Bladder cancer | 1 mappqvlafg lllaaatatf aaaqeecvce nyklavncfv |
| cell | Breast cancer | nnnrqcqcts vgaqntvics |
| adhesion | Colon cancer | 61 klaakclvmk aemngsklgr rakpegalqn ndglydpdcd |
| molecule | Esophagus cancer | esglfkakqc ngtsmcwcvn |
| (EpCAM) | Lung cancer | 121 tagvrrtdkd teitcservr tywiiielkh karekpydsk |
| (NP_002345) | Hepatocellular | slrtalqkei ttryqldpkf |
| SEQ ID NO: 103 | cancer | 181 itsilyennv itidlvqnss qktqndvdia dvayyfekdv |
| | Ovarian cancer | kgeslfhskk mdltvngeql |
| | Pancreas cancer | 241 dldpgqtliy yvdekapefs mqglkagvia vivvvviavv |
| | Prosate cancer | agivvlvisr kkrmakyeka |
| | | 301 eikemgemhr elna |
| Nestin | Prostate cancer | |
| (NP_006608) | Breast cancer | |
| | Pancreatic cancer | |
| | Ovarian cancer | |
| | Cervical cancers | |
| | Glioblastoma | |
| | Lung cancer | |
| | Adenocarcinoma | |
| | Multiple myeloma | |
| Annexin A2 | Leukemia | 1 mgrqlagcgd agkkasfkms tvheilckls legdhstpps |
| (p36) | Breast cancer | aygsvkaytn fdaerdalni |
| (NP_001002858) | Pancreatic cancer | 61 etaiktkgvd evtivniltn rsnaqrqdia fayqrrtkke |
| SEQ ID NO: 104 | Hepatocellular | lasalksals ghletvilgl |
| | cancer | 121 lktpaqydas elkasmkglg tdedslieii csrtnqelqe |
| | Colorectal cancer | inrvykemyk tdlekdiisd |
| | Squamous cell | 181 tsgdfrklmv alakgrraed gsvidyelid qdardlydag |
| | carcinomas | vkrkgtdvpk wisimtersv |
| | Multiple myeloma | 241 phlqkvfdry ksyspydmle sirkevkgdl enaflnlvqc |
| | | iqnkplyfad rlydsmkgkg |
| | | 301 trdkvlirim vsrsevdmlk irsefkrkyg kslyyyiqqd |
| | | tkgdyqkall ylcggdd |
| Tumor | Most sarcomas | 1 mllrlllawa aagptlgqdp waaepraacg psscyalfpr |
| endothelial | Brain tumors | rrtfleawra crelggdlat |
| marker 1 | | 61 prtpeeaqrv dslvgagpas rllwiglqrq arqcqlqrpl |
| (TEM 1) | | rgftwttgdq dtaftnwaqp |
| (NP_065137) | | 121 asggpcpaqr cvaleasgeh rwlegsctla vdgylcqfgf |
| SEQ ID NO: 105 | | egacpalqde agqagpavyt |
| | | 181 tpfhlvstef ewlpfgsvaa vqcqagrgas llcvkqpegg |
| | | vgwsragplc lgtgcspdng |
| | | 241 gcehecveev dghvscrcte gfrlaadgrs cedpcaqapc |
| | | eqqcepggpq gyschcrlgf |
| | | 301 rpaeddphrc vdtdecqiag vcqqmcvnyv ggfecycseg |
| | | heleadgisc spagamgaqa |
| | | 361 sqdlgdelld dgedeedede awkafnggwt empgilwmep |
| | | tqppdfalay rpsfpedrep |
| | | 421 qipypeptwp pplsaprvpy hssvlsvtrp vvvsathptl |
| | | psahqppvip athpalsrdh |
| | | 481 qipviaanyp dlpsayqpgi lsvshsaqpp ahqppmistk |
| | | ypelfpahqs pmfpdtrvag |
| | | 541 tqttthlpgi ppnhaplvtt lgaqlppqap dalvlrtqat |
| | | qlpiiptaqp sltttsrspv |
| | | 601 spahqisvpa atqpaalptl lpsqsptnqt spispthphs |
| | | kapqipredg pspklalwlp |
| | | 661 spaptaapta lgeaglaehs qrddrwllva llvptcvflv |
| | | vllalgivyc trcgphapnk |
| | | 721 ritdcyrwvi hagsksptep mpprgsltgv qtcrtsv |
| Mucin 1 (MUC1) | Ovarian cancer | 1 mtpgtqspff llllltvltv vtgsghasst pggeketsat |
| (NP_002447) | Breast cancer | qrssvpsste knalstgvsf |
| SEQ ID NO: 106 | Lung cancer | 61 fflsfhisnl qfnssledps tdyyqelqrd isemflqiyk |
| | Pancreatic cancer | qggflglsni kfrpgsvvvq |
| | Prostate | 121 ltlafregti nvhdvetqfn qykteaasry nitisdvsvs |
| | adenocarcinoma | dvpfpfsaqs gagvpgwgia |
| | Multiple myeloma | 181 llvlvcvlva laivyliala vcqcrrknyg qldifpardt |
| | | yhpmseypty hthgryvpps |
| | | 241 stdrspyekv sagnggssls ytnpavaats anl |
| Nucleolin | Gastric cancer | 1 mvklakagkn qgdpkkmapp pkeveedsed eemsedeedd |
| (NCL) | Lung cancer | ssgeevvipq kkgkkaaats |
| (NP_005372) | Colorectal cancer | 61 akkvvvsptk kvavatpakk aavtpgkkaa atpakktvtp |
| SEQ ID NO: 107 | Prostate cancer | akavttpgkk gatpgkalva |
| | Breast cancers | 121 tpgkkgaaip akgakngkna kkedsdeeed ddseedeedd |
| | Melanoma | ededededei epaamkaaaa |
| | Glioblastoma | 181 apasededde ddeddedddd deeddseeea mettpakgkk |
| | Gliomas | aakvvpvkak nvaededeee |
| | Osteosarcoma | 241 ddededdddd eddedddded deeeeeeeee epvkeapgkr |
| | Leukemia | kkemakqkaa peakkqkveg |
| | | 301 tepttafnlf vgnlnfnksa pelktgisdv fakndlavvd |
| | | vrigmtrkfg yvdfesaedl |
| | | 361 ekaleltglk vfgneiklek pkgkdskker dartllaknl |
| | | pykvtqdelk evfedaaeir |
| | | 421 lvskdgkskg iayiefktea daektfeekq gteidgrsis |
| | | lyytgekgqn qdyrggknst |
| | | 481 wsgesktlvl snlsysatee tlqevfekat fikvpqnqng |
| | | kskgyafief asfedakeal |
| | | 541 nscnkreieg rairlelqgp rgspnarsqp sktlfvkgls |
| | | edtteetlke sfdgsvrari |
| | | 601 vtdretgssk gfgfvdfnse edakaakeam edgeidgnkv |
| | | tldwakpkge ggfggrgggr |
| | | 661 ggfggrgggr ggrggfggrg rggfggrggf rggrggggdh |
| | | kpqgkktkfe |
| Endoglin | Renal cell | 1 mdrgtlplav alllascsls ptslaetvhc dlqpvgperg |
| (CD105) | carcinoma | evtyttsqvs kgcvaqapna |
| (NP_001108225) | Hepatocellular | 61 ilevhvlfle fptgpsqlel tlqaskqngt wprevllvls |
| SEQ ID NO: 108 | carcinoma | vnssvflhlq algiplhlay |
| | Gastric cancer | 121 nsslvtfqep pgvnttelps fpktqilewa aergpitsaa |
| | Prostate cancer | elndpqsill rlgqaqgsls |
| | Sarcoma | 181 fcmleasqdm grtlewrprt palvrgchle gvaghkeahi |
| | Leukemia | lrvlpghsag prtvtvkvel |
| | | 241 scapgdldav lilqgppyvs wlidanhnmq iwttgeysfk |
| | | ifpeknirgf klpdtpqgll |
| | | 301 gearmlnasi vasfvelpla sivslhassc ggrlqtspap |
| | | iqttppkdtc spellmsliq |
| | | 361 tkcaddamtl vlkkelvahl kctitgltfw dpsceaedrg |
| | | dkfvlrsays scgmqvsasm |
| | | 421 isneavvnil sssspqrkkv hclnmdslsf qlglylsphf |
| | | lqasntiepg qqsfvqvrvs |
| | | 481 psvsefllql dschldlgpe ggtveliqgr aakgncvsll |
| | | spspegdprf sfllhfytvp |
| | | 541 ipktgtlsct valrpktgsq dqevhrtvfm rlniispdls |
| | | gctskglvlp avlgitfgaf |
| | | 601 ligalltaal wyiyshtrsp skrepvvava apassessst |
| | | nhsigstqst pcstssma |
| CD24 | Breast cancer | |
| (NP_001108225), | Brain cancer | |
| CD30 | Colon cancer | |
| (NP_001234), | Gastric cancer | |
| CD44 | Lung cancer | |
| (NP_000601), | Prostate cancers | |
| CD71 | Leukemia | |
| (NP_003225), | Hodgkin's | |
| CD133 | lymphoma | |
| (NP_006008), | | |
| CD166 | | |
| (NP_001139864) | | |
| Integrin α3βv | Pancreatic cancer | |
| (NP_002195/ | Leukemia | |
| NP_002204), | Prostate cancer | |
| α2β1 | Colorectal cancers | |
| (NP_002194/ | Sarcoma | |
| NP_002202) | | |
| CXCR4 | Gliomas | 1 megissiplp llqiytsdny teemgsgdyd smkepcfree |
| (CAA12166) | Renal cell | nanfnkiflp tiysiifltg |
| SEQ ID NO: 109 | carcinoma | 61 ivgnglvilv mgyqkklrsm tdkyrlhlsv adllfvitlp |
| | Lymphoma | fwavdavanw yfgnflckav |
| | Breast cancer | 121 hviytvnlys svlilafisl drylaivhat nsqrprklla |
| | Leukemia | ekvvyvgvwi pallltipdf |
| | Gastric cancer | 181 ifanvseadd ryicdrfypn dlwvvvfqfq himvglilpg |
| | Ovarian cancer | ivilscycii isklshskgh |
| | Colorectal cancer | 241 qkrkalkttv ililaffacw lpyyigisid sfilleiikq |
| | | gcefentvhk wisitealaf |
| | | 301 fhcclnpily aflgakfkts aqhaltsvsr gsslkilskg |
| | | krgghssvst esesssfhss |
| CXCR3 | Breast cancer | 1 mvlevsdhqv lndaevaall enfsssydyg enesdsccts |
| (NP_001495) | Gastric cancer | ppcpqdfsln fdraflpaly |
| SEQ ID NO: 110 | Melanoma | 61 sllfllgllg ngavaavlls rrtalsstdt fllhlavadt |
| | Renal cell | llvltlplwa vdaavqwvfg |
| | carcinoma | 121 sglckvagal fninfyagal llacisfdry lnivhatqly |
| | | rrgpparvtl tclavwglcl |
| | | 181 lfalpdfifl sahhderlna thcqynfpqv grtalrvlql |
| | | vagfllpllv maycyahila |
| | | 241 vllvsrgqrr lramrlvvvv vvafalcwtp yhlvvlvdil |
| | | mdlgalarnc gresrvdvak |
| | | 301 svtsglgymh cclnpllyaf vgvkfrermw mlllrlgcpn |
| | | grglqrqpss srrdsswset |
| | | 361 seasysgl |
| Vascular | Ovarian cancer | |
| endothelial | Breast cancer | |
| growth | Cervical cancer | |
| factor | Lung cancers | |
| receptor | Thyroid cancer | |
| (VEGFR) | Renal cell | |
| (AAC16449) | carcinoma | |
| Platelet | Gastrointestinal | |
| derived | stromal | |
| growth | tumors | |
| factor | Leukemia | |
| receptor | Multiple myeloma | |
| (PDGF-R) | Dermatofibro- | |
| (AAA60049) | sarcoma | |
| | Melanoma | |
| | Glioblastoma | |
| Hepatocyte | Colorectal cancer | |
| growth | Lung cancer | |
| factor | Breast cancer | |
| receptor | Pancreatic cancer | |
| (HGFR/MET) | Ovarian cancer | |
| (AAI30421) | Gastric cancer | |
| | Endometrial | |
| | cancers | |
| Ephrin | Breast cancer | 1 maldylllll lasavaamee tlmdtrtata elgwtanpas |
| type-B | Prostate cancer | gweevsgyde nlntirtyqv |
| receptor 4 | Lung cancers | 61 cnvfepnqnn wllttfinrr gahriytemr ftvrdcsslp |
| (Eph64) | Mesothelioma | nvpgscketf nlyyyetdsv |
| (NP_004432) | Glioblastoma | 121 iatkksafws eapylkvdti aadesfsqvd fggrlmkvnt |
| SEQ ID NO: 111 | | evrsfgpltr ngfylafqdy |
| | | 181 gacmsllsvr vffkkcpsiv qnfavfpetm tgaestslvi |
| | | argtcipnae evdvpiklyc |
| | | 241 ngdgewmvpi grctckpgye pensvackac pagtfkasqe |
| | | aegcshcpsn srspaeaspi |
| | | 301 ctcrtgyyra dfdppevact svpsgprnvi sivnetsiil |
| | | ewhppretgg rddvtyniic |
| | | 361 kkcradrrsc srcddnvefv prqlgltecr vsisslwaht |
| | | pytfdiqain gvsskspfpp |
| | | 421 qhvsvnittn qaapstvpim hqvsatmrsi tlswpqpeqp |
| | | ngiildyeir yyekehnefn |
| | | 481 ssmarsqtnt aridglrpgm vyvvqvrart vagygkfsgk |
| | | mcfqtltddd ykselreqlp |
| | | 541 liagsaaagv vfvvslvais ivcsrkrays keavysdklq |
| | | hystgrgspg mkiyidpfty |
| | | 601 edpneavref akeidvsfvk ieevigagef gevykgrlkl |
| | | pgkreiyvai ktlkagysek |
| | | 661 qrrdflseas imgqfdhpni irlegvvtks rpvmiitefm |
| | | engaldsflr qndgqftviq |
| | | 721 lvgmlrgiaa gmkylaemny vhrdlaarni lvnsnlvckv |
| | | sdfglsrylq ddtsdptyts |
| | | 781 slggkipvrw tapeaiayrk ftsasdvwsy givmwevmsf |
| | | gerpywdmsn qdvinaieqd |
| | | 841 yrlpppmdcp aalhqlmldc wqkdrnsrpr faeivntldk |
| | | mirnpaslkt vatitavpsq |
| | | 901 plldrsipdf tafttvddwl saikmvqyrd sfltagftsl |
| | | qlvtgmtsed llrigitlag |
| | | 961 hqkkilnsih smrvgisgsp tama |
| IL-6 receptor | Colon cancer | 1 mlavgcalla allaapgaal aprrcpaqev argvltslpg |
| (CD126) | Ovarian cancer | dsvtltcpgv epednatvhw |
| (NP_000556) | Pancreatic cancers | 61 vlrkpaagsh psrwagmgrr lllrsvqlhd sgnyscyrag |
| SEQ ID NO: 112 | Multiple myeloma | rpagtvhllv dvppeepqls |
| | Hepatocellular | 121 cfrksplsnv vcewgprstp slttkavllv rkfqnspaed |
| | carcinoma | fqepcqysge sqkfscqlav |
| | | 181 pegdssfyiv smcvassvgs kfsktqtfqg cgilqpdppa |
| | | nitvtavarn prwlsvtwqd |
| | | 241 phswnssfyr lrfelryrae rsktfttwmv kdlqhhcvih |
| | | dawsglrhvv qlraqeefgq |
| | | 301 gewsewspea mgtpwtesrs ppaenevstp mqalttnkdd |
| | | dnilfrdsan atslpvqdss |
| | | 361 svplptflva ggslafgtll ciaivlrfkk twklralkeg |
| | | ktsmhppysl gqlvperprp |
| | | 421 tpvlvplisp pvspsslgsd ntsshnrpda rdprspydis |
| | | ntdyffpr |
| Transforming | Breast cancer | 1 meaavaaprp rllllvlaaa aaaaaallpg atalqcfchl |
| growth | Lung cancer | ctkdnftcvt dglcfvsvte |
| factor β | Colon cancer | 61 ttdkvihnsm ciaeidlipr drpfvcapss ktgsvtttyc |
| receptor | Prostate cancer | cnqdhcnkle lpttvksspg |
| (TGF-β R) | Pancreatic cancer | 121 lgpvelaavi agpvcfvcis lmlmvyichn rtvihhrvpn |
| (ABD46753) | Ductal | eedpsldrpf isegttlkdl |
| SEQ ID NO: 113 | adenocarcinoma | 181 iydmttsgsg sglpllvqrt iartivlqes igkgrfgevw |
| | Hepatocellular | rgkwrgeeva vkifssreer |
| | carcinoma | 241 swfreaeiyq tvmlrhenil gfiaadnkdn gtwtqlwlvs |
| | | dyhehgslfd ylnrytvtve |
| | | 301 gmiklalsta sglahlhmei vgtqgkpaia hrdlksknil |
| | | vkkngtccia dlglavrhds |
| | | 361 atdtidiapn hrvgtkryma pevlddsinm khfesfkrad |
| | | iyamglvfwe iarrcsiggi |
| | | 421 hedyqlpyyd lvpsdpsvee mrkvvceqkl rpnipnrwqs |
| | | cealrvmaki mrecwyanga |
| | | 481 arltalrikk tlsqlsqqeg ikm |
| Transco- | Breast cancer | 1 mgrgllrglw plhivlwtri astipphvqk svnndmivtd |
| balamin II | Pancreatic cancer | nngavkfpql ckfcdvrfst |
| receptor | Ovarian cancer | 61 cdnqkscmsn csitsicekp qevcvavwrk ndenitletv |
| (TCII-R) | Lung cancer | chdpklpyhd filedaaspk |
| (ABG65632) | Renal | 121 cimkekkkpg etffmcscss decndniifs eeyntsnpdl |
| SEQ ID NO: 114 | adenocarcinoma | llvifqvtgi sllpplgvai |
| | Ewing's sarcoma | 181 sviiifycyr vnrqqklsst wetgktrklm efsehcaiil |
| | Glioblastoma | eddrsdisst canninhnte |
| | Leukemia | 241 llpieldtlv gkgrfaevyk aklkqntseq fetvavkifp |
| | Burkitt's | yeeyaswkte kdifsdinlk |
| | lymphoma | 301 henilqflta eerktelgkq ywlitafhak gnlqeyltrh |
| | | viswedlrkl gsslargiah |
| | | 361 lhsdhtpcgr pkmpivhrdl kssnilvknd ltcclcdfgl |
| | | slrldptlsv ddlansgqvg |
| | | 421 tarymapevl esrmnlenve sfkqtdvysm alvlwemtsr |
| | | cnavgevkdy eppfgskvre |
| | | 481 hpcvesmkdn vlrdrgrpei psfwlnhqgi qmvcetltec |
| | | wdhdpearlt aqcvaerfse |
| | | 541 lehldrlsgr scseekiped gslnttk |
| Protein | T-cell acute | |
| tyrosine | lymphoblastic | |
| kinase 7 | leukemia | |
| (PTK7) | Lung cancer | |
| (AAL39062) | Gastric cancer | |
| SEQ ID NO: 115 | Colon carcinoma | |

In some embodiments, the pharmaceutical composition is administered in a liposomal formulation. In some embodiments, toxicity to other cancer therapy is prevented or reduced, such that toxic doses are tolerated in the subject. In some embodiments, the pharmaceutical composition comprises: (a) an active strand nucleotide sequence comprising a sequence at least 80% identical to a mature miRNA; and (b) a separate passenger strand that is at least 60% complementary to the active strand. In some embodiments, the passenger strand of the pharmaceutical composition comprises a 5′ terminal cap. In some embodiments, the 5′ terminal cap is a lower alkylamine. In some embodiments, the 5′ terminal cap is NH2-(CH2)6-0-. In some embodiments, the miRNA domain of the nuclei c acid comprises: miR-26a or a nucleotide sequence with at least about 70%, 75%, 80%. 85%, 90%. 91%, 92%, 93%, 94%, 95%, 96%, 97%, 98% or 99% homologous to the SEQ ID NO:1 and the domain is from about 15 to about 40 nucleotides.

In some embodiments, administration of the effective amount of pharmaceutical composition disclosed herein is not limited to any particular delivery system and includes, without limitation, parenteral (including subcutaneous, intravenous, intramedullary, intraarticular, intramuscular, or intraperitoneal injection), rectal, topical, transdermal, muscoal or oral (for example, in capsules, suspensions, or tablets) administration. In some embodiments, administration to a subject in need thereof occurs in a single dose or in repeat administrations, and in any of a variety of physiologically acceptable salt forms, or with an acceptable pharmaceutical carrier or additive as part of a pharmaceutical composition. In some embodiments, any suitable and physiological acceptable salt forms or standard pharmaceutical formulation techniques, dosages, and excipients are utilized.

In some embodiments, effective dosages achieved in one animal are extrapolated for use in another animal, including humans, using conversion factors known in the art.

In some embodiments, the pharmaceutical composition dosing amount or schedule follows clinically approved, or experimental, guidelines. In some embodiments, the dose of the pharmaceutical composition is about 1, 2, 3, 4, 5, 6, 7, 8, 9, 10, 15, 20, 25, 30, 40, 50, 60, 70, 80, 90, 100, 125, 150, 175, 200, 225, 250 or about 500 mg/kg of the subject per day.

In some embodiments the pharmaceutical composition is administered to the individual in about 1, 2, 3, 4, 5 daily doses over 5 consecutive or non-consecutive days. In some embodiments, the oligonucleotide is administered to the individual in about 1, 2, 3, 4, 5, 6, or 7 daily doses over a single week (7 days). In some embodiments, the pharmaceutical composition is administered to the individual in about 1.2, 3, 4, 5, 6, 7, 8, 9, 10, 11, 12, 13, or 14 daily doses over 14 days. In some embodiments, the pharmaceutical composition is administered to the individual in 1, 2, 3, 4, 5, 6, 7, 8, 9, 10, 11, 12, 13, 14, 15, 16, 17, 18, 19, 20, or 21 daily doses over 21 days. In some embodiments, the pharmaceutical composition is administered to the individual in 1, 2, 3, 4, 5, 6, 7, 8, 9, 10, 11, 12, 13, 14, 15, 16, 17, 18, 19, 20, 21, 22, 23, 24, 25, 26, 27, or 28 daily doses over 28 days.

In some embodiments, the pharmaceutical composition is provided about twice a week of a 21 or a 28 day cycle. In particular embodiments, the pharmaceutical composition is provided on about days 1, 4, 8, 11, 15 and 18 of a 21 day or 28 day cycle.

In some embodiments the pharmaceutical composition is administered for: about 2 weeks (total 14 days); about 1 week with 1 week off (total 14 days); about 3 consecutive weeks (total 21 days); about 2 weeks with 1 week off (total 21 days); about 1 week with 2 weeks off (total 21 days); about 4 consecutive weeks (total 28 days); about 3 consecutive weeks with 1 week off (total 28 days); about 2 weeks with 2 weeks off (total 28 days); about 1 week with 3 consecutive weeks off (total 28 days).

In some embodiments the pharmaceutical composition disclosed herein is administered on day 1 of a 7, 14, 21 or 28 day cycle; administered on days 1 and 15 of a 21 or 28 day cycle; administered on days 1, 8, and 15 of a 21 or 28 day cycle; or administered on days 1, 2, 8, and 15 of a 21 or 28 day cycle. In some embodiments, the pharmaceutical composition is administered once every 1, 2, 3, 4, 5, 6, 7, or 8 weeks. In some embodiments, the pharmaceutical composition (and optionally a combination therapy) is administered for 1, 2, 3, 4, 5, 6, 7, 8, 9, 10, 11, or 12 cycles.

The disclosure also relates to a method of increasing the sensitivity of a cancer cell to one or more chemotherapeutic agents, the method comprising contacting a cancer cell with one or more pharmaceutical compositions disclosed herein.

The disclosure also relates to a method of increasing the sensitivity of a cancer cell in a subject in need thereof to one or more chemotherapeutic agents, the method comprising administering to a subject diagnosed with cancer or suspected of having cancer one or more pharmaceutical compositions disclosed herein. In some embodiments, the cancer in the subject is not responsive to chemotherapeutic agents.

The disclosure also relates to a method of destroying a cancer cell, the method comprising contacting a cancer stem cell with one or more pharmaceutical compositions disclosed herein.

The disclosure also relates to a method of treating or preventing growth and/or proliferation of a cancer cell in a subject diagnosed with or suspected of having cancer, the method comprising administering to a subject diagnosed with cancer or suspected of cancer one or more pharmaceutical compositions disclosed herein.

The disclosure also relates to a method of treating or preventing cancer expressing KIT or any other aptamer targeting protein disclosed herein in a subject diagnosed with or suspected of having a cancer, the method comprising administering to a subject diagnosed with a cancer overexpressing one or more pharmaceutical compositions disclosed herein.

According to one aspect, the disclosure relates to a method of altering a eukaryotic cell comprising: transfecting the eukaryotic cell with a nucleic acid disclosed herein with a miRNA sequence sufficiently complementary to mRNA expressed by the cell such that the miRNA domain hybridizes to the mRNA target sequence of the eukaryotic cell and degrades the mRNA, thereby reducing expression of the one or plurality of mRNA target sequences. According to one aspect, the eukaryotic cell is a yeast cell, a plant cell or a mammalian cell. According to one aspect, the nucleic acid disclosed herein comprises from about 10 to about 250 nucleotides. According to one aspect, the nucleic acid disclosed herein comprises from about 20 to about 100 nucleotides. IN some embodiments, the nucleic acid sequence comprises about two domains, an aptamer domain and a miRNA domain and each domain is no greater than about 35 nucleotide in length.

According to one aspect, a method of altering a human cell is provided including transfecting the human cell with a nucleic acid disclosed herein with a miRNA sequence sufficiently complementary to mRNA of the cell such that the miRNA domain hybridizes to the mRNA target sequence of the human cell and degrades the mRNA, thereby reducing expression of the one or plurality of mRNA target sequences. According to one aspect, the RNA includes between about 10 to about 250 nucleotides. According to one aspect, the RNA includes between about 20 to about 100 nucleotides. The step of transfecting a nucleic acid encoding an RNA may be added to any method disclosed herein so that there is sequential or concurrent transfection of one or a plurality of vectors that carry one or more expressible genes operably linked to a regulatory sequence active in the target cell. In some embodiments, the step of administering one or more of the chemotherapeutic agents.

The disclosure relates to a composition comprising a cell with any one or combination of nucleic acid sequences disclosed herein. In some embodiments, the cell is a plant, insect or mammalian cell. In some embodiments, the cell is a eukaryotic cell or a prokaryotic cell. The cell may be isolated from the body, a component of a culture system, or part of an organism in an in vivo based assay or therapy. The construct(s) containing the nucleic acids can be delivered to a cell using, for example, biolistic bombardment, electrostatic potential or through transformation permeability reagents (reagents known to increase the permeability of the cell wall or cell membrane). Alternatively, the system components can be delivered using Agrobacterium-mediated transformation, insect vectors, grafting, or DNA abrasion, according to methods that are standard in the art, including those described herein. In some embodiments, the system components can be delivered in a viral vector (e.g., a vector from a DNA virus such as, without limitation, geminivirus, AAV, adenovirus, lentiviral strains attenuated for human use, bean yellow dwarf virus, wheat dwarf virus, tomato leaf curl virus, maize streak virus, tobacco leaf curl virus, tomato golden mosaic virus, or Faba bean necrotic yellow virus, or a vector from an RNA virus such as, without limitation, a tobravirus (e.g., tobacco rattle virus, tobacco mosaic virus), potato virus X, or barley stripe mosaic virus.

The disclosure relates to a method of inhibiting myelosuppression in a subject being treated for cancer by administering one or a plurality of nucleic acid sequences to the subject in need thereof in a therapeutically effective amount, the nucleic acid sequence comprising one or a portion of mi-26a miRNA or a salt thereof (or a variant at least 70%, 80%, 85%, 90%. 91%, 92%, 93%, 94%, 95%, 96%, 97%, 98% or 99% homolgous to mi-26a) in its miRNA domain. In some embodiments, the miRNA domain is modified by a glycerol derivative and/or cholesterol. In some embodiments, the nucleic acid sequence comprises a cholesterol molecule on its 3′ terminus and is capable of hybridizing to a complementary mRNA in a cell of the subject, thereby preventing Bak1 related apoptosis.

In some embodiments, the methods of the disclosure relate to a method of preventing Bak1-induced apoptosis by administering to a subject comprising a cell with a dysfunctional apoptosis cycle a therapeutically effective amount of one, two or more pharmaceutical compositions disclosed herein. The nucleic acid disclosed here prevents dyregulated apoptosis of the breast cancer cell or any metastatic cancer derived from a breast cancer cell. The disclosure relates to a method of inhibiting myelosuppression in a subject being treated for cancer by administering one or a plurality of nucleic acid sequences to the subject in need thereof in a therapeutically effective amount, the nucleic acid sequence comprising one or a portion of the following miRNA sequences:

TABLE F
miR-200a
Mouse (SEQ ID NO: 116)

| | |
|---|---|
| 1 | ctgggcctct gtgggcatct taccggacag tgctggattt cttggcttga ctctaacact |
| 61 | gtctggtaac gatgttcaaa ggtgacccac |

Human (SEQ ID NO: 117)

| | |
|---|---|
| 1 | ccgggcccct gtgagcatct taccggacag tgctggattt cccagcttga ctctaacact |
| 61 | gtctggtaac gatgttcaaa ggtgacccgc |

miR-200b
Mouse (SEQ ID NO: 118)

| | |
|---|---|
| 1 | gccgtggcca tcttactggg cagcattgga tagtgtctga tctctaatac tgcctggtaa |
| 61 | tgatgacggc |

Human (SEQ ID NO: 119)

| | |
|---|---|
| 1 | ccagctcggg cagccgtggc catcttactg ggcagcattg gatggagtca ggtctctaat |
| 61 | actgcctggt aatgatgacg gcggagccct gcacg |

miR-200c
Mouse (SEQ ID NO: 120)

| | |
|---|---|
| 1 | ccctcgtctt acccagcagt gtttgggtgc tggttgggag tctctaatac tgccgggtaa |
| 61 | tgatggagg |

Human (SEQ ID NO: 121)

| | |
|---|---|
| 1 | ccctcgtctt acccagcagt gtttgggtgc ggttgggagt ctctaatact gccgggtaat |
| 61 | gatggagg |

miR-141
Mouse (SEQ ID NO: 122)

| | |
|---|---|
| 1 | gggtccatct tccagtgcag tgttggatgg ttgaagtatg aagctcctaa cactgtctgg |
| 61 | taaagatggc cc |

Human (SEQ ID NO: 123)

| | |
|---|---|
| 1 | cggccggccc tgggtccatc ttccagtaca gtgttggatg gtctaattgt gaagctccta |
| 61 | acactgtctg gtaaagatgg ctcccgggtg ggttc |

miR-429
Mouse (SEQ ID NO: 124)

| | |
|---|---|
| 1 | cctgctgatg gatgtcttac cagacatggt tagatctgga tgcatctgtc taatactgtc |
| 61 | tggtaatgcc gtccatccac ggc |

Human (SEQ ID NO: 125)

| | |
|---|---|
| 1 | cgccggccga tgggcgtctt accagacatg gttagacctg gccctctgtc taatactgtc |
| 61 | tggtaaaacc gtccatccgc tgc |

miR-506
Human (SEQ ID NO: 126)

| | |
|---|---|
| 1 | gccaccacca tcagccatac tatgtgtagt gccttattca ggaaggtgtt acttaataga |
| 61 | ttaatatttg taaggcaccc ttctgagtag agtaatgtgc aacatggaca acatttgtgg |
| 121 | tggc |

miR-205
Mouse (SEQ ID NO: 127)

| | |
|---|---|
| 1 | ctcttgtcct tcattccacc ggagtctgtc ttatgccaac cagatttcag tggagtgaag |
| 61 | ctcaggag |

Human (SEQ ID NO: 128)

| | |
|---|---|
| 1 | aaagatcctc agacaatcca tgtgcttctc ttgtccttca ttccaccgga gtctgtctca |
| 61 | tacccaacca gatttcagtg gagtgaagtt caggaggcat ggagctgaca |

miR-21
Mouse (SEQ ID NO: 129)

| | |
|---|---|
| 1 | tgtaccacct tgtcggatag cttatcagac tgatgttgac tgttgaatct catggcaaca |
| 61 | gcagtcgatg ggctgtctga cattttggta tc |

Human (SEQ ID NO: 130)

| | |
|---|---|
| 1 | tgtcgggtag cttatcagac tgatgttgac tgttgaatct catggcaaca ccagtcgatg |
| 61 | ggctgtctga ca |

miR-494
Mouse (SEQ ID NO: 131)

| | |
|---|---|
| 1 | ttgatacttg aaggagaggt tgtccgtgtt gtcttctctt tatttatgat gaaacataca |
| 61 | cgggaaacct cttttttagt atcaa |

Human (SEQ ID NO: 132)

| | |
|---|---|
| 1 | gatactcgaa ggagaggttg tccgtgttgt cttctcttta tttatgatga aacatacacg |
| 61 | ggaaacctct tttttagtat c |

miR-1973
Human (SEQ ID NO: 133)

| | |
|---|---|
| 1 | tatgttcaac ggccatggta tcctgaccgt gcaaaggtag cata |

miR-155
Mouse (SEQ ID NO: 134)

| | |
|---|---|
| 1 | ctgttaatgc taattgtgat aggggttttg gcctctgact gactcctacc tgttagcatt |
| 61 | aacag |

Human (SEQ ID NO: 135)

| | |
|---|---|
| 1 | ctgttaatgc taatcgtgat aggggttttt gcctccaact gactcctaca tattagcatt |
| 61 | aacag |

All variants and/or functional fragments that are at least 70%, 80%, 85%, 90%, 91%, 92%, 93%, 94%, 95%, 96%, 97%, 98% or 99% homolgous to the sequences of Table F are also provided as possible mi-RNA domains contemplated by the nucleic acid sequences or salts disclosed herein.

Methods of vaccinating a subject with a pharmaceutical composition comprising a therapeutically effective amount of a bispecific aptamer domain containing nucleic acid sequences or salts or variants thereof are also provided, wherein such methods comprise administering to a subject in need thereof a therapeutically effective amount of a bispecific aptamer domain containing nucleic acid sequences or salts or variants thereof.

Methods of administration for any method include administration of the composition or pharmaceutical compositions to the subject is accomplished by intradermally, intramucosally, subcutaneously, sublingually, orally, intravaginally, intramuscularly, intracavernously, intraocularly, intranasally, into a sinus, intrarectally, gastrointestinally, intraductally, intrathecally, subdurally, extradurally, intraventricular, intrapulmonary, into an abscess, intra articularly, into a bursa, subpericardially, into an axilla, intrauterinely, into the pleural space, intraperitoneally, or transmucosally.

### Kits

In some embodiments, kits in accordance with the present disclosure may be used to treat or prevent development of a cancer in a subject. In some embodiments, the kits comprise a container comprising one or a plurality of pharmaceutical compositions comprising the nucleic acids, compositions described herein and, optionally, a device used to administer the one or more pharmaceutical compositions. Any nucleic acid, composition, or component thereof disclosed may be arranged in a kit either individually or in combination with any other nucleic acid, composition, or component thereof. The disclosure provides a kit to perform any of the methods described herein. In some embodiments, the kit comprises at least one container comprising a therapeutically effective amount of one or a plurality of oligonucleotides comprising an aptamer domain capable of targeting an apatemer targeting domain on a cell of a subject. In some embodiments, the kit comprises at least one container comprising any of the polypeptides or functional fragments described herein. In some embodiments, the polypeptides are in solution (such as a buffer with adequate pH and/or other necessary additive to minimize degradation of the polypeptides during prolonged storage). In some embodiments, the polypeptides or oligonucleotides are lyophilized for the purposes of resuspension after prolonged storage. In some embodiments, the kit comprises: at least one container comprising one or a plurality of polypeptides comprising or functional fragments disclosed herein and/or oligonucleotides disclosed herein. In some embodiments, the kit optionally comprises instructions to perform any or all steps of any method described herein.

The kit may contain two or more containers, packs, or dispensers together with instructions for preparation of an array. In some embodiments, the kit comprises at least one container comprising the oligonucleotides described herein and a second container comprising a means for maintenance, use, and/or storage of the oligonucleotides such as storage buffer. In some embodiments, the kit comprises a composition comprising any polypeptide disclosed herein in solution or lyophilized or dried and accompanied by a rehydration mixture. In some embodiments, the polypeptides and rehydration mixture may be in one or more additional containers.

The compositions included in the kit may be supplied in containers of any sort such that the shelf-life of the different components are preserved, and are not adsorbed or altered by the materials of the container. For example, suitable containers include simple bottles that may be fabricated from glass, organic polymers, such as polycarbonate, polystyrene, polypropylene, polyethylene, ceramic, metal or any other material typically employed to hold reagents or food; envelopes, that may consist of foil-lined interiors, such as aluminum or an alloy. Other containers include test tubes, vials, flasks, and syringes. The containers may have two compartments that are separated by a readily removable membrane that upon removal permits the components of the compositions to mix. Removable membranes may be glass, plastic, rubber, or other inert material.

Kits may also be supplied with instructional materials. Instructions may be printed on paper or other substrates, and/or may be supplied as an electronic-readable medium, such as a floppy disc, CD-ROM, DVD-ROM, zip disc, videotape, audio tape, or other readable memory storage device. Detailed instructions may not be physically associated with the kit; instead, a user may be directed to an internet web site specified by the manufacturer or distributor of the kit, or supplied as electronic mail.

The disclosure also provides a kit comprising: a nucleic acid sequence disclosed herein; and a vector comprising one or plurality of nucleic acid sequences disclosed herein and a syringe and/or needle. In some embodiments, the kit further comprises at least one of the following: one or a plurality of eukaryotic cells comprising regulatory protein capable of trans-activation of the regulatory element, cell growth media, a volume of fluorescent stain or dye, and a set of instructions, optionally accessible remotely through an electronic medium.

Any and all journal articles, patent applications, issued patents, or other cited references disclosed herein are incorporated by reference in their respective entireties.

### REFERENCES

1. Metcalf D. The Molecular Control of Blood Cells. Cambridge, Mass.: Harvard Univ. Press; 1988.
2. Daniel D, Crawford J. Myelotoxicity from chemotherapy. Semin Oncol 2006; 33:74-85
3. Lyman G H, Dale D C, Crawford J. Incidence and predictors of low dose-intensity in adjuvant breast cancer chemotherapy: a nationwide study of community practices. J Clin Oncol 2003; 21:4524-31
4. Li L, Clevers H. Coexistence of quiescent and active adult stem cells in mammals. Science 2010; 327:542-5
5. Wilson A, Laurenti E, Oser G, van der Wath R C, et al. Hematopoietic stem cells reversibly switch from dormancy to self-renewal during homeostasis and repair. Cell 2008; 135:1118-29
6. Foudi A, Hochedlinger K, Van Buren D, Schindler J W, et al. Analysis of histone 2B-GFP retention reveals slowly cycling hematopoietic stem cells. Nat Biotechnol 2009; 27:84-90
7. Trumpp A, Essers M, Wilson A. Awakening dormant haematopoietic stem cells. Nat Rev Immunol 2010; 10:201-9
8. Wilson A, Laurenti I, Trumpp A. Balancing dormant and self-renewing hematopoietic stem cells. Curr Opin Genet Dev 2009; 19:461-8
9. Jaiswal S, Jamieson C H. Pang W W, Park C Y, et al. CD47 is upregulated on circulating hematopoietic stem cells and leukemia cells to avoid phagocytosis. Cell 2009; 138:271-85
10. Radley J M, Scurfield G. Effects of 5-fluorouracil on mouse bone marrow. Br J Haematol 1979; 43:341-51
11. Van Zant G. Studies of hematopoietic stem cells spared by 5-fluorouracil. J Exp Med 1984; 159:679-90
12. NG T, RP G. Hematopoiesis: Long-Term Effects of Chemotherapy and Radiation. New York, N.Y.: Marcel Dekker; 1988.
13. Zhou L, Seo K H, He H Z, Pacholczyk R, Meng D M, Li C G, et al. Tie2cre-induced inactivation of the miRNA-processing enzyme Dicer disrupts invariant NKT cell development. Proc Natl Acad Sci USA 2009; 106:10266-71
14. Guo S, Lu J, Schlanger R, Zhang H, Wang J Y, Fox M C, et al. MicroRNA miR-125a controls hematopoietic stem cell number. Proc Natl Acad Sci USA 2010; 107:14229-34
15. Ji J, Shi J, Budhu A, Yu Z, Forgues M, Roessler S. et al. MicroRNA expression, survival, and response to interferon in liver cancer. N Engl J Med 2009; 361:1437-47
16. Croce C M, Calin G A. miRNAs, cancer, and stem cell division. Cell 2005; 122:6-7
17. Calin G A, Dumitru C D, Shimizu M. Bichi R, Zupo S, Noch E, et al. Frequent deletions and down-regulation of micro-RNA genes miR15 and miR16 at 13q14 in chronic lymphocytic leukemia. Proc Natl Acad Sci USA 2002; 99:15524-9
18. Esquela-Kerscher A, Slack F J. Oncomirs-microRNAs with a role in cancer. Nat Rev Cancer 2006; 6:259-69
19. Burnett J C, Rossi J J. RNA-based therapeutics: current progress and future prospects. Chem Biol 2012; 19:60-71
20. Rovero S, Amici A, Di Carlo E, Bei R, Nanni P. Quaglino E, et al. DNA vaccination against rat her-2/Neu p185 more effectively inhibits carcinogenesis than transplantable carcinomas in transgenic BALB/c mice. J Immunol 2000; 165:5133-42
21. Zhao N, Pei S N, Qi J, Zeng Z, Iyer S P, Lin P, et al. Oligonucleotide aptamer-drug conjugates for targeted therapy of acute myeloid leukemia. Biomaterials 2015; 67:42-51
22. Meyer S, Maufort J P, Nie J, Stewart R. McIntosh B E, Conti L R, et al. Development of an efficient targeted cell-SELEX procedure for DNA aptamer reagents. PLoS One 2013; 8:e71798
23. Gao S, Dagnaes-Hansen F, et al. The effect of chemical modification and nanoparticle formulation on stability and biodistribution of siRNA in mice. Mol Ther 2009; 17:1225-33
24. Soutschek J, Akine A, Bramlage B, Charisse K, Constien R, et al. Therapeutic silencing of an endogenous gene by systemic administration of modified siRNAs. Nature 2004; 432:173-8
25. Wilson C. Keefe A D. Building oligonucleotide therapeutics using non-natural chemistries. Curr Opin Chem Biol 2006; 10:607-14
26. Orava E W, Cicmil N, Gariepy J. Delivering cargoes into cancer cells using DNA aptamers targeting internalized surface portals. Biochim Biophys Acta 2010; 1798:2190-200
27. Bramsen J B, Laursen M B, Damgaard C K, Lena S W, et al. Improved silencing properties using small internally segmented interfering RNAs. Nucleic Acids Res 2007; 35:5886-97
28. Tang Z, Shangguan D, Wang K, Shi H, Sefah K, et al. Selection of aptamers for molecular recognition and characterization of cancer cells. Anal Chem 2007; 79:4900-7
29. Liu P, Tang H, Chen 13, He Z, et al. miR-26a suppresses tumour proliferation and metastasis by targeting metadherin in triple negative breast cancer. Cancer Lett 2015; 357:384-92
30. Zhang B, et al. Pathologically decreased miR-26a antagonizes apoptosis and facilitates carcinogenesis by targeting MTDH and EZH2 in breast cancer. Carcinogenesis 2011; 32:2-9
31. McNiece I K, Briddell R A. Stem cell factor. J Leukoc Biol 1995; 58:14-22
32. Haraguchi T, Ozaki Y, Iba H. Vectors expressing efficient RNA decoys achieve the long-term suppression of specific microRNA activity in mammalian cells. Nucleic Acids Res 2009; 37:e43
33. Russell E S, Neufeld E F, Higgins C T. Comparison of normal blood picture of young adults from 18 inbred strains of mice. Proc Soc Exp Biol Med 1951; 78:761-6
34. von Minckwitz G, Schneeweiss A, Loibl S, Salat C, Denkert C, Rezai M, et al. Neoadjuvant carboplatin in patients with triple-negative and HER2-positive early breast cancer (GeparSixto; GBG 66): a randomised phase 2 trial. Lancet Oncol 2014; 15:747-56
35. Gao J, Li L, Wu M, Liu M, Xie X, Guo J, et al. MiR-26a inhibits proliferation and migration of breast cancer through repression of MCL-1. PLoS One 2013; 8:e65138
36. Dang X, Ma A, Yang L, Hu H, Zhu B, Shang D, et al. MicroRNA-26a regulates tumorigenic properties of EZH2 in human lung carcinoma cells. Cancer Genet 2012; 205:113-23
37. Deng J, He M, Chen L, Chen C, Zheng J, Cai Z. The loss of miR-26a-mediated post-transcriptional regulation of cyclin E2 in pancreatic cancer cell proliferation and decreased patient survival. PLoS One 2013; 8:e76450
38. Fu X, Meng Z, Liang W, Tian Y, Wang X, Han W, et al. miR-26a enhances miRNA biogenesis by targeting Lin28B and Zcchc11 to suppress tumor growth and metastasis.

Oncogene 2014; 33:4296-306
39. Chau W K, Ip C K, Mak A S, Lai H C, Wong A S. c-Kit mediates chemoresistance and tumor-initiating capacity of ovarian cancer cells through activation of Wnt/beta-catenin-ATP-binding cassette G2 signaling. Oncogene 2013; 32:2767-81
40. Di Lorenzo G, Autorino R, D'Armiento F P, Mignogna C, De Laurentiis M, et al. Expression of proto-oncogene c-kit in high risk prostate cancer. Eur J Surg Oncol 2004; 30:987-92
41. Hirota S, Isozaki K, Moriyama Y, Hashimoto K, Nishida T, Ishiguro S, et al. Gain-of-function mutations of c-kit in human gastrointestinal stromal tumors. Science 1998; 279:577-80
42. Plummer H, 3rd, Catlett J. Leftwich J, et al. c-myc expression correlates with suppression of c-kit protooncogene expression in small cell lung cancer cell lines. Cancer Res 1993; 53:4337-42
43. Puputti M, Tynninen O, Sihto H, Blom T, Maenpaa H, Isola J, et al. Amplification of KIT, PDGFRA, VEGFR2, and EGFR in gliomas. Mol Cancer Res 2006; 4:927-34
44. Wang C, Curtis J E. Geissler E N, McCulloch E A, Minden M D. The expression of the proto-oncogene C-kit in the blast cells of acute myeloblastic leukemia. Leukemia 1989; 3:699-702
45. Zhang L, Smyrk T C, Oliveira A M, Lohse C M, Zhang S, Johnson M R. et al. KIT is an independent prognostic marker for pancreatic endocrine tumors: a finding derived from analysis of islet cell differentiation markers. Am J Surg Pathol 2009; 33:1562-9
46. Huse J T, Brennan C, et al The PTEN-regulating microRNA miR-26a is amplified in high-grade glioma and facilitates gliomagenesis in vivo. Genes Dev 2009; 23:1327-37
47. Salvatori B, Iosue I, et al. The microRNA-26a target E2F7 sustains cell proliferation and inhibits monocytic differentiation of acute myeloid leukemia cells. Cell Death Dis 2012; 3:e413
48. Shen W, Song M, Liu J, Qiu G, Li T, Hu Y, et al. MiR-26a promotes ovarian cancer proliferation and tumorigenesis. PLoS One 2014; 9:e86871
49. Kota J, Chivukula R R, O'Donnell K A, Wentzel E A, et al. Therapeutic microRNA delivery suppresses tumorigenesis in a murine liver cancer model. Cell 2009; 137:1005-17
50. Sun H I, Zhu X, Lu P Y, Rosato R R, Tan W, Zu Y. Oligonucleotide aptamers: new tools for targeted cancer therapy. Mol Ther Nucleic Acids 2014; 3:e182

Representative Sequences from Table D and E. All amino acid sequences encoded by these nucleic acid sequences are also contemplated by this disclosure as well as plasmid sequences comprising any one or plurality of expressible nucleic acid sequences that are at least 70%, 80%, 85%, 90%, 95%, 96%, 97%, 98%, 99% or 100% homologous to the sequences below. Amino acid variants and full-length protein sequences are contemplated by the disclosure and can be considered payloads for this disclosure in addition to the nucleic acid sequences.

| SERP2463 | |
|---|---|
| (SEQ ID NO: 136) | |
| 1 | atgaaagatg ttatttatgt agaaaatcat tactttgtta ctgcgaagga aaatagcatt |
| 61 | aaatttagaa atgtcataga taaaagtgaa aaattttatt tgtttgaaga aattgaagct |
| 121 | attatttttg atcattataa aagctatttt tcacataaat tggtaattaa atgtatagag |
| 181 | aatgatattg cgattatatt ttgtgacaaa aagcattctc cagttacaca gctcatttct |
| 241 | tcatatggca tggttaatcg tcttaaaaga attcaaagtc aatttcagtt atcgggaaga |
| 301 | acaaaagata gaatttggaa aaagattgtt ataaataaaa tttttaacca aacacgatgt |
| 361 | cttgaaaata atttacataa tgaaaatgtt aagctgatgt taggttttgc aaaagaagtg |
| 421 | agttcaggag acaaaagtaa taaagaagca catgctactc gtatttattt taaagattta |
| 481 | tttggcaaac aatttaaacg tggacgctat aatgatgtta tcaattcagg attgaattat |
| 541 | ggttattcaa ttctcagatc ttttatcaac aaagagttag ctattcatgg atttgaaatg |
| 601 | agcctaggta ttaaacatca gtcaaaagaa aatccattta atttagcaga tgatatcatt |
| 661 | gaagtttttc gtccttttgt agataatata gtgtacgaga tagtttttaa gaaaaatatt |
| 721 | gatacatttg atataaacga aaagaaatta ttattaaatg ttttgtatga aaggtgcatt |
| 781 | atagataaaa aagtagtgcg attacttgat agtgtgaaga tagttattca atcgctaatt |
| 841 | agatgttatg aagaaaatac acctacttat ttattactac ctaagatgat agaggtggga |
| 901 | aactaa |
| SPy1047 | |
| (SEQ ID NO: 137) | |
| 1 | atggctggtt ggcgtactgt tgtggtaaat acccactcga aattatccta taagaataat |
| 61 | catctgattt ttaaggatgc ctataaaacg gagctgatcc atttatcaga aattgatatt |
| 121 | ttgttattag aaacgaccga tattgtcttg tccactatgc tggtaaaacg gctagtggat |
| 181 | gagaatgtcc ttgtcatatt ctgtgatgat aaacgattac caacagctat gctgatgcct |
| 241 | ttttatggtc gtcatgattc gagtttacag cttgggaaac aaatgtcctg gtcagaaaca |
| 301 | gtcaaatcgc aggtttggac gacgattatt gctcaaaaga ttttgaatca atcttgctat |
| 361 | ctaggagcat gctcctattt tgaaaaatcc caatctatta tggatttata tcatggtttg |
| 421 | gaaaattttg atccgagtaa tcgagaaggg catgcagcga gaatttattt taatacactt |
| 481 | tttgggaacg atttctcaag agatttggag catccaatca atgcaggtct ggattatggt |
| 541 | tatactttat tattgagtat gtttgcgcgt gaagtggttg tgtctggatg tatgactcag |
| 601 | tttgggctta aacacgctaa tcagtttaat cagttcaatt ttgctagcga tattatggaa |
| 661 | ccatttaggc ctttagtgga taagattgtt tatgaaaatc gaaatcagcc ttttcccaaa |
| 721 | ataaagagag agttatttac tttgttttca gatacatttt catataatgg taaagagatg |
| 781 | tatctcacga atattattag cgattatact aaaaaagttg tcaaagctct gaataatgaa |
| 841 | gggaaaggag ttcctgaatt taggatatga |
| FTN_0757 | |
| (SEQ ID NO: 138) | |
| 1 | atgaatttca aaatattgcc aatagcaata gatttaggtg ttaaaaatac tggtgtcttt |
| 61 | agcgcatttt atcaaaaagg aacttctctt gagagattgg ataataaaaa tggcaaagta |
| 121 | tatgaactat caaaagattc ttatacttta ttgatgaata atagaacagc aagaagacat |
| 181 | caaagaagag ggatagatag aaagcagcta gtcaaaaggc tctttaagct tatttggaca |
| 241 | gagcagctaa atttagagtg ggataaagac actcaacaag caattagctt tttatttaat |
| 301 | cgtagaggtt ttagttttat tactgatggt tattcgcctg aatatttaaa tattgttcca |
| 361 | gagcaagtaa aagcgatact tatggatata tttgatgatt acaacggtga agatgattta |
| 421 | gacagttatt taaaattagc tactgagcaa gaaagcaaaa tttctgaaat ttataacaag |
| 481 | ctaatgcaaa aaatattaga gtttaaatta atgaaattat gtactgatat taaggatgat |
| 541 | aaagtaagta ctaaaacgct taaagaaatc acaagctatg aatttgagtt attagctgat |
| 601 | tatttagcaa actatagcga gagtttaaaa acacaaaaat ttagttatac agataaacaa |
| 661 | ggtaatttaa aagagctaag ctactatcat catgataaat ataatattca agaatttcta |
| 721 | aagcgacatg ctactataaa tgatcgaatt ttagatactc ttttaactga tgatttagat |
| 781 | atttggaatt ttaattttga gaaatttgat tttgataaga atgaagaaaa gcttcagaat |
| 841 | caggaagata aagatcatat acaagcgcat ttacatcatt ttgtttttgc agtaaataaa |
| 901 | ataaaaagtg aaatggcaag tggtggtcgt catcgtagcc aatattttca agagataaca |
| 961 | aatgtgctag atgaaaataa tcatcaagag ggatatctca agaatttctg tgaaaatttg |
| 1021 | cataataaaa aatattcaaa tttaagtgtt aaaaatttag ttaatctaat tggtaaccta |
| 1081 | agtaatttag agctaaaacc gctaagaaaa tattttaatg acaaaattca cgcaaaagct |
| 1141 | gatcattggg atgagcaaaa gtttacagaa acttattgcc actggatatt aggagagtgg |
| 1201 | cgagtaggtg tcaaagatca agataagaaa gatggcgcta aatatagtta taaagatctg |
| 1261 | tgtaatgaat taaaacaaaa agttactaag gctggtttgg tagatttttt attagagtta |
| 1321 | gatccatgta gaactatacc accatatctg gataacaata accgtaaacc accaaaatgt |
| 1381 | caaagtttga ttttaaatcc gaagttttta gataatcaat atccaaactg gcaacaatat |
| 1441 | ttacaagaat taaagaaact acaaagtatt caaaattatt tagacagttt tgaaactgat |
| 1501 | ttaaaagtct taaagtcaag taaagatcaa ccatattttg ttgaatacaa gagttcaaat |
| 1561 | cagcaaatag caagtggtca aagagattat aaagatttag atgctcgaat attacagttt |
| 1621 | atatttgata gggtaaaagc tagtgatgag ttgcttttga atgagattta ttttcaggct |
| 1681 | aaaaaactta aacaaaaagc tagctctgag ttagaaaaac tcgagtcgag caaaaagcta |
| 1741 | gatgaagtta tagcaaatag tcaactatca cagatactaa agtctcaaca tacaaatggt |
| 1801 | atttttgaac agggtacttt tttgcatttg gtttgtaaat attataaaca aagacaaaga |
| 1861 | gcgagagact ctaggctata tattatgcct gaatatcgtt atgataaaaa actacataaa |
| 1921 | tataacaata caggcaggtt tgatgatgat aatcagctgc taacatattg taatcataag |
| 1981 | ccaagacaaa aaagatacca attgttaaat gatttagctg gggtgttgca ggtatcacct |
| 2041 | aattttttga aagataaaat tggttctgat gatgatctat ttattagcaa atggttggta |
| 2101 | gagcatatta gaggatttaa aaaagcttgt gaagatagtt taaaaataca aaaagacaat |
| 2161 | agaggattat taaatcataa aataaatata gctaggaata caaaaggcaa atgtgaaaaa |
| 2221 | gaaatattta atttaatatg taaaatagaa ggttcagaag ataaaaaagg taattacaag |
| 2281 | catggtttag cttacgaatt aggagtactt ttatttggtg aacctaatga agctagtaaa |
| 2341 | cctgagttcg atagaaaaat taaaaaattt aactcaatat acagttttgc acagattcaa |
| 2401 | caaattgctt ttgcagagcg taaaggcaat gctaacactt gtgcagtttg tagtgctgat |
| 2461 | aatgctcata gaatgcaaca aattaagatc actgagcctg tagaggacaa taaagataag |
| 2521 | ataatcttaa gtgccaaagc tcagagacta ccagcgattc caactagaat agttgacggt |
| 2581 | gcggttaaga aaatggcaac tatattagct aaaaatatag ttgatgataa ttggcagaat |
| 2641 | atcaaacaag ttttatcagc aaaacatcag ttacatatac ctattatcac agaatcaaat |
| 2701 | gcttttgagt ttgaaccagc attagctgat gtaaaaggta agagcctaaa agataggaga |
| 2761 | aaaaaagcat tagagagaat aagtcctgaa aatatattca aggataaaaa caatagaata |
| 2821 | aaagaatttg ctaaaggtat atcagcatat agtggtgcta atttaactga tggcgatttt |
| 2881 | gatggtgcaa aagaagaatt agatcatata atacctcgtt cacataaaaa atacggtact |
| 2941 | ctaaatgatg aagcaaatct aatttgtgta actcgtggtg ataataaaaa taaaggtaat |
| 3001 | agaattttct gcctacgtga tcttgcagat aactataaac taaaacagtt tgagacaact |
| 3061 | gatgatttag aaattgaaaa gaagatagct gatacaatct gggatgctaa caagaaagat |
| 3121 | tttaaatttg gtaattatcg tagttttatt aacctaacac cacaagagca gaaagcattt |
| 3181 | cgtcacgcgc tatttctggc tgatgaaaat cctatcaaac aagcagtcat aagagcgata |
| 3241 | aataatcgta atcgtacatt tgtaaatggc actcaacgct attttgcaga agtactggca |
| 3301 | aacaatatct atctaagggc taaaaaagaa aatctaaata cagataaaat ttcatttgat |
| 3361 | tattttggta ttccaactat aggtaatggt agaggtattg ctgaaatccg tcaactttat |
| 3421 | gaaaaagttg atagtgatat acaagcttat gcaaaaggtg ataaacctca agctagctac |
| 3481 | tctcacctaa tagatgcgat gctggctttt tgtattgctg ctgatgaaca cagaaatgat |
| 3541 | ggaagtatag gtctagaaat cgataaaaat tatagtttat atccattaga taaaaataca |
| 3601 | ggagaagtct ttaccaaaga tatttttagt caaattaaaa ttactgataa tgagtttagc |
| 3661 | gataaaaaat tagtaagaaa aaaagctata gagggcttta acacgcatag acaaatgact |
| 3721 | agagatggca tttatgcaga aaattaccta ccaatactaa tccataaaga actaaatgaa |
| 3781 | gttagaaaag gctatacttg gaaaaatagt gaagaaataa aaatattcaa aggtaaaaag |
| 3841 | tacgatatac aacaattgaa taaccttgtg tattgtctaa aatttgtaga taaacctata |
| 3901 | tctatagata tacaaattag taccttagaa gagttaagaa atatattaac aacaaataat |
| 3961 | atagctgcta cagcagaata ctattatata aatctaaaaa cccaaaaatt acatgagtat |
| 4021 | tatatcgaaa actataatac tgccttaggt tataaaaaat acagtaaaga aatggagttt |
| 4081 | ttgagaagct tagcttatcg tagcgaaagg gtaaaaatta aatcaataga tgatgtaaag |
| 4141 | caggttttgg ataaggatag taactttatc atcggtaaga ttactttacc atttaaaaaa |
| 4201 | gagtggcaaa gactatatcg tgagtggcaa aatacaacta tcaaagatga ttatgagttt |
| 4261 | ttaaaatcat tctttaatgt taaaagtatt actaagttgc ataaaaaagt tagaaaagat |
| 4321 | ttctctttac ctatttctac aaatgaaggt aaattcctgg tcaaaagaaa aacatgggat |
| 4381 | aacaatttta tctatcagat attaaatgat tctgattcta gagcagacgg aacaaagcca |
| 4441 | tttattccag cttttgacat ttctaaaaat gaaatagtcg aagccataat tgattcattt |
| 4501 | acatcaaaaa atattttttg gctgcctaaa aatatagaat tacaaaaggt ggataataaa |
| 4561 | aacatttttg ctatagatac tagtaaatgg ttcgaagtag aaacacctag tgatcttaga |
| 4621 | gacattggaa tagcaacaat tcaatacaag atagataata attctcgccc taaagtcaga |
| 4681 | gttaaacttg attatgttat cgatgatgat agtaagataa attattttat gaatcattct |
| 4741 | ttattaaaat caagatatcc tgacaaagtt ttagaaattt taaaacaatc aactattata |
| 4801 | gaatttgaaa gttcaggttt taataaaact atcaaagaaa tgcttggtat gaaattagca |
| 4861 | ggtatttata atgaaacatc taataattag |
| Spy1046 | |
| (SEQ ID NO: 139) | |
| 1 | atggataaga aatactcaat aggcttagat atcggcacaa atagcgtcgg atgggcggtg |
| 61 | atcactgatg aatataaggt tccgtctaaa aagttcaagg ttctgggaaa tacagaccgc |
| 121 | cacagtatca aaaaaaatct tataggggct cttttatttg acagtggaga gacagcggaa |
| 181 | gcgactcgtc tcaaacggac agctcgtaga aggtatacac gtcggaagaa tcgtatttgt |
| 241 | tatctacagg agattttttc aaatgagatg gcgaaagtag atgatagttt ctttcatcga |
| 301 | cttgaagagt cttttttggt ggaagaagac aagaagcatg aacgtcatcc tatttttgga |
| 361 | aatatagtag atgaagttgc ttatcatgag aaatatccaa ctatctatca tctgcgaaaa |
| 421 | aaattggtag attctactga taaagcggat ttgcgcttaa tctatttggc cttagcgcat |
| 481 | atgattaagt ttcgtggtca ttttttgatt gagggagatt taaatcctga taatagtgat |
| 541 | gtggacaaac tatttatcca gttggtacaa acctacaatc aattatttga agaaaaccct |
| 601 | attaacgcaa gtggagtaga tgctaaagcg attctttctg cacgattgag taaatcaaga |
| 661 | cgattagaaa atctcattgc tcagctcccc ggtgagaaga aaaatggctt atttgggaat |
| 721 | ctcattgctt tgtcattggg tttgacccct aattttaaat caaattttga tttggcagaa |
| 781 | gatgctaaat tacagctttc aaaagatact tacgatgatg atttagataa tttattggcg |
| 841 | caaattggag atcaatatgc tgatttgttt ttggcagcta agaatttatc agatgctatt |
| 901 | ttactttcag atatcctaag agtaaatact gaaataacta aggctcccct atcagcttca |
| 961 | atgattaaac gctacgatga acatcatcaa gacttgactc ttttaaaagc tttagttcga |
| 1021 | caacaacttc cagaaaagta taaagaaatc ttttttgatc aatcaaaaaa cggatatgca |
| 1081 | ggttatattg atgggggagc tagccaagaa gaattttata aatttatcaa accaatttta |
| 1141 | gaaaaaatgg atggtactga ggaattattg gtgaaactaa atcgtgaaga tttgctgcgc |
| 1201 | aagcaacgga cctttgacaa cggctctatt ccccatcaaa ttcacttggg tgagctgcat |
| 1261 | gctattttga gaagacaaga agacttttat ccatttttaa aagacaatcg tgagaagatt |
| 1321 | gaaaaaatct tgacttttcg aattccttat tatgttggtc cattggcgcg tggcaatagt |
| 1381 | cgttttgcat ggatgactcg gaagtctgaa gaaacaatta ccccatggaa ttttgaagaa |
| 1441 | gttgtcgata aaggtgcttc agctcaatca tttattgaac gcatgacaaa ctttgataaa |
| 1501 | aatcttccaa atgaaaaagt actaccaaaa catagtttgc tttatgagta ttttacggtt |
| 1561 | tataacgaat tgacaaaggt caaatatgtt actgaaggaa tgcgaaaacc agcatttctt |
| 1621 | tcaggtgaac agaagaaagc cattgttgat ttactcttca aaacaaatcg aaaagtaacc |
| 1681 | gttaagcaat taaaagaaga ttatttcaaa aaaatagaat gttttgatag tgttgaaatt |
| 1741 | tcaggagttg aagatagatt taatgcttca ttaggtacct accatgattt gctaaaaatt |
| 1801 | attaaagata aagatttttt ggataatgaa gaaaatgaag atatcttaga ggatattgtt |
| 1861 | ttaacattga ccttatttga agatagggag atgattgagg aaagacttaa aacatatgct |
| 1921 | cacctctttg atgataaggt gatgaaacag cttaaacgtc gccgttatac tggttgggga |
| 1981 | cgtttgtctc gaaaattgat taatggtatt agggataagc aatctggcaa aacaatatta |
| 2041 | gattttttga aatcagatgg ttttgccaat cgcaatttta tgcagctgat ccatgatgat |
| 2101 | agtttgacat ttaaagaaga cattcaaaaa gcacaagtgt ctggacaagg cgatagttta |
| 2161 | catgaacata ttgcaaattt agctggtagc cctgctatta aaaaaggtat tttacagact |
| 2221 | gtaaaagttg ttgatgaatt ggtcaaagta atggggcggc ataagccaga aaatatcgtt |
| 2281 | attgaaatgg cacgtgaaaa tcagacaact caaaagggcc agaaaaattc gcgagagcgt |
| 2341 | atgaaacgaa tcgaagaagg tatcaaagaa ttaggaagtc agattcttaa agagcatcct |
| 2401 | gttgaaaata ctcaattgca aaatgaaaag ctctatctct attatctcca aaatggaaga |
| 2461 | gacatgtatg tggaccaaga attagatatt aatcgtttaa gtgattatga tgtcgatcac |
| 2521 | attgttccac aaagtttcct taaagacgat tcaatagaca ataaggtctt aacgcgttct |
| 2581 | gataaaaatc gtggtaaatc ggataacgtt ccaagtgaag aagtagtcaa aaagatgaaa |
| 2641 | aactattgga gacaacttct aaacgccaag ttaatcactc aacgtaagtt tgataattta |
| 2701 | acgaaagctg aacgtggagg tttgagtgaa cttgataaag ctggttttat caaacgccaa |
| 2761 | ttggttgaaa ctcgccaaat cactaagcat gtggcacaaa ttttggatag tcgcatgaat |
| 2821 | actaaatacg atgaaaatga taaacttatt cgagaggtta aagtgattac cttaaaatct |
| 2881 | aaattagttt ctgacttccg aaaagatttc caattctata aagtacgtga gattaacaat |
| 2941 | taccatcatg cccatgatgc gtatctaaat gccgtcgttg gaactgcttt gattaagaaa |
| 3001 | tatccaaaac ttgaatcgga gtttgtctat ggtgattata aagtttatga tgttcgtaaa |
| 3061 | atgattgcta agtctgagca agaaataggc aaagcaaccg caaaatattt cttttactct |
| 3121 | aatatcatga acttcttcaa aacagaaatt acacttgcaa atggagagat tcgcaaacgc |
| 3181 | cctctaatcg aaactaatgg ggaaactgga gaaattgtct gggataaagg gcgagatttt |
| 3241 | gccacagtgc gcaaagtatt gtccatgccc caagtcaata ttgtcaagaa aacagaagta |
| 3301 | cagacaggcg gattctccaa ggagtcaatt ttaccaaaaa gaaattcgga caagcttatt |
| 3361 | gctcgtaaaa aagactggga tccaaaaaaa tatggtggtt ttgatagtcc aacggtagct |
| 3421 | tattcagtcc tagtggttgc taaggtggaa aaagggaaat cgaagaagtt aaaatccgtt |
| 3481 | aaagagttac tagggatcac aattatggaa agaagttcct ttgaaaaaaa tccgattgac |
| 3541 | tttttagaag ctaaaggata taaggaagtt aaaaaagact taatcattaa actacctaaa |
| 3601 | tatagtcttt ttgagttaga aaacggtcgt aaacggatgc tggctagtgc cggagaatta |
| 3661 | caaaaaggaa atgagctggc tctgccaagc aaatatgtga attttttata tttagctagt |
| 3721 | cattatgaaa agttgaaggg tagtccagaa gataacgaac aaaaacaatt gtttgtggag |
| 3781 | cagcataagc attatttaga tgagattatt gagcaaatca gtgaattttc taagcgtgtt |
| 3841 | attttagcag atgccaattt agataaagtt cttagtgcat ataacaaaca tagagacaaa |
| 3901 | ccaatacgtg aacaagcaga aaatattatt catttattta cgttgacgaa tcttggagct |
| 3961 | cccgctgctt ttaaatattt tgatacaaca attgatcgta aacgatatac gtctacaaaa |
| 4021 | gaagttttag atgccactct tatccatcaa tccatcactg gtctttatga aacacgcatt |
| 4081 | gatttgagtc agctaggagg tgactga |
| y1724 | |
| (SEQ ID NO: 140) | |
| 1 | atgtcggacg aaggattagc ggcgtttatt gtctcttata tcaaaagccg cgagcagcca |
| 61 | aagcttgagg cttttgataa agaggcagag aaaaggctgg cagggctaac tcaggcggaa |
| 121 | gatattgcac tggcccagca agagattgcc caacaacggc aggagttgat agcccgttat |
| 181 | gaggtgcgca attggttaac cgatgcggct aaccgtgccg ggcaaataaa gttagcaact |
| 241 | catgcaccta aatacactca tagtgattca aaaagtagtg gtattttgaa tattgagtta |
| 301 | caaagtaaga aaaaggatta tttctccagc gtagacctcg ctgaacaagc cagtgatgtt |
| 361 | attggtaatg cagccgtatt tgatcttgta aagctattgc agagtgaatg tgaaggcggc |
| 421 | tcattaataa agtgtttaga gcaaggtgat aattcagtac tgaaactatt ctcaaatgat |
| 481 | gacgagttag ttgaagagtg gactacaaaa tttaaaagca ttttgaataa tgagaaattc |
| 541 | acgtcacata aactggccaa gcaattttat ttccctgtgg ggccagatca ataccacttg |
| 601 | ttaagtccac ttttctcctc atcattggcg caggccatgc atcaacgtat tattgaagcc |
| 661 | cgtttcagtg accaatcgaa agaggccaag gctgcccata aagcagggcg atggcatcct |
| 721 | acggtcaggg tcttgtacct tgatacggcg gtgcaacata ttggtggtac caaaccccaa |
| 781 | aatatctctt acctaaacag cgtccgtggc ggaaaagttt ggttgctgcc ctgtggcgca |
| 841 | ccgtcttgga aaaatattca gaaaccgcca ataaaatata gatctatttt tcatgatcgc |
| 901 | agtgaattta ctgtactggc ccgcaataac ctgtggcaaa tgcagctata tttgctgggt |
| 961 | gtcaaacccc tgagcaatac gatggacatc cgcgccgctc gtttggcctg ctgcgatgaa |
| 1021 | atcattgata ttctgtttaa ttatgtggct gaaattcaaa atctggacgg taccaatggc |
| 1081 | tggagtgacg ctgaagattg taagttaaaa cgctcagagc agctttggct tgacccgacc |
| 1141 | cgtgctatgg atgaccccgt atttaagctc gaacgtgaga aggaggattg gaaacaagag |
| 1201 | gtcagccaag acttcggtta ctggttaaat cgttggttac agcatgatga actggtcttt |
| 1261 | ggctatgttg aacagcggga gtggtcaagc ttatttaagc agcggctacg tgaatttgaa |
| 1321 | caaggtagcc tggagacatc gtcatga |
| Balac_1306 | |
| (SEQ ID NO: 141) | |
| 1 | atgcgcaaac tcacagtaca agacttgaat gaagcggcaa aaattggggg ctcgaatgcg |
| 61 | ctgacggaag taacgtcgct cgcaccggca gcaggtatgg gtagtatcgt agctcctgca |
| 121 | aaatacactg ctggtaatgg gtcgacgtat gtgtacgaga agcgctgggt gaatgatgag |
| 181 | tgtgttgata ctgtgctcat tgattctaga acatcacagg ccaatcgttt ggaagactac |
| 241 | atcagccgag cgattgaagt tggccatccg attttcagta agatgcccca agttcgcgta |
| 301 | cgttatgaga tgatcccagg agacgaaagc agcgtcaggt atttcgatga tgttcaattg |
| 361 | ccacatcgtg cggtggacgc gcatattcgc atcgctgaat tcagtgaatc cgacaaagtc |
| 421 | aagtacatgg cggcacgaaa ctcctccttg gaggatttgt ctgcaatgct ggcgatttcc |
| 481 | ccagtgacag tgatgtttgg gtgctgggac tcaacacgaa acaagaacca actgcgtatt |
| 541 | cccgccagct tcaacggtga gatttatgcg gtgcttgctg atcagacgca tgagtctcca |
| 601 | attcatcgtg caggcgcgcg tattgatcca gttgcagccg gagtgcatct aaccaaaaat |
| 661 | gaagccaaga aaattgctga gcgcatcaaa ggaacaatga atgacaagaa gctcagcaaa |
| 721 | ttcgcgagta gcggggatgg ctcaactatc gtcattggtg cgattccccc atcaactgat |
| 781 | gcaaatgctc ttgatggcat cgcagttcgt agtattacgc gcacacatgt cctgagtttc |
| 841 | tccatgcttc gggctatgcg cttcggtaaa gggccagagg gtgatgaagc tatccgtgta |
| 901 | ctgctggctg cagcgttgat caatgcgatg gttggaagca atgcggaatt gcatctacgg |
| 961 | gaaaactgct tccttgtgga agctgatgag ccgaaaaccg ttctggatcg tcgtggcggt |
| 1021 | aaacatgatg atttggagat gcttacgctg gaagacgccg atgagctgtt ggctcaggca |
| 1081 | tatgcacaag ctcagaaaaa ggcaggcatt gattggcatg ggcaaattat cactgtgcaa |
| 1141 | ggcgaccctg cagtgattga atctgcaagc gctgctgacg acgatgatag atga |
| GSU0053 | |
| (SEQ ID NO: 142) | |
| 1 | atgaacgatc tcgtgcagaa gtatgaccat tggttggaaa actccggacc tgcggcactg |
| 61 | gttattcgcg aacaactgat gcccgtcgag ggacgtgacg gtgtgctgtt tccagcgacc |
| 121 | tttgccgata ccggctacaa catcgacaaa ttcgacgatg gcggcaatgt ctgcctgatc |
| 181 | gacagtgtcg ggtcccaggc aaacaggatc gagccgatct tcatgactaa ggattacgct |
| 241 | ggccttgtcc cccaaatagt ggtccaggcg ggaaacaaaa aagtaaatct tctcgaagca |
| 301 | gggcatcgag ccggggacgc gattattcgc tgttctgagt tgcagcaaac ccttagggct |
| 361 | gcgttcaaca acgttctgaa tggcaatgca gagccactag cccgtatagc acccacctcg |
| 421 | cttgtgtttg gcgtgtggga ttcacgagat acccaagcca aattgcccag actcgttgcc |
| 481 | tcgaccataa gggcctacaa tgttcgccct ctcacccgct ctgcccagta tgtgccggct |
| 541 | gttgactaca acgccgaagg gcttttggaa gagcccggtg acttgcgaga tgctgaaggc |
| 601 | aaagtcaaga gcaagcaccc gtttgcccaa cgcgggtttg tgcatgtccc ggcgacaggt |
| 661 | gctctcggcg gcgtaatcgc caccgggggg attcgccgtg acgccacact ccaccttgcc |
| 721 | gcgctccgct tgctttcggc aggccaagac gaagcaaagt ccaaggccct tcgccgctat |
| 781 | atactcagtc ttgccttaac agcatttact gtgcctgtaa ctggctatct gcgtcagggc |
| 841 | tgcaatcttg tgctcgaccc tgaaaacccc cttgagttta aagaggtttt taatgatggg |
| 901 | acgcgcaatg acgtcggtat tacgcacacc gaagcgattg tctatgcaaa ggcagttgca |
| 961 | aaggagtttg gcattgaccc cgagcgtaac cttgacgaaa aaaaagcccc ggatcgagaa |
| 1021 | gtaccgtttg acaaggtact ggcgaaaaaa gatgtgagcg atgccggagg ctctaagaaa |
| 1081 | aaagcaaaat ga |
| MJ1666 | |
| (SEQ ID NO: 143) | |
| 1 | atgcaaaaaa tattaattgc tccatgggga aatttttcaa gttggaaaaa agttatctac |
| 61 | tcatttaatg gagttgaaaa agaatcaaaa agctctttat ctgccattta tgataaaata |
| 121 | aacccagata aagtttatat attggtttta gatactttat ctaatttaga atcagaaaat |
| 181 | tatggagata ttgtaaaaga agttaaagaa aagacagaga attttataaa agaaaattta |
| 241 | aacattgata attacgaggt aattgtatgt cctggagttg ggacatttta taacaaagat |
| 301 | tttgaaaaat actttaaatt ttatgggaat ttgactgatt attattcttt tgccctttat |
| 361 | gaattgtcta aaagattgga tggagatttg gaagttcatt tggacttaac acatggatta |
| 421 | aattacatgc ctgtcttaac ctatagagta attaaagacc tcttagaaat tttagcaata |
| 481 | aaaaataagg ttagattagt tgtttataac tcagacccct atgttggaag agaaaaagaa |
| 541 | atattaaaca tccacactgt ggaagatgtg attataaaac cgtcctatga cattaaaggt |
| 601 | atgactttgg attttttaga cgcaaccaaa tttgtagata aaaaagaaat aggaaaaata |
| 661 | aaaaaagaaa ttaacatgaa tccaaagata aaagaattaa gaataatgaa acaaaatata |
| 721 | aatgcattta tagcttctat tgtttatgct ctacctttag tttattcaac attctttgta |
| 781 | aagaaagata aaattgagat ttatttaaat gaacttattg gagcatttat ttcaaatata |
| 841 | aaaattaatc cagaagataa aatattaaaa agatacttat atttcggaga aggatttaat |
| 901 | agcttggtta aagcatattt tgcttcaaag attagcgaaa ttcctcaatt gataaaagac |
| 961 | gagctatctt tagaagagat tgatgaatta aaaaatacct tattcaaaga aaatccaaac |
| 1021 | tctcaatata tcaaaaatga gatttcatcc ctttataaca taataaacac caaatataaa |
| 1081 | gaagaagaac ttagtgaaat cttaggaaat tggactccaa tatataaaat tagaagggag |
| 1141 | aatattgaca aattcaagat taggaatttc ttagcacatg ctgggtttga aaaaagtgta |
| 1201 | actgaaattt atatttccgt agaaaataaa aatggaaaaa ttgaacttag tgaaaaaact |
| 1261 | tcgcttagat ataataagaa ctacatagaa gaaaaaaatg gaatcaaaag gttcatattt |
| 1321 | aaatataagg acaaaaatgg aaaagtagag gagataaata tcttagaaaa aattgaagag |
| 1381 | attctactaa acaaataa |
| NE0113 | |
| (SEQ ID NO: 144) | |
| 1 | atgccagagc cgctccaacc ccatgaatat ccgcaccgca tactgttatg tgttacgggg |
| 61 | ctgtcaccgc agattgtcac tgaaacgctg tacgctctgg ctgtggcacg ggccacgccc |
| 121 | tttattccga cagagataca tctgctgacc actaccgatg gagcacggtt ggcgcgggca |
| 181 | gcattgctgc accccgatgg tggacatttt catgcgctgt tgaatgacca gccacagatc |
| 241 | ggcctccccc gttttgatga agattgcatc catatcatca gccatcacca ggaaaaactc |
| 301 | gccgacattc gcacaccggc cgaaaatgcc gcagcggctg acacaatcac ggcacttgtt |
| 361 | gcccaactta ctgaggatgc tgacgcggcg ctgcatgttt cgattgccgg cgggcgcaag |
| 421 | accatgggtt tttatctggg gtatgccttt tcactgtttg cccggccgca ggataatctc |
| 481 | tcccatgttc tggtttcatc accctttgaa ggtcatccgg atttttttta cccgccacgt |
| 541 | cagccacgcc gcctggtaac acgagacggg catcatattg atactgccga ggccattgtt |
| 601 | acactggcag aaattcctgt ggtacggttg cggcacgggc tgccggctac tctgattgcc |
| 661 | ggccgcgccg ggttcagtga aacggtagtt accttgcagc aaagttttgc accaccatgc |
| 721 | ctgctgattg atctggagca gcgaaacgtc gtctgtggca ctactgcagt tgccatgaaa |
| 781 | ccgcaactgc ttgcatggct ggcgtggtgg gctacgctag cccgacaggg gcggcctgaa |
| 841 | acaacctggc gtgaagccga tgccagatta tttctcgata tttaccggac agtggttggt |
| 901 | attgatgcca ttgattatga gaaaaccgcc gagctgctcg gcaacggcat ggagaaggag |
| 961 | ttttttcaga ccaaaaacgc gaaactggaa cgtgtgctga aagacacact tggaccggca |
| 1021 | gctgccccct atctgctaac gactacgggc aaacgcccgc atacacggcg tggtctcaca |
| 1081 | ctgcctcccg agcgtattcg tatcgttggc acaggcagta aatga |
| PF1127 | |
| (SEQ ID NO: 145) | |
| 1 | atgggaatga gagttttggt aactacatgg ggtaatccct tccagtggga accaataaca |
| 61 | tatgaataca gaggaatcaa agttaaaagc agaaatacct tgccaattct agtcaagact |
| 121 | cttgagccag agaggattct aatccttgtg gctgatacaa tggccaacta ctatgattca |
| 181 | ggaaaaaata agccagaaat agaagaaaaa tcgttttcgt cttattcgga agttgtggaa |
| 241 | gatacaaaag aaaggatact atggcacata aaagaggagg tcattgaaga actccgtgag |
| 301 | gaagatcctg agcttgctaa gaaaattgag aatatgttaa aagatgaaag aattacaatt |
| 361 | gaagttcttc ccggcgttgg agtctttggc aacattacag tagagggaga aatgcttgac |
| 421 | ttctattatt atgccacata caagttggcc gaatggttgc cagttcagaa caatttagag |
| 481 | gtttacttag acctaactca tgggataaat ttcatgccca cctttactta cagagcccta |
| 541 | agaaacttgc ttggattgtt ggcctacttg tacaatgtaa agtttgagat agttaattca |
| 601 | gaaccttatc ccctgggggt ttcacaagaa ataagggagg acacaattct ccatattagg |
| 661 | gaaattggag agggagtagt tcgtcctaga ccacagtatt ctccagtaga aggaaagctt |
| 721 | tactggaatg catttataag ctctgtagcc aatggcttcc cgttagtctt tgccagcttt |
| 781 | tatccaaata ttcgggacgt agaagattac cttaacaaaa agcttgagga attcctggtg |
| 841 | ggaattgagg ttggggagag agaagatgga aaaccttatg ttaaaagaga gaaagctctt |
| 901 | gacaggagct ttaagaatgc ttctaagctc tactatgctt taagagtgtt caatacaaaa |
| 961 | ttccaaaact atccaaaaaa agaagttcct attgaagaaa taatggagat atcaaagata |
| 1021 | ttcgagtctc ttcccaggat tggaattatt ttagagaggc aagtagagtg gctaagaaat |
| 1081 | ttagtatatg gaagattatg gtatgaaaat ggagaacaga aaataaagaa gggtctttta |
| 1141 | gagattatca aggataagaa ggataaaagg aaagaggccg aagctcttaa aaaagggaag |
| 1201 | acaatatctt tagccgaagc tgcaaagctt acaagaatat tttctccgag tggagaaaga |
| 1261 | atagagacaa tagaatctcc aaatgttgtt cgtaacttta tagcacattc tggatttgag |
| 1321 | tataacattg tctatgtgaa atatgataga ctaagtgata ggctgtactt tttctataag |
| 1381 | gataaagaaa aagctgcaaa tctcgcttat gaagcccttt tatatagggg tgaaaaagaa |
| 1441 | tga |
| TM1812 | |
| (SEQ ID NO: 146) | |
| 1 | atgaatttcc ttgtaagaaa ccttgtggaa aatttggaag aaggggacag agttatactg |
| 61 | gatgttactc attcgttcag aagtattcca ttgatggcaa gtgtagtagc cctgtacctt |
| 121 | aaagaggcca aagatgttaa tgtgagtgta gtttacggca agtacaataa agaaacaaaa |
| 181 | gtaacagagt gcgaagattt gaccccacta actaaagcta catcatggat atatgctgtt |
| 241 | cgattgttca aagagtatgg atatgcgaaa gaactcgctg atttgataaa aaagagaaat |
| 301 | gaagaaatat acaggagaag tcaaagttcg aaaaaaccaa agctacttgg ttctatgtct |
| 361 | caaaaacttc aggatctttc gtcttctata cgtcttggat ccatagtagc cataaggaag |
| 421 | aatttaacca atttcttcaa ttttattgat agaaataaag caagaataag agaggagacg |
| 481 | gaggtttttg ttccagagat tgcggctctc ttagatggga tcgaaaaaag atacagggtt |
| 541 | atacatgtga aatcggagaa ttttgaactg agcgaaaaag aattggaatc tgaaaaagag |
| 601 | ttactggatt tctatcttca aacaggagat ttaggtatgg ctcttcgttt ggcaagagaa |
| 661 | tatcttataa atgtctattt gatgtctgga ggggagaaga gtgatttctt ggatagaaat |
| 721 | gttagagagt ctgtgagcat ttcaacgttt ggttatgata ccatccttca ggcaagaaat |
| 781 | catgttgctc atttcggatt caacaaactc cagcttccct ccttgaaaaa gatagaagat |
| 841 | cacctgaaag tgcttgttca aaccccaccg gaacaacttc tggagtctgc gagaaaaact |
| 901 | cagcgaaaca gaaaaagagc tcttctcact cctctcggta cgacaaaagg tgctttatac |
| 961 | accgttctga aaaaaatttc acctgatcta ctcctggtta taacttcaaa acagggaaaa |
| 1021 | gctattcttt cagaaattct ggagaaagct gaattcaagg gtgaatttag ggtaatcctt |
| 1081 | ctcgaagacc cttttatggg tgtaagtgaa atagatcgag tagtatccga aatcaaagaa |
| 1141 | catctttctg acgtggatga agtgatcgtc aatctcacag gtggaaccac ttttcttacg |
| 1201 | tacgttatcg agcgtgccaa aaatcaaatt agatacggaa gaaaagtaaa aactatcctg |
| 1261 | gctgtggaca agagaactta cgaagaacaa aagcagaatc cttttgtggt gggtgaaatt |
| 1321 | ctggagcttg attga |

### EXAMPLES

### Example 1

Due. to the highly regenerative nature of hematopoietic system (1), commonly used cytotoxic chemotherapy often causes myelosuppression. Life-threatening myelosuppression include neutropenia and thrombocytopenia. In addition, more than 50% of patients undergoing chemotherapy have severe anemia (2). Since myelosuppression is the cause of dose-limiting toxicity in many although not all chemotherapies, myelosuppression by conventional chemotherapy limits the intensities of chemotherapy for multiple cancer types and thus likely contribute the reduced therapeutic effect (3).

Based on analysis of hematopoietic stem cell (HSC) niches (4) and the kinetics of HSC proliferation (5,6), it has been suggested that distinct HSC are responsible for homeostatic and injury-induced hematopoiesis (4,5,7,8). Thus, it was postulated that the actively cycling HSC mediate homeostatic hematopoiesis, while the dormant HSC are responsible for injury-induced hematopoiesis (4,5,7,8). Consistent with this notion, HSC responsible for injury-induced but not homeostatic hematopoiesis are vulnerable to destruction by macrophages unless protected by CD47 (9). However, it is unclear whether the HSC that mediates homeostatic versus injury-induced homeostatic proliferation were maintained by distinct molecular programs. Since chemotherapeutic drugs (10-12) cause massive cell loss and trigger injury-induced hematopoiesis, understanding regulators that selectively regulate injury-induced hematopoiesis may lead to new approaches in minimizing myelosuppression associated with chemotherapy. In this context, it is of great interest to consider the role for miRNA. On one hand, with exception of NKT cells, most lineages of hematopoietic cells developed normally when the Dicer1 gene, a critical regulator for most miRNA, was deleted in both vascular endothelial stem cells and HSC (13). On the other hand, under conditions that could elicit injury-induced hematopoiesis, including treatment that induce inflammation and bone marrow transplantation, Dicer1 gene as well as specific miRNA, such as miR125, plays major role in hematopoiesis (14). It is therefore of interest to explore if injury-induced hematopoiesis under the condition of cancer chemotherapy can be protected by manipulation of specific miRNA.

In this study, miR-26a mediated a converging pathway in chemotherapy-related myelosuppression and tumor suppression and miRNA-aptamer was shown to be a platform to deliver miRNA to enhance therapeutic effect of chemotherapy while abrogating myelosuppression.

### Bioinformatics Analyses

Level 3 data of miRNA sequencing (miRNAseq) and RNA sequencing (RNASeq) Version 2 measured by Illumina HiSeq and clinical annotation tables of breast tumors from The Cancer Genome Atlas (TCGA) were downloaded from the UCSC Cancer Genomics Browser (https://genome-cancer.ucsc.edu/proj/site/hgHeatmap/). miRNA expression value was measured as counts normalized to reads per million mapped reads (RPM) and wilcoxon rank sum tests were used for comparisons between Basal-like breast tumors and normal breast tissues with FDR<0.05 and Log2-Ratio>0.5 (<0.5) considered significantly up-(down-)regulated. The up- or down-regulated miRNAs with mean of RPM in Basal-like breast tumors (normal breast tissues) less than 30 were deleted. RNAseq expression value was measured as counts normalized by RNA-Seq by Expectation-Maximization (RSEM) and the genes with mean of RSEM in Basal-like breast tumors less than 30 were deleted. The genes were identified as specifically upregulated in Basal-like breast tumors if differences in expression within Basal-like tumor and all other subsets (LumA, lumB and Her2) of breast tumors were significant (wilcox test, FDR<0.01) and difference relative to the Basal-like breast tumors was at least 2-fold. The overall survival curves were estimated using the Kaplan-Meier method and compared with the log-rank test, which were performed in R software (http://cran.r-project.org) using the survival package. The target prediction of miR-26a was performed using TargetScan 3.0. (http://www.targetscan.org/) and miRBase (http://microrna.sanger.ac.uk/).

### Cell Culture

No cell lines used in this study were listed in the database of cross-contaminated or misidentified cell lines suggested by International Cell Line Authentification Committee (ICLAC). Human breast cancer cell line (MDA-MB-231) and mouse TUBO cell line derived from BA LB/c mice transgenic for the transforming rat HER2/neu oncogene (BALB-NeuT) (20) were gifted from Dr. Yang-Xin Fu at University of Texas at Dallas South Western Medical Center (Dallas, Tex., USA). These cell lines were not authenticated but were regularly tested for mycoplasma contamination as required by in-house policies. These cells were cultured at 37C, 5% CO₂ in DMEM (Dulbecco's Modified Eagle Medium) (Thermo Scientific, Waltham, Mass. USA) supplemented with 25 mM HEPES and 10% heat-inactivated fetal bovine serum.

### Animals

Xenograft model were generated 8-weeks old female immune-deficient NOD-scid IL2Rgamma^{null} (NSG™, Jackson Laboratories, Bar Harbar, Me., USA) using human breast cancer cell line. Syngeneic TUBO tumors were established in 8-weeks old female BALB/c mice (from Charles River Animal Facility, Frederick, Md., USA). These tumor-bearing mice were randomly assigned to each treatment group. Eight-week old male C57BL/6 mice and C57BL/6 ly5.2 mice (from Charles River Animal Facility, Frederick, Md., USA) were used for bone marrow transplantation assay. The mouse groups assigned for each treatment were not blinded. No statistical methods were used to pre-determined sample size for the analysis of mouse experiments. All the mice were maintained in the Research Animal Facility at Children's National Institute, Children's National Medical Center. The committee on the Use and Care of Animal at Children's National Institute approved all procedures involving experimental animals.

Aptamer and miRNA Chimera Preparation

DNA sequences for anti-human KIT aptamer (21) and mouse c-Kit aptamers (22) conjugated with biotin at 3′ were functionalized by short denaturation-renaturation step (95° C. 10 min, 5 min snap-cooling on ice). These DNA sequences were 5′-GAGGCATACCAGCTTATTCAAGGGGCCGGGGCAAGGGGGGGGTACCGTGGTAGGAC ATAGTAAGTGCAATCTGCGAA-3′ (SEQ ID NO: 2) for human KIT, 5′-GCTCAACGCGGGACGGCTCTCCCATTGAC-3′ (SEQ ID NO:3) for mouse c-Kit. These activated aptamers were used for binding assay to the cell lines by flow cytometry. For chimera preparation, human KIT-aptamer or mouse c-Kit-aptamer miR-26a chimera was assembled by three compartments of DNA/RNA hybrid sequences. The sequences were 5′-GAGGCATACCAGCTTATTCAAGGGGCCGGGGCAAGGGGGGGGGTACCGTGGTAGGAC ATAGTAAGTGCAATCTGCGAA/C3 (SEQ ID NO:4) spacer/CCUAUUCUGG-3′(SEQ ID NO:5) for human KIT aptamer+the part of passenger sequence for miR-26a-5p, 5′-GCTCAACGCGGGACGGCTCTCCCATTGAC/C3 (SEQ ID NO:6) spacer/CCUAUUCUGG-3′ (SEQ ID NO:7) for mouse cKit aptamer+the part of passenger sequence for miR-26a-5p, 5′-GUUACUUGCACG/TEG (triethylene glycol)-Cholesterol-3′ (SEQ ID NO:8) for the part of sequence for miR-26a-5p+cholesterol, and 5′-UUCAAGUAAUCCAGGAUAGGCU-3′ (SEQ ID NO:9) for guide sequence for miR-26a-5p (RNA sequences were represented as italic). The guide sequence for scramble control was GGUUCGUACGUACACUGUUCA (SEQ ID NO:10). To protect the oligonucleotide sequences from nuclease degradation, phosphorothioate bonds were introduced between the last 3 nucleotides at the 5′-end of DNA aptamers. The RNA sequences were modified with 2′-fluoro-uridines. The conjugation with cholesterol at 3′ oligonucleotide improved in vivo pharmacokinetic properties, enhanced the permeation of cellular membranes, and protected the RNA from in vivo degradation (23-25). KIT receptor targeting aptamer enhanced the up-take of chimera via receptor-mediated internalization (26). Generating an internal nicking in complementary sequence of miR-26a prevented non-specific miRNA targeting by RISC complex (27). All oligonucleotides with these modifications were synthesized and purified by Integrated DNA Technologies (Coralville, Iowa, USA). For assembling there components to generate the chimeras, the DNA aptamers were initially subjected to the short denaturation-renaturation step (95° C. 10 min, 5 min snap-cooling on ice), and then equal molar of aptamer+passenger sequence for miR-26a, passenger sequence for miR-26a+cholesterol, and guide sequence were incubated in annealing buffer (30 mM HEPES, 100 mM potassium acetate, p117.5) (Integrated DNA Technologies) at 95° C. for 2 min, 55° C. 20 min, and then 37° C. for 30 min.

### Chimera Treatment In Vivo

For xenograft models, the NSG mice and BALB/c mice were subcutaneously injected with 2×10⁶ viable MDA-MB-231 cells and TUBO cells in their right hind limbs, respectively. After the tumor grew to 1 cm in diameter, mice were randomly divided into the groups for each treatment. For no treatment control, 100 μl saline with 1% mouse serum was intravenously injected through tail vein. For treatment groups, 670 pmol/20 g chimeras and/or 5(0 mg/kg 5-fluorouracil (5-FU), or 120 mg/kg carboplatin were also intravenously injected. Tumor sizes were measured in two dimensions every 3 days. Tumor volume (V) was calculated using the following formula: V=(½) S²×L (S. the shortest dimension; L, the longest dimension). Animals were euthanized when the tumor size was over 2 cm diameters, tumor ulcerated, or became necrotic. For chimera treatment in normal mice, C57BL/6 mice were treated with miR-26a chimera (670 pmol/20 g) everyday for 3 days by intravenous injection. At day 2, 150 mg/kg 5-FU and/or 120 mg/kg carboplatin was injected together with the chimera. Peripheral bloods were collected at day 5 and day 10 after the 5-FU treatment. Complete blood counts were measured by Hemavet 950FS (Drew Scientific, Miami Lakes, Fla., USA).

### Flow Cytometry and Cell Sorting

For aptamer binding assay, streptavidin-APC (Cat #554067) (B31) Bioscience, San Jose, Calif., USA) was used for detecting the biotin-conjugated aptamers by flow cytometry. Other cell targeting aptamer specific for Ramos cells (28) was used for negative control. For phenotype analysis, mice were sacrificed by CO₂. Bone marrow (13M) cells were flashed out from the long bones (tibiae and femurs) by 25-gauge needle with 1× Hanks Balanced Saline Solution (HBSS) (Thermo Fischer Scientific), supplemented with 2% heat-inactivated fetal bovine serum (FBS). Splenocytes and thymocytes were collected after spleen and thymus ground with frosted slides. Peripheral blood was obtained from the tail veins of recipients at the time points indicated in the figures, and red blood cells (RBC) were lysed by ammonium chloride-potassium bicarbonate buffer before staining. CD3e (clone 145-2C11), B220 (clone RA3-6B2), CD11b (clone MI/70), Gr-1 (clone RB6-8C5), Ter-119 (clone Ter-119), Sca-1 (clone D7), c-Kit (clone 2B8), CD150 (clone TC15-12F12.2), and CD48 (clone HM48-1) antibodies were purchased from BD Bioscience, antibodies were diluted in 1×PBS (phosphate-buffered saline) containing 0.5% FBS and 2 mM ethylenediaminetetraacetic acid (EDTA) and stained was performed on ice for 20 minutes. Annexin V staining was performed using AnnexinV apoptosis detection kit (BD Bioscience) according to the manufacture's instruction. Flow cytometry analysis was performed on FACS Canto II (BD Bioscience) and the data were analyzed with FlowJo (FLOWJO, Ashland, Oreg., USA). KIT/c-Kit⁺ cells from tumors were isolated with CD117 micro beads kits (human or mouse) (Miltenyi biotec, San Diego, Calif., USA) by autoMACS pro separator (Miltenyi biotec). LSK population (CD3e⁻/B220⁻/CD11b⁻/Gr-1⁻/Ter119⁻/Sca-1 ⁺/c-Kit⁺) and HSCs (CD3e⁻/B220⁻/CD11b⁻/Gr-1⁻/Ter119⁺/Sca-1 ⁺/c-Kit⁺/CD48⁻/CD150⁺) harvested from bone marrows were isolated by BD Influx cell sorter (BD) Biosciences).

### Real-Time PCR

Total RNAs from cell lines and mouse tissues were extracted by RNeasy Plus Mini kit (Qiagen, Valencia, Calif., USA). miR-26a levels were quantified by Taqman microRNA assay (assay ID; 000405) that covered both has-miR-26a-5p and mmu-miR-26a-5p (Thermo Fisher Scientific) according to manufacturer's protocol. U6 snRNA (Taqman microRNA assay, assay ID; 001973) was used as endogenous control. For qPCR of other genes, cDNAs were synthesized from the purified total RNA with qScript cDNA supermix (Quanta Bio, Gaithersburg, Md., USA). Real-time PCR was performed with Power SYBR Green PCR master mix (Thermo Fisher Scientific) on 7500 Real Time PCR system (Thermo Fisher Scientific). Sequence primers for mouse Bak were TCTCCACCAAGACCTGAAAAAT (forward) (SEQ ID NO: 11) and CTTCGAAAGACCTCCTCTGTGT (reverse) (SEQ ID NO:12). For mouse Ezh2, TTGCTAAGAGGGCTATCCAGAC (forward) (SEQ ID NO:13) and TGTCAAGGGATTTCCATTTCTC (reverse) (SEQ ID NO:14). For mouse Actb, AGGAGTCCTGTTGATGTTGCCAGT (forward) (SEQ ID NO:15) and GGGACGCAGCAACTGACATTTCTA (reverse) (SEQ ID NO:16) were used as endogenous control.

### Western Blot

The cell lysate from MDA-MB-231 cells treated with 167 nM chimeras for 2 days were fractionated by SDS-PAGE and then transferred to PVDF membrane (EMD Millipore, Billerica, Mass., USA). Following blocking with 5% FBS in tris-buffered saline, the membrane was incubated with primary antibody against EZH2 (D2C2) and Histone H3 (Cat #9715) (Cell Signaling Technologies, Danvers, Mass., USA) overnight at 4° C. and then incubated with horseradish peroxidase-conjugated anti-mouse and rabbit IgGs (Thermo Fisher Scientific) for 1 hr. respectively. The antigen-antibody immunoreactivity was detected in ChemiDoc Touch Imaging System (Bio-Rad Hercules, Calif., USA) using SuperSignal West Pico Chemiluminescent Substrate (Thermo Fisher Scientific).

### Lentivirus Production

For miR-26a overexpression, the sequence of mouse miR-26a-1 stem loop structure and 400 base pairs of upstream and downstream flanking genomic sequence was cloned into a pGIPZ lentiviral vector (GE Dharmacon, Lafayette, Colo., USA). For miR-26a inhibition, the miR-26 TuD (tough-decoy) inhibitor sequence (GACGGCGCTAGGATCATCAACAGCCTATCCTGGTCTCATTACTTGAACAAGATTTCT GGTCACAGAATACAACAGCCTATCCTGGTCTCATTACTTGAACAAGATGATCCTAGC GCCGTCTTTTTT) (SEQ ID NO:17) was cloned into pLL3.7 lentiviral vector (Addgene plasmid #11795, Addgene, Cambridge, Mass., USA) that expresses the RNAs under mouse U6 promoter. Viral production was performed in 293T cells following manufacture recommendation (Lenti-X Lentiviral expression system) (Clontech, Mountain View, Calif.). Similarly, control vector was made expressing non-cording TuD. The sequences (CATCAACTATCGCGAGTATCGACGTCGAGGCCCAAGTATTCTGGTCACAGAATACA ACTATCGCGAGTATCGACGTCGAGGCCCAAG) (SEQ ID NO:18) were cloned into the lentiviral vector.

### Luciferase Assay

Predicted miR-26a targeting sequence (730 bp) from mouse Bak1 3′UTR was inserted into the downstream of the firefly luciferase gene in pmirGLO Dual-Luciferase miRNA Target Expression Vector (Promega, Madison, Wis., USA). The deletion of the targeting sequence on the Bak1 3′UTR was inserted into the vector as the mutated clone. HEK293 cells were transduced with the lentivirus inducing miR-26a over-expression or inhibition. Two day after the transduction, the luciferase plasmids were transfected by Lipofectamine LTX reagent (Thermo Fisher Scientific). Next day, luciferase assays were performed with dual-luciferase reporter assay system (Promega) using a luminometer (FLUOstar Optima, BMG Labtech, Cary, N.C., USA). The value of relative luminescence denotes the firefly luciferase activity normalized to renilla luciferase activity for each assay.

### Bone Marrow Transplantation

The c-Kit⁺ cells harvested from bone marrow (BM) of 8-week old C57BL/6 donor mice (CD45.2) were transduced with miR-26 TuD or ctrl lentivirus in StemMACS HSC Expansion Medium (Miltenyi biotec) supplemented with 100 ng/ml SCF, 100 ng/ml TPO, 40 ng/ml Flt3 ligand (Miltenyi biotec), and 4 μg/ml polybrene (Sigma-Aldrich, St. Louis, Mo., USA) by spinoculation (800×g, 30 min, 32° C.) and further incubation in 37° C. overnight. Lethally irradiated (8.5Gy) mice were transplanted with the virus-transduced BM cells (5×10⁵ cells). For competitive BM transplantation, the virus-transduced donor c-Kit⁺ BM cells (CD45.2⁺), mixed with same number (5×10⁵ cells) of recipient-type competitive c-Kit⁺ BM cells (CD45.1⁺), were transplanted into recipients through the tail vein within 24 hours after irradiation. Reconstitutions in the recipients' BM, spleen, thymus and peripheral blood by donor-derived cells were measured by flow cytometry at the time points indicated.

### Statistics

For statistical test selection, distribution fitting and variance testing was determined to justify test selection. The specific tests used to analyze each set of experiments were indicated in the figure legends. The majority of the data had similar variance, and all data met the assumptions of the statistical test. Data were analyzed using a Student's t test to compare between two groups, and two-way repeated-measures ANOVA, followed by the Bonferroni post-hoc procedure for follow-up pairwise comparison. Survival data were analyzed by a Kaplan-Meier survival analysis with log-rank test. Sample sizes were chosen with adequate statistical power on the basis of the literature and past experience, and mice were allocated to experimental groups according to age, gender and genotype. No samples were excluded from analysis, and experiments were not randomized except what was specified. Statistical calculations were performed using GraphPad Prism software (GraphPad Software, San Diego, Calif.) and R Software (https://www.r-project.org/). Error bars stand for standard deviation. *P<0.05, **P<0.01, ***P<0.001.

### Example 2

Identification and Validation of KIT-Targeting miR-26a Chimera that Inhibits Human Breast Cancer Growth In Vitro and In Vivo

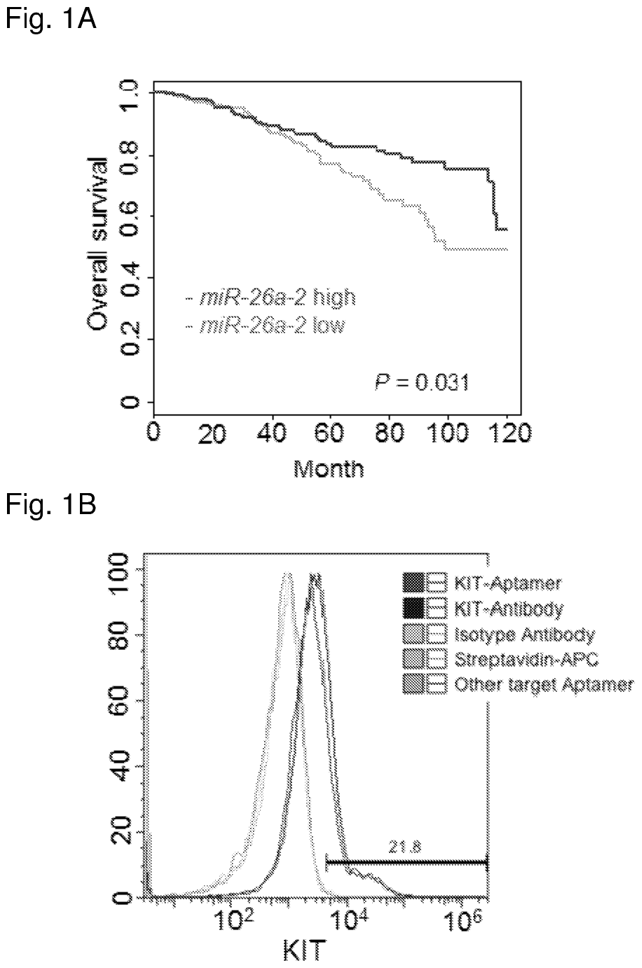

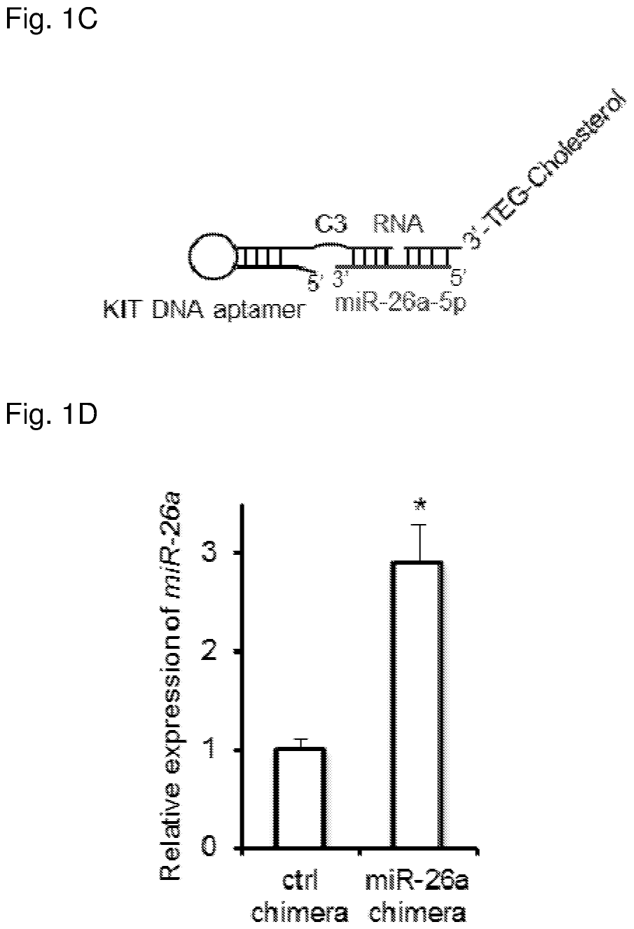

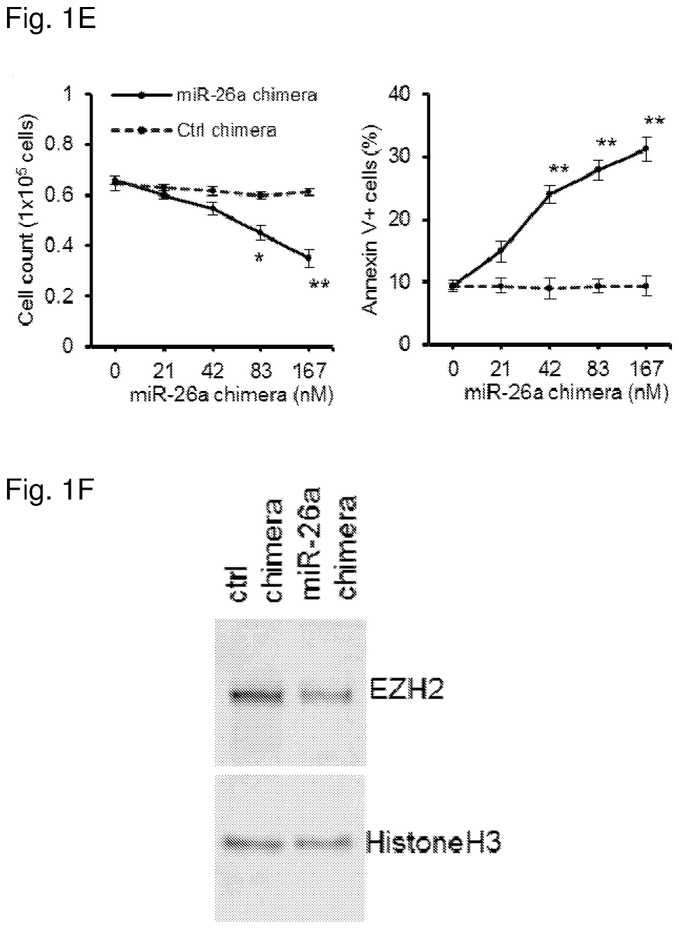

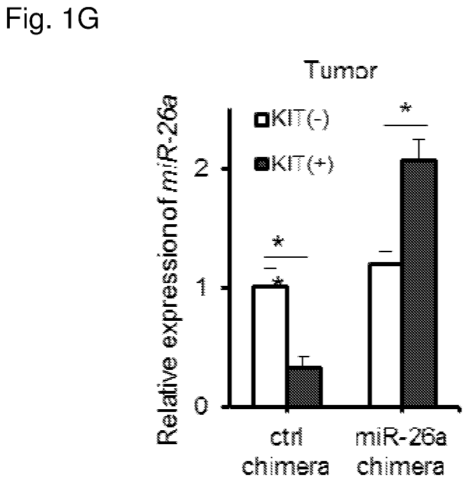

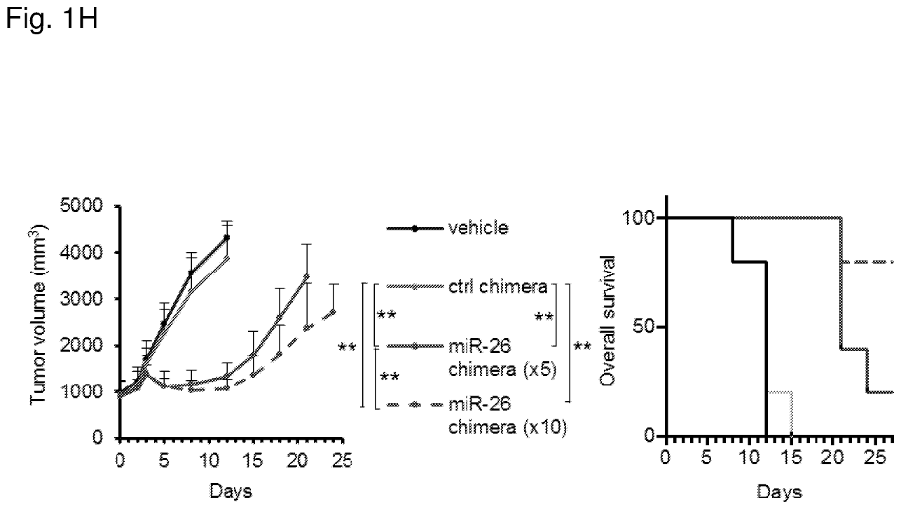

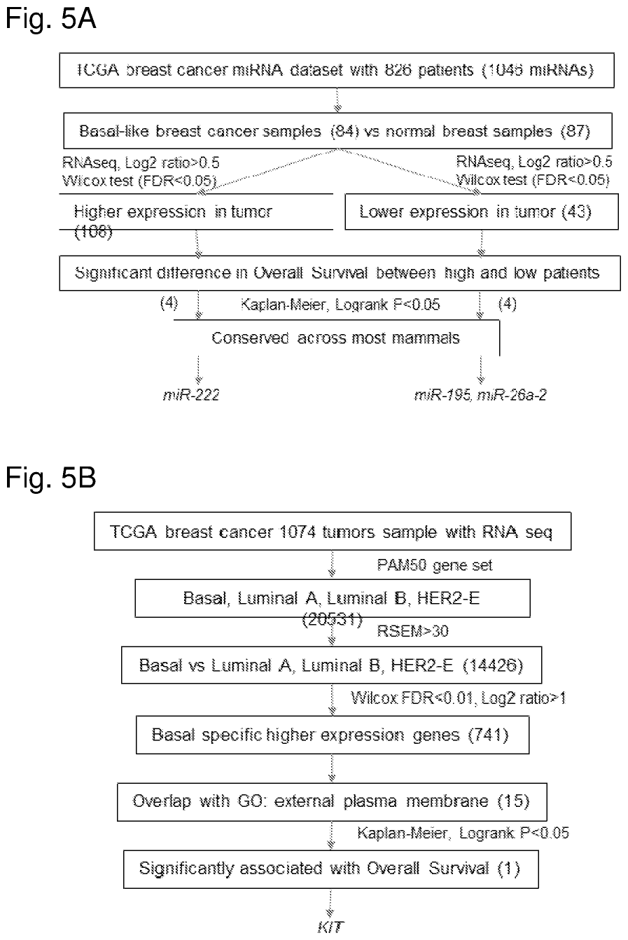

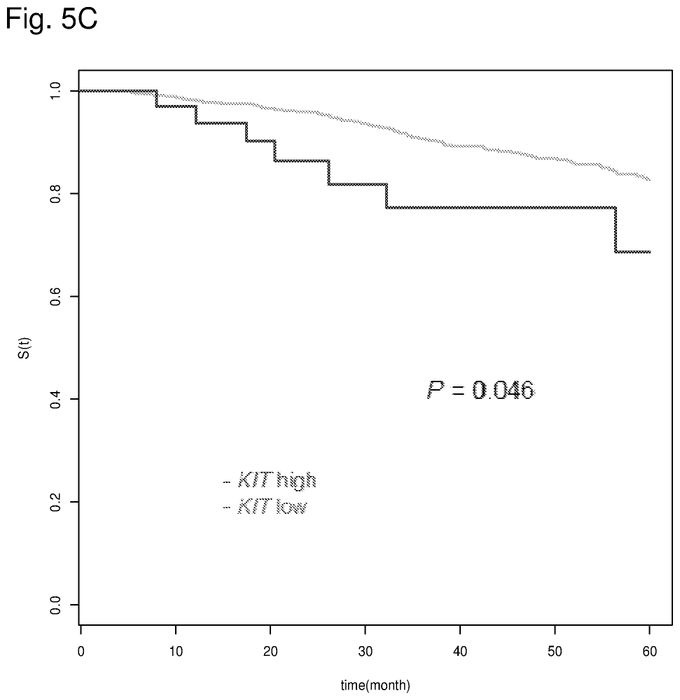

Accumulating data have shown that alternation of miRNAs is involved in cancer initiation and progression [1-3,8] and that manipulating expression level of miRNAs in cancer cells may offer potential therapeutic effect [9]. To identify a miRNA for treatment of advanced breast cancer, the breast cancer miRNA dataset was analyzed with information from 826 patients in The Cancer Genome Atlas (TCGA) program. In silico analysis revealed that miR-26a-2 (mature miR-26a-1 and miR-26a-2 have the same sequence despite encoded by distinct genes, and the mature sequence is referred as miR-26a) was significantly down-regulated in basal-like breast cancer in comparison to paired normal tissue, and that the down-regulation was significantly associated with shorter overall survival of basal-like breast cancer patients (FIG. 1A and and FIG. 5). Data were consistent with recent reports that miR-26a was down-regulated in the triple-negative breast cancer that includes 50-75% of basal-like breast cancer, and its expression levels were associated with metastasis and poor overall survival of the triple-negative breast cancer patients (10,11).

Since the ectopic expression of miR-26a inhibited the proliferation and metastasis of basal-like breast cancer cells (29.30), a method to deliver miR-26a into cancer cells was developed. To deliver miR-26a mimic selectively to the basal-like breast cancer, the cell surface proteins were enriched on the basal-like breast cancer cells. Bioinformatics analysis using the TCGA database identified the KIT gene as the most highly expressed cell surface protein on the basal-like breast cancer cells compared to other subtypes of breast cancer (FIG. 5B). The higher expression of the KIT gene was also significantly associated with poor clinical outcomes in the patients with breast cancer (FIG. 5C). Therefore basal-like breast cancer was targeted using DNA aptamers against the KIT proteins (21.22). To evaluate the binding ability of the human KIT aptamer to basal-like breast cancer cells, flow cytometry analysis was performed, and this confirmed the binding to human basal-like breast cancer cell line (MDA-MB-231). As shown in FIG. 1B, the anti-KIT antibody and aptamer revealed nearly identical distribution of KIT expression among cancer cells, with higher levels on a small subset of cancer cells. Using this aptamer, a KIT-targeting miR-26a chimera was designed comprising (1) miR-26a-5p RNA mimic sequence, (2) anti-KIT DNA aptamer linked with a part of miR-26a complementary sequence, and (3) TEG (triethylene glycol)-cholesterol linked with the rest of the complementary sequence (FIG. 1C). The KIT receptor was chosen as this receptor targeting aptamer enhanced the up-take of chimera via receptor-mediated internalization (26). The conjugation of TEG-cholesterol improved in vivo pharmacokinetic properties, enhanced the permeation of cellular membranes, and protected the RNA from in vivo degradation (23-25), while an internal nicking in complementary sequence of miR-26a prevented non-specific miRNA targeting by RISC (RNA-induced silencing complex) complex (27). Using this system, the chimera successfully delivered miR-26a into the MDA-MB-231 cells (FIG. 1D). Importantly, the chimera consisting of miR-26a, but not scrambled sequence, inhibited cell growth by the induction of apoptosis in vitro (FIG. 1E). Correspondingly, the chimera treatment in MDA-MD-231 cells significantly suppressed EZH2 protein expression (FIG. 1F), which is a major oncogene for the basal-like breast cancer (30).

To evaluate the therapeutic potential of miR-26a chimera, the chimera were injected intravenously into immune-compromised NSG™ mice bearing large MDA-MB-231 xenograft tumors once every day for either 5 or 10 times. To confirm specific targeting, single-cell suspension from the tumors were isolated at 3 days after single injection, sorted into KIT⁺ and KIT⁺ populations, and the miR-26a levels measured by quantitative PCR (qPCR). As shown in FIG. 1G, significant elevation of miR-26a was detected in KIT⁺ tumor cells isolated from mice that received miR-26a chimera. No increase of miR-26a was observed in control chimera-treated KIT⁺ tumor cells and in KIT tumor cells from mice that received either miR-26a or control chimera. Therefore, the aptamer-based delivery allowed specific target of KIT⁺ tumor cells. Remarkably, 5 or 10 injections with miR-26a chimera, but not control chimera, significantly suppressed the tumor growth and extended their survival (FIG. 1H). These results suggest the therapeutic potential of miR-26a chimera for basal-like breast cancer treatment.

miR-26a Protects Hematopoiesis from Chemotherapeutic Agent-Induced Myelosuppression

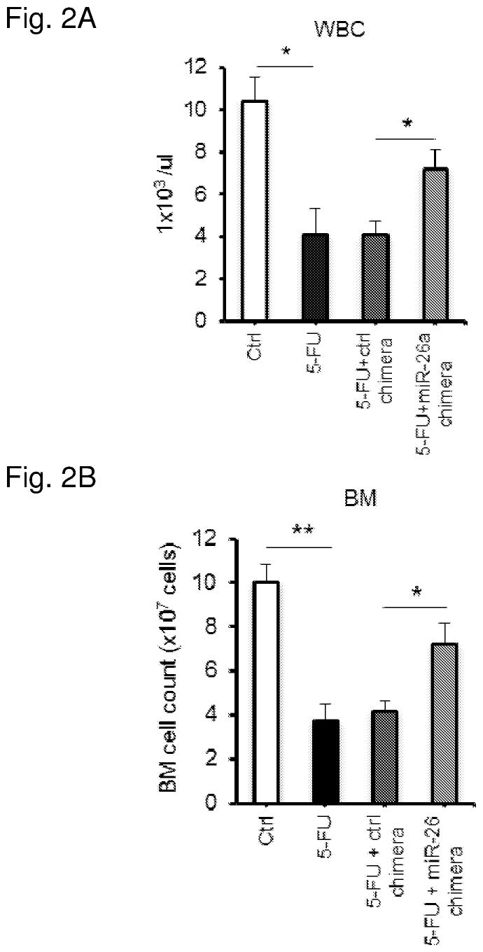

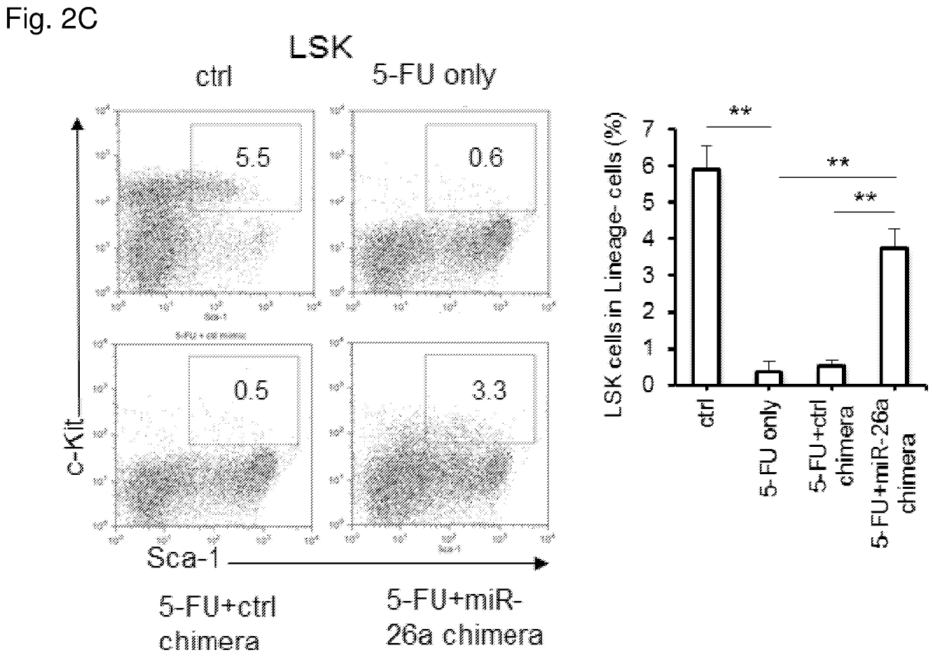

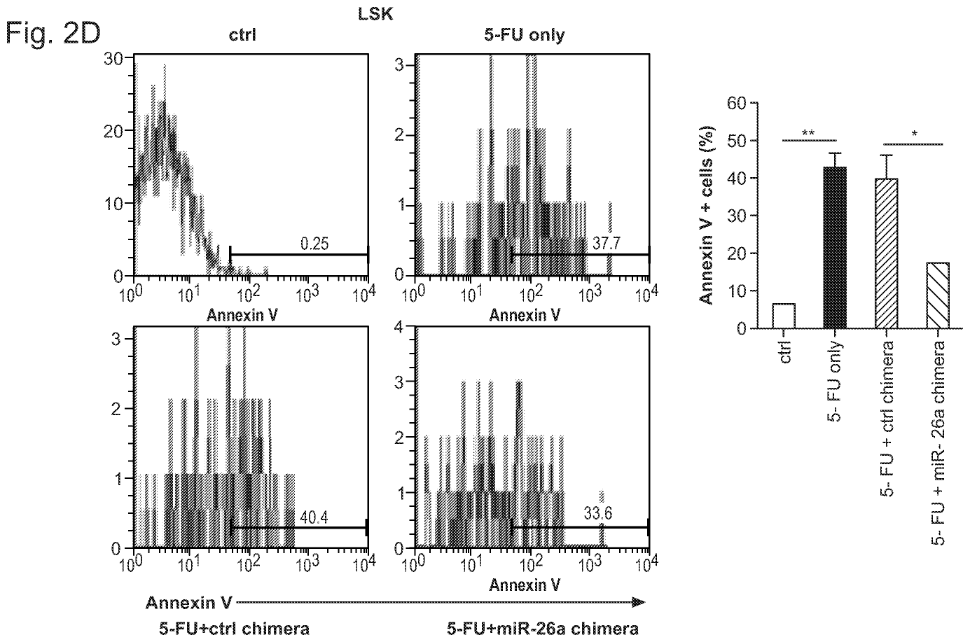

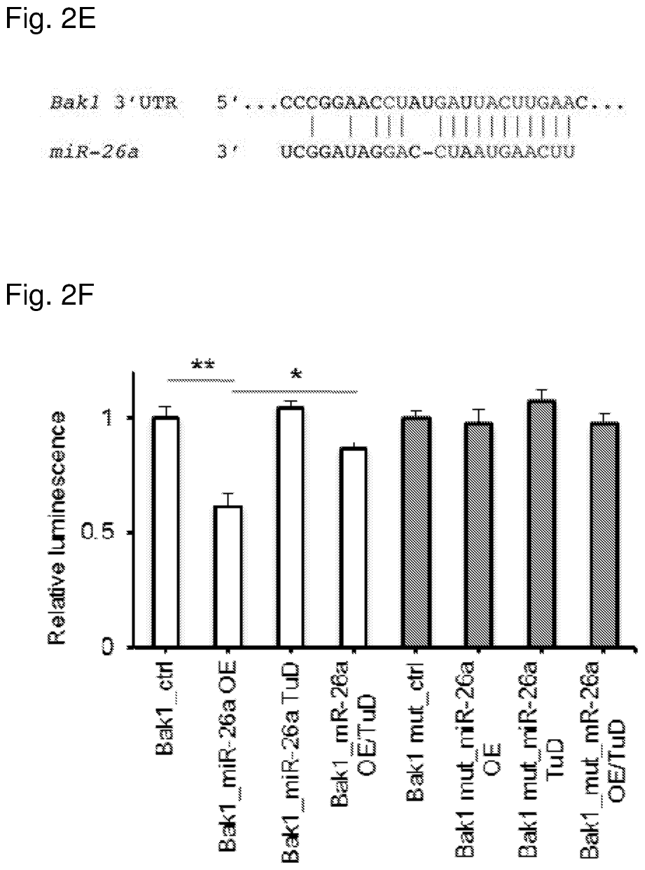

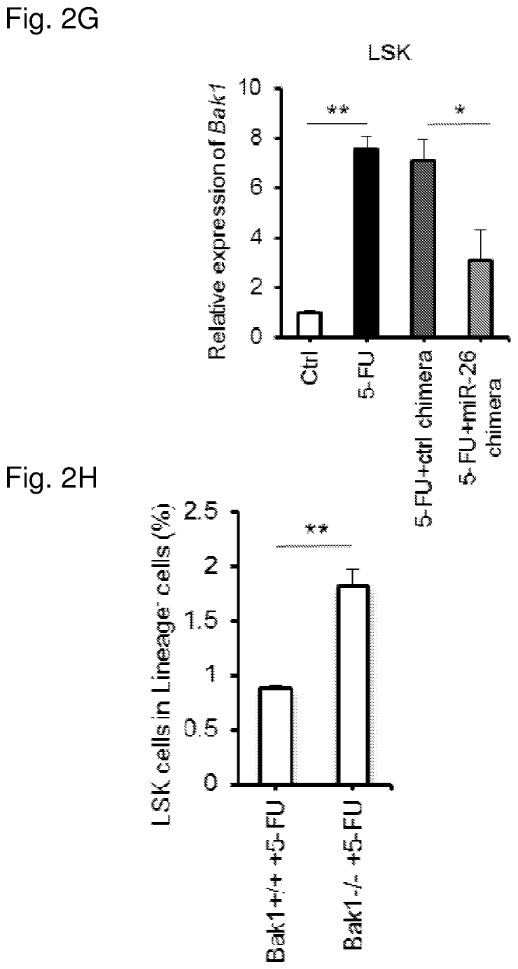

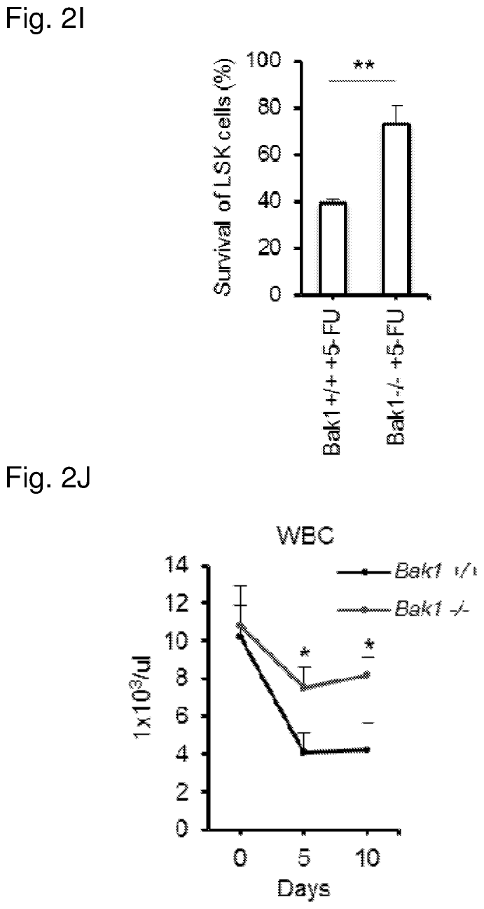

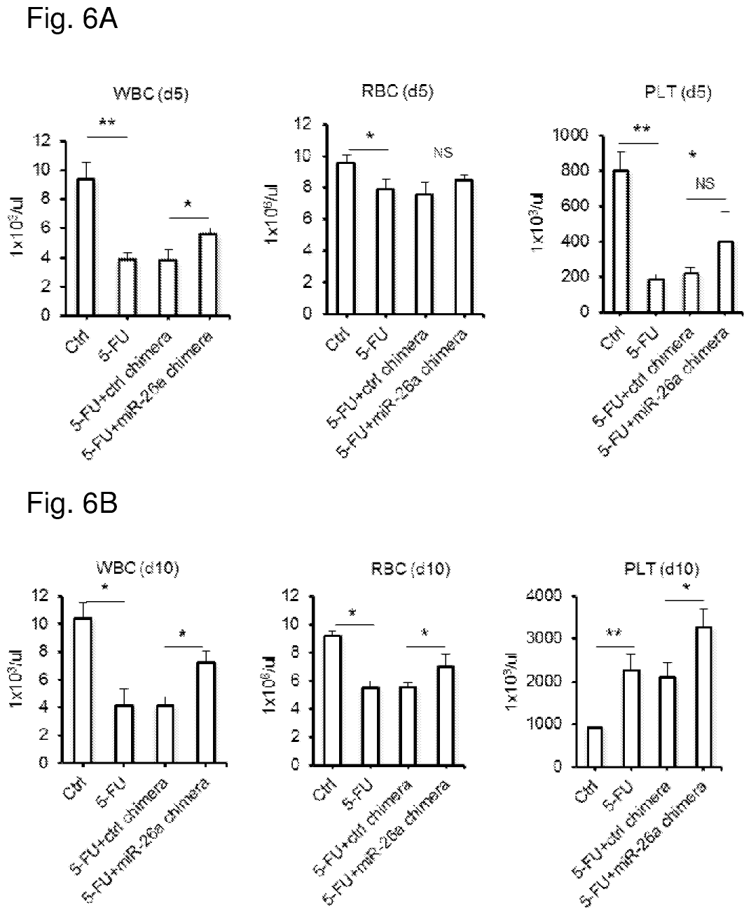

Myelosuppression limits the intensities of chemotherapy regimen using 5-FU against the advanced breast cancer (3). Since c-Kit is expressed at high levels in HSPC (31), the effect of miR-26a on myelosuppression was tested by 5-FU. As expected, 5-FU induced significant defects in hematopoiesis, as revealed by reduction in leukocytes (WBC) (FIG. 2A and FIG. 6) and total bone marrow (BM) cellularity (FIG. 2B). Remarkably, miR-26a chimera significantly ameliorated myelosuppression as revealed by increased leukocyte counts and bone marrow cellularity.

Given the preferential expression of c-Kit in the HSPC, the protective effect of miR-26 on the HSPC (LSK population) was evaluated against 5-FU. As shown in FIG. 2C, 5-FU caused nearly 10-fold-reduction in the LSK population (CD3e⁻/B220⁻/CD11b⁻/Gr-1⁻/Ter119⁻/Sca-1⁺/c-Kit⁺) at 5 days after treatment. This was largely prevented by miR-26a chimera (P=0.0063) but not control chimera. Correspondingly, the percent of apoptotic LSK was massively increased by 5-FU and specifically protected by the miR-26a- but not the control chimera (FIG. 2D).

Bak1 is a Target of miR-26a for its Myeloprotection from Chemotherapeutic Agent

miR-26a target genes that potentially regulate apoptosis were searched using in silico approach. Among them, Bak1 (Bcl-2 antagonist/killer1) was a putative target (FIG. 2E). To directly test whether miR-26a targets Bak1, the 3′UTR of Bak1 downstream sequence was cloned into a luciferase reporter vector, and co-transfected with miR-26a precursors transducing lenti-virus (miR-26 OE) or miR-26a Tough-Decoy inhibitor transducing virus (miR-26 TuD) that inhibits microRNA function by acting like a sponge for microRNAs (32). The luciferase activity was significantly down-regulated by the over-expression of miR-26a. Further co-transfection of miR-26 TuD inhibitor significantly rescued the down-regulation of luciferase activity caused by miR-26a over-expression. The depletion of miR-26a binding site on the Bak1 3′UTR abolished the inhibitory effects of miR-26a OE on the luciferase expressions, indicating that Bak1 is a direct target of miR-26a (FIG. 2F).

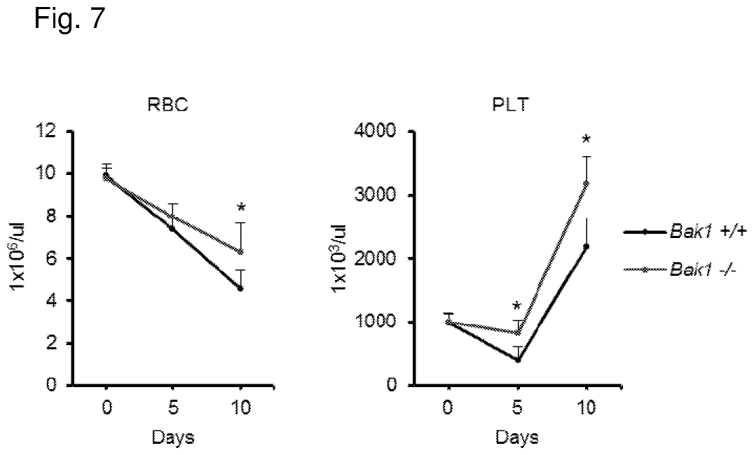

To test the in vivo effect, Bak1 levels were compared among LSK cells in mice that received 5-FU in conjunction with or without miR-26a or control chimeras. As shown in FIG. 2G, Bak1 expression was massively induced by 5-FU treatment in LSK cells. miR-26a but not control chimera treatment significantly diminished the 5-FU-induced Bak elevation in vivo (FIG. 2G). To determine the role of Bak in chemo-induced hematopoietic injury, Bak1 KO mice and wild type mice were treated with 5-FU and the frequency of LSK in bone marrow was analyzed at five days after treatment. As shown in FIG. 2H, 5-FU treated WT mouse BM had significantly lower percent of LSK when compared with that of Bak1 KO mice. When normalized against the percent of LSK in bone marrow of the untreated mice, it is clear that 5-FU caused more dramatic reduction of Bak1^{+/+} LSK than Bak1^{-/-} LSK (FIG. 2I). The myeloprotective effect against 5-FU by Bak1 deletion was further revealed by increased leukocyte counts and platelet counts in peripheral blood of the 5-FU-treated Bak1^{-/-} mice (FIG. 2J and FIG. 7). These results demonstrate that exogenously delivered miR-26a mediates myeloprotection from chemotherapy at least in part by inhibiting Bak1-induced pro-apoptotic signaling.

miR-26a Plays an Essential Role in Hematopoietic Reconstitution after BM Transplantation

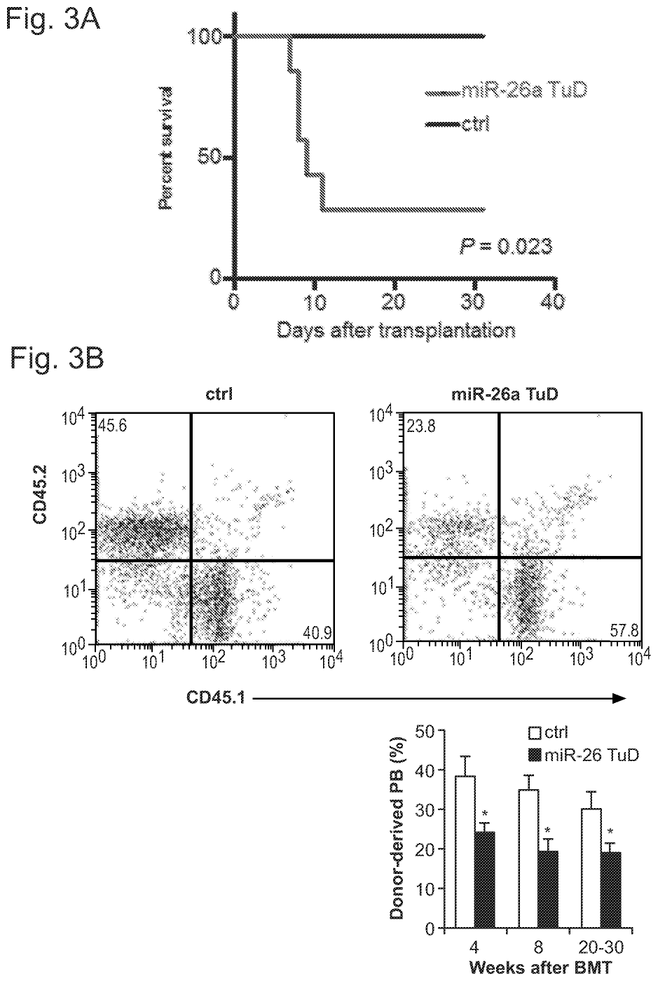

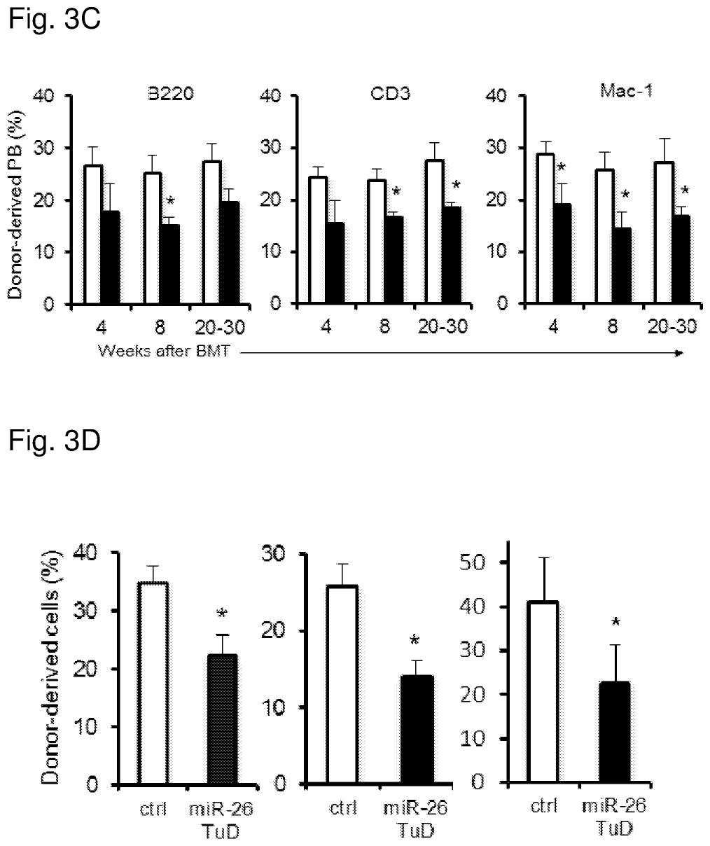

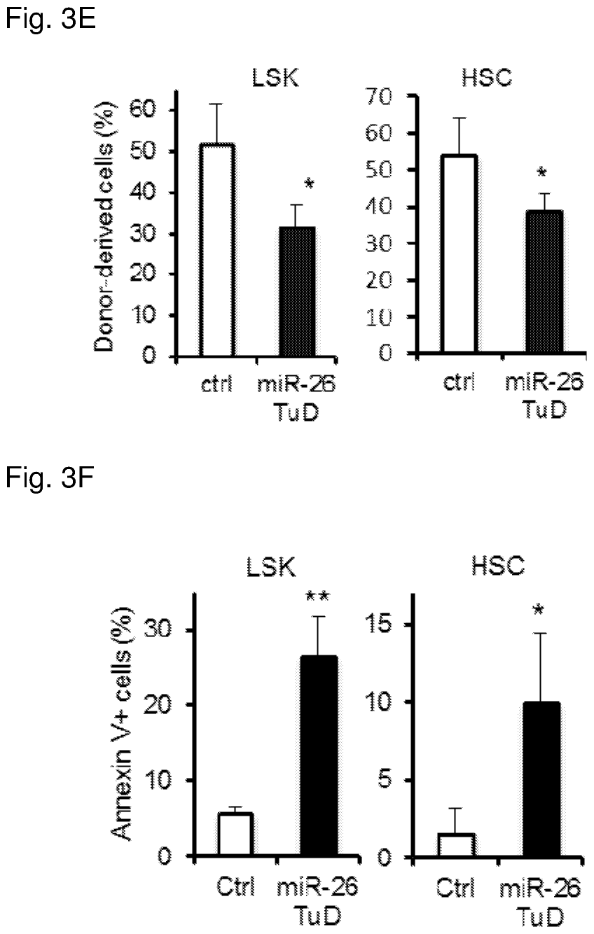

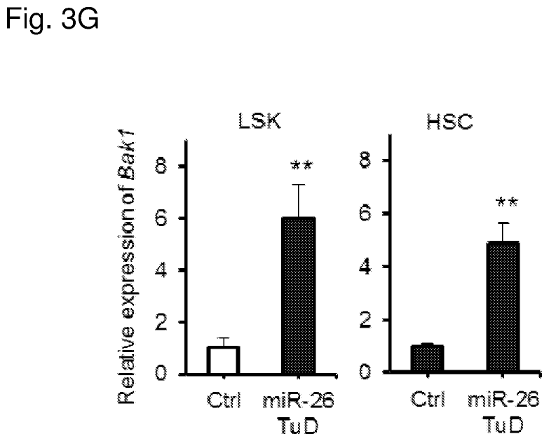

To further investigate the role of endogenous miR-26a on stress hematopoiesis, miR-26a function was inhibited in BM cells using the miR-26a TuD inhibitor their radioprotective activity tested. While mice that received control inhibitor-treated BM were radioprotected, more than 70% of mice transplanted with miR-26a TuD BM cells died between 7-10 days after BM transplantation (FIG. 3A), suggesting that miR-26a plays a major role in radioprotection during the BM transplantation (BMT). Furthermore, miR-26a TuD-transduced and control inhibitor-transduced BM cells were compared for their ability to compete with recipient type BM. Briefly, donor-type BM cells (CD45.2) were transduced with control inhibitor or miR-26 TuD, mixed with equal number of recipient-type BM cells (CD45.1), and then transplanted into lethally irradiated CD45.1 recipients. At 8 weeks, control inhibitor-transduced BM cells contributed roughly 40% of leukocytes in the peripheral blood, while miR-26a TuD-transduced BM cells contributed only about 20% (FIG. 3B, left panel). The significant reduction was observed throughout 20 weeks of observation period (FIG. 3B, right panel). Similar defects were observed in B cells, T cells, and myeloid cells (FIG. 3C). Likewise, the miR-26 TuD BM-derived leukocytes were significantly reduced in BM, spleen, and thymus (FIG. 31)). The severe defects were reflected among LSK cells (CD3e⁻/B220⁻/CD11b⁻/Gr-1⁻/Ter119⁻/Sca-1⁺/c-Kit⁺) and hematopoietic stem cells (HSC) (CD3e⁻/B220⁻/CD11b⁻/Gr-1⁻/Ter119/Sca-1⁺/c-Kit⁺/CD48 ⁻/CD150⁺) (FIG. 3E). As shown in FIG. 3F, Annexin V⁺ cells were significantly increased both in miR-26a TuD-treated LSK and HSC populations. As expected, Bak1 expression levels were significantly increased in these populations (FIG. 3G). These results suggest that miR-26a plays an essential role against hematopoietic stresses by inhibiting an apoptotic pathway.

miR-26a Chimera Inhibits Mouse Breast Cancer Growth and Protects from Chemo-Induced Myelosuppression

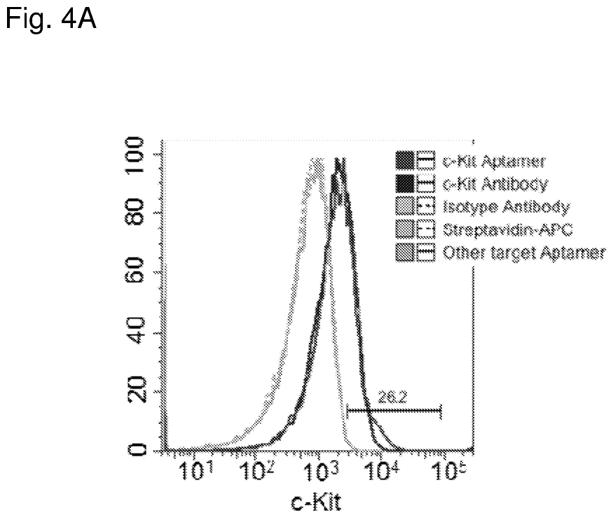

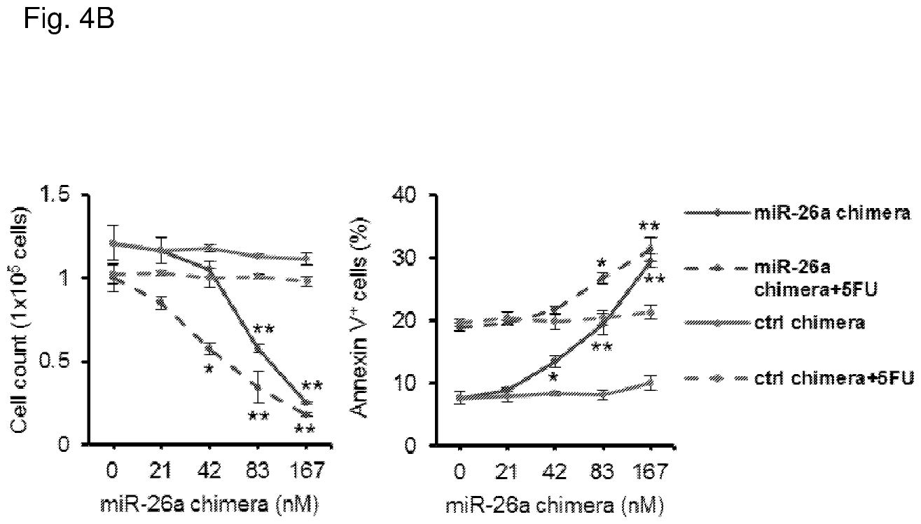

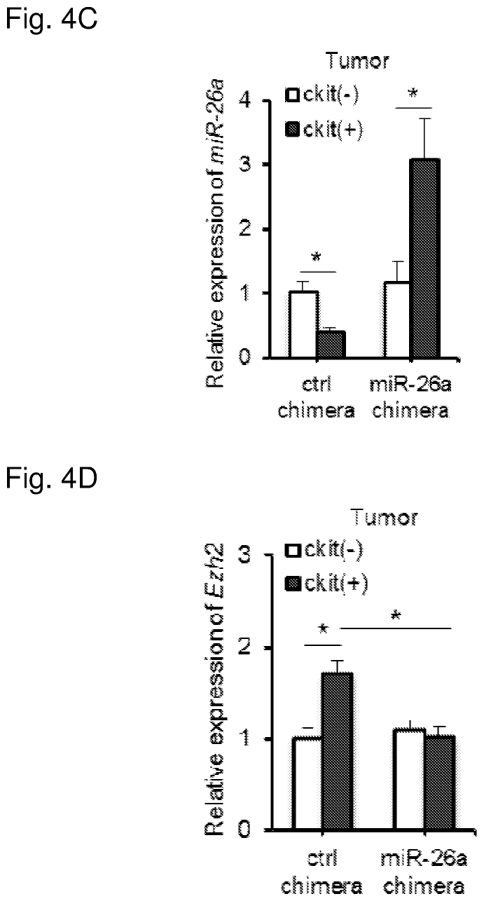

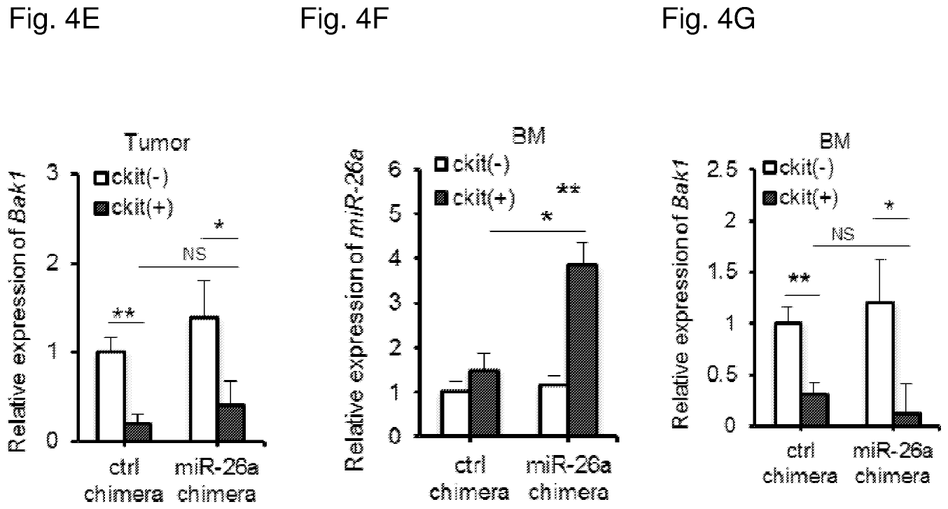

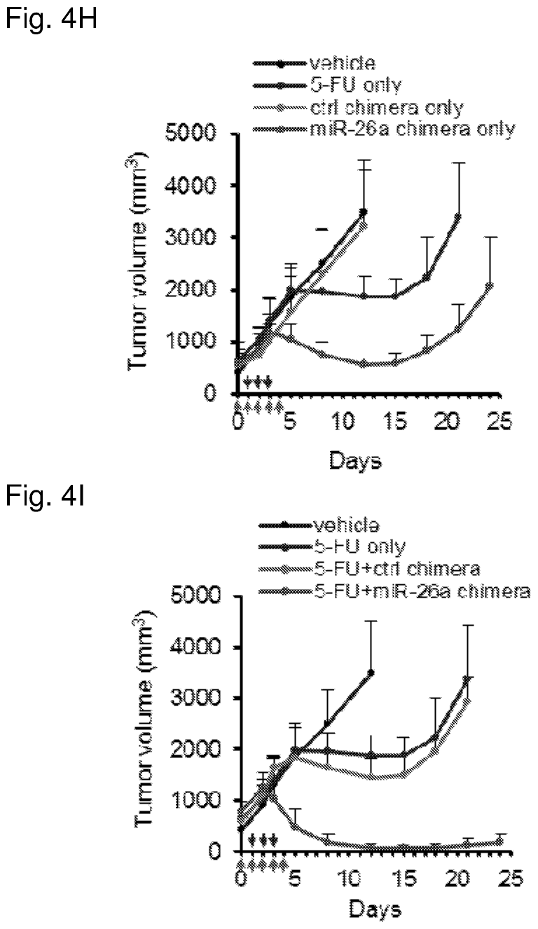

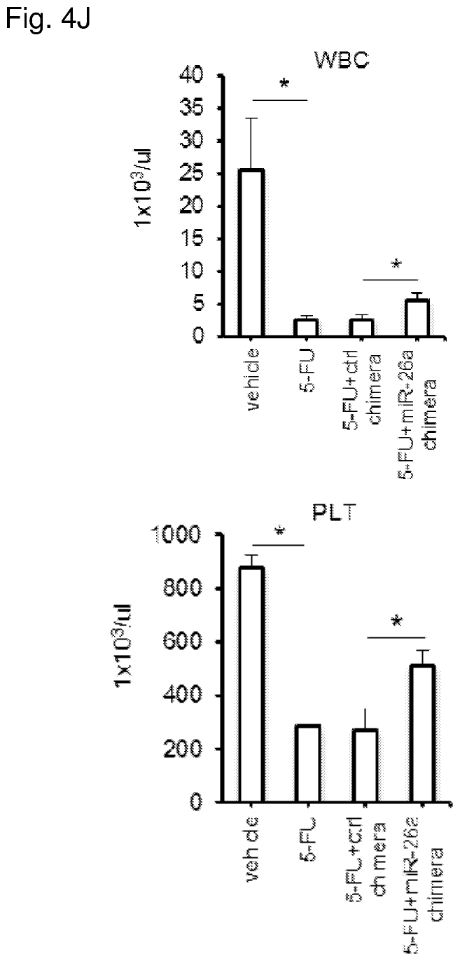

A mouse TUBO breast cancer model was used to evaluate the therapeutic potential of miR-26a chimera for the myeloprotection and anti-tumor growth in breast cancer chemotherapy. Binding ability of an aptamer targeting mouse c-Kit to the TUBO cells was first tested by flow cytometry. As shown in FIG. 4A, biotinylated miR-26a chimera bound to TUBO cells at nearly identical levels comparable to an anti-c-Kit antibody. To determine whether the mouse c-Kit-miR-26a chimera can inhibit the growth of mouse breast cancer cells in vitro, the TUBO cells were treated with increasing doses of miR-26a- or control-chimeras, and the viable cells counted under microscope at 2 days after the treatment. As shown in FIG. 48, left panel, miR-26a chimera alone caused a dose-dependent reduction in tumor cell number with IC₅₀ of approximately 83 nM. This is considerably more effective than 7 μM of 5-FU alone, which caused less than 20% reduction in tumor cell number. In combination, 5-FU and miR-26a chimera synergistically reduced tumor cells. The reduction was due to induction of apoptosis as the percentage of apoptotic TUBO cells were increased by both 5-FU and miR-26a chimera and their combination (FIG. 4B, right panel).

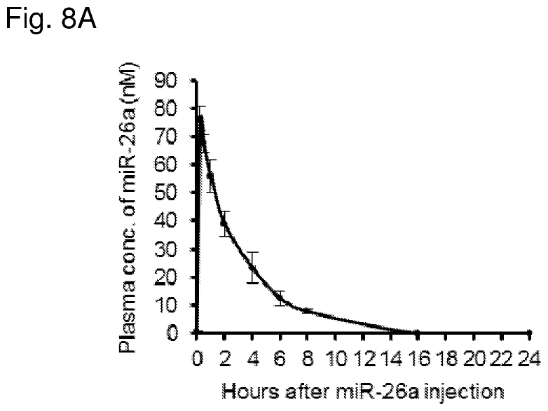

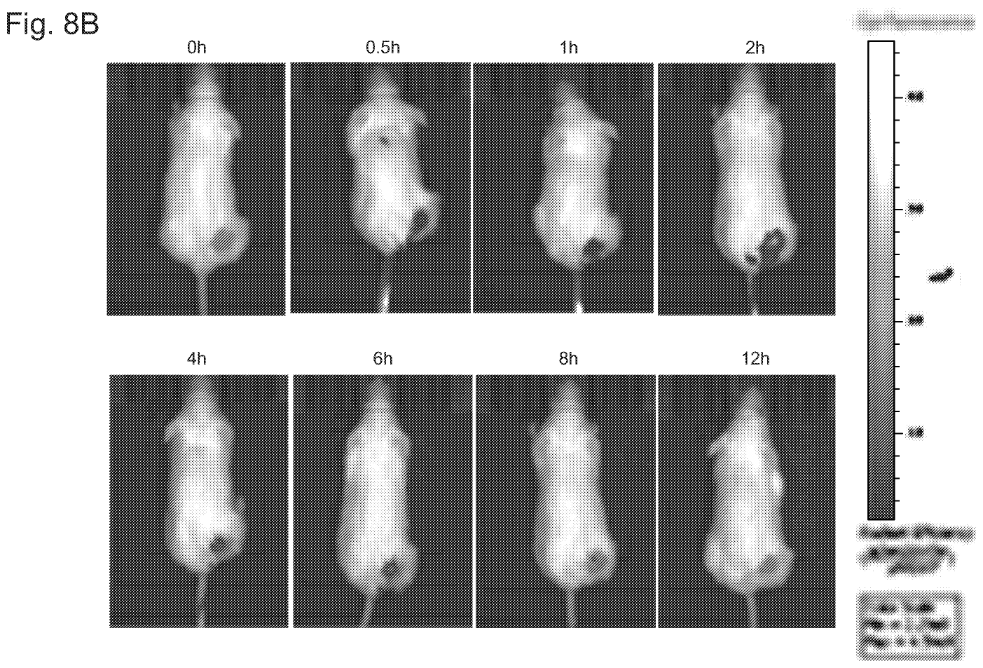

To test the therapeutic effect of miR-26a chimera, miR-26a- or control chimera was injected intravenously into TUBO-tumor-bearing mice. As shown in FIG. 8A, significant levels of miR-26a chimera could be detected in the blood of tumor bearing mice that received 670 pmol of chimera up till 8 hours after injection. Moreover, the accumulation of miR-26a chimera into the tumor was visualized by in vivo imaging using Alexa Fluor 647 dye-conjugated miR-26a chimera (FIG. 8B). To further confirm the targeting delivery of miR-26a in vivo, levels of miR-26a among sorted c-Kit+ and c-Kit− TUBO cells were measured at 3 days after injection by qPCR. As shown in FIG. 4C, significant increase of miR-26a was detected among c-Kit+ but not the c-Kit− tumor cells, thus confirming specific delivery of the miR-26a to the c-Kit+ tumor cells. Functional delivery was further confirmed by the specific decrease in the miR-26a target gene Ezh2 among the c-Kit+ tumor cells (FIG. 4D). In contrast, Bak was barely expressed among c-Kit+ tumor cells and such expression was not significantly elevated by miR-26a chimera (P=0.49) (FIG. 4E). Consistent with non-tumor-bearing C57BL/6 mice, a selective increase in miR-26a levels was observed in c-Kit+BM cells from the mice treated with miR-26a chimera on day 3 after single treatment (FIG. 4F). Interestingly, in mice that received no 5-FU-treatment, c-Kit+ HSPC also expressed very low levels of Bak1, and its expression was not significantly down-regulated by miR-26a chimera (FIG. 40). Therefore, it appears that miR-26a regulates Bak1 expression only during stress-induced hematopoiesis, such as within hosts that received irradiation or chemotherapy21.

To test the anti-tumor and myeloprotective effects by miR-26a chimera in vivo, mice bearing mammary tumors (TUBO) were treated with multiple injections of 5-FU and miR-26a chimera. A significant decrease of tumor size was observed after 5 daily injections of miR-26a chimera (FIG. 4H). Although either 5-FU or miR-26a chimera monotherapy was sufficient to cause growth retardation, miR-26a chimera appeared more effective than 5-FU treatment (FIG. 4H). Remarkably, combining 5-FU with miR-26a chimera achieved most effective growth retardation (FIG. 4I).

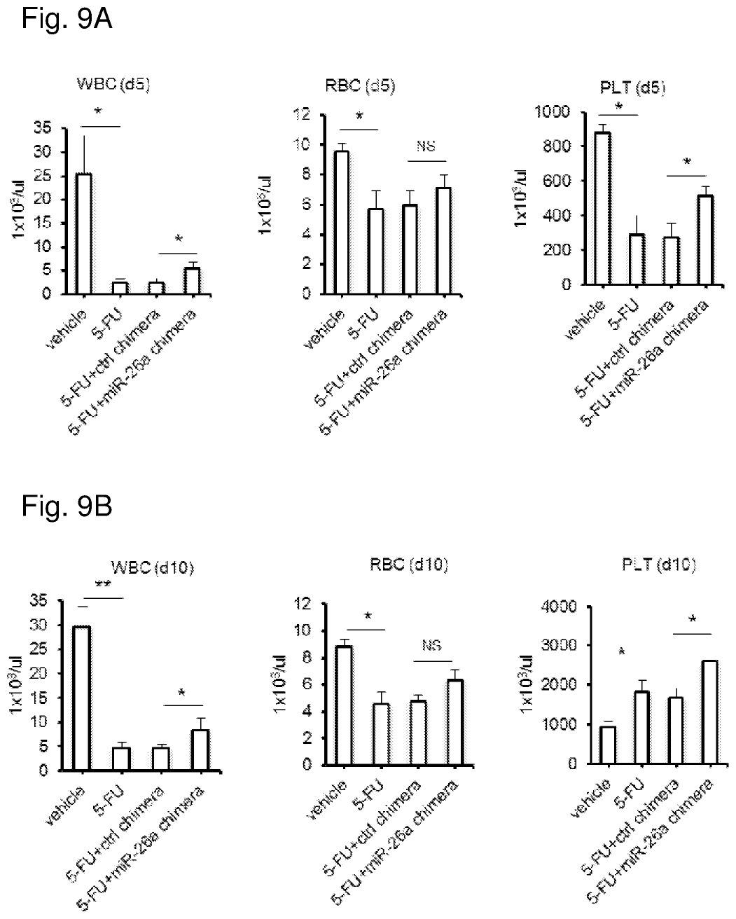

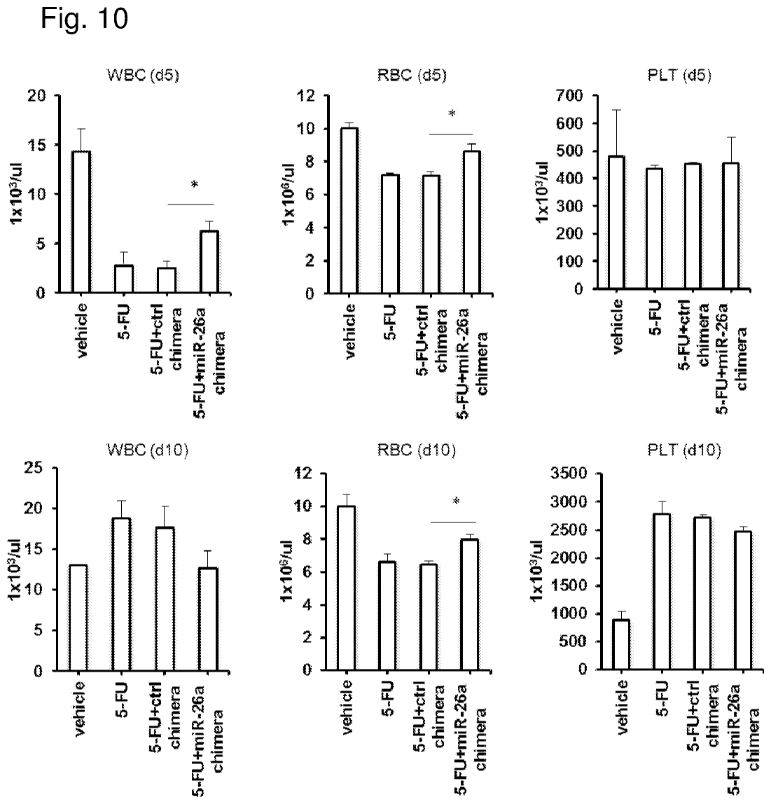

Notably, the number of leukocytes in TUBO-tumor bearing mice were nearly 3-times the normal range of 8.05+/−1.04×10³/μl (33), which is consistent with a tumor-induced leukocytosis. As expected, 5-FU treatment not only eliminated leukocytosis, but also caused significant leukopenia and thrombocytopenia (FIG. 4J and FIG. 9). Importantly, combination treatment with miR-26a chimera nearly doubled the number of leukocytes (WBC) and thrombocytes (PLT) and largely prevented leukopenia (FIG. 4J and FIG. 9).

miR-26a Protects Mice Against Hematopoietic Toxicity from Carboplatin

The addition of neoadjuvant carboplatin to the regimen of taxane and anthracycline significantly increases the proportion of patients achieving a pathological complete response (34). However, adverse effects such as anemia, neutropenia, thrombocytopenia (grade 3 or 4 hematological events) occurred more frequently in patient group given carboplatin. To evaluate if miR-26a broadly protect mice against chemotherapy-related hematological toxicity, we tested the effect of miR-26a on myelosuppression by carboplatin treatment. As expected, high-dose of carboplatin treatment induced significant defects in hematopoiesis. Thus, compared with vehicle, a single dose of carboplatin significantly reduced WBC and platelet counts as early as day 5 (FIG. 6A). The defect persisted and exacerbated at least for 10 days, when the reduction of red blood cells was also observed (FIG. 6B). Remarkably, 3 daily injections of miR-26a chimera, but not control chimera, prevented reduction in WBC and RBC. While reduction of platelet on day 5 was not prevented, miR-26a chimera prevented further drop of platelets from day 5 to day 10. Therefore the myeloprotective effect of the miR-26a chimera is not limited to 5-FU.

Taken together, based on the finding that miR-26a mediated a converging pathway in cancer progression and 5-FU-induced myelosuppression, a new combination therapy was developed that improved efficacy of 5-FU while ameliorating its main adverse effect. The dual benefit of combination therapy will likely extend to other chemotherapies, as miR-26a has been shown to act synergistically with Paclitaxel in killing breast cancer cells in vitro (35). Furthermore, while this study focused on breast cancer models, miR-26a as a tumor suppressor has been observed in other cancer types, including prostate cancer, pancreatic cancer, and lung cancer (36-38). Likewise, KIT is also widely expressed among human cancers including gastrointestinal stromal tumors, myeloid leukemia, small cell lung cancer, prostate cancer, pancreatic cancer, ovarian cancer and glioblastoma (39-45). Therefore, the clinical significance of the miR-26a chimera targeting KIT⁺ cancers would extend well beyond breast cancer. However, it should be noted that miR-26a demonstrates its opposing functions as an oncogene in acute myeloid leukemia, ovarian cancer, and glioma (46-48), careful consideration should be given to the designing of cancer therapies using the combination of miR-26a with KIT.

Apart from 5-FU, this data demonstrated remarkable efficacy of miR-26a chimera in protecting mice against hematopoietic toxicity from carboplatin. Carboplatin is a platinum-based and inter-strand cross-linking antineoplastic agent. Platinum-based compounds remain in use for chemotherapy drugs despite its toxicity (34). By preventing its hematopoietic toxicity, this new approach may allow even broad use of this class of chemotherapeutic drugs.

Although general and specific inactivation of miRNA play major role in cancer pathogenesis (15-17), difficulties in miRNA delivery to cancer cells has limited potential utility of miRNA in cancer therapy. While an adenovirus-associated virus-based miRNA delivery system has been reported for miR-26a administration in a liver cancer model (49), it is unclear whether this is generally applicable to other cancer types. Here it was demonstrated that aptamer can be used for specific delivery of miRNA to targeted cells, providing the advantages of being effective at lower dose, low immunogenic, and highly scalable method for miRNA delivery with no risk of genomic integration (50). Given the existence of large bank of aptamers for cell surface markers, the new approach described herein will likely have a broad impact for studying biological function of miRNA in vivo and for cancer therapy.

### Example 3

Embodiments of the disclosure relate to a composition comprising a nucleic acid sequence having at least two domains: an aptamer domain and an exosome targeting sequence domain. Such embodiments are made and tested for effectiveness as follows.

Exosome Preparation from Cell Culture
1. Culture cell line (ex. human HEK293, mouse JAWSII) in 60-cm dish with 10 ml culture medium (Exosome-free FBS) for 3 days.
2. Collect 10 ml culture medium and spin at 400×g, 5 min.
3. Transfer the supernatant to new 15 ml tube.
4. Add 2 ml volume of ExoQuick-TC (SBI) into the 10 ml supernatant and mix well.
5. Refrigerate (4C) overnight.
6. Centrifuge at 1500×g, 30 min, 4 C.
7. Aspirate the supernatant, and spin down at 1500×g, 5 min.
8. Remove all supernatant carefully, and resuspend the pellet in 1 ml sterilized PBS.
9. Aliquot 30 ul (approx. 1×10\hat{ }7 particles, 50-300 ug exosome protein) into 33 eppendorf tubes and keep in −20 C.
   1-5×10\hat{ }8 particles/mi exosome from 293T cells.
      Exosome Preparation from Serum
1. Collect 1 ml peripheral blood into an eppendorf tube.
2. Leave it in a fridge (4C) over night.
3. Centrifuge at 2000×g, 10 min, 4 C.
4. Transfer 500 ul serum to a new eppendorf tube.
5. Add 125 ul ExoQuick (SBI) into the serum and mix well.
6. Refrigerate (4C) overnight.
7. Centrifuge at 1500×g, 30 min, 4C.
8. Aspirate the supernatant, and spin down at 1500×g, 5 min.
9. Remove all supernatant carefully, and resuspend the pellet in 300 ul sterilized PBS.
10. Aliquot 30 ul (>1×10\hat{ }7 particle/30 ul) into 10 eppendorf tubes and keep in −20 C
   1.0-4.0×10\hat{ }8 particle/ml human plasma.

### CD63-Target Chimera Preparation

1. Prepare 5 uM targeting-DNA aptamer solution (ex. ckit) and CD63 aptamer solution in 100 ul annealing buffer (30 mM HEPES, 100 mM Potassium acetate, p1H7.5) by adding 5 ul of 100 uM stock solution into 90 ul buffer, individually.
2. Add 5 ul of 50 mM MgCl₂.
3. Heat at 95C for 10 min
4. Snap cool on ice for 10 min
5. Mix two solutions (CD63 and targeting) in one tube (total 200 ul) and aliquot into 2 PCR tubes (100 ul/tube).
6. Incubate in thermal cycler
   55C 20 min
   37C 60 min
   4C
7. Pool the solutions (5 uM [5 pmol/ul] target+CD63 chimera)

CD63-Apt linker
(SEQ ID NO: 147)
C*A*C*CCCACCTCGCTCCCGTGACACTAATGCTA/iSpC3/
TAATGAA*C*T*T
Mouse eKit-Apt linker
(SEQ ID NO: 148)
G*C*T*CAACGCGGGACGGCTCTCCCATTGAC/iSpC3/
AAGTTCA*T*T*A

### Exo-Fect Reaction

1. Add by following order in 1.5 ml sterilized tube.

| | | |
|---|---|---|
| Exo-Fect solution (SBI) | 5 | ul |
| Plasmid DNA (2.5 ug) | | ul |
| or mRNA (1 ug) (<1 kbp) | | ul |
| or si/miRNA (200 pmol) | | ul |
| or small molecule (>100 ug) | | |
| Sterilized PBS | | ul |
| Exosome (60-120 ug, 1 × 10\hat{ }7 particles) | 30 | ul |

75 ul (per one transfect to
a well of 12 well plate)
*SBI; System Biosciences
* For in vivo (mouse, iv injection), prepare >120 ug exosome

2. Mix the components well by flicking/inversion 3 times. No vortex!
3. Incubate 37C for 10 min.
4. Add 15 ul Exo-Quick-TC and mix by inverting 6 times. No vortex!
5. Incubate at 4C for 30 min.
6. Centrifuge at 15,000 rpm, 5 min, 4C and carefully remove the supernatant.
7. Resuspend the pellet in 100 ul PBS.
8. Add 5 ul target-CD63 aptamer chimera (5 uM).
9. Incubate for 30 min on ice.
10. Add 650 ul PBS and 200 ul Exo-Quick-TC and mix by inverting 6 times. No vortex.
11. Incubate for 30 min on ice.
12. Centrifuge at 15,000 rpm, 5 min, 4C and carefully remove the supernatant.
13. Spin again and carefully remove the supernatant.
14. Add 50 ul PBS and resuspend the pellet.
15. Add the 50 ul transfected exosome into 2×10\hat{ }5 cells/500 ul medium/well (12 or 24-well).
16. For in vivo, use 3× volume of targeting exosome for iv injection per day. Repeat 2 more times. * prepare fresh exosome for every injection
17. Incubate for overnight-3 days to see the delivery.

### Targeting Exosome Gene-Protein Delivery System

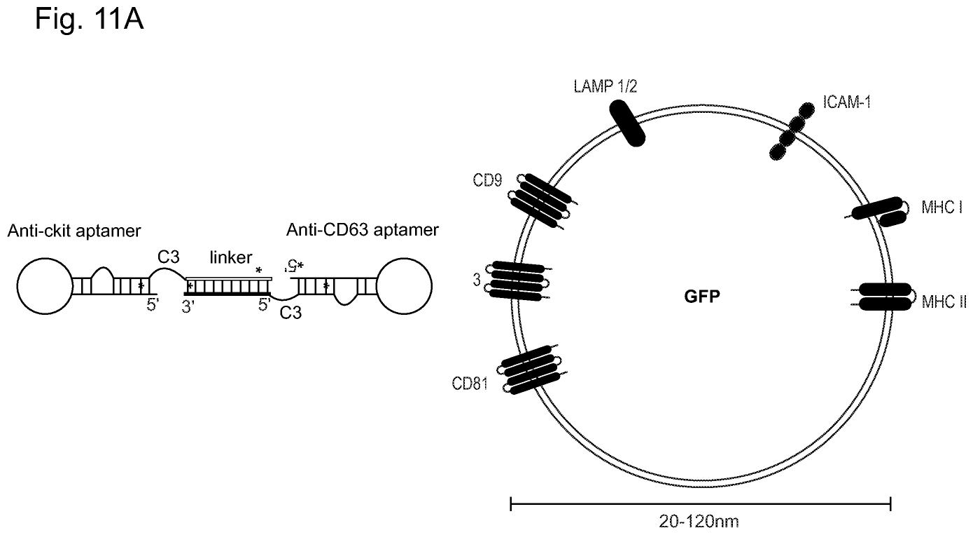

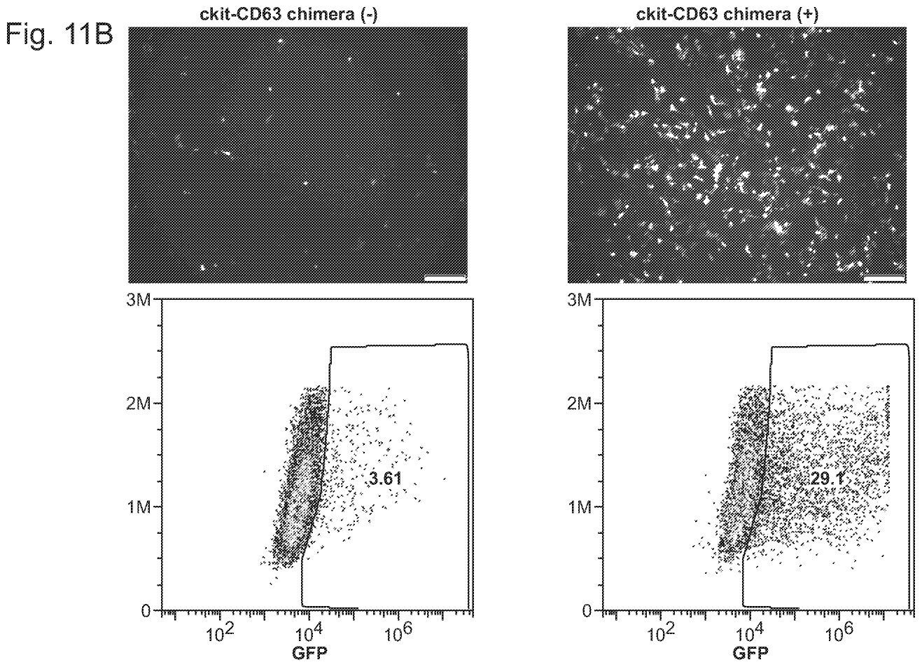

2.5 ug of pmaxGFP plasmid was transfected into HEK293 cell-derived exosome (1×10\hat{ }7 particles) by Exo-fect (FIG. 11A). This was added into the 0.5 ml culture medium of ckit over-expressed MEF cells (1×10\hat{ }5 cells in a 24 well plate). After 2 days, GFP expression was tested by fluorescence (FIG. 11B). The targeting system enhanced the delivery efficiency of the exosome.

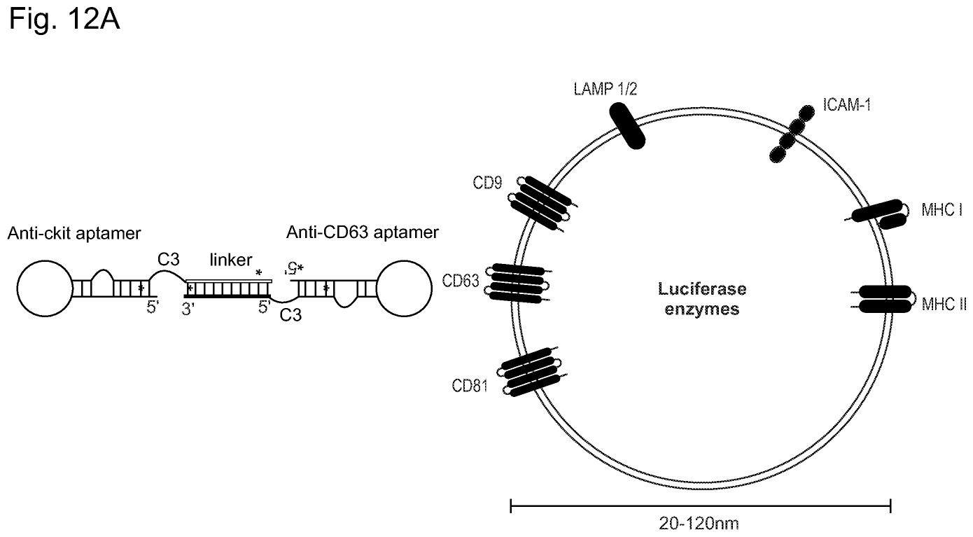

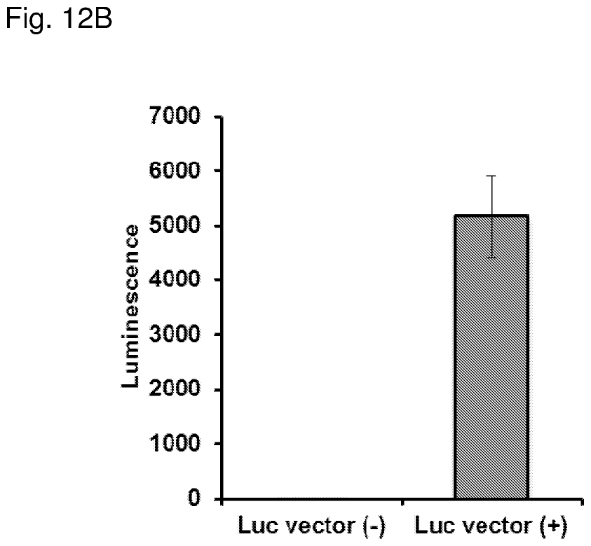

A luciferase expression vector (pmirGLo) was transfected into 1-EK293 cell-derived exosome (FIG. 12A). The exosome was added into the 0.5 ml culture medium of ckit over-expressed MEF cells (1×10\hat{ }5 cells in a 24 well plate). After 3 days, luminescence from luciferase was shown to be significantly increased (FIG. 12B)

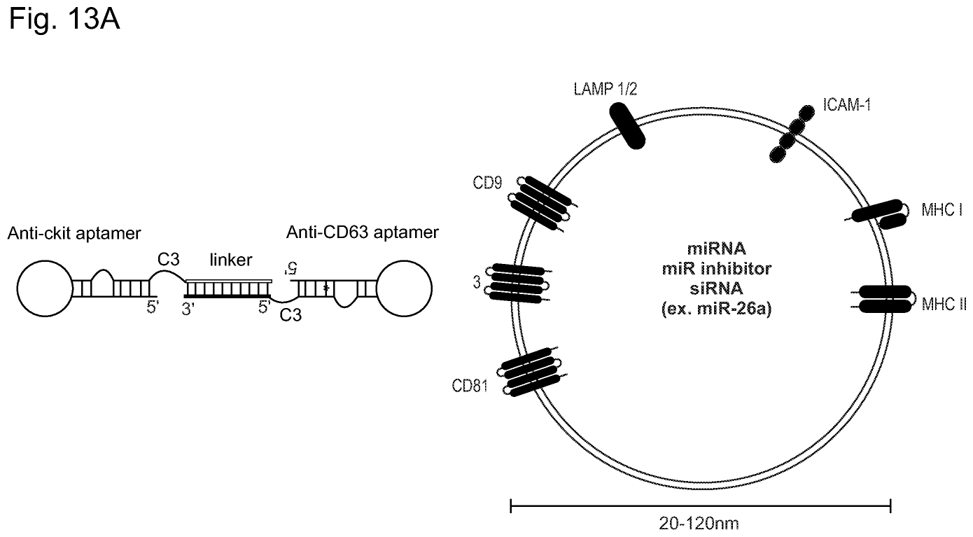

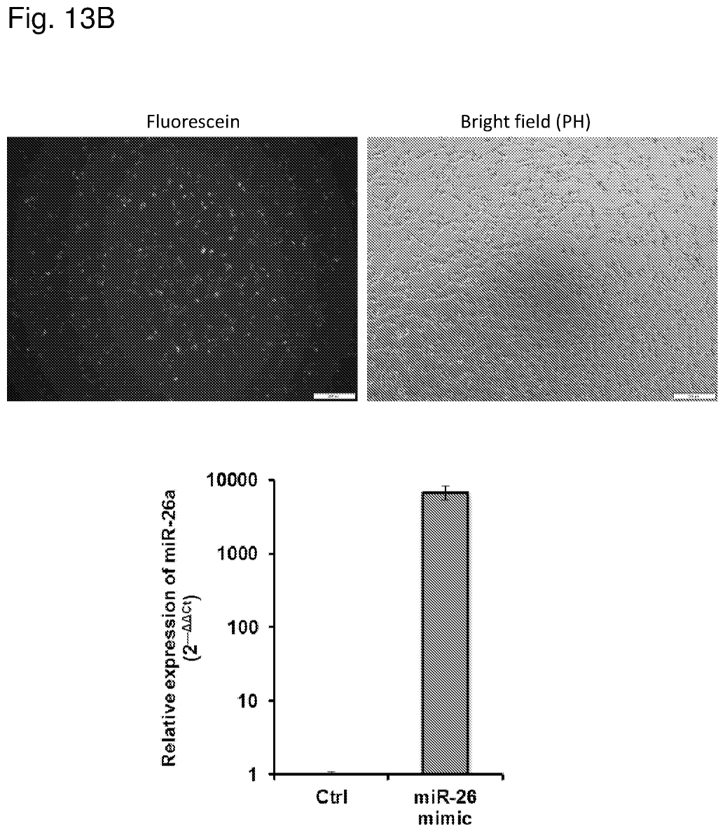

A large amount of small RNA can be delivered into target cells using the exosome delivery method, such as miRNA and siRNA (FIG. 13A). One application of this method is the delivery of miRNA inhibitors into cancer cells that express large amounts of miRNAs. This method may also be used to deliver different miRNAs at one time. For example, miR-26a and miR-195 can be delivered into target cells together. 250 pmol of fluorescein-conjugated miRNA mimic (miR-26a-5p) was transfected into HEK293 cell-derived exosome (1×10\hat{ }7 particles) by Exo-fect. This was added to ckit over-expressed MEF cells (1×10\hat{ }5 cells in a 24 well plate). One day later, cells were shown to express fluorescein (FIG. 13B, top). After 2 days, expression of miR-26a was increased dramatically (FIG. 13B, bottom).

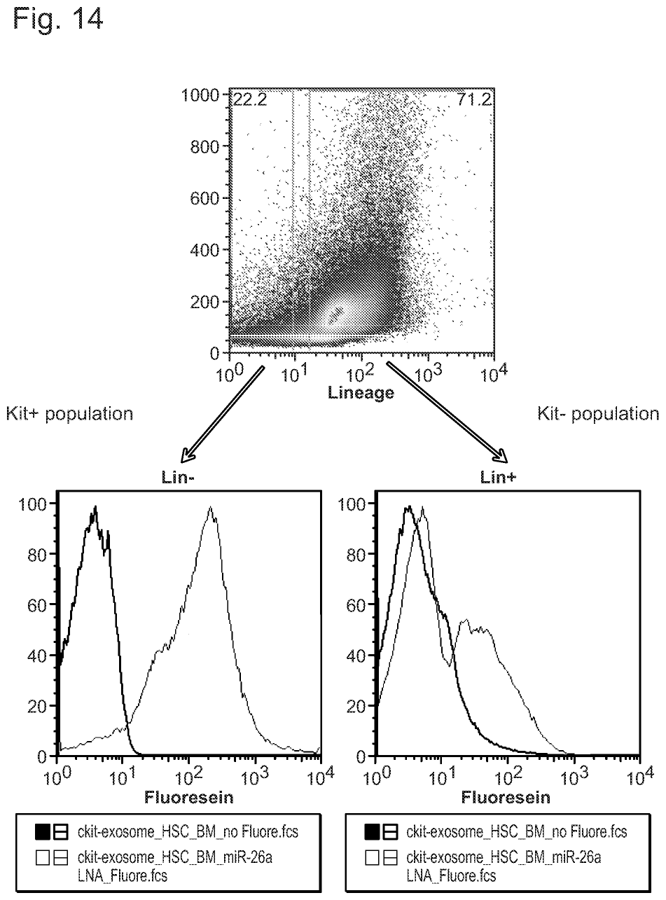

In another experiment, 250 pmol of fluorescein-conjugated miRNA mimic (miR-26a-5p) was again transfected into HEK293 cell-derived exosome (1×10\hat{ }7 particles) by Exo-fect. The exosome was then added into in vitro cultured bone marrow cells (1×10\hat{ }7 cells in a 24 well plate). One day later, flow cytometry analysis revealed significant fluorescence in the ckit-positive (Kit+) population of cells (FIG. 14).

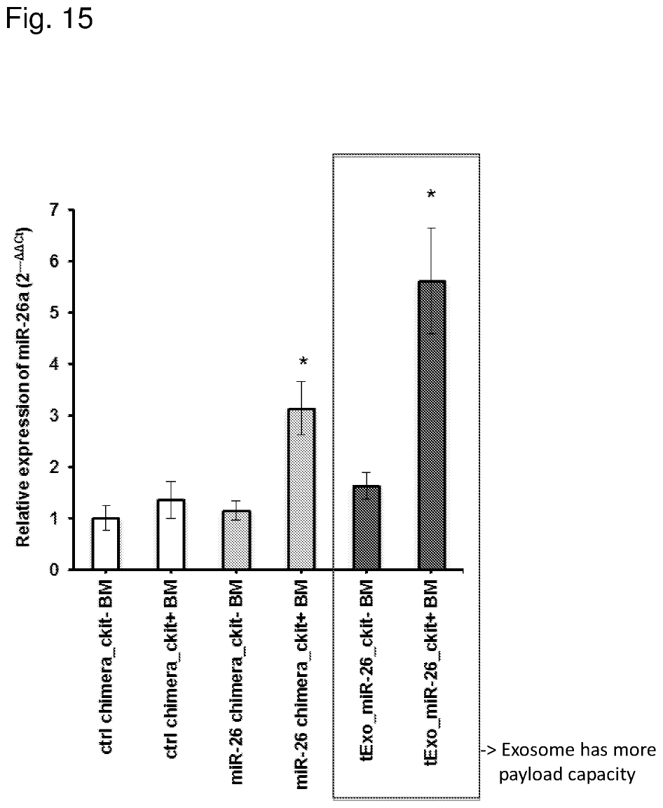

To test the ckit targeting exosome in vivo, 250 pmol of fluorescein-conjugated miRNA mimic (miR-26a-5p) was transfected into JAWSII cell-derived exosome (1×10\hat{ }7 particles) by Exo-fect. 2 days after the exosome was given to via intravenous injection, mouse bone marrow cells were harvested. The cells were divided into ckit+ and ckit− using magnetic-activated cell sorting (MACS). qPCR showed increased expression of miR-26a in ckit+ cells, and a further significant increase in ckit+ cells when the exosome was made with a higher payload capacity (FIG. 15).

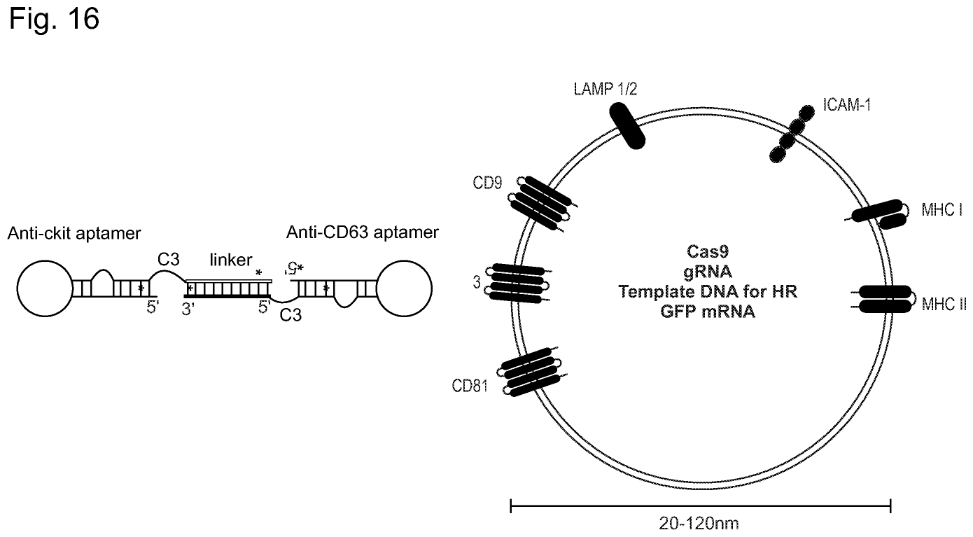

Many CRISPR components can be loaded into exosome by one-time ExoFect for a targeting genome editing tool, even though Cas9 mRNA/protein itself is too large (FIG. 16). This allows the exosome system to target several genes at a time. Additionally, template DNA can be loaded for homologous recombination, whereas use of a virus cannot.

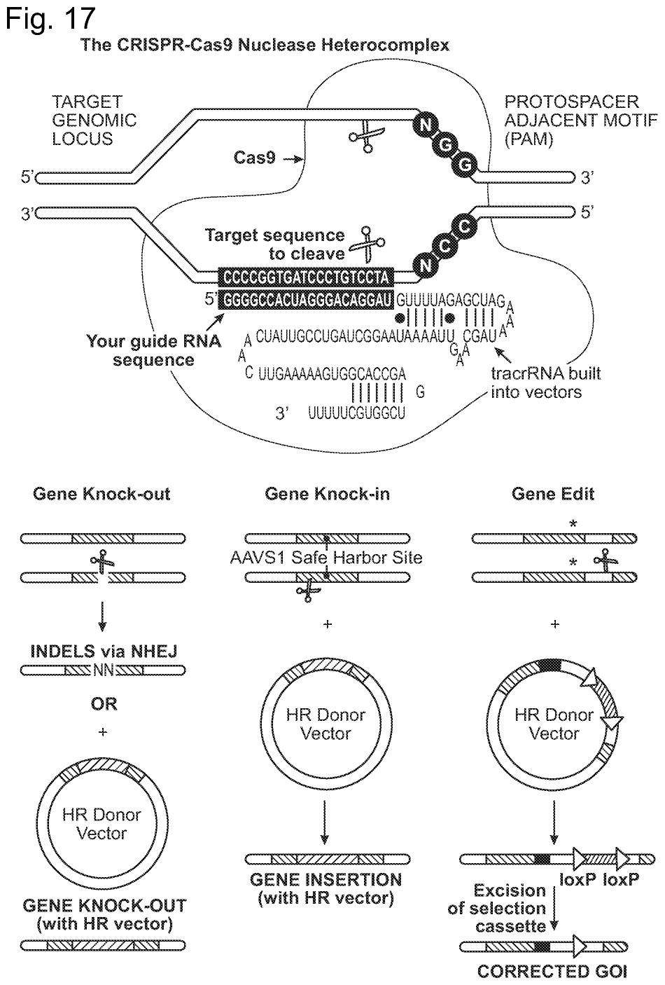

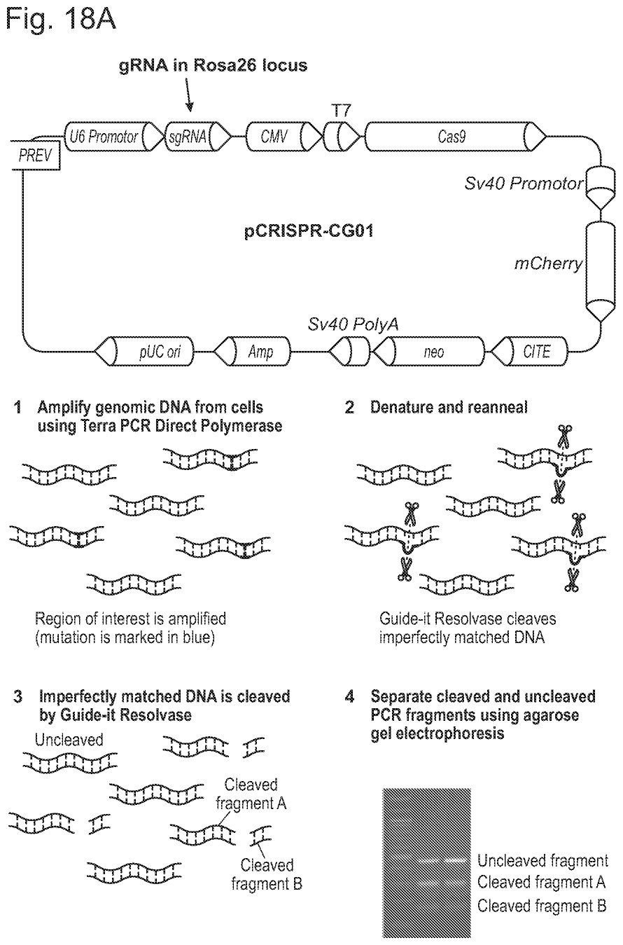

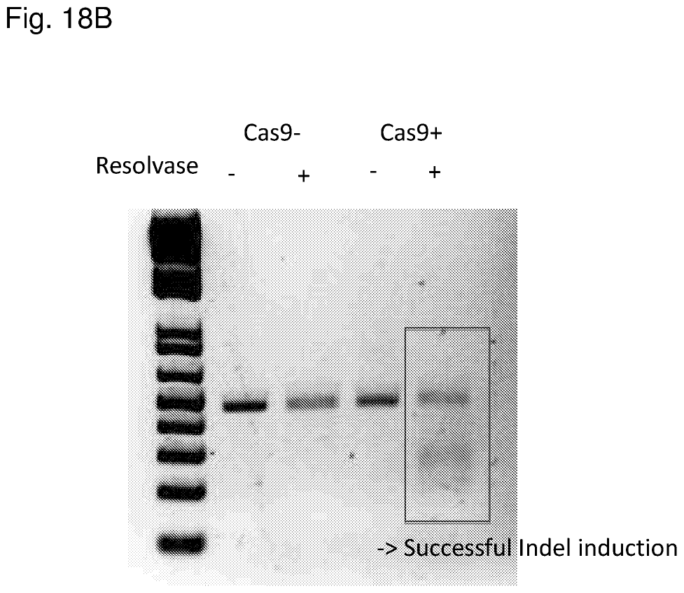

A gene knock-out was created in vitro by targeting exosome containing Cas9 gRNA (FIG. 17). 2.5 μg of Cas9-Rosa26 gRNA plasmid (mCherry reporter; FIG. 18A, top) was transfected into 293T cell-derived exosome (1×10\hat{ }7 particles) by Exo-fect (5 μl) with 5 μl 5 μM ckit-CD63 aptamer. The exosome was then added into ckit over-expressed MEF cells (1×10\hat{ }5 cells). 3 days later, cells were sorted for mCherry, and 10 days later, cells were subjected to the Indel detection assay (Clontech; FIG. 18A, bottom). The assay showed successful indel induction in mCherry-positive cells (FIG. 18B).

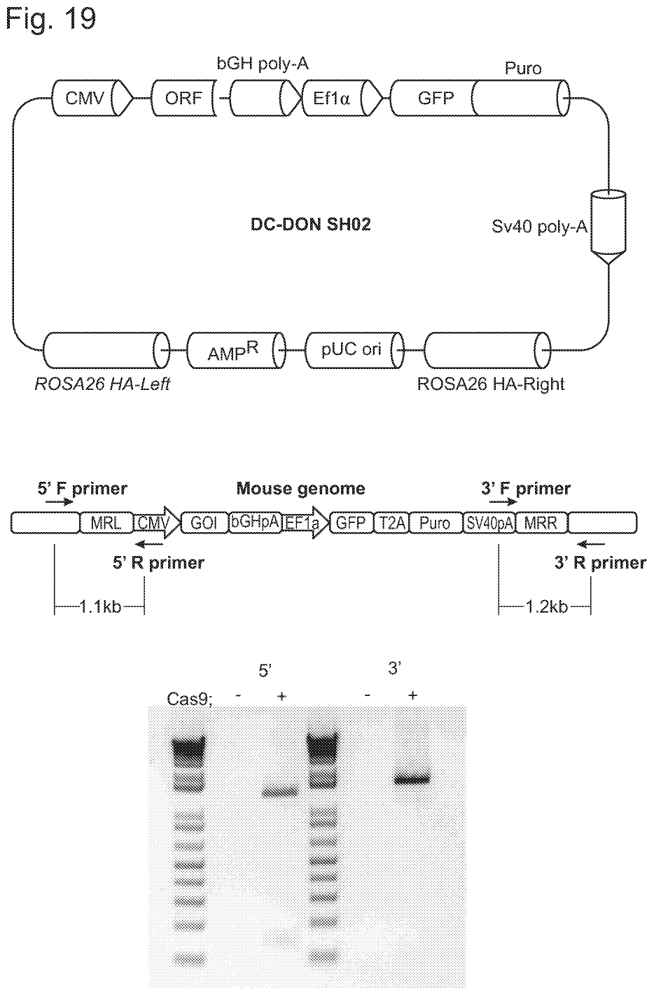

A gene knock-in was created in vitro by targeting exosome with homologous recombination vector. 1.25 μg of Cas9-Rosa26 gRNA plasmid (mCherry) was transfected into 293T cell-derived exosome (1×10\hat{ }7 particles) by Exo-fect (5 μl) with 5 μl 5 μM ckit-CD63 aptamer and 1.25 μg Donor vector (RFP/GFP/Puro). The exosome was then added into ckit over-expressed MEF cells (1×10\hat{ }5 cells). 3 days later, cells were subjected to 2 μg/ml Puromycin selection. 10 days later, Junction PCR was used for an integration check. It was found that template sequence integration was successfully detected by PCR, demonstrating that homologous recombination occurred (FIG. 19).

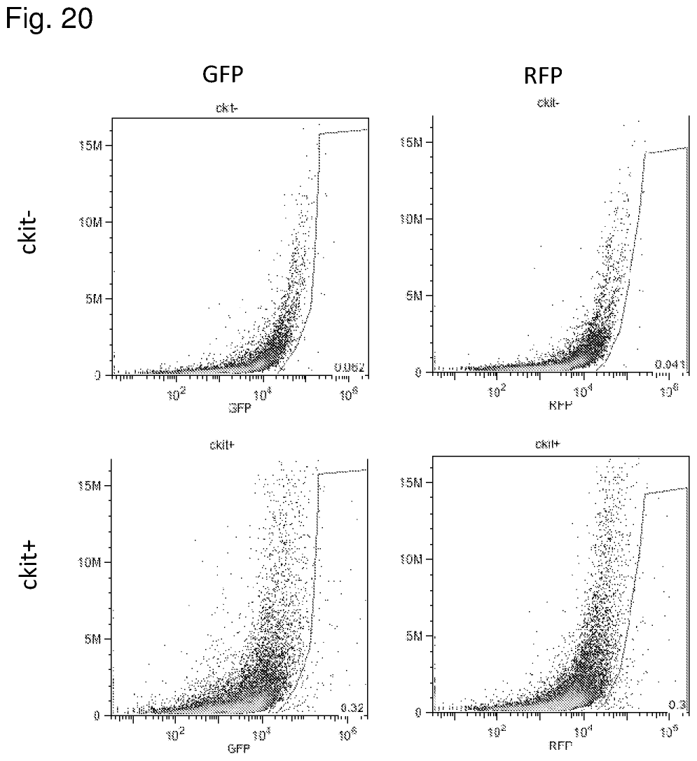

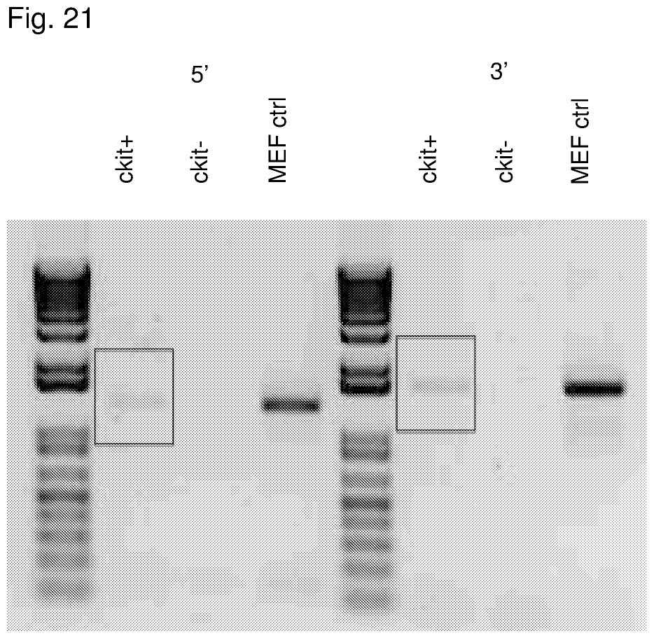

A gene knock-in was created by targeting exosome to mouse bone marrow cells in vivo. Rosa26 gRNA-Cas9 vector with template DNA vector (RFP/GFP/Puro) was transfected into JAWSII dendritic cell-derived exosome by Exo-fect. 120 μg of exosomes were injected twice intravenously into a BL6 mouse. 11 days later, bone marrow was harvested and underwent lineage depletion. ckit+ selection was performed by MACS (FIG. 20), but was not able to be detected with Junction PCR. With ex vivo cell culture and 1 μg/ml puromycin selection over 4 days, Rosa locus Junction PCR successfully detected template sequence integration, demonstrating that homologous recombination occurred (FIG. 21).

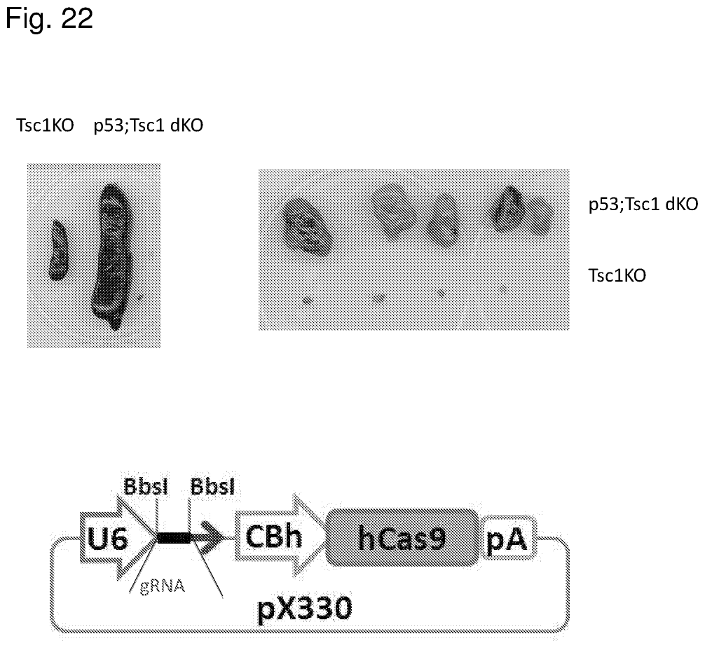

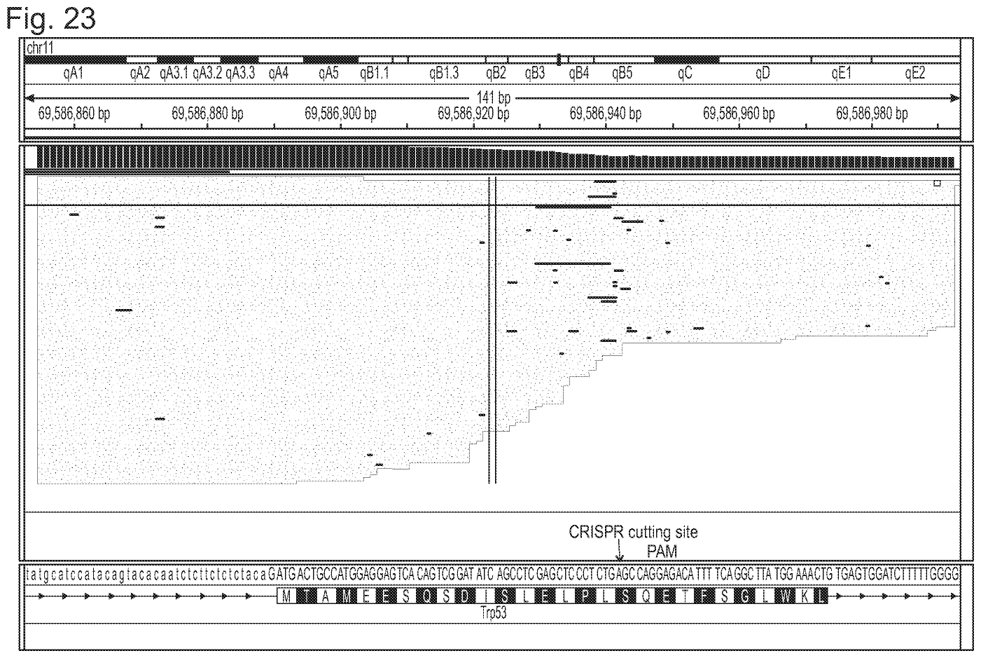

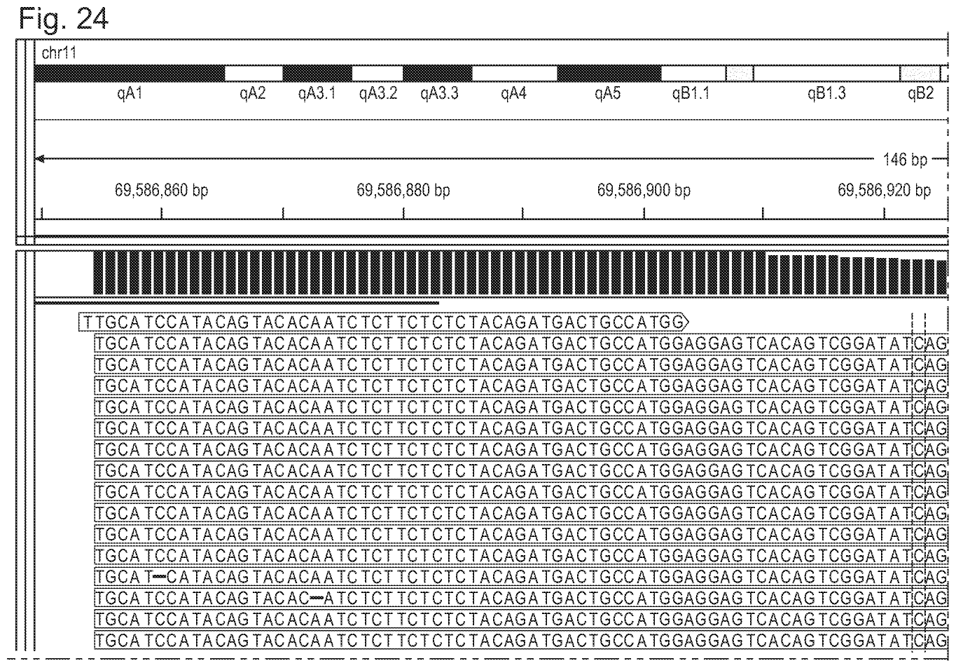

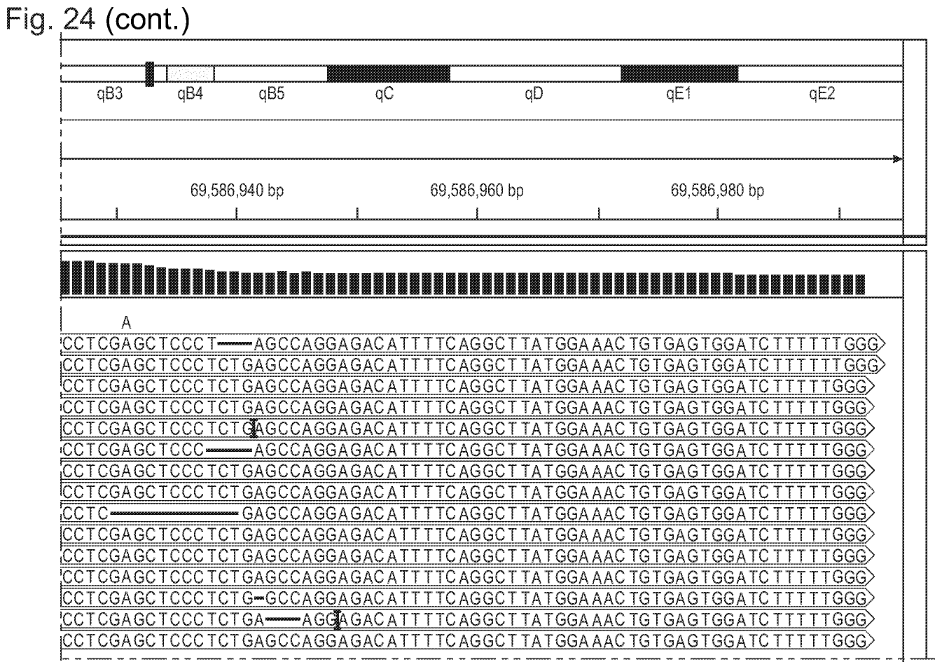

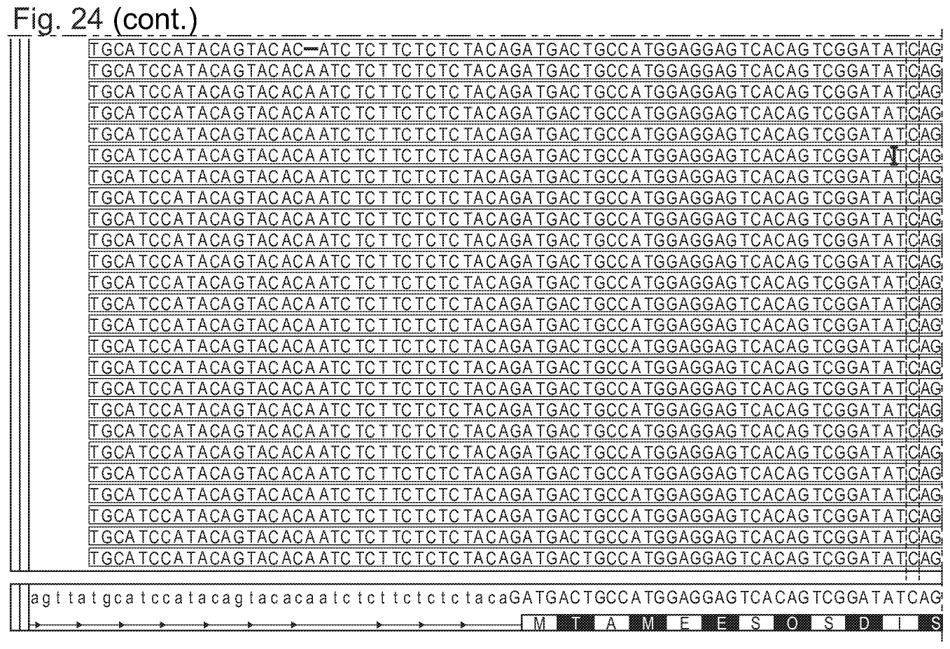

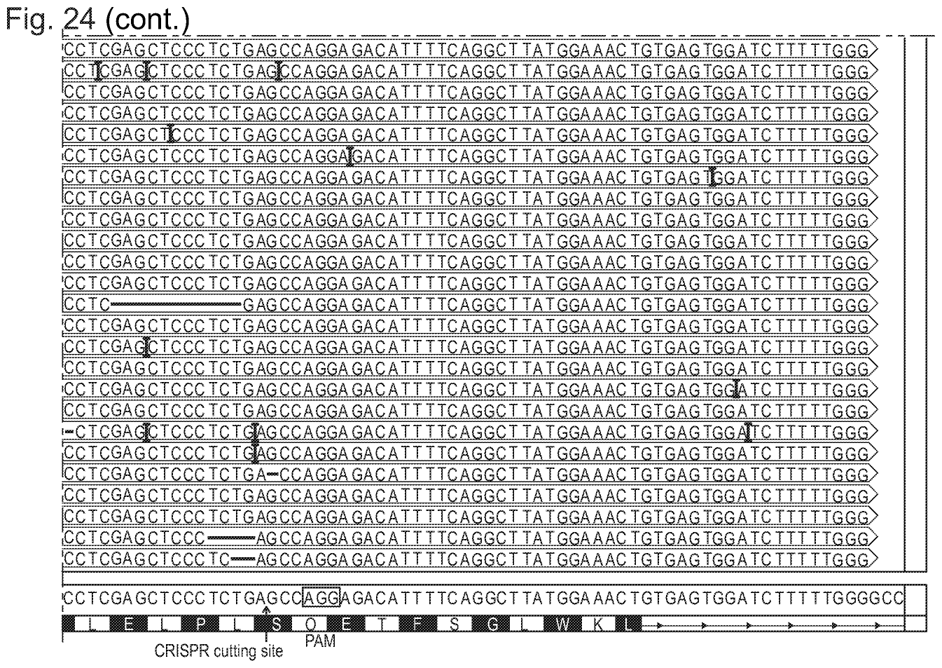

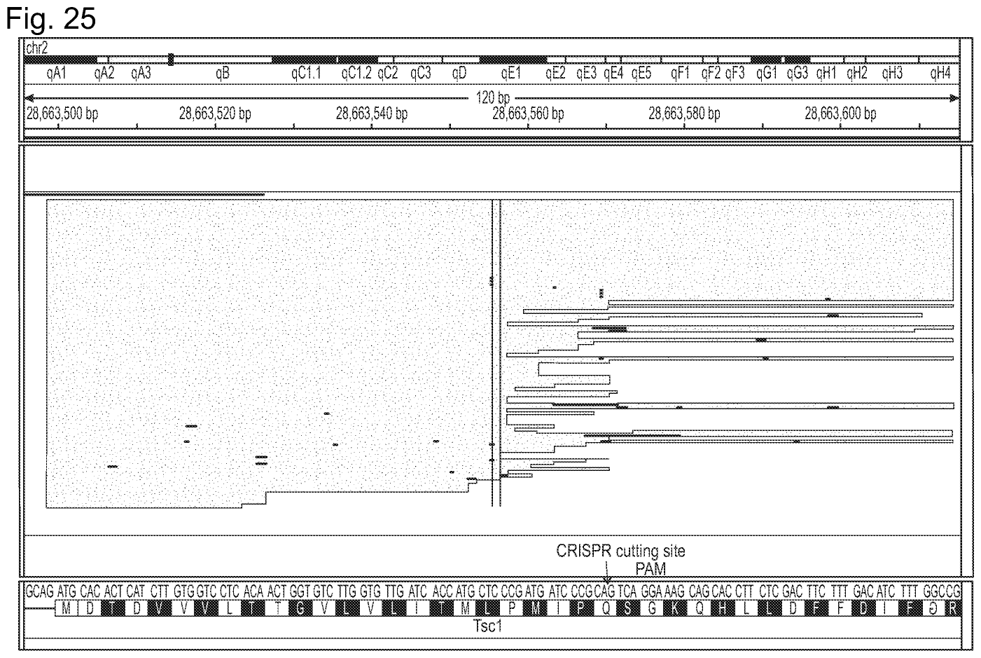

A gene knock-out was created by targeting exosome to mouse bone marrow cells, making a leukemia mouse model. CRISPR gRNAs for mouse p53 gene (pX330; FIG. 22, bottom) and Tsc1 gene (pX458; Cas9-GFP) were used in vitro. 1.25 μg p53 plasmid and 1.25 μg Tsc1 plasmid with template DNA vector (RFP/GFP/Puro) was transfected into JAWSII cell-derived exosome (1×10\hat{ }7 particles) by Exo-fect (5 μl) and activated with 5 μl 5 μM ckit-CD63 aptamer. The exosome was then added into ckit over-expressed MEF cells (1×10\hat{ }5 cells). 3 days later, cells were GFP sorted. 10 days later, genomic DNA was isolated, and IonTorrent PGM next generation sequencing showed successful induction by Exo-CRISPR as indication by indel formation (FIG. 23-FIG. 25).

### Example 4

Targeting Short RNA Delivery System (Ckit-Aptamer miR-26a Mimic)

Embodiments of the disclosure relate to a composition comprising a miRNA-aptamer chimera. In some embodiments, the chimera contains miR-26a mimic and c-Kit-targeting aptamer. Such embodiments are made and tested for effectiveness as follows.

3 Components were Designed for Aptamer-miRNA Chimera
1. Followed sequence from original miRNA stem loop sequence from miRBAse.
2. Aptamer-C3 linker-sense strand RNA 10 bp (3′ end needs 2-Fluoro, all U and C need 2′-Fluoro modifications).
3. 2nd sense strand RNA 12 bp with TEG-Cholesterol (5′ end needs 2-Fluoro, all uracil needs 2′-Fluoro modification).
4. miRNA 22 bp sequence (3′ end needs 2-Fluoro modification).
5. RNA oligos were ordered with RNase free HPLC purification.
6. Cholesterol passenger needs >250 nmole RNA Oligo.

### Assembled Delivery Components

1. Prepare 10 uM DNA aptamer solution in 100 ul annealing buffer (30 mM HEPES, 100 mM Potassium acetate, pH7.5, IDT) by adding 10 ul of 100 uM stock solution into 85 ul buffer.
2. Add 5 ul of 50 mM MgCl₂ solution.
3. Heat at 95c for 10 min
4. Snap cool on ice for 10 min
5. Prepare 10 uM miR-26a mimic and 10 uM 3′-RNA-TEG-Cholesterol in the 200 ul annealing buffer.
6. Mix these 3 components in one tube (total 300 ul) and aliquot into 3 PCR tubes (100 ul/tube).
7. Incubate in thermal cycler
   95C 5 min
   55C 20 min
   37C 30 min
   4C
8. Keep at −80C (3.3 uM (3.3 pmol/ul) [330 pmol/100 ul/tube])

### In Vitro Transfection

1. Prepare 2×10\hat{ }5 cells in 450 ul culture medium in 24 well plate.
2. Mix 50 ul Aptamer-miRNA chimera with 1 ul serum (FBS) and incubate for 15 min.
3. Add the Aptamer-miRNA chimera into the culture medium.
4. Incubate for 24 hrs.
5. Harvest cells and perform qPCR for miRNA with U6.
6. Harvest cells at day2 for qPCR with target genes.

### In Vivo Administration

1. Inject 200 ul (660 pmol) of chimera with 2% mouse serum (0.22 um filtered) into a mouse intravenously.
2. Inject two more times (inject per day).
3. 2-6 hrs later for localization with AF647 chimera by IVIS.
4. 1 day later for miRNA qPCR
5. 2 days later for miRNA target genes qPCR.
   Secondary Structure miR-26a (miRBase) (SEQ ID NO: 149)

```
            g u c --g ca
5′ gug ccucgu caaguaauc aggauaggcu ug g
      ||| |||||| ||||||||| |||||||||| || g
3′ cgc ggggca guucauugg ucuuauccgg ac u
            a c u gua cc
mouse ckit-apt passenger (SEQ ID NO: 150)
5′-
G*C*TCAACGCGGGACGGCTCTCCCATTGAC/iSpC3/i2FC//i2FC//
i2FU/rA/i2FU//i2FU//i2FC//i2FU/rGrG-3′
human CD117-apt passenger (SEQ ID NO: 151)
5′-
G*A'G*GCATACCAGCTTATTATTGGGGCCGGGGCAAGGGGGGGGTACCGTGGTAGG
ACAGATAGTAAGTGCAATCTGCGAA/iSpC3/i2FC//i2FC//i2FU/rA/i2FU//i2FU//
i2FC//I2FU/rGrG-3′
human CD117 core-apt passenger (SEQ ID NO: 152)
5′-
A*T*T*GGGGCCGGGGCAAGGGGGGGGTACCGTGGTAGGAC/iSpC3/i2FC//i2FC//i2FU/r
A/i2FU//i2FU//i2FC//i2U/rGrG-3′
Chol-passenger (SEQ ID NO: 153)
5′-rG/i2FU//i2FU/rA/i2FC//i2FU//i2FU/rG/i2FC/rA/i2FC/rG/3CholTEG/-3′
miR-26a-5p mimic (SEQ ID NO: 154)
5′-rUrUrCrArArGrUrArArUrCrCrArGrGrArUrArG/i2FG//i2FC/rU-3′
miR-26a mimic AF647 (or AF488) (SEQ ID NO: 155)
5′-/5AlexF647/rUrUrCrArArGrUrArArUrCrCrArGrGrArUrArG/i2FG//i2FC/rU-3′
5′ uucaaguaauccaggauaggcu (SEQ ID NO: 156) 3′
                            | ||||||||||||||||||
3′ Chol-TEG-gcacguucauugggucuuaucc-C3-ckit-apt 5′
                                    (SEQ ID NO: 157) [symbol]
                                      10^{th}-11^{th} bp has nick
                                                  ↓
5′ ckit-apt-C3-ccuauucuggguuacuugcacg-TEG-Chol (SEQ ID NO: 158) 3′
                                |||||||||||||||||| |
3′ ucggauaggaccuaaugaacuu 5′ (SEQ ID NO: 159)
Bold: 2′-F1uoro base modifications
```

### Phosphorothioate Bonds Modifications (*)

The phosphorothioate (PS) bond substitutes a sulfur atom for a non-bridging oxygen in the phosphate backbone of an oligo. This modification renders the internucleotide linkage resistant to nuclease degradation. Phosphorothioate bonds can be introduced between the last 3-5 nucleotides at the 5′- or 3′-end of the oligo to inhibit exonuclease degradation. Including phosphorothioate bonds throughout the entire oligo will help reduce attack by endonucleases as well.

/iSpC3/ Int C3 Spacer (MW 138.1)

The C3 Spacer phosphoramidite can be incorporated internally or at the 5′-end of the oligo. Multiple C3 spacers can be added at either end of an oligo to introduce a long hydrophilic spacer arm for the attachment of fluorophores or other pendent groups.

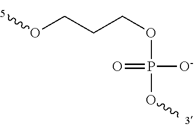

/i2FC/ Int Fluoro C (MW 307.2)

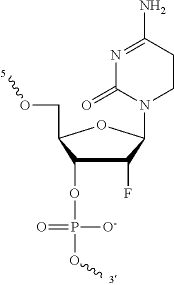

### Fluoro Bases

/i2FU/ Int Fluoro U (MW 308.2)

### Fluoro Bases

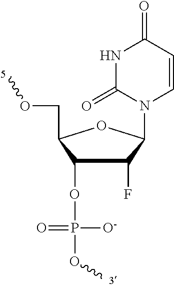

### /3CholTEG/ 3′ Cholesteryl-TEG (MW 756)

Cholesterol can be conjugated to oligonucleotides and can facilitate uptake into cells. It has been used as a transfection aid for antisense oligos and siRNAs, both in vitro and in vivo. Cholesterol is a very hydrophobic modification that is best purified using RP-HPLC.

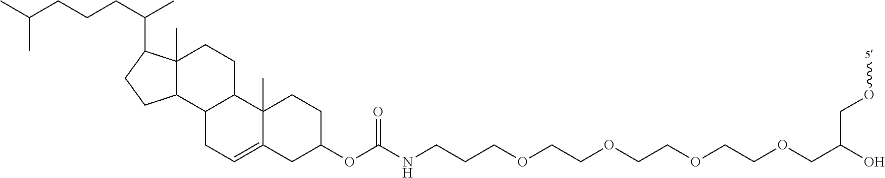

### Example 5

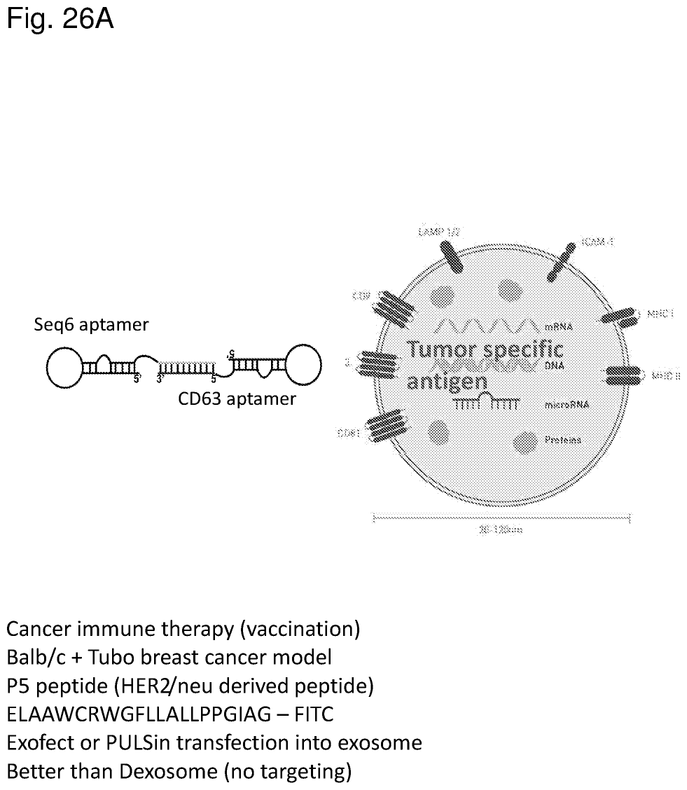

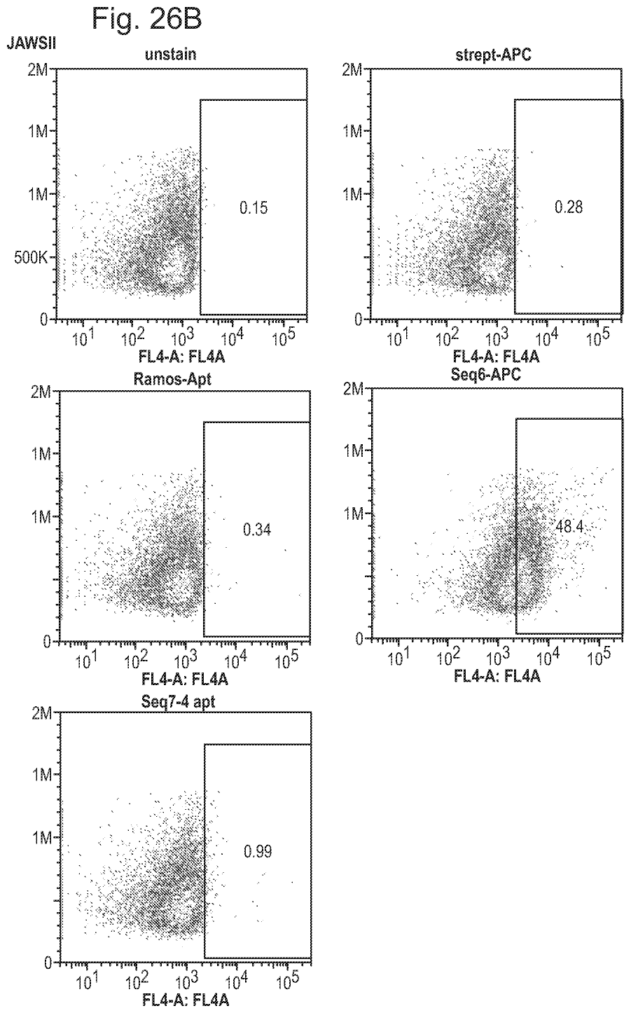

Exosomes of the present invention may also be loaded with tumor-specific antigens (FIG. 26A-B). To activate adaptive immune responses against tumor cells, tumor specific antigens have to be engulfed and processed by APCs, preferably dendritic cells. Therefore, small peptide delivery targeted to dendritic cells may be beneficial. The small peptides include tumor specific antigens to be delivered to dendritic cells using Seq6, CD205, CD40, or CD11c aptamers binding to the exosome via CD63 aptamer-linker. Non-limiting examples of tumor-specific antigens are shown in Table 1.

1. A composition comprising a nucleic acid sequence comprising two domains in the 5′ to 3′ orientation: an aptamer domain and tumor suppressor domain; the aptamer domain comprising from about 20 to about 50 contiguous ribonucleotides or modified ribonucleotides; and the tumor suppressor domain comprising a ribonucleic acid sequence comprising from about 18 to about 27 nucleotides sufficiently complementary to cancer-associated nucleic acid.

2. The composition of claim 1, wherein the aptamer domain from about 20 to about 50 contiguous ribonucleotides or modified ribonucleotides that targets the nucleic acid sequence into a cancer cell when exposed to the cancer cell in a therapeutically effective amount; and wherein the tumor suppressor domain comprises from about 18 to about 27 nucleotides sufficiently complementary to cancer-associated nucleic acid capable of inhibiting expression of the cancer-associated nucleic acid when exposed to the cancer cell in a therapeutically effective amount.

3. The composition of claim 1, wherein the tumor suppressor domain is at least 70% homologous to SEQ ID NO: 1 or comprises a genus of tumor suppressors other than mi-RNA26a.

4. The composition of claim 1, wherein the aptamer domain comprises a ribonucleic acid sequence at least 70% homologous to a ribonucleic acid sequence capable of binding c-kit receptor and/or T-cell receptor.

5-6. (canceled)

7. The composition of claim 1 further comprising a chemotherapeutic agent.

8. The composition of claim 1, wherein the aptamer domain and the tumor suppressor domain are contiguous and the aptamer domain consists of SEQ ID NO: 2 and the tumor suppressor domain consists of SEQ ID NO: 1.

9. A pharmaceutical composition comprising:
a pharmaceutically effective amount of the composition of claim 1 or a salt thereof; and
a pharmaceutically acceptable carrier.

10. The pharmaceutical composition of claim 9 further comprising a chemotherapeutic agent.

11. The pharmaceutical composition of claim 9 further comprising a nanoparticle that encapsulates the pharmaceutically effective amount of the ribonucleic acid.

12. The pharmaceutical composition of claim 9, wherein the pharmaceutically effective amount is from about 0.001 micrograms/mL to about 10 micrograms/mL.

13. (canceled)

14. A method of treating and/or preventing cancer in a subject in need thereof, the method comprising administering a pharmaceutically effective amount of the composition of claim 1 or a pharmaceutical composition comprising said composition and a pharmaceutically acceptable carrier.

15. The method of claim 14, wherein the cancer is selected from bone cancer, breast cancer, ovarian cancer, and prostate cancer.

16. (canceled)

17. The method of claim 14 further comprising repeating the step of administering the composition or the pharmaceutical composition once a day, once every other day, once a week, once every other week or once a month.

18. The method of claim 14 further comprising administering a therapeutically effective amount of at least one chemotherapeutic agent prior to, simultaneously with, or subsequent to administering the composition or the pharmaceutical composition.

19. The method of claim 14 further comprising administering a toxic amount of at least one chemotherapeutic agent prior to, simultaneously with, or subsequent to administering the composition or the pharmaceutical composition.

20. (canceled)

21. The method of claim 18, wherein the at least one chemotherapeutic agent is administered once a day, once every other day, once a week, once every other week or once a month.

22. The method of claim 14, wherein the step of administering comprises administering a dose of the composition or the pharmaceutical composition of from about 0.1 micrograms/mL to about 500 micrograms/mL.

23. (canceled)

24. A method of reducing the toxicity of radiation therapy in a subject in need thereof, the method comprising administering a therapeutically effective amount of the composition of claim 1 or a pharmaceutical composition comprising said composition and a pharmaceutically acceptable carrier.

25-30. (canceled)

31. A method of potentiating the effect of a chemotherapeutic agent, the method comprising administering to a subject, exposed to the chemotherapeutic agent or expected to be exposed to the chemotherapeutic agent, the composition of claim 1 or a pharmaceutical composition comprising said composition and a pharmaceutically acceptable carrier.

32. (canceled)

33. The method of claim 31, wherein the subject is suffering from or suspected of having breast cancer, breast cancer, ovarian cancer, bone cancer, pancreatic cancer, lung cancer, or prostate cancer.

34-41. (canceled)

42. A method of preventing myelosuppression in a subject, the method comprising administering a pharmaceutically effective amount of the composition of claim 1 or a pharmaceutical composition comprising said composition and a pharmaceutically acceptable carrier.

43-44. (canceled)

45. The method of claim 42 further comprising repeating the step of administering the composition or the pharmaceutical composition once a day, once every other day, once a week, once every other week or once a month.

46. The method of claim 42 further comprising administering a therapeutically effective amount of at least one chemotherapeutic agent prior to, simultaneously with, or subsequent to administering the composition or the pharmaceutical composition.

47-51. (canceled)

52. The method of claim 42, wherein the myelosuppression is 5′-fluorouracil (5-FU)-induced myelosuppression.

53-70. (canceled)


SEQUENCE LISTING (166 sequences)

SEQ ID NO: 1 - RNA, 22 nt, Artificial Sequence
  Description of Artificial Sequence: Synthetic oligonucleotide
uucaaguaauccaggauaggcu

SEQ ID NO: 2 - DNA, 77 bp, Artificial Sequence
  Description of Artificial Sequence: Synthetic oligonucleotide
gaggcataccagcttattcaaggggccggggcaaggggggggtaccgtggtaggacatagtaagtgcaatctgcgaa

SEQ ID NO: 3 - DNA, 29 bp, Artificial Sequence
  Description of Artificial Sequence: Synthetic oligonucleotide
gctcaacgcgggacggctctcccattgac

SEQ ID NO: 4 - DNA, 77 bp, Artificial Sequence; identical to SEQ ID NO: 2
  Description of Artificial Sequence: Synthetic oligonucleotide

SEQ ID NO: 5 - RNA, 10 nt, Artificial Sequence
  Description of Artificial Sequence: Synthetic oligonucleotide
ccuauucugg

SEQ ID NO: 6 - DNA, 29 bp, Artificial Sequence
  Description of Artificial Sequence: Synthetic oligonucleotide
gctcaacgcgggacggctctcccattgac

SEQ ID NO: 7 - RNA, 10 nt, Artificial Sequence
  Description of Artificial Sequence: Synthetic oligonucleotide
ccuauucugg

SEQ ID NO: 8 - RNA, 12 nt, Artificial Sequence
  Description of Artificial Sequence: Synthetic oligonucleotide
guuacuugcacg

SEQ ID NO: 9 - RNA, 22 nt, Artificial Sequence
  Description of Artificial Sequence: Synthetic oligonucleotide
uucaaguaauccaggauaggcu

SEQ ID NO: 10 - RNA, 21 nt, Artificial Sequence
  Description of Artificial Sequence: Synthetic oligonucleotide
gguucguacguacacuguuca

SEQ ID NO: 11 - DNA, 22 bp, Artificial Sequence
  Description of Artificial Sequence: Synthetic primer
tctccaccaagacctgaaaaat

SEQ ID NO: 12 - DNA, 22 bp, Artificial Sequence
  Description of Artificial Sequence: Synthetic primer
cttcgaaagacctcctctgtgt

SEQ ID NO: 13 - DNA, 22 bp, Artificial Sequence
  Description of Artificial Sequence: Synthetic primer
ttgctaagagggctatccagac

SEQ ID NO: 14 - DNA, 22 bp, Artificial Sequence
  Description of Artificial Sequence: Synthetic primer
tgtcaagggatttccatttctc

SEQ ID NO: 15 - DNA, 24 bp, Artificial Sequence
  Description of Artificial Sequence: Synthetic primer
aggagtcctgttgatgttgccagt

SEQ ID NO: 16 - DNA, 24 bp, Artificial Sequence
  Description of Artificial Sequence: Synthetic primer
gggacgcagcaactgacatttcta

SEQ ID NO: 17 - DNA, 126 bp, Artificial Sequence
  Description of Artificial Sequence: Synthetic polynucleotide
gacggcgctaggatcatcaacagcctatcctggtctcattacttgaacaagtattctggtcacagaatacaacagcctatcctggtctcattacttgaacaagatgatcctagcgccgtctttttt

SEQ ID NO: 18 - DNA, 87 bp, Artificial Sequence
  Description of Artificial Sequence: Synthetic oligonucleotide
catcaactatcgcgagtatcgacgtcgaggcccaagtattctggtcacagaatacaactatcgcgagtatcgacgtcgaggcccaag

SEQ ID NO: 19 - RNA, 24 nt, Mus sp.
cccggaaccuaugauuacuugaac

SEQ ID NO: 20 - RNA, 8 nt, Artificial Sequence
  Description of Artificial Sequence: Synthetic oligonucleotide
uacuugaa

SEQ ID NO: 21 - RNA, 22 nt, Unknown
  Description of Unknown: mir-26a
uucaaguaauccaggauaggcu

SEQ ID NO: 22 - DNA, 20 bp, Artificial Sequence
  Description of Artificial Sequence: Synthetic oligonucleotide
atggtgtccctagtggcccc

SEQ ID NO: 23 - RNA, 20 nt, Artificial Sequence
  Description of Artificial Sequence: Synthetic oligonucleotide
ggggccacuagggacaggau

SEQ ID NO: 24 - RNA, 101 nt, Artificial Sequence
  Description of Artificial Sequence: Synthetic polynucleotide
ggggccacuagggacaggauguuuuagagcuagaaauagcaaguuaaaauaaggcuaguccguuaucaacuugaaaaaguggcaccgagucggugcuuuuu

SEQ ID NO: 25 - DNA, 140 bp, Mus sp.
tatgctccatacagtacacaatctcttctctctacagatgactgccatggaggagtcacagtcggatatcagcctcgagctccctctgagccaggagacattttcaggcttatggaaactgtgagtggatctttttgggg

SEQ ID NO: 26 - protein, 28 aa, Mus sp.
MTAMEESQSDISLELPLSQETFSGLWKL

SEQ ID NO: 27 - DNA, 50 bp, Mus sp.
ttgcatccatacagtacacaatctcttctctctacagatgactgccatgg

SEQ ID NO: 28 - DNA, 136 bp, Mus sp.
tgcatccatacagtacacaatctcttctctctacagatgactgccatggaggagtcacagtcggatatcagcctcgagctccctagccaggagacattttcaggcttatggaaactgtgagtggatcttttttggg

SEQ ID NO: 29 - DNA, 139 bp, Mus sp.
tgcatccatacagtacacaatctcttctctctacagatgactgccatggaggagtcacagtcggatatcagcctcgagctccctctgagccaggagacattttcaggcttatggaaactgtgagtggatcttttttggg

SEQ ID NO: 30 - DNA, 138 bp, Mus sp.
tgcatccatacagtacacaatctcttctctctacagatgactgccatggaggagtcacagtcggatatcagcctcgagctccctctgagccaggagacattttcaggcttatggaaactgtgagtggatctttttggg

SEQ ID NO: 31 - DNA, 138 bp, Mus sp.; identical to SEQ ID NO: 30

SEQ ID NO: 32 - DNA, 138 bp, Mus sp.; identical to SEQ ID NO: 30

SEQ ID NO: 33 - DNA, 134 bp, Mus sp.
tgcatccatacagtacacaatctcttctctctacagatgactgccatggaggagtcacagtcggatatcagcctcgagctcccagccaggagacattttcaggcttatggaaactgtgagtggatctttttggg

SEQ ID NO: 34 - DNA, 138 bp, Mus sp.; identical to SEQ ID NO: 30

SEQ ID NO: 35 - DNA, 138 bp, Mus sp.; identical to SEQ ID NO: 30

SEQ ID NO: 36 - DNA, 127 bp, Mus sp.
tgcatccatacagtacacaatctcttctctctacagatgactgccatggaggagtcacagtcggatatcagcctcgagccaggagacattttcaggcttatggaaactgtgagtggatctttttggg

SEQ ID NO: 37 - DNA, 138 bp, Mus sp.; identical to SEQ ID NO: 30

SEQ ID NO: 38 - DNA, 138 bp, Mus sp.; identical to SEQ ID NO: 30

SEQ ID NO: 39 - DNA, 137 bp, Mus sp.
tgcatcatacagtacacaatctcttctctctacagatgactgccatggaggagtcacagtcggatatcagcctcgagctccctctgagccaggagacattttcaggcttatggaaactgtgagtggatctttttggg

SEQ ID NO: 40 - DNA, 136 bp, Mus sp.
tgcatccatacagtacacatctcttctctctacagatgactgccatggaggagtcacagtcggatatcagcctcgagctccctctggccaggagacattttcaggcttatggaaactgtgagtggatctttttggg

SEQ ID NO: 41 - DNA, 135 bp, Mus sp.
tgcatccatacagtacacaatctcttctctctacagatgactgccatggaggagtcacagtcggatatcagcctcgagctccctctgaaggagacattttcaggcttatggaaactgtgagtggatctttttggg

SEQ ID NO: 42 - DNA, 138 bp, Mus sp.; identical to SEQ ID NO: 30

SEQ ID NO: 43 - DNA, 137 bp, Mus sp.
tgcatccatacagtacacatctcttctctctacagatgactgccatggaggagtcacagtcggatatcagcctcgagctccctctgagccaggagacattttcaggcttatggaaactgtgagtggatctttttggg

SEQ ID NO: 44 - DNA, 138 bp, Mus sp.; identical to SEQ ID NO: 30

SEQ ID NO: 45 - DNA, 138 bp, Mus sp.; identical to SEQ ID NO: 30

SEQ ID NO: 46 - DNA, 138 bp, Mus sp.; identical to SEQ ID NO: 30

SEQ ID NO: 47 - DNA, 138 bp, Mus sp.; identical to SEQ ID NO: 30

SEQ ID NO: 48 - DNA, 138 bp, Mus sp.; identical to SEQ ID NO: 30

SEQ ID NO: 49 - DNA, 138 bp, Mus sp.; identical to SEQ ID NO: 30

SEQ ID NO: 50 - DNA, 138 bp, Mus sp.; identical to SEQ ID NO: 30

SEQ ID NO: 51 - DNA, 138 bp, Mus sp.; identical to SEQ ID NO: 30

SEQ ID NO: 52 - DNA, 138 bp, Mus sp.; identical to SEQ ID NO: 30

SEQ ID NO: 53 - DNA, 138 bp, Mus sp.; identical to SEQ ID NO: 30

SEQ ID NO: 54 - DNA, 138 bp, Mus sp.; identical to SEQ ID NO: 30

SEQ ID NO: 55 - DNA, 127 bp, Mus sp.; identical to SEQ ID NO: 36

SEQ ID NO: 56 - DNA, 138 bp, Mus sp.; identical to SEQ ID NO: 30

SEQ ID NO: 57 - DNA, 137 bp, Mus sp.
tgcatccatacagtacacaatctcttctctctacagatgactgccatggaggagtcacagtcggatatcagcctcgagctccctctggccaggagacattttcaggcttatggaaactgtgagtggatctttttggg

SEQ ID NO: 58 - DNA, 138 bp, Mus sp.; identical to SEQ ID NO: 30

SEQ ID NO: 59 - DNA, 138 bp, Mus sp.; identical to SEQ ID NO: 30

SEQ ID NO: 60 - DNA, 138 bp, Mus sp.; identical to SEQ ID NO: 30

SEQ ID NO: 61 - DNA, 137 bp, Mus sp.
tgcatccatacagtacacaatctcttctctctacagatgactgccatggaggagtcacagtcggatatcagctcgagctccctctgagccaggagacattttcaggcttatggaaactgtgagtggatctttttggg

SEQ ID NO: 62 - DNA, 138 bp, Mus sp.; identical to SEQ ID NO: 30

SEQ ID NO: 63 - DNA, 137 bp, Mus sp.
tgcatccatacagtacacaatctcttctctctacagatgactgccatggaggagtcacagtcggatatcagcctcgagctccctctgaccaggagacattttcaggcttatggaaactgtgagtggatctttttggg

SEQ ID NO: 64 - DNA, 138 bp, Mus sp.; identical to SEQ ID NO: 30

SEQ ID NO: 65 - DNA, 138 bp, Mus sp.
tgcatccatacagtacacaatctcttctctctacagatgactgccatggaggagtcacagtcggatatcagcctcgagctccctctgagccaggagacattttcaggcttatgaaaactgtgagtggatctttttggg

SEQ ID NO: 66 - DNA, 134 bp, Mus sp.; identical to SEQ ID NO: 33

SEQ ID NO: 67 - DNA, 136 bp, Mus sp.
tgcatccatacagtacacaatctcttctctctacagatgactgccatggaggagtcacagtcggatatcagcctcgagctccctcagccaggagacattttcaggcttatggaaactgtgagtggatctttttggg

SEQ ID NO: 68 - DNA, 146 bp, Mus sp.
agttatgcatccatacagtacacaatctcttctctctacagatgactgccatggaggagtcacagtcggatatcagcctcgagctccctctgagccaggagacattttcaggcttatggaaactgtgagtggatctttttggggcc

SEQ ID NO: 69 - DNA, 120 bp, Mus sp.
gcagatggacactgatgttgtggtcctcacaactggtgtcttggtgttgatcaccatgctcccgatgatcccgcagtcagggaagcagcaccttctcgacttctttgacatctttggccg

SEQ ID NO: 70 - protein, 40 aa, Mus sp.
MDTDVVVLTTGVLVLITMLPMIPQSGKQHLLDFFDIIFGR

SEQ ID NO: 71 - DNA, 29 bp, Mus sp.
gctcaacgcgggacggctctcccattgac

SEQ ID NO: 72 - DNA, 79 bp, Unknown
  Description of Unknown: CD117 sequence
gaggcataccagcttattattggggccggggcaaggggggggtaccgtggtaggacagatagtaagtgcaatctgcgaa

SEQ ID NO: 73 - DNA, 38 bp, Unknown
  Description of Unknown: CD117 (core) sequence
attggggccggggcaaggggggggtaccgtggtaggac

SEQ ID NO: 74 - DNA, 73 bp, Unknown
  Description of Unknown: CD3 sequence
cagcgtggaggataaaagttagctgtttcctcgtggcagaaggaacagaccaccgtactttagggtgtgtcgt

SEQ ID NO: 75 - DNA, 39 bp, Unknown
  Description of Unknown: CD4 sequence
gtgacgtcctgatcgattgtgcattcggtgtgacgatct

SEQ ID NO: 76 - DNA, 29 bp, Unknown
  Description of Unknown: CD8 sequence
ctacagcttgctatgctccccttggggta

SEQ ID NO: 77 - DNA, 41 bp, Unknown
  Description of Unknown: CD16 sequence
ccactgcgggggtctatacgtgaggaagaagtgggcaggtc

SEQ ID NO: 78 - RNA, 77 nt, Unknown
  Description of Unknown: CD28 sequence
gggagagaggaagagggauggggauuagaccauaggcucccaacccccauaacccagaggucgauaguacuggaucc

SEQ ID NO: 79 - RNA, 65 nt, Unknown
  Description of Unknown: CD40 sequence
gggagagacgaugcggccaacgaguaggcgauagcgcguggcagagcgucgcugaggauccgaga

SEQ ID NO: 80 - DNA, 30 bp, Unknown
  Description of Unknown: CD44 sequence
ccaaggcctgcaagggaaccaaggacacag

SEQ ID NO: 81 - RNA, 80 nt, Mus sp.
gggaggacgaugcggcagucugcaucguaggaaucgccaccguauacuuucccaccagacgacucgcugaggauccgaga

SEQ ID NO: 82 - RNA, 54 nt, Unknown
  Description of Unknown: CD134 sequence
gggaugcggaaaaaagaacacuuccgauuagggcccacccuaacggccgcagac

SEQ ID NO: 83 - RNA, 15 nt, Unknown
  Description of Unknown: CD133 sequence
cccuccuacauaggg

SEQ ID NO: 84 - RNA, 96 nt, Unknown
  Description of Unknown: CD137 sequence
gggagagaggaagagggaugggcgaccgaacgugcccuucaaagccguucacuaaccaguggcauaacccagaggucgauaguacuggaucccccc

SEQ ID NO: 85 - RNA, 35 nt, Mus sp.
gggagagaggaagagggaugggccgacgugccgca

SEQ ID NO: 86 - RNA, 42 nt, Unknown
  Description of Unknown: CD205 sequence
gggagguguguuagcacacgauucauaaucagcuacccuccc

SEQ ID NO: 87 - DNA, 75 bp, Mus sp.
gacgatagcggtgacggcacagacggctactgtacatcacgcctctcccccgtatgccgcttccgtccgtcgctc

SEQ ID NO: 88 - RNA, 62 nt, Mus sp.
gggagaggaccauguagucacuauggucuuggagcuagcggcagagcgucgcggucccuccc

SEQ ID NO: 89 - DNA, 40 bp, Unknown
  Description of Unknown: EpCAM sequence
aacagagggacaaacgggggaagatttgacgtcgacgaca

SEQ ID NO: 90 - DNA, 40 bp, Unknown
  Description of Unknown: EGFR sequence
tgccgtttcttctctttcgctttttttgcttttgagcatg

SEQ ID NO: 91 - DNA, 79 bp, Unknown
  Description of Unknown: Seq6 Mouse macrophage sequence
gctgtgtgactcctgcaatgtccagtctcggccctaccttcgacttcctaagtcgcatcggcagctgtatcttgtctcc

SEQ ID NO: 92 - DNA, 38 bp, Unknown
  Description of Unknown: Nucleolin (NUC) sequence
ggtggtggtggttgtggtggtggtggtttttttttttt

SEQ ID NO: 93 - RNA, 56 nt, Unknown
  Description of Unknown: PSMA sequence
gggaggacgaugcggaucagccauguuuacgucacuccuugucaauccucaucggc

SEQ ID NO: 94 - DNA, 84 bp, Unknown
  Description of Unknown: Her2 sequence
aaccgcccaaatccctaagagtctgcacttgtcattttgtatatgtttggtttttggctctcacagacacactacacacgcaca

SEQ ID NO: 95 - DNA, 25 bp, Unknown
  Description of Unknown: Nestin sequence
gcagttgatcctttggataccctgg

SEQ ID NO: 96 - DNA, 25 bp, Unknown
  Description of Unknown: Mucin1 sequence
gcagttgatcctttggataccctgg

SEQ ID NO: 97 - RNA, 26 nt, Unknown
  Description of Unknown: VEGFR sequence
cggaaucagugaaugcuauacauccg

SEQ ID NO: 98 - DNA, 41 bp, Unknown
  Description of Unknown: PDGF-BB sequence
caggctacggcacgtagagcatcaccatgatcctgtttttt

SEQ ID NO: 99 - DNA, 84 bp, Unknown
  Description of Unknown: c-Met sequence
ggagggaaaagttatcaggctggatggtagctcggtcggggtgggtgggttggcaagtctgattagttttggagtactcgctcc

SEQ ID NO: 100 - protein, 976 aa, Artificial Sequence [sequence omitted]

SEQ ID NO: 101 - protein, 750 aa, Artificial Sequence
  Description of Artificial Sequence: Synthetic polypeptide
MWNLLHETDSAVATARRPRWLCAGALVLAGGFFLLGFLFGWFIKSSNEATNITPKHNMKAFLDELKAENIKKFLYNFTQIPHLAGTEQNFQLAKQIQSQWKEFGLDSVELAHYDVLLSYPNKTHPNYISIINEDGNEIFNTSLFEPPPPGYENVSDIVPPFSAFSPQGMPEGDLVYVNYARTEDFFKLERDMKINCSGKIVIARYGKVFRGNKVKNAQLAGAKGVILYSDPADYFAPGVKSYPDGWNLPGGGVQRGNILNLNGAGDPLTPGYPANEYAYRRGIAEAVGLPSIPVHPIGYYDAQKLLEKMGGSAPPDSSWRGSLKVPYNVGPGFTGNFSTQKVKMHIHSTNEVTRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVHNLTKELKSPDEGFEGKSLYESWTKKSPSPEFSGMPRISKLGSGNDFEVFFQRLGIASGRARYTKNWETNKFSGYPLYHSVYETYELVEKFYDPMFKYHLTVAQVRGGMVFELANSIVLPFDCRDYAVVLRKYADKIYSISMKHPQEMKTYSVSFDSLFSAVKNFTEIASKFSERLQDFDKSNPIVLRMMNDQLMFLERAFIDPLGLPDRPFYRHVIYAPSSHNKYAGESFPGIYDALFDIESKVDPSKAWGEVKRQIYVAAFTVQAAAETLSEVA

SEQ ID NO: 102 - protein, 1255 aa, Artificial Sequence [sequence omitted]

SEQ ID NO: 103 - protein, 314 aa, Artificial Sequence
  Description of Artificial Sequence: Synthetic polypeptide
MAPPQVLAFGLLLAAATATFAAAQEECVCENYKLAVNCFVNNNRQCQCTSVGAQNTVICSKLAAKCLVMKAEMNGSKLGRRAKPEGALQNNDGLYDPDCDESGLFKAKQCNGTSMCWCVNTAGVRRTDKDTEITCSERVRTYWIIIELKHKAREKPYDSKSLRTALQKEITTRYQLDPKFITSILYENNVITIDLVQNSSQKTQNDVDIADVAYYFEKDVKGESLFHSKKMDLTVNGEQLDLDPGQTLIYYVDEKAPEFSMQGLKAGVIAVIVVVVIAVVAGIVVLVISRKKRMAKYEKAEIKEMGEMHRELNA

SEQ ID NO: 104 - protein, 357 aa, Artificial Sequence
  Description of Artificial Sequence: Synthetic polypeptide
MGRQLAGCGDAGKKASFKMSTVHEILCKLSLEGDHSTPPSAYGSVKAYTNFDAERDALNIETAIKTKGVDEVTIVNILTNRSNAQRQDIAFAYQRRTKKELASALKSALSGHLETVILGLLKTPAQYDASELKASMKGLGTDEDSLIEIICSRTNQELQEINRVYKEMYKTDLEKDIISDTSGDFRKLMVALAKGRRAEDGSVIDYELIDQDARDLYDAGVKRKGTDVPKWISIMTERSVPHLQKVFDRYKSYSPYDMLESIRKEVKGDLENAFLNLVQCIQNKPLYFADRLYDSMKGKGTRDKVLIRIMVSRSEVDMLKIRSEFKRKYGKSLYYYIQQDTKGDYQKALLYLCGGDD

SEQ ID NO: 105 - protein, 757 aa, Artificial Sequence
  Description of Artificial Sequence: Synthetic polypeptide
MLLRLLLAWAAAGPTLGQDPWAAEPRAACGPSSCYALFPRRRTFLEAWRACRELGGDLATPRTPEEAQRVDSLVGAGPASRLLWIGLQRQARQCQLQRPLRGFTWTTGDQDTAFTNWAQPASGGPCPAQRCVALEASGEHRWLEGSCTLAVDGYLCQFGFEGACPALQDEAGQAGPAVYTTPFHLVSTEFEWLPFGSVAAVQCQAGRGASLLCVKQPEGGVGWSRAGPLCLGTGCSPDNGGCEHECVEEVDGHVSCRCTEGFRLAADGRSCEDPCAQAPCEQQCEPGGPQGYSCHCRLGFRPAEDDPHRCVDTDECQIAGVCQQMCVNYVGGFECYCSEGHELEADGISCSPAGAMGAQASQDLGDELLDDGEDEEDEDEAWKAFNGGWTEMPGILWMEPTQPPDFALAYRPSFPEDREPQIPYPEPTWPPPLSAPRVPYHSSVLSVTRPVVVSATHPTLPSAHQPPVIPATHPALSRDHQIPVIAANYPDLPSAYQPGILSVSHSAQPPAHQPPMISTKYPELFPAHQSPMFPDTRVAGTQTTTHLPGIPPNHAPLVTTLGAQLPPQAPDALVLRTQATQLPIIPTAQPSLTTTSRSPVSPAHQISVPAATQPAALPTLLPSQSPTNQTSPISPTHPHSKAPQIPREDGPSPKLALWLPSPAPTAAPTALGEAGLAEHSQRDDRWLLVALLVPTCVFLVVLLALGIVYCTRCGPHAPNKRITDCYRWVIHAGSKSPTEPMPPRGSLTGVQTCRTSV

SEQ ID NO: 106 - protein, 273 aa, Artificial Sequence
  Description of Artificial Sequence: Synthetic polypeptide
MTPGTQSPFFLLLLLTVLTVVTGSGHASSTPGGEKETSATQRSSVPSSTEKNALSTGVSFFFLSFHISNLQFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANL

SEQ ID NO: 107 - protein, 710 aa, Artificial Sequence
  Description of Artificial Sequence: Synthetic polypeptide
MVKLAKAGKNQGDPKKMAPPPKEVEEDSEDEEMSEDEEDDSSGEEVVIPQKKGKKAAATSAKKVVVSPTKKVAVATPAKKAAVTPGKKAAATPAKKTVTPAKAVTTPGKKGATPGKALVATPGKKGAAIPAKGAKNGKNAKKEDSDEEEDDDSEEDEEDDEDEDEDEDEIEPAAMKAAAAAPASEDEDDEDDEDDEDDDDDEEDDSEEEAMETTPAKGKKAAKVVPVKAKNVAEDEDEEEDDEDEDDDDDEDDEDDDDEDDEEEEEEEEEEPVKEAPGKRKKEMAKQKAAPEAKKQKVEGTEPTTAFNLFVGNLNFNKSAPELKTGISDVFAKNDLAVVDVRIGMTRKFGYVDFESAEDLEKALELTGLKVFGNEIKLEKPKGKDSKKERDARTLLAKNLPYKVTQDELKEVFEDAAEIRLVSKDGKSKGIAYIEFKTEADAEKTFEEKQGTEIDGRSISLYYTGEKGQNQDYRGGKNSTWSGESKTLVLSNLSYSATEETLQEVFEKATFIKVPQNQNGKSKGYAFIEFASFEDAKEALNSCNKREIEGRAIRLELQGPRGSPNARSQPSKTLFVKGLSEDTTEETLKESFDGSVRARIVTDRETGSSKGFGFVDFNSEEDAKAAKEAMEDGEIDGNKVTLDWAKPKGEGGFGGRGGGRGGFGGRGGGRGGRGGFGGRGRGGFGGRGGFRGGRGGGGDHKPQGKKTKFE

SEQ ID NO: 108 - protein, 658 aa, Artificial Sequence
  Description of Artificial Sequence: Synthetic polypeptide
MDRGTLPLAVALLLASCSLSPTSLAETVHCDLQPVGPERGEVTYTTSQVSKGCVAQAPNAILEVHVLFLEFPTGPSQLELTLQASKQNGTWPREVLLVLSVNSSVFLHLQALGIPLHLAYNSSLVTFQEPPGVNTTELPSFPKTQILEWAAERGPITSAAELNDPQSILLRLGQAQGSLSFCMLEASQDMGRTLEWRPRTPALVRGCHLEGVAGHKEAHILRVLPGHSAGPRTVTVKVELSCAPGDLDAVLILQGPPYVSWLIDANHNMQIWTTGEYSFKIFPEKNIRGFKLPDTPQGLLGEARMLNASIVASFVELPLASIVSLHASSCGGRLQTSPAPIQTTPPKDTCSPELLMSLIQTKCADDAMTLVLKKELVAHLKCTITGLTFWDPSCEAEDRGDKFVLRSAYSSCGMQVSASMISNEAVVNILSSSSPQRKKVHCLNMDSLSFQLGLYLSPHFLQASNTIEPGQQSFVQVRVSPSVSEFLLQLDSCHLDLGPEGGTVELIQGRAAKGNCVSLLSPSPEGDPRFSFLLHFYTVPIPKTGTLSCTVALRPKTGSQDQEVHRTVFMRLNIISPDLSGCTSKGLVLPAVLGITFGAFLIGALLTAALWYIYSHTRSPSKREPVVAVAAPASSESSSTNHSIGSTQSTPCSTSSMA

SEQ ID NO: 109 - protein, 360 aa, Artificial Sequence [sequence omitted]

SEQ ID NO: 110 - protein, 368 aa, Artificial Sequence
  Description of Artificial Sequence: Synthetic polypeptide
MVLEVSDHQVLNDAEVAALLENFSSSYDYGENESDSCCTSPPCPQDFSLNFDRAFLPALYSLLFLLGLLGNGAVAAVLLSRRTALSSTDTFLLHLAVADTLLVLTLPLWAVDAAVQWVFGSGLCKVAGALFNINFYAGALLLACISFDRYLNIVHATQLYRRGPPARVTLTCLAVWGLCLLFALPDFIFLSAHHDERLNATHCQYNFPQVGRTALRVLQLVAGFLLPLLVMAYCYAHILAVLLVSRGQRRLRAMRLVVVVVVAFALCWTPYHLVVLVDILMDLGALARNCGRESRVDVAKSVTSGLGYMHCCLNPLLYAFVGVKFRERMWMLLLRLGCPNQRGLQRQPSSSRRDSSWSETSEASYSGL

SEQ ID NO: 111 - protein, 984 aa, Artificial Sequence
  Description of Artificial Sequence: Synthetic polypeptide
MALDYLLLLLLASAVAAMEETLMDTRTATAELGWTANPASGWEEVSGYDENLNTIRTYQVCNVFEPNQNNWLLTTFINRRGAHRIYTEMRFTVRDCSSLPNVPGSCKETFNLYYYETDSVIATKKSAFWSEAPYLKVDTIAADESFSQVDFGGRLMKVNTEVRSFGPLTRNGFYLAFQDYGACMSLLSVRVFFKKCPSIVQNFAVFPETMTGAESTSLVIARGTCIPNAEEVDVPIKLYCNGDGEWMVPIGRCTCKPGYEPENSVACKACPAGTFKASQEAEGCSHCPSNSRSPAEASPICTCRTGYYRADFDPPEVACTSVPSGPRNVISIVNETSIILEWHPPRETGGRDDVTYNIICKKCRADRRSCSRCDDNVEFVPRQLGLTECRVSISSLWAHTPYTFDIQAINGVSSKSPFPPQHVSVNITTNQAAPSTVPIMHQVSATMRSITLSWPQPEQPNGIILDYEIRYYEKEHNEFNSSMARSQTNTARIDGLRPGMVYVVQVRARTVAGYGKFSGKMCFQTLTDDDYKSELREQLPLIAGSAAAGVVFVVSLVAISIVCSRKRAYSKEAVYSDKLQHYSTGRGSPGMKIYIDPFTYEDPNEAVREFAKEIDVSFVKIEEVIGAGEFGEVYKGRLKLPGKREIYVAIKTLKAGYSEKQRRDFLSEASIMGQFDHPNIIRLEGVVTKSRPVMIITEFMENGALDSFLRQNDGQFTVIQLVGMLRGIAAGMKYLAEMNYVHRDLAARNILVNSNLVCKVSDFGLSRYLQDDTSDPTYTSSLGGKIPVRWTAPEAIAYRKFTSASDVWSYGIVMWEVMSFGERPYWDMSNQDVINAIEQDYRLPPPMDCPAALHQLMLDCWQKDRNSRPRFAEIVNTLDKMIRNPASLKTVATITAVPSQPLLDRSIPDFTAFTTVDDWLSAIKMVQYRDSFLTAGFTSLQLVTQMTSEDLLRIGITLAGHQKKILNSIHSMRVQISQSPTAMA

SEQ ID NO: 112 - protein, 468 aa, Artificial Sequence
  Description of Artificial Sequence: Synthetic polypeptide
MLAVGCALLAALLAAPGAALAPRRCPAQEVARGVLTSLPGDSVTLTCPGVEPEDNATVHWVLRKPAAGSHPSRWAGMGRRLLLRSVQLHDSGNYSCYRAGRPAGTVHLLVDVPPEEPQLSCFRKSPLSNVVCEWGPRSTPSLTTKAVLLVRKFQNSPAEDFQEPCQYSQESQKFSCQLAVPEGDSSFYIVSMCVASSVGSKFSKTQTFQGCGILQPDPPANITVTAVARNPRWLSVTWQDPHSWNSSFYRLRFELRYRAERSKTFTTWMVKDLQHHCVIHDAWSGLRHVVQLRAQEEFGQGEWSEWSPEAMGTPWTESRSPPAENEVSTPMQALTTNKDDDNILFRDSANATSLPVQDSSSVPLPTFLVAGGSLAFGTLLCIAIVLRFKKTWKLRALKEGKTSMHPPYSLGQLVPERPRPTPVLVPLISPPVSPSSLGSDNTSSHNRPDARDPRSPYDISNTDYFFPR

SEQ ID NO: 113 - protein, 503 aa, Artificial Sequence
  Description of Artificial Sequence: Synthetic polypeptide
MEAAVAAPRPRLLLLVLAAAAAAAAALLPGATALQCFCHLCTKDNFTCVTDGLCFVSVTETTDKVIHNSMCIAEIDLIPRDRPFVCAPSSKTGSVTTTYCCNQDHCNKIELPTTVKSSPGLGPVELAAVIAGPVCFVCISLMLMVYICHNRTVIHHRVPNEEDPSLDRPFISEGTTLKDLIYDMTTSGSGSGLPLLVQRTIARTIVLQESIGKGRFGEVWRGKWRGEEVAVKIFSSREERSWFREAEIYQTVMLRHENILGFIAADNKDNGTWTQLWLVSDYHEHGSLFDYLNRYTVTVEGMIKLALSTASGLAHLHMEIVGTQGKPAIAHRDLKSKNILVKKNGTCCIADLGLAVRHDSATDTIDIAPNHRVGTKRYMAPEVLDDSINMKHFESFKRADIYAMGLVFWEIARRCSIGGIHEDYQLPYYDLVPSDPSVEEMRKVVCEQKLRPNIPNRWQSCEALRVMAKIMRECWYANGAARLTALRIKKTLSQLSQQEGIKM

SEQ ID NO: 114 - protein, 567 aa, Artificial Sequence
  Description of Artificial Sequence: Synthetic polypeptide
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 115 - skipped

SEQ ID NO: 116 - DNA, 90 bp, Mus sp.
ctgggcctctgtgggcatcttaccggacagtgctggatttcttggcttgactctaacactgtctggtaacgatgttcaaaggtgacccac

SEQ ID NO: 117 - DNA, 90 bp, Homo sapiens
ccgggcccctgtgagcatcttaccggacagtgctggatttcccagcttgactctaacactgtctggtaacgatgttcaaaggtgacccgc

SEQ ID NO: 118 - DNA, 70 bp, Mus sp.
gccgtggccatcttactgggcagcattggatagtgtctgatctctaatactgcctggtaatgatgacggc

SEQ ID NO: 119 - DNA, 95 bp, Homo sapiens
ccagctcgggcagccgtggccatcttactgggcagcattggatggagtcaggtctctaatactgcctggtaatgatgacggcggagccctgcacg

SEQ ID NO: 120 - DNA, 69 bp, Mus sp.
ccctcgtcttacccagcagtgtttgggtgctggttgggagtctctaatactgccgggtaatgatggagg

SEQ ID NO: 121 - DNA, 68 bp, Homo sapiens
ccctcgtcttacccagcagtgtttgggtgcggttgggagtctctaatactgccgggtaatgatggagg

SEQ ID NO: 122 - DNA, 72 bp, Mus sp.
gggtccatcttccagtgcagtgttggatggttgaagtatgaagctcctaacactgtctggtaaagatggccc

SEQ ID NO: 123 - DNA, 95 bp, Homo sapiens
cggccggccctgggtccatcttccagtacagtgttggatggtctaattgtgaagctcctaacactgtctggtaaagatggctcccgggtgggttc

SEQ ID NO: 124 - DNA, 83 bp, Mus sp.
cctgctgatggatgtcttaccagacatggttagatctggatgcatctgtctaatactgtctggtaatgccgtccatccacggc

SEQ ID NO: 125 - DNA, 83 bp, Homo sapiens
cgccggccgatgggcgtcttaccagacatggttagacctggccctctgtctaatactgtctggtaaaaccgtccatccgctgc

SEQ ID NO: 126 - DNA, 124 bp, Homo sapiens
gccaccaccatcagccatactatgtgtagtgccttattcaggaaggtgttacttaatagattaatatttgtaaggcacccttctgagtagagtaatgtgcaacatggacaacatttgtggtggc

SEQ ID NO: 127 - DNA, 68 bp, Mus sp.
ctcttgtccttcattccaccggagtctgtcttatgccaaccagatttcagtggagtgaagctcaggag

SEQ ID NO: 128 - DNA, 110 bp, Homo sapiens
aaagatcctcagacaatccatgtgcttctcttgtccttcattccaccggagtctgtctcatacccaaccagatttcagtggagtgaagttcaggaggcatggagctgaca

SEQ ID NO: 129 - DNA, 92 bp, Mus sp.
tgtaccaccttgtcggatagcttatcagactgatgttgactgttgaatctcatggcaacagcagtcgatgggctgtctgacattttggtatc

SEQ ID NO: 130 - DNA, 72 bp, Homo sapiens
tgtcgggtagcttatcagactgatgttgactgttgaatctcatggcaacaccagtcgatgggctgtctgaca

SEQ ID NO: 131 - DNA, 85 bp, Mus sp.
ttgatacttgaaggagaggttgtccgtgttgtcttctctttatttatgatgaaacatacacgggaaacctcttttttagtatcaa

SEQ ID NO: 132 - DNA, 81 bp, Homo sapiens
gatactcgaaggagaggttgtccgtgttgtcttctctttatttatgatgaaacatacacgggaaacctcttttttagtatc

SEQ ID NO: 133 - DNA, 44 bp, Homo sapiens
tatgttcaacggccatggtatcctgaccgtgcaaaggtagcata

SEQ ID NO: 134 - DNA, 65 bp, Mus sp.
ctgttaatgctaattgtgataggggttttggcctctgactgactcctacctgttagcattaacag

SEQ ID NO: 135 - DNA, 65 bp, Homo sapiens
ctgttaatgctaatcgtgataggggtttttgcctccaactgactcctacatattagcattaacag

SEQ ID NO: 136 - DNA, 906 bp, Unknown
  Description of Unknown: SERP2463 polynucleotide
atgaaagatgttatttatgtagaaaatcattactttgttactgcgaaggaaaatagcattaaatttagaaatgtcatagataaaagtgaaaaattttatttgtttgaagaaattgaagctattatttttgatcattataaaagctatttttcacataaattggtaattaaatgtatagagaatgatattgcgattatattttgtgacaaaaagcattctccagttacacagctcatttcttcatatggcatggttaatcgtcttaaaagaattcaaagtcaatttcagttatcgggaagaacaaaagatagaatttggaaaaagattgttataaataaaatttttaaccaaacacgatgtcttgaaaataatttacataatgaaaatgttaagctgatgttaggttttgcaaaagaagtgagttcaggagacaaaagtaataaagaagcacatgctactcgtatttattttaaagatttatttggcaaacaatttaaacgtggacgctataatgatgttatcaattcaggattgaattatggttattcaattctcagatcttttatcaacaaagagttagctattcatggatttgaaatgagcctaggtattaaacatcagtcaaaagaaaatccatttaatttagcagatgatatcattgaagtttttcgtccttttgtagataatatagtgtacgagatagtttttaagaaaaatattgatacatttgatataaacgaaaagaaattattattaaatgttttgtatgaaaggtgcattatagataaaaaagtagtgcgattacttgatagtgtgaagatagttattcaatcgctaattagatgttatgaagaaaatacacctacttatttattactacctaagatgatagaggtgggaaactaa

SEQ ID NO: 137 - DNA, 870 bp, Unknown
  Description of Unknown: Spy1047 polynucleotide
atggctggttggcgtactgttgtggtaaatacccactcgaaattatcctataagaataatcatctgatttttaaggatgcctataaaacggagctgatccatttatcagaaattgatattttgttattagaaacgaccgatattgtcttgtccactatgctggtaaaacggctagtggatgagaatgtccttgtcatattctgtgatgataaacgattaccaacagctatgctgatgcctttttatggtcgtcatgattcgagtttacagcttgggaaacaaatgtcctggtcagaaacagtcaaatcgcaggtttggacgacgattattgctcaaaagattttgaatcaatcttgctatctaggagcatgctcctattttgaaaaatcccaatctattatggatttatatcatggtttggaaaattttgatccgagtaatcgagaagggcatgcagcgagaatttattttaatacactttttgggaacgatttctcaagagatttggagcatccaatcaatgcaggtctggattatggttatactttattattgagtatgtttgcgcgtgaagtggttgtgtctggatgtatgactcagtttgggcttaaacacgctaatcagtttaatcagttcaattttgctagcgatattatggaaccatttaggcctttagtggataagattgtttatgaaaatcgaaatcagccttttcccaaaataaagagagagttatttactttgttttcagatacattttcatataatggtaaagagatgtatctcacgaatattattagcgattatactaaaaaagttgtcaaagctctgaataatgaagggaaaggagttcctgaatttaggatatga

SEQ ID NO: 138 - DNA, 4890 bp, Unknown
  Description of Unknown: FTN_0757 polynucleotide
atgaatttcaaaatattgccaatagcaatagatttaggtgttaaaaatactggtgtctttagcgcattttatcaaaaaggaacttctcttgagagattggataataaaaatggcaaagtatatgaactatcaaaagattcttatactttattgatgaataatagaacagcaagaagacatcaaagaagagggatagatagaaagcagctagtcaaaaggctctttaagcttatttggacagagcagctaaatttagagtgggataaagacactcaacaagcaattagctttttatttaatcgtagaggttttagttttattactgatggttattcgcctgaatatttaaatattgttccagagcaagtaaaagcgatacttatggatatatttgatgattacaacggtgaagatgatttagacagttatttaaaattagctactgagcaagaaagcaaaatttctgaaatttataacaagctaatgcaaaaaatattagagtttaaattaatgaaattatgtactgatattaaggatgataaagtaagtactaaaacgcttaaagaaatcacaagctatgaatttgagttattagctgattatttagcaaactatagcgagagtttaaaaacacaaaaatttagttatacagataaacaaggtaatttaaaagagctaagctactatcatcatgataaatataatattcaagaatttctaaagcgacatgctactataaatgatcgaattttagatactcttttaactgatgatttagatatttggaattttaattttgagaaatttgattttgataagaatgaagaaaagcttcagaatcaggaagataaagatcatatacaagcgcatttacatcattttgtttttgcagtaaataaaataaaaagtgaaatggcaagtggtggtcgtcatcgtagccaatattttcaagagataacaaatgtgctagatgaaaataatcatcaagagggatatctcaagaatttctgtgaaaatttgcataataaaaaatattcaaatttaagtgttaaaaatttagttaatctaattggtaacctaagtaatttagagctaaaaccgctaagaaaatattttaatgacaaaattcacgcaaaagctgatcattgggatgagcaaaagtttacagaaacttattgccactggatattaggagagtggcgagtaggtgtcaaagatcaagataagaaagatggcgctaaatatagttataaagatctgtgtaatgaattaaaacaaaaagttactaaggctggtttggtagattttttattagagttagatccatgtagaactataccaccatatctggataacaataaccgtaaaccaccaaaatgtcaaagtttgattttaaatccgaagtttttagataatcaatatccaaactggcaacaatatttacaagaattaaagaaactacaaagtattcaaaattatttagacagttttgaaactgatttaaaagtcttaaagtcaagtaaagatcaaccatattttgttgaatacaagagttcaaatcagcaaatagcaagtggtcaaagagattataaagatttagatgctcgaatattacagtttatatttgatagggtaaaagctagtgatgagttgcttttgaatgagatttattttcaggctaaaaaacttaaacaaaaagctagctctgagttagaaaaactcgagtcgagcaaaaagctagatgaagttatagcaaatagtcaactatcacagatactaaagtctcaacatacaaatggtatttttgaacagggtacttttttgcatttggtttgtaaatattataaacaaagacaaagagcgagagactctaggctatatattatgcctgaatatcgttatgataaaaaactacataaatataacaatacaggcaggtttgatgatgataatcagctgctaacatattgtaatcataagccaagacaaaaaagataccaattgttaaatgatttagctggggtgttgcaggtatcacctaattttttgaaagataaaattggttctgatgatgatctatttattagcaaatggttggtagagcatattagaggatttaaaaaagcttgtgaagatagtttaaaaatacaaaaagacaatagaggattattaaatcataaaataaatatagctaggaatacaaaaggcaaatgtgaaaaagaaatatttaatttaatatgtaaaatagaaggttcagaagataaaaaaggtaattacaagcatggtttagcttacgaattaggagtacttttatttggtgaacctaatgaagctagtaaacctgagttcgatagaaaaattaaaaaatttaactcaatatacagttttgcacagattcaacaaattgcttttgcagagcgtaaaggcaatgctaacacttgtgcagtttgtagtgctgataatgctcatagaatgcaacaaattaagatcactgagcctgtagaggacaataaagataagataatcttaagtgccaaagctcagagactaccagcgattccaactagaatagttgacggtgcggttaagaaaatggcaactatattagctaaaaatatagttgatgataattggcagaatatcaaacaagttttatcagcaaaacatcagttacatatacctattatcacagaatcaaatgcttttgagtttgaaccagcattagctgatgtaaaaggtaagagcctaaaagataggagaaaaaaagcattagagagaataagtcctgaaaatatattcaaggataaaaacaatagaataaaagaatttgctaaaggtatatcagcatatagtggtgctaatttaactgatggcgattttgatggtgcaaaagaagaattagatcatataatacctcgttcacataaaaaatacggtactctaaatgatgaagcaaatctaatttgtgtaactcgtggtgataataaaaataaaggtaatagaattttctgcctacgtgatcttgcagataactataaactaaaacagtttgagacaactgatgatttagaaattgaaaagaagatagctgatacaatctgggatgctaacaagaaagattttaaatttggtaattatcgtagttttattaacctaacaccacaagagcagaaagcatttcgtcacgcgctatttctggctgatgaaaatcctatcaaacaagcagtcataagagcgataaataatcgtaatcgtacatttgtaaatggcactcaacgctattttgcagaagtactggcaaacaatatctatctaagggctaaaaaagaaaatctaaatacagataaaatttcatttgattattttggtattccaactataggtaatggtagaggtattgctgaaatccgtcaactttatgaaaaagttgatagtgatatacaagcttatgcaaaaggtgataaacctcaagctagctactctcacctaatagatgcgatgctggctttttgtattgctgctgatgaacacagaaatgatggaagtataggtctagaaatcgataaaaattatagtttatatccattagataaaaatacaggagaagtctttaccaaagatatttttagtcaaattaaaattactgataatgagtttagcgataaaaaattagtaagaaaaaaagctatagagggctttaacacgcatagacaaatgactagagatggcatttatgcagaaaattacctaccaatactaatccataaagaactaaatgaagttagaaaaggctatacttggaaaaatagtgaagaaataaaaatattcaaaggtaaaaagtacgatatacaacaattgaataaccttgtgtattgtctaaaatttgtagataaacctatatctatagatatacaaattagtaccttagaagagttaagaaatatattaacaacaaataatatagctgctacagcagaatactattatataaatctaaaaacccaaaaattacatgagtattatatcgaaaactataatactgccttaggttataaaaaatacagtaaagaaatggagtttttgagaagcttagcttatcgtagcgaaagggtaaaaattaaatcaatagatgatgtaaagcaggttttggataaggatagtaactttatcatcggtaagattactttaccatttaaaaaagagtggcaaagactatatcgtgagtggcaaaatacaactatcaaagatgattatgagtttttaaaatcattctttaatgttaaaagtattactaagttgcataaaaaagttagaaaagatttctctttacctatttctacaaatgaaggtaaattcctggtcaaaagaaaaacatgggataacaattttatctatcagatattaaatgattctgattctagagcagacggaacaaagccatttattccagcttttgacatttctaaaaatgaaatagtcgaagccataattgattcatttacatcaaaaaatattttttggctgcctaaaaatatagaattacaaaaggtggataataaaaacatttttgctatagatactagtaaatggttcgaagtagaaacacctagtgatcttagagacattggaatagcaacaattcaatacaagatagataataattctcgccctaaagtcagagttaaacttgattatgttatcgatgatgatagtaagataaattattttatgaatcattctttattaaaatcaagatatcctgacaaagttttagaaattttaaaacaatcaactattatagaatttgaaagttcaggttttaataaaactatcaaagaaatgcttggtatgaaattagcaggtatttataatgaaacatctaataattag

SEQ ID NO: 139 - DNA, 4107 bp, Unknown
  Description of Unknown: Spy1046 polynucleotide
atggataagaaatactcaataggcttagatatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactga

SEQ ID NO: 140 - DNA, 1347 bp, Unknown
  Description of Unknown: y1724 polynucleotide
atgtcggacgaaggattagcggcgtttattgtctcttatatcaaaagccgcgagcagccaaagcttgaggcttttgataaagaggcagagaaaaggctggcagggctaactcaggcggaagatattgcactggcccagcaagagattgcccaacaacggcaggagttgatagcccgttatgaggtgcgcaattggttaaccgatgcggctaaccgtgccgggcaaataaagttagcaactcatgcacctaaatacactcatagtgattcaaaaagtagtggtattttgaatattgagttacaaagtaagaaaaaggattatttctccagcgtagacctcgctgaacaagccagtgatgttattggtaatgcagccgtatttgatcttgtaaagctattgcagagtgaatgtgaaggcggctcattaataaagtgtttagagcaaggtgataattcagtactgaaactattctcaaatgatgacgagttagttgaagagtggactacaaaatttaaaagcattttgaataatgagaaattcacgtcacataaactggccaagcaattttatttccctgtggggccagatcaataccacttgttaagtccacttttctcctcatcattggcgcaggccatgcatcaacgtattattgaagcccgtttcagtgaccaatcgaaagaggccaaggctgcccataaagcagggcgatggcatcctacggtcagggtcttgtaccttgatacggcggtgcaacatattggtggtaccaaaccccaaaatatctcttacctaaacagcgtccgtggcggaaaagtttggttgctgccctgtggcgcaccgtcttggaaaaatattcagaaaccgccaataaaatatagatctatttttcatgatcgcagtgaatttactgtactggcccgcaataacctgtggcaaatgcagctatatttgctgggtgtcaaacccctgagcaatacgatggacatccgcgccgctcgtttggcctgctgcgatgaaatcattgatattctgtttaattatgtggctgaaattcaaaatctggacggtaccaatggctggagtgacgctgaagattgtaagttaaaacgctcagagcagctttggcttgacccgacccgtgctatggatgaccccgtatttaagctcgaacgtgagaaggaggattggaaacaagaggtcagccaagacttcggttactggttaaatcgttggttacagcatgatgaactggtctttggctatgttgaacagcgggagtggtcaagcttatttaagcagcggctacgtgaatttgaacaaggtagcctggagacatcgtcatga

SEQ ID NO: 141 - DNA, 1194 bp, Unknown
  Description of Unknown: Balac_1306 polynucleotide
atgcgcaaactcacagtacaagacttgaatgaagcggcaaaaattgggggctcgaatgcgctgacggaagtaacgtcgctcgcaccggcagcaggtatgggtagtatcgtagctcctgcaaaatacactgctggtaatgggtcgacgtatgtgtacgagaagcgctgggtgaatgatgagtgtgttgatactgtgctcattgattctagaacatcacaggccaatcgtttggaagactacatcagccgagcgattgaagttggccatccgattttcagtaagatgccccaagttcgcgtacgttatgagatgatcccaggagacgaaagcagcgtcaggtatttcgatgatgttcaattgccacatcgtgcggtggacgcgcatattcgcatcgctgaattcagtgaatccgacaaagtcaagtacatggcggcacgaaactcctccttggaggatttgtctgcaatgctggcgatttccccagtgacagtgatgtttgggtgctgggactcaacacgaaacaagaaccaactgcgtattcccgccagcttcaacggtgagatttatgcggtgcttgctgatcagacgcatgagtctccaattcatcgtgcaggcgcgcgtattgatccagttgcagccggagtgcatctaaccaaaaatgaagccaagaaaattgctgagcgcatcaaaggaacaatgaatgacaagaagctcagcaaattcgcgagtagcggggatggctcaactatcgtcattggtgcgattcccccatcaactgatgcaaatgctcttgatggcatcgcagttcgtagtattacgcgcacacatgtcctgagtttctccatgcttcgggctatgcgcttcggtaaagggccagagggtgatgaagctatccgtgtactgctggctgcagcgttgatcaatgcgatggttggaagcaatgcggaattgcatctacgggaaaactgcttccttgtggaagctgatgagccgaaaaccgttctggatcgtcgtggcggtaaacatgatgatttggagatgcttacgctggaagacgccgatgagctgttggctcaggcatatgcacaagctcagaaaaaggcaggcattgattggcatgggcaaattatcactgtgcaaggcgaccctgcagtgattgaatctgcaagcgctgctgacgacgatgatagatga

SEQ ID NO: 142 - DNA, 1092 bp, Unknown
  Description of Unknown: GSU0053 polynucleotide
atgaacgatctcgtgcagaagtatgaccattggttggaaaactccggacctgcggcactggttattcgcgaacaactgatgcccgtcgagggacgtgacggtgtgctgtttccagcgacctttgccgataccggctacaacatcgacaaattcgacgatggcggcaatgtctgcctgatcgacagtgtcgggtcccaggcaaacaggatcgagccgatcttcatgactaaggattacgctggccttgtcccccaaatagtggtccaggcgggaaacaaaaaagtaaatcttctcgaagcagggcatcgagccggggacgcgattattcgctgttctgagttgcagcaaacccttagggctgcgttcaacaacgttctgaatggcaatgcagagccactagcccgtatagcacccacctcgcttgtgtttggcgtgtgggattcacgagatacccaagccaaattgcccagactcgttgcctcgaccataagggcctacaatgttcgccctctcacccgctctgcccagtatgtgccggctgttgactacaacgccgaagggcttttggaagagcccggtgacttgcgagatgctgaaggcaaagtcaagagcaagcacccgtttgcccaacgcgggtttgtgcatgtcccggcgacaggtgctctcggcggcgtaatcgccaccggggggattcgccgtgacgccacactccaccttgccgcgctccgcttgctttcggcaggccaagacgaagcaaagtccaaggcccttcgccgctatatactcagtcttgccttaacagcatttactgtgcctgtaactggctatctgcgtcagggctgcaatcttgtgctcgaccctgaaaacccccttgagtttaaagaggtttttaatgatgggacgcgcaatgacgtcggtattacgcacaccgaagcgattgtctatgcaaaggcagttgcaaaggagtttggcattgaccccgagcgtaaccttgacgaaaaaaaagccccggatcgagaagtaccgtttgacaaggtactggcgaaaaaagatgtgagcgatgccggaggctctaagaaaaaagcaaaatga

SEQ ID NO: 143 - DNA, 1398 bp, Unknown
  Description of Unknown: MJ1666 polynucleotide
atgcaaaaaatattaattgctccatggggaaatttttcaagttggaaaaaagttatctactcatttaatggagttgaaaaagaatcaaaaagctctttatctgccatttatgataaaataaacccagataaagtttatatattggttttagatactttatctaatttagaatcagaaaattatggagatattgtaaaagaagttaaagaaaagacagagaattttataaaagaaaatttaaacattgataattacgaggtaattgtatgtcctggagttgggacattttataacaaagattttgaaaaatactttaaattttatgggaatttgactgattattattcttttgccctttatgaattgtctaaaagattggatggagatttggaagttcatttggacttaacacatggattaaattacatgcctgtcttaacctatagagtaattaaagacctcttagaaattttagcaataaaaaataaggttagattagttgtttataactcagacccctatgttggaagagaaaaagaaatattaaacatccacactgtggaagatgtgattataaaaccgtcctatgacattaaaggtatgactttggattttttagacgcaaccaaatttgtagataaaaaagaaataggaaaaataaaaaaagaaattaacatgaatccaaagataaaagaattaagaataatgaaacaaaatataaatgcatttatagcttctattgtttatgctctacctttagtttattcaacattctttgtaaagaaagataaaattgagatttatttaaatgaacttattggagcatttatttcaaatataaaaattaatccagaagataaaatattaaaaagatacttatatttcggagaaggatttaatagcttggttaaagcatattttgcttcaaagattagcgaaattcctcaattgataaaagacgagctatctttagaagagattgatgaattaaaaaataccttattcaaagaaaatccaaactctcaatatatcaaaaatgagatttcatccctttataacataataaacaccaaatataaagaagaagaacttagtgaaatcttaggaaattggactccaatatataaaattagaagggagaatattgacaaattcaagattaggaatttcttagcacatgctgggtttgaaaaaagtgtaactgaaatttatatttccgtagaaaataaaaatggaaaaattgaacttagtgaaaaaacttcgcttagatataataagaactacatagaagaaaaaaatggaatcaaaaggttcatatttaaatataaggacaaaaatggaaaagtagaggagataaatatcttagaaaaaattgaagagattctactaaacaaataa

SEQ ID NO: 144 - DNA, 1125 bp, Unknown
  Description of Unknown: NE0113 polynucleotide
atgccagagccgctccaaccccatgaatatccgcaccgcatactgttatgtgttacggggctgtcaccgcagattgtcactgaaacgctgtacgctctggctgtggcacgggccacgccctttattccgacagagatacatctgctgaccactaccgatggagcacggttggcgcgggcagcattgctgcaccccgatggtggacattttcatgcgctgttgaatgaccagccacagatcggcctcccccgttttgatgaagattgcatccatatcatcagccatcaccaggaaaaactcgccgacattcgcacaccggccgaaaatgccgcagcggctgacacaatcacggcacttgttgcccaacttactgaggatgctgacgcggcgctgcatgtttcgattgccggcgggcgcaagaccatgggtttttatctggggtatgccttttcactgtttgcccggccgcaggataatctctcccatgttctggtttcatcaccctttgaaggtcatccggattttttttacccgccacgtcagccacgccgcctggtaacacgagacgggcatcatattgatactgccgaggccattgttacactggcagaaattcctgtggtacggttgcggcacgggctgccggctactctgattgccggccgcgccgggttcagtgaaacggtagttaccttgcagcaaagttttgcaccaccatgcctgctgattgatctggagcagcgaaacgtcgtctgtggcactactgcagttgccatgaaaccgcaactgcttgcatggctggcgtggtgggctacgctagcccgacaggggcggcctgaaacaacctggcgtgaagccgatgccagattatttctcgatatttaccggacagtggttggtattgatgccattgattatgagaaaaccgccgagctgctcggcaacggcatggagaaggagttttttcagaccaaaaacgcgaaactggaacgtgtgctgaaagacacacttggaccggcagctgccccctatctgctaacgactacgggcaaacgcccgcatacacggcgtggtctcacactgcctcccgagcgtattcgtatcgttggcacaggcagtaaatga

SEQ ID NO: 145 - DNA, 1443 bp, Unknown
  Description of Unknown: PF1127 polynucleotide
atgggaatgagagttttggtaactacatggggtaatcccttccagtgggaaccaataacatatgaatacagaggaatcaaagttaaaagcagaaataccttgccaattctagtcaagactcttgagccagagaggattctaatccttgtggctgatacaatggccaactactatgattcaggaaaaaataagccagaaatagaagaaaaatcgttttcgtcttattcggaagttgtggaagatacaaaagaaaggatactatggcacataaaagaggaggtcattgaagaactccgtgaggaagatcctgagcttgctaagaaaattgagaatatgttaaaagatgaaagaattacaattgaagttcttcccggcgttggagtctttggcaacattacagtagagggagaaatgcttgacttctattattatgccacatacaagttggccgaatggttgccagttcagaacaatttagaggtttacttagacctaactcatgggataaatttcatgcccacctttacttacagagccctaagaaacttgcttggattgttggcctacttgtacaatgtaaagtttgagatagttaattcagaaccttatcccctgggggtttcacaagaaataagggaggacacaattctccatattagggaaattggagagggagtagttcgtcctagaccacagtattctccagtagaaggaaagctttactggaatgcatttataagctctgtagccaatggcttcccgttagtctttgccagcttttatccaaatattcgggacgtagaagattaccttaacaaaaagcttgaggaattcctggtgggaattgaggttggggagagagaagatggaaaaccttatgttaaaagagagaaagctcttgacaggagctttaagaatgcttctaagctctactatgctttaagagtgttcaatacaaaattccaaaactatccaaaaaaagaagttcctattgaagaaataatggagatatcaaagatattcgagtctcttcccaggattggaattattttagagaggcaagtagagtggctaagaaatttagtatatggaagattatggtatgaaaatggagaacagaaaataaagaagggtcttttagagattatcaaggataagaaggataaaaggaaagaggccgaagctcttaaaaaagggaagacaatatctttagccgaagctgcaaagcttacaagaatattttctccgagtggagaaagaatagagacaatagaatctccaaatgttgttcgtaactttatagcacattctggatttgagtataacattgtctatgtgaaatatgatagactaagtgataggctgtactttttctataaggataaagaaaaagctgcaaatctcgcttatgaagcccttttatataggggtgaaaaagaatga

SEQ ID NO: 146 - DNA, 1335 bp, Unknown
  Description of Unknown: TM1812 polynucleotide
atgaatttccttgtaagaaaccttgtggaaaatttggaagaaggggacagagttatactggatgttactcattcgttcagaagtattccattgatggcaagtgtagtagccctgtaccttaaagaggccaaagatgttaatgtgagtgtagtttacggcaagtacaataaagaaacaaaagtaacagagtgcgaagatttgaccccactaactaaagctacatcatggatatatgctgttcgattgttcaaagagtatggatatgcgaaagaactcgctgatttgataaaaaagagaaatgaagaaatatacaggagaagtcaaagttcgaaaaaaccaaagctacttggttctatgtctcaaaaacttcaggatctttcgtcttctatacgtcttggatccatagtagccataaggaagaatttaaccaatttcttcaattttattgatagaaataaagcaagaataagagaggagacggaggtttttgttccagagattgcggctctcttagatgggatcgaaaaaagatacagggttatacatgtgaaatcggagaattttgaactgagcgaaaaagaattggaatctgaaaaagagttactggatttctatcttcaaacaggagatttaggtatggctcttcgtttggcaagagaatatcttataaatgtctatttgatgtctggaggggagaagagtgatttcttggatagaaatgttagagagtctgtgagcatttcaacgtttggttatgataccatccttcaggcaagaaatcatgttgctcatttcggattcaacaaactccagcttccctccttgaaaaagatagaagatcacctgaaagtgcttgttcaaaccccaccggaacaacttctggagtctgcgagaaaaactcagcgaaacagaaaaagagctcttctcactcctctcggtacgacaaaaggtgctttatacaccgttctgaaaaaaatttcacctgatctactcctggttataacttcaaaacagggaaaagctattctttcagaaattctggagaaagctgaattcaagggtgaatttagggtaatccttctcgaagacccttttatgggtgtaagtgaaatagatcgagtagtatccgaaatcaaagaacatctttctgacgtggatgaagtgatcgtcaatctcacaggtggaaccacttttcttacgtacgttatcgagcgtgccaaaaatcaaattagatacggaagaaaagtaaaaactatcctggctgtggacaagagaacttacgaagaacaaaagcagaatccttttgtggtgggtgaaattctggagcttgattga

SEQ ID NO: 147 - DNA, 32 bp, Artificial Sequence
  Description of Artificial Sequence: Synthetic oligonucleotide
caccccacctcgctcccgtgacactaatgcta

SEQ ID NO: 148 - DNA, 29 bp, Artificial Sequence
  Description of Artificial Sequence: Synthetic oligonucleotide
gctcaacgcgggacggctctcccattgac

SEQ ID NO: 149 - RNA, 77 nt, Homo sapiens
guggccucguucaaguaauccaggauaggcugugcaggucccaaugggccuauucuugguuacuugcacggggacgc

SEQ ID NO: 150 - DNA, 29 bp, Artificial Sequence
  Description of Artificial Sequence: Synthetic oligonucleotide
gctcaacgcgggacggctctcccattgac

SEQ ID NO: 151 - DNA, 79 bp, Artificial Sequence; identical to SEQ ID NO: 72
  Description of Artificial Sequence: Synthetic oligonucleotide

SEQ ID NO: 152 - DNA, 38 bp, Artificial Sequence; identical to SEQ ID NO: 73
  Description of Artificial Sequence: Synthetic oligonucleotide

SEQ ID NO: 153 - RNA, 12 nt, Artificial Sequence
  Description of Artificial Sequence: Synthetic oligonucleotide
guuacuugcacg

SEQ ID NO: 154 - RNA, 22 nt, Artificial Sequence
  Description of Artificial Sequence: Synthetic oligonucleotide
uucaaguaauccaggauaggcu

SEQ ID NO: 155 - RNA, 22 nt, Artificial Sequence
  Description of Artificial Sequence: Synthetic oligonucleotide
uucaaguaauccaggauaggcu

SEQ ID NO: 156 - RNA, 22 nt, Artificial Sequence
  Description of Artificial Sequence: Synthetic oligonucleotide
uucaaguaauccaggauaggcu

SEQ ID NO: 157 - RNA, 22 nt, Artificial Sequence
  Description of Artificial Sequence: Synthetic oligonucleotide
ccuauucuggguuacuugcacg

SEQ ID NO: 158 - RNA, 22 nt, Artificial Sequence
  Description of Artificial Sequence: Synthetic oligonucleotide
ccuauucuggguuacuugcacg

SEQ ID NO: 159 - RNA, 22 nt, Artificial Sequence
  Description of Artificial Sequence: Synthetic oligonucleotide
uucaaguaauccaggauaggcu

SEQ ID NO: 160 - DNA, 10 bp, Artificial Sequence
  Description of Artificial Sequence: Synthetic oligonucleotide
taatgaactt

SEQ ID NO: 161 - DNA, 10 bp, Artificial Sequence
  Description of Artificial Sequence: Synthetic oligonucleotide
aagttcatta

SEQ ID NO: 162 - RNA, 10 nt, Artificial Sequence
  Description of Artificial Sequence: Synthetic oligonucleotide
ccuauucugg

SEQ ID NO: 163 - RNA, 10 nt, Artificial Sequence
  Description of Artificial Sequence: Synthetic oligonucleotide
ccuauucugg

SEQ ID NO: 164 - RNA, 10 nt, Artificial Sequence
  Description of Artificial Sequence: Synthetic oligonucleotide
ccuauucugg

SEQ ID NO: 165 - DNA, 145 bp, Mus sp.
agttatgctccatacagtacacaatctcttctctctacagatgactgccatggaggagtcacagtcggatatcagcctcgagctccctctgagccaggagacattttcaggcttatggaaactgtgagtggatctttttggggcc

SEQ ID NO: 166 - protein, 21 aa, Artificial Sequence
  Description of Artificial Sequence: Synthetic peptide
ELAAWCRWGFLLALLPPGIAG